(19) United States
(12) Patent Application Publication
(10) Pub. No.: US 2025/0136620 A1
(43) Pub. Date: May 1, 2025

(54) BTK DEGRADER

(71) Applicant: Newave Pharmaceutical Inc., Pleasanton, CA (US)

(72) Inventor: Yi Chen, Pleasanton, CA (US)

(21) Appl. No.: 18/729,223

(22) PCT Filed: Jan. 17, 2023
(86) PCT No.: PCT/US2023/010944

§ 371 Date: Jul. 16, 2024

Related U.S. Application Data
(60) Provisional application No. 63/300,117, filed on Jan. 17, 2022.

(51) Int. Cl. C07D 519/00; A61K 31/5383

(52) U.S. Cl. CPC C07D 519/00; A61K 31/5383

(57) ABSTRACT

The disclosure includes compounds of any one of Formulae (0)-(5), (11)-(24), (A)-(E), and (1)-(V) comprising an E3 ubiquitin ligase binding moiety that binds an E3 ubiquitin ligase, as described herein. Also disclosed pharmaceutical compositions comprising compounds, and a method for treating a neoplastic disease, autoimmune disease, and inflammatory disorder with these compounds.

### REFERENCE TO RELATED APPLICATIONS

This application claims priority to and the benefit of the filing date of U.S. Provisional Patent Application No. 63/300,117, filed on Jan. 17, 2022, the entire contents of which are incorporated herein by reference.

### BACKGROUND OF THE INVENTION

Bruton tyrosine kinase (Btk) is a Tec family non-receptor protein kinase, expressed in most hematopoietic cells such as B cells, mast cells, and macrophages but not in T cells, natural killer cells, and plasma cells [Smith, C. I. et al. Journal of Immunology (1994), 152 (2), 557-65]. Btk is a crucial part of the BCR and FcR signaling pathway, and the targeted inhibition of Btk is a novel approach for treating many different human diseases such as B-cell malignancies, autoimmune disease, and inflammatory disorders [Uckun, Fatih M. et al, Anti-Cancer Agents in Medicinal Chemistry (2007), Shinohara et al, Cell 132 (2008) pp794-806; Pan, Zhengying, Drug News & Perspectives (2008), 21 (7); 7 (6), 624-632; Gilfillan et al, Immunological Reviews 288 (2009) pp 149-169; Davis et al, Nature, 463 (2010) pp 88-94].

Covalent Bruton's tyrosine kinase (BTK) inhibitors including ibrutinib and acalabrutinib have transformed the treatment landscape of several BTK dependent B-cell malignancies, including chronic lymphocytic leukemia, Waldenstrom's macroglobulinemia, mantle cell lymphoma and marginal zone lymphoma. Despite impressive clinical response of ibrutinib in B-cell malignancies, cases of primary and secondary resistance have emerged with poor outcomes and limited treatment options.

Removal of BTK protein would eliminate BTK kinase activity as well as any protein interaction or scaffolding function of BTK. Specific degradation of BTK could be accomplished using heterobifunctional small molecules to recruit BTK to a ubiquitin ligase and thus promoting ubiquitylation and proteasomal degradation of BTK. Thalidomide derivatives, such as lenalidomide or pomalidomide, can be used to recruit potential substrates to cereblon (CRBN), a component of a ubiquitin ligase complex. This unique therapeutic approach could present a mechanism of action for interfering with BTK activity and BCR signaling that is distinct from the mechanism of stoichiometric BTK inhibition. Furthermore, this degradative approach could effectively target the C481S mutated form of BTK, which mutation has been clinically observed and confers resistance to inhibition by ibrutinib (Woyach, et al. Blood. 120(6): 1175-1184. 2012).

### SUMMARY OF THE INVENTION

In one aspect, this invention relates to a compound of Formula (0), or an N-oxide thereof, or a pharmaceutically acceptable salt, solvate, polymorph, tautomer, stereoisomer, an isotopic form, or a prodrug of said compound of Formula (0) or N-oxide thereof:

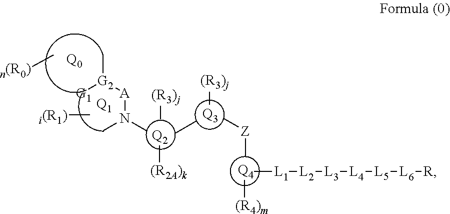

wherein
   R is a small molecule (e.g., molecular weight less than about 1,500 Da, 1,200 Da, 900 Da, 500 Da or less) E3 ubiquitin ligase binding moiety that binds an E3 ubiquitin ligase;
   each of L₁, L₂, L₃, L₄, L₅, and L₆, independently, is absent, a bond, N(Rₐ), O, S, C(O), S(O₂), OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, cycloalkyl, cycloalkenyl, heterocycloalkyl, heterocycloalkenyl, spiro-heterocyclic, fused-heterocyclic, bridged-heterocyclic, aryl, or heteroaryl is optionally substituted with one or more R_{d};
   Q₀ is a 5-9 membered aryl or heteroaryl;
   Q₁ is a 5-7 membered heterocycloalkyl;
   Q₀ and Q₁ taken together form a fused-heterocyclic with two or more shared/border atoms between Q₀ and Q₁, including G₁ and G₂, wherein each said shared/border atoms can be carbon or heteroatom;
   Q₂ is a 5-9 membered cycloalkyl, heterocycloalkyl, aryl or heteroaryl;
   Q₃ is a 5-9 membered aryl or heteroaryl;
   Q₄ is a 5-9 membered aryl or heteroaryl;
   A is in Q₁ and is —C(O)—, —P(O)(RₐR_{b})—, or —S(O₂)—;
   Z is NH or O;
   each of R₀, R₁, R_{2A}, R₃, and R₄, independently, is H, D, alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, or heteroaryl is optionally substituted with one or more R_{d};
   R₂ is H, halo, alkyl, —C(RₐR_{b}R_{c}), haloalkyl, or hydroxyalkyl;
   two of R₀ groups, taken together with the atom to which they are attached, may optionally form a cycloalkyl, heterocycloalkyl, heterocycloalkenyl, aryl, or heteroaryl, each optionally substituted with one or more R_{d};
   two of R₁ groups, taken together with the atom to which they are attached, may optionally form a cycloalkyl, heterocycloalkyl, heterocycloalkenyl, aryl, or heteroaryl, each optionally subsitiuted with one or more R_{d};
   two of R_{2A} groups, taken together with the atom to which they are attached, may optionally form a cycloalkyl, heterocycloalkyl, heterocycloalkenyl, aryl, or heteroaryl, each optionally subsitiuted with one or more R_{d};
   two of R₃ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₄ groups, taken together with the atom to which they are attached, may optionally form a cycloalkyl, heterocycloalkyl, heterocycloalkenyl, aryl, or heteroaryl, each optionally subsitiuted with one or more R_{d};
   each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more Rₑ;
   each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{f}; and
   each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl;
   Rₐ and R_{b}, taken together with the atom to which they are attached, may optionally form a cycloalkyl, heterocycloalkyl, heterocycloalkenyl, aryl, or heteroaryl, each optionally subsitiuted with one or more Rₑ;
   R_{b} and R_{c}, taken together with the atom to which they are attached, may optionally form a cycloalkyl, heterocycloalkyl, heterocycloalkenyl, aryl, or heteroaryl, each optionally subsitiuted with one or more Rₑ;
   two of R_{d} groups, taken together with the atom to which they are attached, may optionally form a cycloalkyl, heterocycloalkyl, heterocycloalkenyl, aryl, or heteroaryl, each optionally subsitiuted with one or more Rₑ;
   two of Rₑ groups, taken together with the atom to which they are attached, may optionally form a cycloalkyl, heterocycloalkyl, heterocycloalkenyl, aryl, or heteroaryl, each optionally subsitiuted with one or more R_{f}; and, two of R_{f} groups, taken together with the atom to which they are attached, may optionally form a cycloalkyl, heterocycloalkyl, heterocycloalkenyl, spiro-heterocyclic, fused-heterocyclic, bridged-heterocyclic, aryl, or heteroaryl, each optionally subsitiuted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl,
   each of i, j, k, m, n, p, and q, independently, is 0, 1, 2, 3, or 4.

In some embodiments, said E3 ubiquitin ligase is Cereblon, Von Hippel-Lindau, mouse double-minute homolog 2, or IAP.

In another aspect, this invention relates to a compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof:

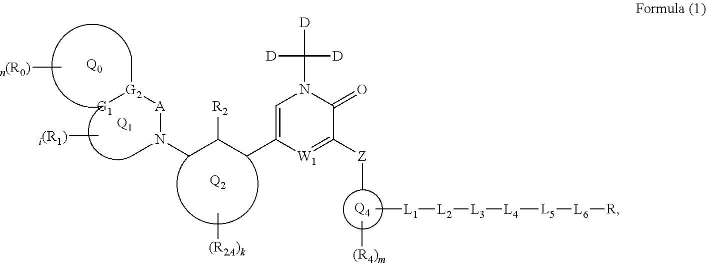

wherein
   R is a small molecule (e.g., molecular weight less than about 1,500 Da, 1,200 Da, 900 Da, 500 Da or less) E3 ubiquitin ligase binding moiety that binds an E3 ubiquitin ligase;
   each of L₁, L₂, L₃, L₄, L₅, and L₆, independently, is absent, a bond, N(Rₐ), O, S, C(O), S(O₂), OC(O), C(O)O, OSO₂, S(O₂O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, cycloalkyl, cycloalkenyl, heterocycloalkyl, spiro-heterocyclic, fused-heterocyclic, bridged-heterocyclic, heterocycloalkenyl, aryl, or heteroaryl is optionally subsitiuted with one or more R_{d};
   Q₀ is a 5-9 membered aryl or heteroaryl;
   Q₁ is a 5-7 membered heterocycloalkyl;
   Q₀ and Q₁ taken together form a fused-heterocyclic with two or more shared/border atoms between Q₀ and Q₁, including G₁ and G₂, wherein each said shared/border atoms can be carbon or heteroatom;
   Q₂ is a 5-9 membered cycloalkyl, heterocycloalkyl, aryl or heteroaryl;
   Q₄ is a 5-9 membered aryl or heteroaryl;
   A is in Q₁ and is —C(O)—, —P(O)(RₐR_{b})—, or —S(O₂)—;
   Z is NH or O;
   W₁ is N or CH;
   each of R₀, R₁, R_{2A}, and R₄, independently, is H, D, alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₂ is H, halo, alkyl, —C(RₐR_{b}R_{c}), haloalkyl, or hydroxyalkyl;
   two of R₀ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₁ groups, taken together with the atom to which they are attached, may optionally form a cycloalkyl, heterocycloalkyl, heterocycloalkenyl, aryl, or heteroaryl, each optionally subsitiuted with one or more R_{d};
   two of R_{2A} groups, taken together with the atom to which they are attached, may optionally form a cycloalkyl, heterocycloalkyl, heterocycloalkenyl, aryl, or heteroaryl, each optionally subsitiuted with one or more R_{d};
   two of R₄ groups, taken together with the atom to which they are attached, may optionally form a cycloalkyl, heterocycloalkyl, heterocycloalkenyl, aryl, or heteroaryl, each optionally subsitiuted with one or more R_{d};
   each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more Rₑ;
   each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{f}; and
   each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl;
   Rₐ and R_{b}, taken together with the atom to which they are attached, may optionally form a cycloalkyl, heterocycloalkyl, heterocycloalkenyl, aryl, or heteroaryl, each optionally subsitiuted with one or more Rₑ;
   R_{b} and R_{c}, taken together with the atom to which they are attached, may optionally form a cycloalkyl, heterocycloalkyl, heterocycloalkenyl, aryl, or heteroaryl, each optionally subsitiuted with one or more Rₑ;
   two of R_{d} groups, taken together with the atom to which they are attached, may optionally form a cycloalkyl, heterocycloalkyl, heterocycloalkenyl, aryl, or heteroaryl, each optionally subsitiuted with one or more Rₑ;
   two of Rₑ groups, taken together with the atom to which they are attached, may optionally form a cycloalkyl, heterocycloalkyl, heterocycloalkenyl, aryl, or heteroaryl, each optionally subsitiuted with one or more R_{f}; and two of R_{f} groups, taken together with the atom to which they are attached, may optionally form a cycloalkyl, heterocycloalkyl, heterocycloalkenyl, aryl, or heteroaryl, each optionally subsitiuted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl.
   each of i, k, m, n, and p, independently, is 0, 1, 2, 3, or 4.

In some embodiments, said E3 ubiquitin ligase is Cereblon, Von Hippel-Lindau, mouse double-minute homolog 2, or IAP.

In some embodiments, the compound is represented by Formula (2):

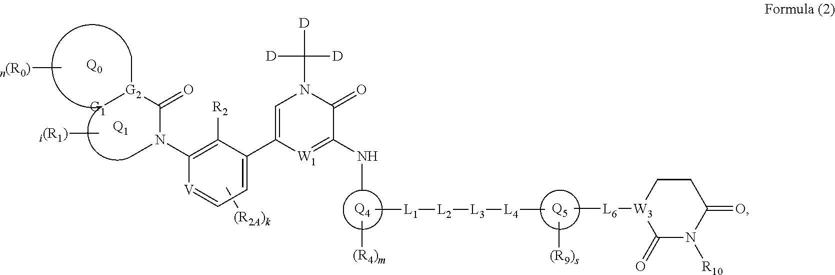

wherein
   R₁₀ is H, D, -alkyl-O—P(O)(Rₐ)(R_{b}), or -alkyl-OC(O)—Rₐ;
   L₆ is absent, NH, CONH, or O;
   W₁ is N or CH;
   W₃ is N or CH;
   Q₅ is absent, cycloalkyl, cycloalkenyl, heterocycloalkyl, heterocycloalkenyl, spiro-heterocyclic, fused-heterocyclic, bridged-heterocyclic, aryl, or heteroaryl;
   R₉ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₉ and L₄ groups, taken together with the atoms to which they are attached, may optionally form a cycloalkyl, heterocycloalkyl, heterocycloalkenyl, aryl, or heteroaryl optionally subsitiuted with one or more R_{d}; and
   V is C(Rₐ) or N; and
   s is 0, 1, 2, 3, or 4,
   while the remaining groups are as defined in Formula (1).

In a further embodiment, the compound is represented by Formula (3):

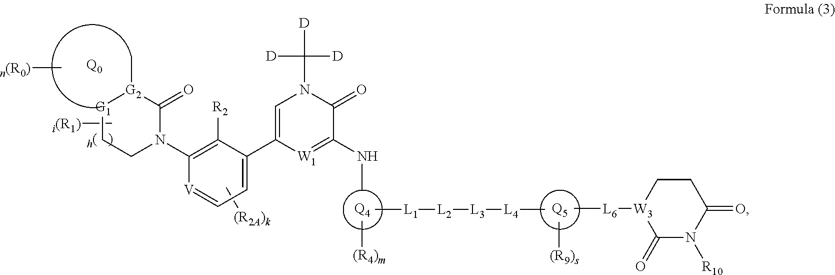

wherein h is 0, 1 or 2, each of the border atoms between Q₀ and Q₁ including G₁ and G₂, can be carbon or heteroatom, while the remaining groups are as defined in Formula (2).

In a further embodiment, the compound is represented by Formula (4):

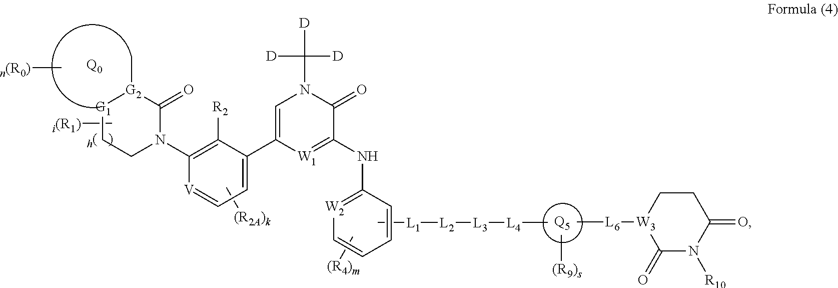

wherein W₁ is CH and W₂ is N, or W₁ is N and W₂ is CH, each of the border atoms between Q₀ and Q₁ including G₁ and G₂, can be carbon or heteroatom, while the remaining groups are as defined in Formula (3).

In a further embodiment, the compound is represented by Formula (5):

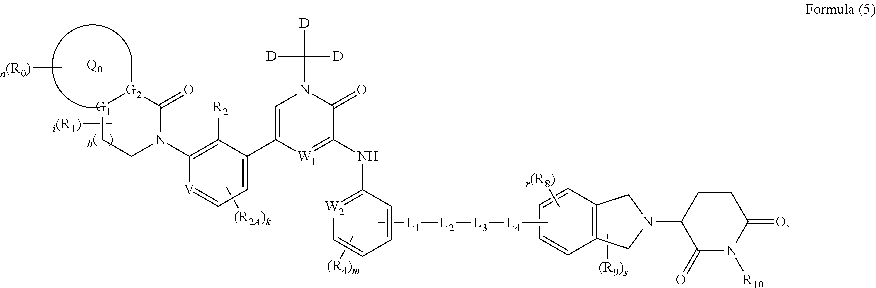

R₈ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
R₈ and L₄ groups, taken together with the atom to which they are attached, may optionally form a cycloalkyl, heterocycloalkyl, heterocycloalkenyl, aryl, or heteroaryl optionally subsitiuted with one or more R_{d}; and
r is 0, 1, 2, 3, or 4,
each of the border atoms between Q₀ and Q₁ including G₁ and G₂, can be carbon or heteroatom,
while the remaining groups are as defined in Formula (4).

In another aspect, this invention relates to a compound of Formula (A), or an N-oxide thereof, or a pharmaceutically acceptable salt, solvate, polymorph, tautomer, stereoisomer, an isotopic form, or a prodrug of said compound of Formula (A) or N-oxide thereof:

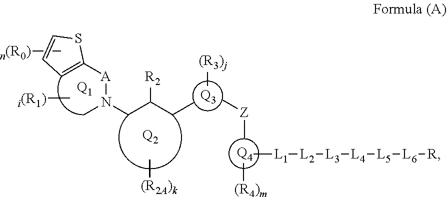

wherein
   R is a small molecule (e.g., molecular weight less than about 1,500 Da, 1,200 Da, 900 Da, 500 Da or less) E3 ubiquitin ligase binding moiety that binds an E3 ubiquitin ligase;
   each of L₁, L₂, L₃, L₄, L₅, and L₆, independently, is absent, a bond, N(Rₐ), O, S, C(O), S(O₂), OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   Q₁ is a 5-7 membered heterocycloalkyl;
   Q₂ is a 5-9 membered cycloalkyl, heterocycloalkyl, aryl or heteroaryl;
   Q₃ is a 5-9 membered aryl or heteroaryl;
   Q₄ is a 5-9 membered aryl or heteroaryl;
   A is —C(O)—, —P(O)(RₐR_{b})—, or —S(O₂)—;
   Z is NH or O;
   each of R₀, R₁, R_{2A}, R₃, and R₄, independently, is H, D, alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₂ is H, halo, alkyl, —C(RₐR_{b}R_{c}), haloalkyl, or hydroxyalkyl;
   two of R₀ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₁ groups, taken together with the atom to which they are attached, may optionally form a cycloalkyl, heterocycloalkyl, heterocycloalkenyl, aryl, or heteroaryl, each optionally subsitiuted with one or more R_{d};
   two of R_{2A} groups, taken together with the atom to which they are attached, may optionally form a cycloalkyl, heterocycloalkyl, heterocycloalkenyl, aryl, or heteroaryl, each optionally subsitiuted with one or more R_{d};
   two of R₃ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₄ groups, taken together with the atom to which they are attached, may optionally form a cycloalkyl, heterocycloalkyl, heterocycloalkenyl, aryl, or heteroaryl, each optionally subsitiuted with one or more R_{d};
   each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more Rₑ;
   each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{f}; and
   each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl;
   Rₐ and R_{b}, taken together with the atom to which they are attached, may optionally form a cycloalkyl, heterocycloalkyl, heterocycloalkenyl, spiro-heterocyclic, fused-heterocyclic, bridged-heterocyclic, aryl, or heteroaryl, each optionally subsitiuted with one or more Rₑ;
   R_{b} and R_{c}, taken together with the atom to which they are attached, may optionally form a cycloalkyl, heterocycloalkyl, heterocycloalkenyl, aryl, or heteroaryl, each optionally subsitiuted with one or more Rₑ;
   two of R_{d} groups, taken together with the atom to which they are attached, may optionally form a cycloalkyl, heterocycloalkyl, heterocycloalkenyl, aryl, or heteroaryl, each optionally subsitiuted with one or more Rₑ;
   two of Rₑ groups, taken together with the atom to which they are attached, may optionally form a cycloalkyl, heterocycloalkyl, heterocycloalkenyl, aryl, or heteroaryl, each optionally subsitiuted with one or more R_{f}; and
   two of R_{f} groups, taken together with the atom to which they are attached, may optionally form a cycloalkyl, heterocycloalkyl, heterocycloalkenyl, spiro-heterocyclic, fused-heterocyclic, bridged-heterocyclic, aryl, or heteroaryl, each optionally subsitiuted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl,
   each of i, j, k, m, n, and p, independently, is 0, 1, 2, 3, or 4.

In some embodiments, said E3 ubiquitin ligase is Cereblon, Von Hippel-Lindau, mouse double-minute homolog 2, or IAP.

In other embodiments, the compound is represented by Formula (B):

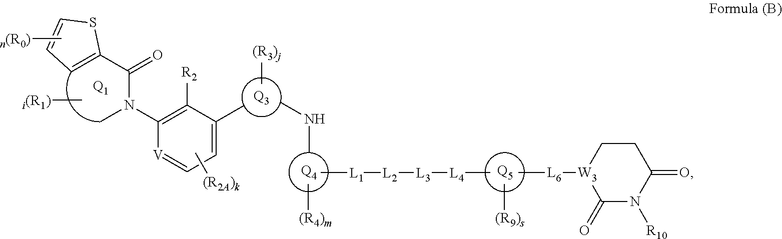

wherein
   R₁₀ is H, D, -alkyl-O—P(O)(Rₐ)(R_{b}), or -alkyl-OC(O)—Rₐ;
   L₆ is absent, NH, CONH, or O;
   W₃ is N or CH;
   Q₅ is absent, cycloalkyl, cycloalkenyl, heterocycloalkyl, heterocycloalkenyl, spiro-heterocyclic, fused-heterocyclic, bridged-heterocyclic, aryl, or heteroaryl;
   R₉ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₉ and L₄ groups, taken together with the atoms to which they are attached, may optionally form a cycloalkyl, heterocycloalkyl, heterocycloalkenyl, aryl, or heteroaryl, each optionally subsitiuted with one or more R_{d}; and
   V is C(Rₐ) or N; and
   s is 0, 1, 2, 3, or 4,
   while the remaining groups are as defined in Formula (A).

In another embodiment, the compound is represented by Formula (C)

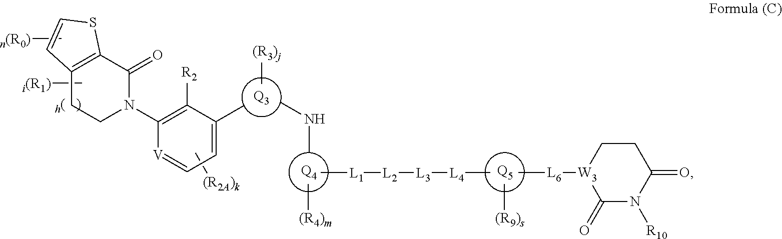

wherein h is 0, 1, or 2, while the remaining groups are as defined in Formula (B).

In another embodiment, the compound is represented by Formula (D):

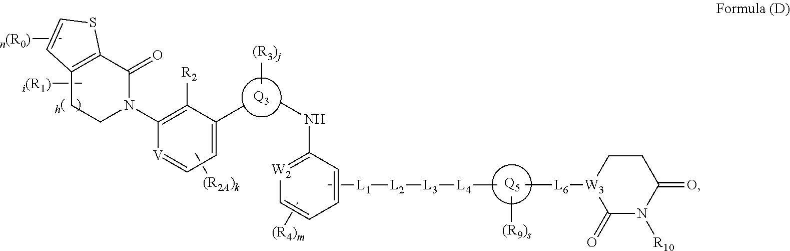

wherein W₂ is C(Rₐ) or N, while the remaining groups are as defined in Formula (C).

In another embodiment, the compound is represented by Formula (E):

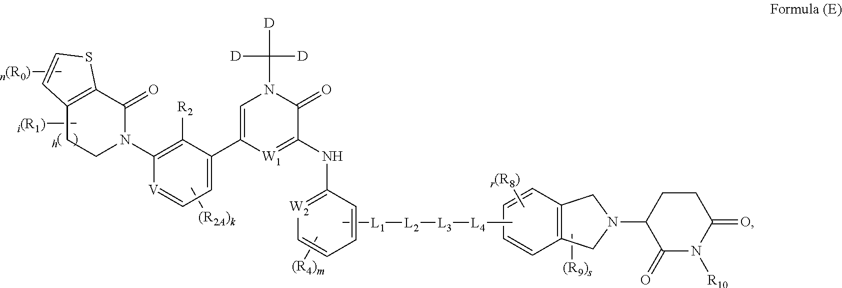

wherein
   W₁ is CH and W₂ is N, or W₁ is N and W₂ is CH;
   R₈ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₈ and L₄ groups, taken together with the atom to which they are attached, may optionally form a cycloalkyl, heterocycloalkyl, heterocycloalkenyl, aryl, or heteroaryl, each optionally subsitiuted with one or more R_{d}; and
   r is 0, 1, 2, 3, or 4,
   while the remaining groups are as defined in Formula (D).

In another aspect, this invention relates to a compound of Formula (I), or an N-oxide thereof, or a pharmaceutically acceptable salt, solvate, polymorph, tautomer, stereoisomer, an isotopic form, or a prodrug of said compound of Formula (I) or N-oxide thereof:

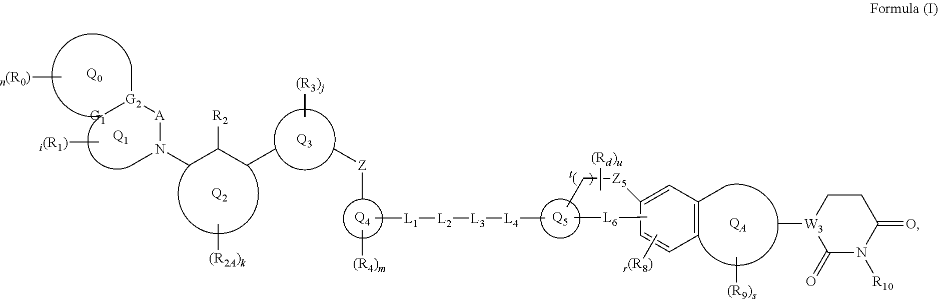

wherein
   Z₅ is absent, a bond, O, S, SO₂, C(Rₐ)(R_{b}), or N(Rₐ);
   each of t, and u, independently, is 0, 1, 2, or 3;
   each of L₁, L₂, L₃, L₄, and L₆, independently, is absent, a bond, N(Rₐ), O, S, C(O), S(O₂), OC(O), C(O)O, OSO₂, S(O₂O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   Q₀ is a 5-9 membered aryl or heteroaryl;
   Q₁ is a 5-7 membered heterocycloalkyl;
   Q₀ and Q₁ taken together form a fused-heterocyclic with two or more shared/border atoms between Q₀ and Q₁, including G₁ and G₂, wherein each said shared/border atoms can be carbon or heteroatom;
   Q₂ is a 5-9 membered cycloalkyl, heterocycloalkyl, aryl or heteroaryl;
   Q₃ is a 5-9 membered aryl or heteroaryl;
   Q₄ is a 5-9 membered aryl or heteroaryl;
   Q_{A} is a cycloalkyl, cycloalkenyl, heterocycloalkyl, aryl or heteroaryl;
   Q₅ is cycloalkyl, cycloalkenyl, heterocycloalkyl, heterocycloalkenyl, spiro-heterocyclic, fused-heterocyclic, bridged-heterocyclic, aryl, or heteroaryl, in which each of cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   A is in Q₁ and is —C(O)—, —P(O)(RₐR_{b})—, or —S(O₂)—;
   W₃ is N or CH;
   Z is NH or O;
   each of R₀, R₁, R_{2A}, R₃, R₄, R₈, and R₉, independently, is H, D, alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₂ is H, halo, alkyl, —C(RₐR_{b}R_{c}), haloalkyl, or hydroxyalkyl;
   R₁₀ is H, D, -alkyl-O—P(O)(Rₐ)(R_{b}), or -alkyl-OC(O)—Rₐ;
   two of R₀ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₁ groups, taken together with the atom to which they are attached, may optionally form a cycloalkyl, heterocycloalkyl, heterocycloalkenyl, aryl, or heteroaryl, each optionally subsitiuted with one or more R_{d};
   two of R_{2A} groups, taken together with the atom to which they are attached, may optionally form a cycloalkyl, heterocycloalkyl, heterocycloalkenyl, aryl, or heteroaryl, each optionally subsitiuted with one or more R_{d};
   two of R₃ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₄ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₈ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₉ groups, taken together with the atom to which they are attached, may optionally form a cycloalkyl, heterocycloalkyl, heterocycloalkenyl, aryl, or heteroaryl, each optionally subsitiuted with one or more R_{d};
   each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more Rₑ;
   each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{f}; and
   each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl;
   Rₐ and R_{b}, taken together with the atom to which they are attached, may optionally form a cycloalkyl, heterocycloalkyl, heterocycloalkenyl, aryl, or heteroaryl, each optionally subsitiuted with one or more Rₑ;
   R_{b} and R_{c}, taken together with the atom to which they are attached, may optionally form a cycloalkyl, heterocycloalkyl, heterocycloalkenyl, aryl, or heteroaryl, each optionally subsitiuted with one or more Rₑ;
   two of R_{d} groups, taken together with the atom to which they are attached, may optionally form a cycloalkyl, heterocycloalkyl, heterocycloalkenyl, aryl, or heteroaryl, each optionally subsitiuted with one or more Rₑ;
   two of Rₑ groups, taken together with the atom to which they are attached, may optionally form a cycloalkyl, heterocycloalkyl, heterocycloalkenyl, aryl, or heteroaryl, each optionally subsitiuted with one or more R_{f}; and
   two of R_{f} groups, taken together with the atom to which they are attached, may optionally form a cycloalkyl, heterocycloalkyl, heterocycloalkenyl, aryl, or heteroaryl, each optionally subsitiuted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl,
   each of i, j, k, m, n, r, s, u, p, and q, independently, is 0, 1, 2, 3, or 4.

In some embodiments, the compound is represented by Formula (II):

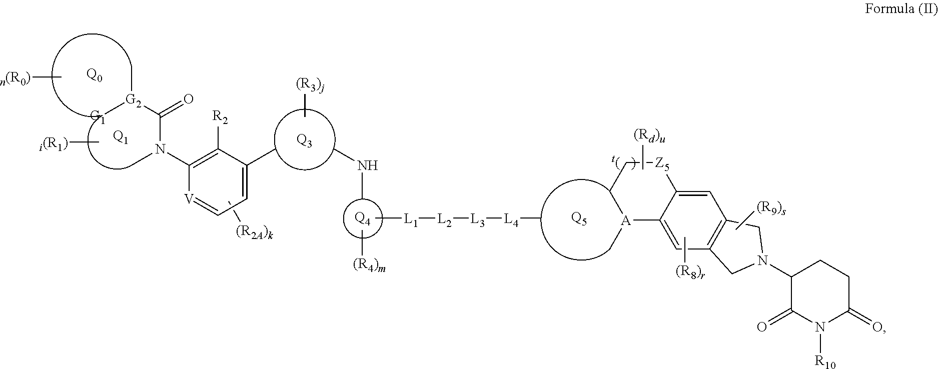

wherein
   A is shared between Q₅ and the ring with Z₅, and is N or C(Rₐ); and
   V is N or C(Rₐ),
   G₁ and G₂ are border atoms between Q₀ and Q₁, and are each independently a carbon or a heteroatom,
   while the remaining groups are as defined in formula (I).

In some embodiments, the compound is represented by Formula (III):

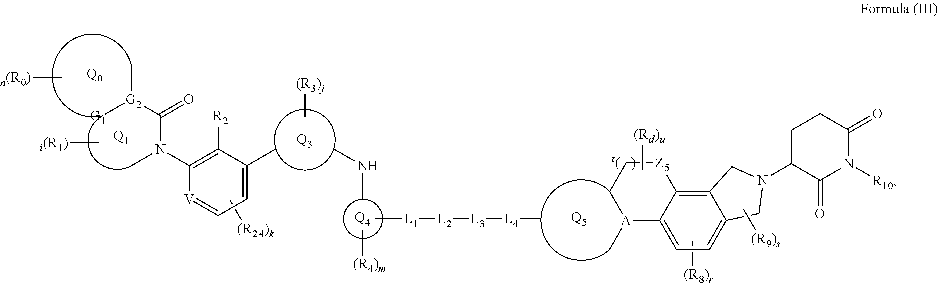

wherein
   A is shared between Q₅ and the ring with Z₅, and is N or C(Rₐ); and
   V is N or C(Rₐ),
   G₁ and G₂ are border atoms between Q₀ and Q₁ and are each independently a carbon or a heteroatom,
   while the remaining groups are as defined in formula (I).

In some embodiments, the compound is represented by Formula (IV):

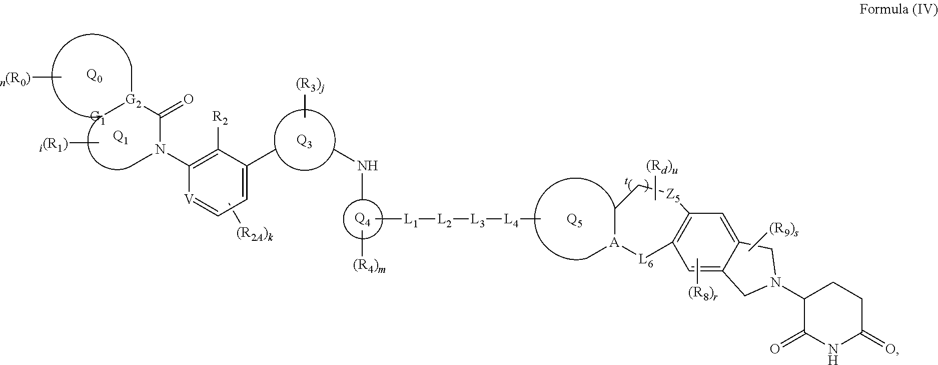

wherein
   A is shared between Q₅ and the ring with Z₅, and is N or C(Rₐ);
   L₆ is not absent;
   V is N or C(Rₐ);
   G₁ and G₂ are border atoms between Q₀ and Q₁ and are each independently a carbon or a heteroatom,
   while the remaining groups are as defined in formula (I).

In some embodiments, the compound is represented by Formula (V):

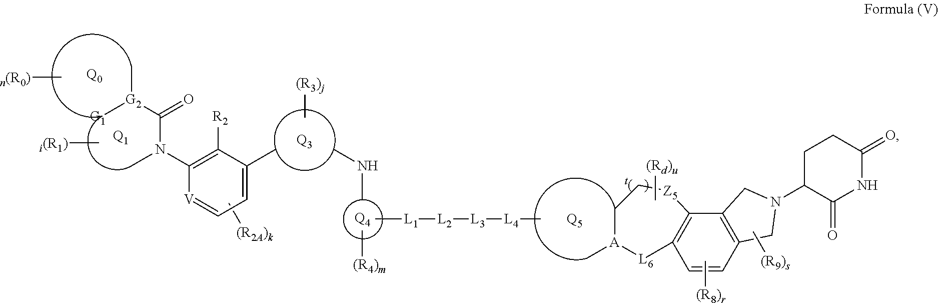

wherein
   A is shared between Q₅ and the ring with Z₅, and is N or C(Rₐ);
   L₆ is not absent;
   V is N or C(Rₐ);
   G₁ and G₂ are border atoms between Q₀ and Q₁ and are each independently a carbon or a heteroatom,
   while the remaining groups are as defined in formula (I).

In another aspect, this invention relates to a compound of Formula (11), or an N-oxide thereof, or a pharmaceutically acceptable salt, solvate, polymorph, tautomer, stereoisomer, an isotopic form, or a prodrug of said compound of Formula (11) or N-oxide thereofIn a more preferred embodiment, the compound is represented by Formula (11):

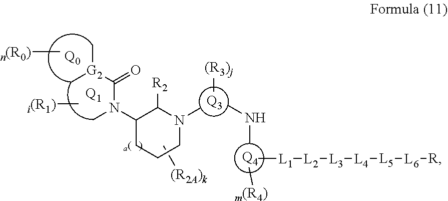

wherein
   R is a small molecule (e.g., molecular weight less than about 1,500 Da, 1,200 Da, 900 Da, 500 Da or less) E3 ubiquitin ligase binding moiety that binds an E3 ubiquitin ligase;
   each of L₁, L₂, L₃, L₄, L₅, and L₆, independently, is absent, a bond, N(Rₐ), O, S, C(O), S(O₂), OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   Q₀ is a 5-9 membered aryl or heteroaryl;
   Q₁ is a 5-7 membered heterocycloalkyl;
   G₁ and G₂ are border atoms between Q₀ and Q₁ and can each independently be either carbon or N,
   Q₃ is a 5-9 membered aryl or heteroaryl;
   Q₄ is a 5-9 membered aryl or heteroaryl;
   each of R₀, R₁, R_{2A}, R₃, and R₄, independently, is H, D, alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₂ is H, halo, alkyl, —C(RₐR_{b}R_{c}), haloalkyl, or hydroxyalkyl;
   two of R₀ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₁ groups, taken together with the atom to which they are attached, may optionally form a cycloalkyl, heterocycloalkyl, heterocycloalkenyl, aryl, or heteroaryl, each optionally subsitiuted with one or more R_{d};
   two of R_{2A} groups, taken together with the atom to which they are attached, may optionally form a cycloalkyl, heterocycloalkyl, heterocycloalkenyl, aryl, or heteroaryl, each optionally subsitiuted with one or more R_{d};
   two of R₃ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₄ groups, taken together with the atom to which they are attached, may optionally form a cycloalkyl, heterocycloalkyl, heterocycloalkenyl, aryl, or heteroaryl, each optionally subsitiuted with one or more R_{d};
   each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, heteroaryl is optionally subsitiuted with one or more Rₑ;
   each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, heteroaryl is optionally subsitiuted with one or more R_{f}; and
   each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl;
   Rₐ and R_{b}, taken together with the atom to which they are attached, may optionally form a cycloalkyl, heterocycloalkyl, heterocycloalkenyl, aryl, or heteroaryl, each optionally subsitiuted with one or more Rₑ;
   R_{b} and R_{c}, taken together with the atom to which they are attached, may optionally form a cycloalkyl, heterocycloalkyl, heterocycloalkenyl, aryl, or heteroaryl, each optionally subsitiuted with one or more Rₑ;
   two of R_{d} groups, taken together with the atom to which they are attached, may optionally form a cycloalkyl, heterocycloalkyl, heterocycloalkenyl, aryl, or heteroaryl, each optionally subsitiuted with one or more Rₑ;
   two of Rₑ groups, taken together with the atom to which they are attached, may optionally form a cycloalkyl, heterocycloalkyl, heterocycloalkenyl, aryl, or heteroaryl, each optionally subsitiuted with one or more R_{f}; and
   two of R_{f} groups, taken together with the atom to which they are attached, may optionally form a cycloalkyl, heterocycloalkyl, heterocycloalkenyl, aryl, or heteroaryl, each optionally subsitiuted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl,
   a is 0, 1 or 2;
   each of i, j, k, m, n, p, and q, independently, is 0, 1, 2, 3, or 4.

In some embodiments, said E3 ubiquitin ligase is Cereblon, Von Hippel-Lindau, mouse double-minute homolog 2, or IAP.

In some embodiments, the compound is represented by Formula (12):

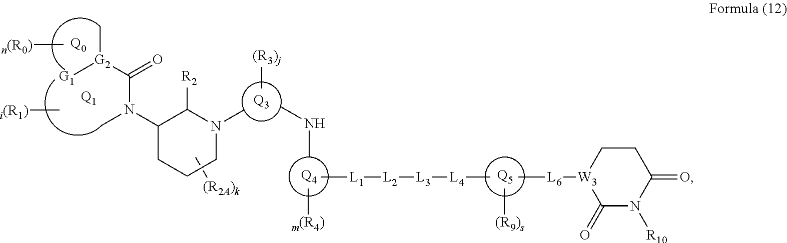

wherein
   R₁₀ is H, D, -alkyl-O—P(O)(Rₐ)(R_{b}), or -alkyl-OC(O)—Rₐ;
   W₃ is N or CH;
   L₆ is absent, NH, CONH, or O;
   Q₅ is absent, cycloalkyl, cycloalkenyl, heterocycloalkyl, heterocycloalkenyl, spiro-heterocyclic, fused-heterocyclic, bridged-heterocyclic, heterocycloalkenyl, aryl, or heteroaryl;
   R₉ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₉ and L₄ groups, taken together with the atoms to which they are attached, may optionally form a cycloalkyl, heterocycloalkyl, heterocycloalkenyl, aryl, or heteroaryl, each optionally subsitiuted with one or more R_{d}; and
   s is 0, 1, 2, 3, or 4,
   G₁ and G₂ are border atoms between Q₀ and Q₁ and are each independently a carbon or a heteroatom,
   while the remaining groups are as defined in formula (11).

In one embodiment, the compound is represented by Formula (13):

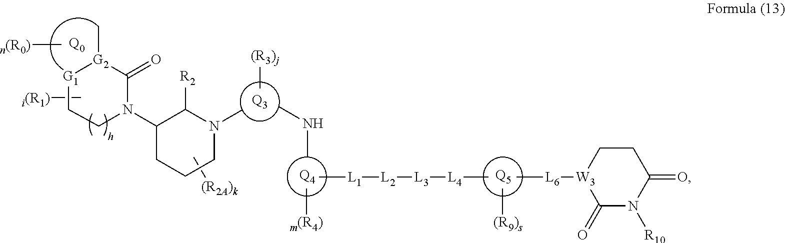

wherein h is 0, 1 or 2, G₁ and G₂ are border atoms between Q₀ and Q₁ and are each independently a carbon or a heteroatom, while the remaining groups are as defined in Formula (12).

In one embodiment, the compound is represented by Formula (14):

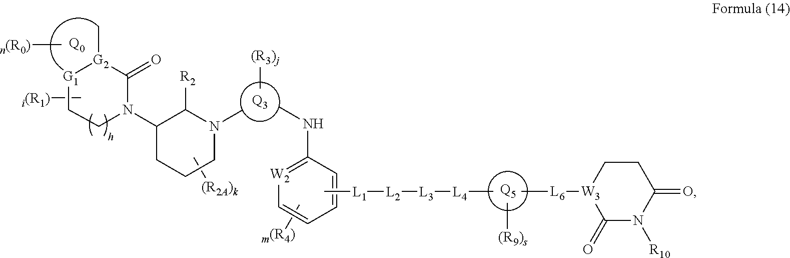

wherein W₂ is N, or CH, G₁ and G₂ are border atoms between Q₀ and Q₁ and are each independently a carbon or a heteroatom, while the remaining groups are as defined in Formula (12).

In one embodiment, the compound is represented by Formula (15):

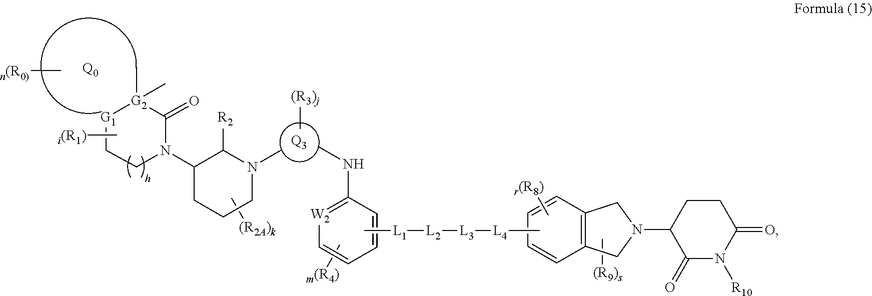

wherein
   R₈ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₈ and L₄ groups, taken together with the atom to which they are attached, may optionally form a cycloalkyl, heterocycloalkyl, heterocycloalkenyl, aryl, or heteroaryl optionally subsitiuted with one or more R_{d}; and
   r is 0, 1, 2, 3, or 4,
   G₁ and G₂ are border atoms between Q₀ and Q₁ and are each independently a carbon or a heteroatom,
   while the remaining groups are as defined in Formula (12).

In another aspect, this invention relates to a compound of Formula (16), or an N-oxide thereof, or a pharmaceutically acceptable salt, solvate, polymorph, tautomer, stereoisomer, an isotopic form, or a prodrug of said compound of Formula (16) or N-oxide thereof:

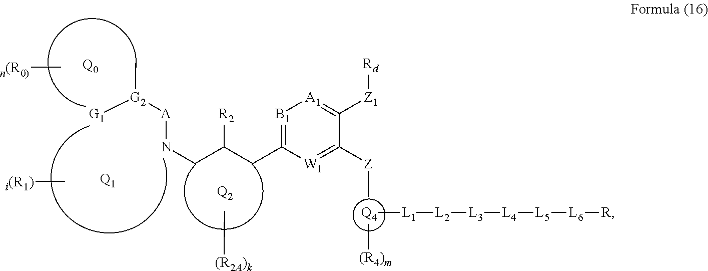

wherein
   R is a small molecule (e.g., molecular weight less than about 1,500 Da, 1,200 Da, 900 Da, 500 Da or less) E3 ubiquitin ligase binding moiety that binds an E3 ubiquitin ligase;
   each of L₁, L₂, L₃, L₄, L₅, and L₆, independently, is absent, a bond, N(Rₐ), O, S, C(O), S(O₂), OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   Q₀ is a 5-9 membered aryl or heteroaryl;
   Q₁ is a 5-7 membered heterocycloalkyl;
   Q₀ and Q₁ taken together form a fused-heterocyclic with two or more shared/border atoms between Q₀ and Q₁, including G₁ and G₂, wherein each said shared/border atoms can be carbon or heteroatom;
   Q₂ is a 5-9 membered cycloalkyl, heterocycloalkyl, aryl or heteroaryl;
   Q₄ is a 5-9 membered aryl or heteroaryl;
   A is in Q₁ and is —C(O)—, —P(O)(RₐR_{b})—, or —S(O₂)—;
   Z is NH or O;
   Z₁ is O, N(Rₐ), C(O)N(Rₐ)(R_{b}), or S(O₂);
   A₁ is N or C(R_{d});
   B₁ is N or C(R_{d});
   each of R₀, R₁, R_{2A}, and R₄, independently, is H, D, alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, ═NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₂ is H, halo, alkyl, —C(RₐR_{b}R_{c}), haloalkyl, or hydroxyalkyl;
   two of R₀ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₁ groups, taken together with the atom to which they are attached, may optionally form a cycloalkyl, heterocycloalkyl, heterocycloalkenyl, aryl, or heteroaryl, each optionally subsitiuted with one or more R_{d};
   two of R_{2A} groups, taken together with the atom to which they are attached, may optionally form a cycloalkyl, heterocycloalkyl, heterocycloalkenyl, aryl, or heteroaryl, each optionally subsitiuted with one or more R_{d};
   two of R₃ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₄ groups, taken together with the atom to which they are attached, may optionally form a cycloalkyl, heterocycloalkyl, heterocycloalkenyl, aryl, or heteroaryl, each optionally subsitiuted with one or more R_{d};
   each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, heteroaryl is optionally subsitiuted with one or more Rₑ;
   each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{f}; and
   each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl;
   Rₐ and R_{b}, taken together with the atom to which they are attached, may optionally form a cycloalkyl, heterocycloalkyl, heterocycloalkenyl, aryl, or heteroaryl, each optionally subsitiuted with one or more Rₑ;
   R_{b} and R_{c}, taken together with the atom to which they are attached, may optionally form a cycloalkyl, heterocycloalkyl, heterocycloalkenyl, aryl, or heteroaryl, each optionally subsitiuted with one or more Rₑ;
   two of R_{d} groups, taken together with the atom to which they are attached, may optionally form a cycloalkyl, heterocycloalkyl, heterocycloalkenyl, aryl, or heteroaryl, each optionally subsitiuted with one or more Rₑ;
   two of Rₑ groups, taken together with the atom to which they are attached, may optionally form a cycloalkyl, heterocycloalkyl, heterocycloalkenyl, aryl, or heteroaryl, each optionally subsitiuted with one or more R_{f}; and
   two of R_{f} groups, taken together with the atom to which they are attached, may optionally form a cycloalkyl, heterocycloalkyl, heterocycloalkenyl, aryl, or heteroaryl, each optionally subsitiuted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl,
   each of i, k, m, n, p, and q, independently, is 0, 1, 2, 3, or 4.

In some embodiments, said E3 ubiquitin ligase is Cereblon, Von Hippel-Lindau, mouse double-minute homolog 2, or IAP.

In some embodiments, the compound is represented by Formula (17):

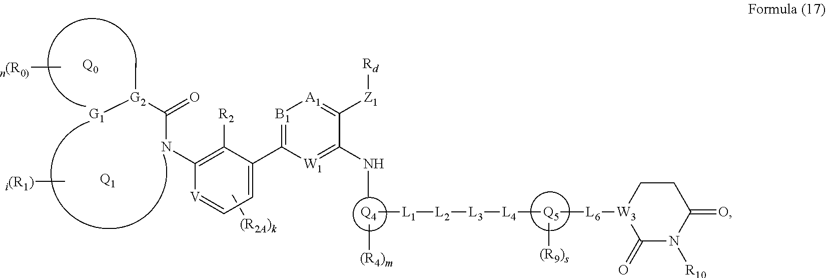

wherein
   R₁₀ is H, D, -alkyl-O—P(O)(Rₐ)(R_{b}), or -alkyl-OC(O)—Rₐ;
   L₆ is absent, NH, CONH, or O;
   W₁ is N or CH;
   W₃ is N or CH;
   Q₅ is absent, cycloalkyl, cycloalkenyl, heterocycloalkyl, heterocycloalkenyl, spiro-heterocyclic, fused-heterocyclic, bridged-heterocyclic, aryl, or heteroaryl;
   R₉ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₉ and L₄ groups, taken together with the atom to which they are attached, may optionally form a cycloalkyl, heterocycloalkyl, heterocycloalkenyl, aryl, or heteroaryl optionally subsitiuted with one or more R_{d}; and
   V is C(Rₐ) or N; and
   s is 0, 1, 2, 3, or 4,
   G₁ and G₂ are border atoms between Q₀ and Q₁ and can each independently be either carbon or N,
   while the remaining groups are as defined in Formula (16).

In one embodiment, the compound is represented by Formula (18)

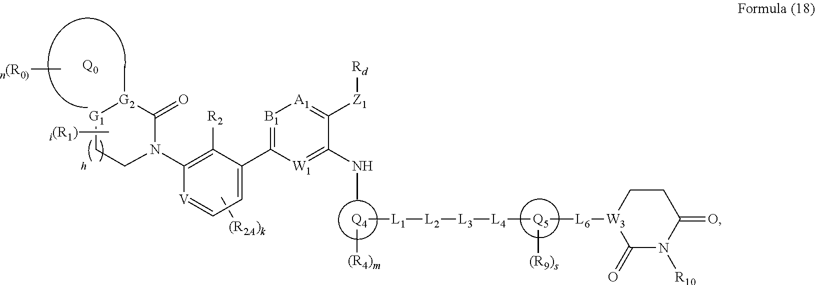

wherein h is 0, 1 or 2, G₁ and G₂ are border atoms between Q₀ and Q₁ and are each indeoendently a carbon or N, while the remaining groups are as defined in Formula (17).

In one embodiment, the compound is represented by Formula (19):

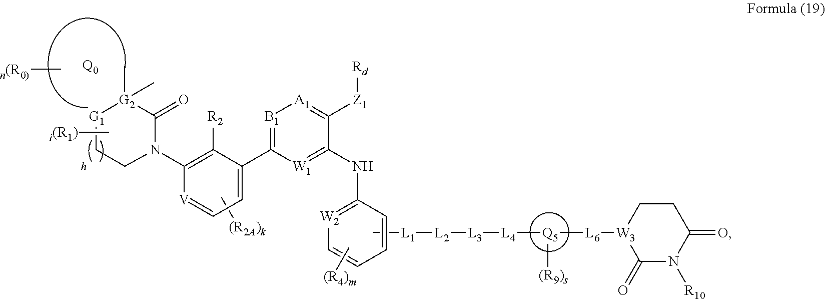

wherein W₁ is CH and W₂ is N, or W₁ is N and W₂ is CH, G₁ and G₂ are border atoms between Q₀ and Q₁ and are each independently carbon or N, while the remaining groups are as defined in Formula (18).

In one embodiment, the compound is represented by Formula (20):

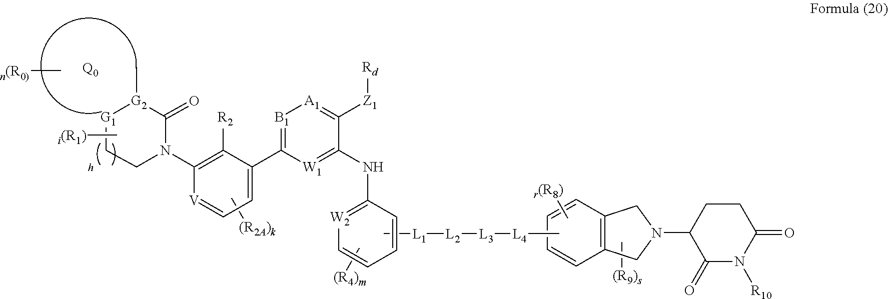

wherein
   R₈ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₈ and L₄ groups, taken together with the atom to which they are attached, may optionally form a cycloalkyl, heterocycloalkyl, heterocycloalkenyl, aryl, or heteroaryl optionally subsitiuted with one or more R_{d}; and
   r is 0, 1, 2, 3, or 4,
   G₁ and G₂ are border atoms between Q₀ and Q₁ and are each independently a carbon or N, while the remaining groups are as defined in Formula (19).

In another aspect, this invention relates to a compound of Formula (20), or an N-oxide thereof, or a pharmaceutically acceptable salt, solvate, polymorph, tautomer, stereoisomer, an isotopic form, or a prodrug of said compound of Formula (20) or N-oxide thereof:

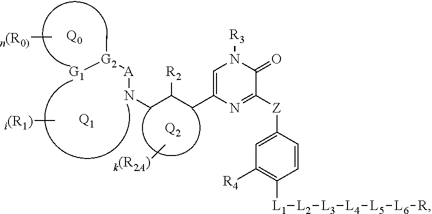

wherein
   R is a small molecule (e.g., molecular weight less than about 1,500 Da, 1,200 Da, 900 Da, 500 Da or less) E3 ubiquitin ligase binding moiety that binds an E3 ubiquitin ligase;
   each of L₁, L₂, L₃, L₄, L₅, and L₆, independently, is absent, a bond, N(Rₐ), O, S, C(O), S(O₂), OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, cycloalkyl, cycloalkenyl, heterocycloalkyl, spiro-heterocyclic, fused-heterocyclic, bridged-heterocyclic, heterocycloalkenyl, aryl, or heteroaryl is optionally subsitiuted with one or more R_{d};
   Q₀ is a 5-9 membered aryl or heteroaryl;
   Q₁ is a 5-7 membered heterocycloalkyl;
   Q₀ and Q₁ taken together form a fused-heterocyclic with two or more shared/border atoms between Q₀ and Q₁, including G₁ and G₂, wherein each said shared/border atoms can be carbon or heteroatom;
   Q₂ is a 5-9 membered cycloalkyl, heterocycloalkyl, aryl or heteroaryl;
   Q₄ is a 5-9 membered aryl or heteroaryl;
   A is in Q₁ and is —C(O)—, —P(O)(RₐR_{b})—, or —S(O₂)—;
   Z is NH or O;
   each of R₀, R₁, R_{2A}, and R₃, independently, is H, D, alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, =NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₂ is H, halo, alkyl, —C(RₐR_{b}R_{c}), haloalkyl, or hydroxyalkyl;
   R₄ is alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, =NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, heteroaryl is optionally subsitiuted with one or more R_{d};
   two of R₀ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₁ groups, taken together with the atom to which they are attached, may optionally form a cycloalkyl, heterocycloalkyl, heterocycloalkenyl, aryl, or heteroaryl, each optionally subsitiuted with one or more R_{d};
   two of R_{2A} groups, taken together with the atom to which they are attached, may optionally form a cycloalkyl, heterocycloalkyl, heterocycloalkenyl, aryl, or heteroaryl, each optionally subsitiuted with one or more R_{d};
   two of R₃ groups, taken together with the atom to which they are attached, may optionally form a cycloalkyl, heterocycloalkyl, heterocycloalkenyl, aryl, or heteroaryl, each optionally subsitiuted with one or more R_{d};
   two of R₄ groups, taken together with the atom to which they are attached, may optionally form a cycloalkyl, heterocycloalkyl, heterocycloalkenyl, aryl, or heteroaryl, each optionally subsitiuted with one or more R_{d};
   each of Rₐ, R_{b}, and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more Rₑ;
   R_{c} is alkyl, cycloalkyl, heterocycloalkyl, spiro-heterocyclic, fused-heterocyclic, bridged-heterocyclic, aryl, heteroaryl;
   each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{f}; and
   each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl;
   Rₐ and R_{b}, taken together with the atom to which they are attached, may optionally form a cycloalkyl, heterocycloalkyl, heterocycloalkenyl, aryl, or heteroaryl, each optionally subsitiuted with one or more Rₑ;
   R_{b} and R_{c}, taken together with the atom to which they are attached, may optionally form a cycloalkyl, heterocycloalkyl, heterocycloalkenyl, aryl, or heteroaryl, each optionally subsitiuted with one or more Rₑ;
   two of R_{d} groups, taken together with the atom to which they are attached, may optionally form a cycloalkyl, heterocycloalkyl, heterocycloalkenyl, aryl, or heteroaryl, each optionally subsitiuted with one or more Rₑ;
   two of Rₑ groups, taken together with the atom to which they are attached, may optionally form a cycloalkyl, heterocycloalkyl, heterocycloalkenyl, aryl, or heteroaryl, each optionally subsitiuted with one or more R_{f}; and
   two of R_{f} groups, taken together with the atom to which they are attached, may optionally form a cycloalkyl, heterocycloalkyl, heterocycloalkenyl, aryl, or heteroaryl, each optionally subsitiuted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl.
   each of i, j, k, m, n, p, and q, independently, is 0, 1, 2, 3, or 4.

In some embodiments, said E3 ubiquitin ligase is Cereblon, Von Hippel-Lindau, mouse double-minute homolog 2, or IAP.

In some embodiments the compound is represented by Formula (21):

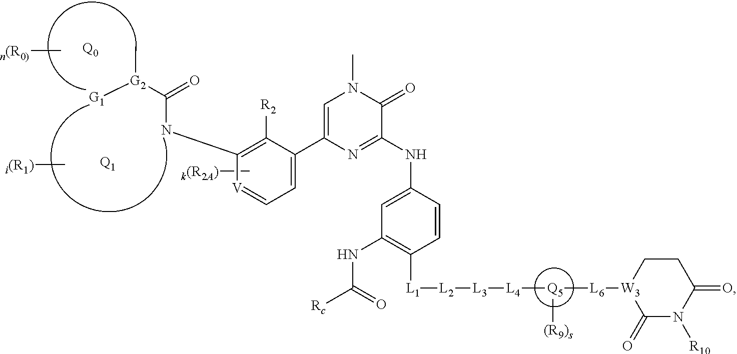

wherein
   R₁₀ is H, D, -alkyl-O—P(O)(Rₐ)(R_{b}), or -alkyl-OC(O)—Rₐ;
   L₆ is absent, NH, CONH, or O;
   W₁ is N or CH;
   W₃ is N or CH;
   Q₅ is absent, cycloalkyl, cycloalkenyl, heterocycloalkyl, heterocycloalkenyl, spiro-heterocyclic, fused-heterocyclic, bridged-heterocyclic, aryl, or heteroaryl;
   R₉ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
   R₉ and L₄ groups, taken together with the atoms to which they are attached, may optionally form a cycloalkyl, heterocycloalkyl, heterocycloalkenyl, aryl, or heteroaryl optionally subsitiuted with one or more R_{d}; and
   V is C(Rₐ) or N; and
   s is 0, 1, 2, 3, or 4,
   while the remaining groups are as defined in Formula (20).

In a further embodiment, the compound is represented by Formula (22):

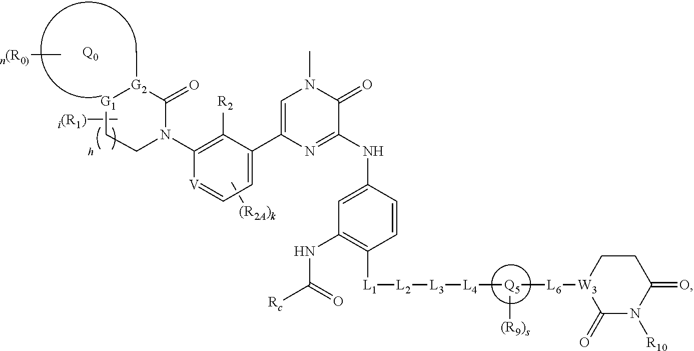

wherein h is 0, 1 or 2, each of the border atoms between Q₀ and Q₁ including G₁ and G₂, can be carbon or heteroatom, while the remaining groups are as defined in Formula (20).

In a further embodiment, the compound is represented by Formula (23):

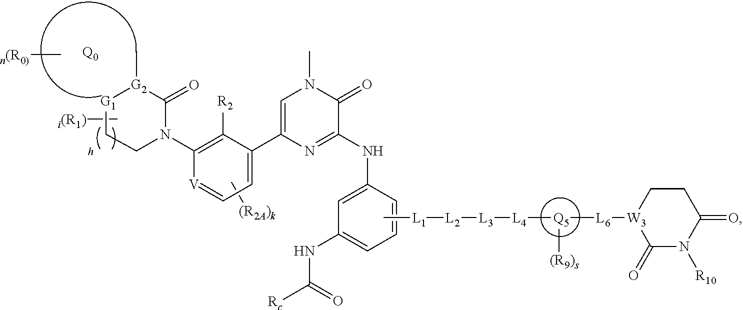

wherein W₁ is CH and W₂ is N, or W₁ is N and W₂ is CH, each of the border atoms between Q₀ and Q₁ including G₁ and G₂, can be carbon or heteroatom, while the remaining groups are as defined in Formula (20).

In a further embodiment, the compound is represented by Formula (24):

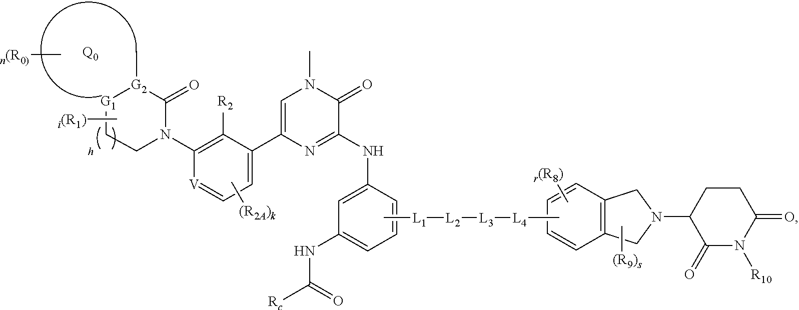

R₈ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
R₈ and L₄ groups, taken together with the atom to which they are attached, may optionally form a cycloalkyl, heterocycloalkyl, heterocycloalkenyl, aryl, or heteroaryl optionally subsitiuted with one or more R_{d}; and
r is 0, 1, 2, 3, or 4,
each of the border atoms between Q₀ and Q₁ including G₁ and G₂, can be carbon or heteroatom,
while the remaining groups are as defined in Formula (20).

Compounds of the invention may contain one or more asymmetric carbon atoms. Accordingly, the compounds may exist as diastereomers, enantiomers, or mixtures thereof. Each of the asymmetric carbon atoms may be in the R or S configuration, and both of these configurations are within the scope of the invention.

A modified compound of any one of such compounds including a modification having an improved (e.g., enhanced, greater) pharmaceutical solubility, stability, bioavailability, and/or therapeutic index as compared to the unmodified compound is also contemplated. Exemplary modifications include (but are not limited to) applicable prodrug derivatives, and deuterium-enriched compounds.

It should be recognized that the compounds of the present invention may be present and optionally administered in the form of salts or solvates. The invention encompasses any pharmaceutically acceptable salts and solvates of any one of the above-described compounds and modifications thereof.

Also within the scope of this invention is a pharmaceutical composition containing one or more of the compounds, modifications, and/or salts and thereof described above for use in treating a neoplastic disease, autoimmune disease, and inflammatory disorders, therapeutic uses thereof, and use of the compounds for the manufacture of a medicament for treating the disease/disorder.

This invention also relates to a method of treating a neoplastic disease, particularly the B-cell malignancy including but not limited to B-cell lymphoma, lymphoma (including Hodgkin's and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia, by administering to a subject in need thereof an effective amount of one or more of the compounds, modifications, and/or salts, and compositions thereof described above.

Autoimmune and/or inflammatory diseases that can be affected using compounds and compositions according to the invention include, but are not limited to: psoriasis, allergy, Crohn's disease, irritable bowel syndrome, Sjogren's disease, tissue graft rejection, and hyperacute rejection of transplanted organs, asthma, systemic lupus erythematosus (and associated glomerulonephritis), dermatomyositis, multiple sclerosis, scleroderma, vasculitis (ANCA-associated and other vasculitides), autoimmune hemolytic and thrombocytopenic states, Goodpasture's syndrome (and associated glomerulonephritis and pulmonary hemorrhage), atherosclerosis, rheumatoid arthritis, chronic Idiopathic thrombocytopenic purpura (ITP), Addison's disease, Parkinson's disease, Alzheimer's disease, diabetes, septic shock, and myasthenia gravis.

The details of one or more embodiments of the invention are set forth in the description below. Other features, objects, and advantages of the invention will be apparent from the description and from the claims. It should be understood that all embodiments/features of the invention (compounds, pharmaceutical compositions, methods of make/use, etc) described herein, including any specific features described in the examples and original claims, can combine with one another unless not applicable or explicitly disclaimed.

### DETAILED DESCRIPTION OF THE INVENTION

Exemplary compounds described herein include, but are not limited to, the following:
5-((S)-7-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-6-methyl-2,7-diazaspiro[3.5]nonan-2-yl)-2-(2,6-dioxopiperidin-3-yl)isoindoline-1,3-dione,
5-((5R,7S)-8-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-7-methyl-2,8-diazaspiro[4.5]decan-2-yl)-2-(2,6-dioxopiperidin-3-yl)isoindoline-1,3-dione,
5-((5S,7S)-8-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-7-methyl-2,8-diazaspiro[4.5]decan-2-yl)-2-(2,6-dioxopiperidin-3-yl)isoindoline-1,3-dione,
5-(9-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3,9-diazaspiro[5.5]undecan-3-yl)-2-(2,6-dioxopiperidin-3-yl)isoindoline-1,3-dione,
5-((S)-9-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-8-methyl-3,9-diazaspiro[5.5]undecan-3-yl)-2-(2,6-dioxopiperidin-3-yl)isoindoline-1,3-dione,
5-((2S,6R)-3-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2-methyl-3,9-diazaspiro[5.6]dodecan-9-yl)-2-(2,6-dioxopiperidin-3-yl)isoindoline-1,3-dione,
5-((2S,6S)-3-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2-methyl-3,9-diazaspiro[5.6]dodecan-9-yl)-2-(2,6-dioxopiperidin-3-yl)isoindoline-1,3-dione,
5-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)azetidin-1-yl)-2-(2,6-dioxopiperidin-3-yl)isoindoline-1,3-dione,
5-(3-((2R)-1-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2-isopropylpiperidin-4-yl)azetidin-1-yl)-2-(2,6-dioxopiperidin-3-yl)isoindoline-1,3-dione,
5-(3-((S)-3-(tert-butyl)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)piperazin-1-yl)azetidin-1-yl)-2-(2,6-dioxopiperidin-3-yl)isoindoline-1,3-dione,
5-(3-(4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-4-methylpiperidin-1-yl)azetidin-1-yl)-2-(2,6-dioxopiperidin-3-yl)isoindoline-1,3-dione,
5-((S)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)pyrrolidin-1-yl)-2-(2,6-dioxopiperidin-3-yl)isoindoline-1,3-dione,
5-((R)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)pyrrolidin-1-yl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
5-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione,
5-((2S,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione,
5-((2R,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione,
5-((2R,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione,
1-(3-(5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)piperidin-1-yl)-1,3-dioxoisoindolin-2-yl)-2,6-dioxopiperidin-1-yl)ethyl dihydrogen phosphate,
1-((2′-(7,7-dimethyl-1-oxo-1,3,4,6,7,8-hexahydro-2H-cyclopenta[4,5]pyrrolo[1,2-a]pyrazin-2-yl)-5-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)-1-methyl-6-oxo-1,6-dihydro-[3,4′-bipyridin]-3′-yl)methoxy)ethyl dihydrogen phosphate,
(2′-(7,7-dimethyl-1-oxo-1,3,4,6,7,8-hexahydro-2H-cyclopenta[4,5]pyrrolo[1,2-a]pyrazin-2-yl)-5-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)-1-methyl-6-oxo-1,6-dihydro-[3,4′-bipyridin]-3′-yl)methyl dihydrogen phosphate,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-isopropylpiperazin-1-yl)-2-isopropylpiperidin-1-yl)-2-(2,6-dioxopiperidin-3-yl)isoindoline-1,3-dione,
5-((S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)azepan-1-yl)-2-(2,6-dioxopiperidin-3-yl)isoindoline-1,3-dione,
5-((R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)azepan-1-yl)-2-(2,6-dioxopiperidin-3-yl)isoindoline-1,3-dione,
5-((4S)-4-((2S)-1-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2-methylpiperidin-4-yl)azepan-1-yl)-2-(2,6-dioxopiperidin-3-yl)isoindoline-1,3-dione,
5-((4R)-4-((2S)-1-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2-methylpiperidin-4-yl)azepan-1-yl)-2-(2,6-dioxopiperidin-3-yl)isoindoline-1,3-dione,
5-(4-((2S)-1-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2-methylpiperidin-4-yl)piperazin-1-yl)-2-(2,6-dioxopiperidin-3-yl)isoindoline-1,3-dione,
5-((S)-1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-6-azaspiro[2.5]octan-6-yl)-2-(2,6-dioxopiperidin-3-yl)isoindoline-1,3-dione,
5-((1S)-1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-isopropylpiperazin-1-yl)-5-isopropyl-6-azaspiro[2.5]octan-6-yl)-2-(2,6-dioxopiperidin-3-yl)isoindoline-1,3-dione,
5-((R)-1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-6-azaspiro[2.5]octan-6-yl)-2-(2,6-dioxopiperidin-3-yl)isoindoline-1,3-dione,
5-(2-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-7-azaspiro[3.5]nonan-7-yl)-2-(2,6-dioxopiperidin-3-yl)isoindoline-1,3-dione,
5-(6-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-azaspiro[3.3]heptan-2-yl)-2-(2,6-dioxopiperidin-3-yl)isoindoline-1,3-dione,
5-(2-((S)-3-(tert-butyl)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)piperazin-1-yl)-6-methyl-7-azaspiro[3.5]nonan-7-yl)-2-(2,6-dioxopiperidin-3-yl)isoindoline-1,3-dione,
5-((S)-2-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-6-methyl-7-azaspiro[3.5]nonan-7-yl)-2-(2,6-dioxopiperidin-3-yl)isoindoline-1,3-dione,
5-((S)-2-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-azaspiro[4.5]decan-8-yl)-2-(2,6-dioxopiperidin-3-yl)isoindoline-1,3-dione,
5-((R)-2-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-azaspiro[4.5]decan-8-yl)-2-(2,6-dioxopiperidin-3-yl)isoindoline-1,3-dione,
5-(9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-azaspiro[5.5]undecan-3-yl)-2-(2,6-dioxopiperidin-3-yl)isoindoline-1,3-dione,
5-(4-((1R,3R,5S)-6-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-5-methyl-6-azaspiro[2.5]octan-1-yl)piperazin-1-yl)-2-(2,6-dioxopiperidin-3-yl)isoindoline-1,3-dione,
5-((S)-4-((1R,3R,5R)-5-(tert-butyl)-6-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-6-azaspiro[2.5]octan-1-yl)-2-methylpiperazin-1-yl)-2-(2,6-dioxopiperidin-3-yl)isoindoline-1,3-dione,
5-(4-((1R,3S,5S)-6-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-5-methyl-6-azaspiro[2.5]octan-1-yl)piperazin-1-yl)-2-(2,6-dioxopiperidin-3-yl)isoindoline-1,3-dione,
5-(4-((S)-7-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-6-methyl-7-azaspiro[3.5]nonan-2-yl)piperazin-1-yl)-2-(2,6-dioxopiperidin-3-yl)isoindoline-1,3-dione,
5-((2S,3R)-4-((R)-6-(tert-butyl)-7-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-7-azaspiro[3.5]nonan-2-yl)-2,3-dimethylpiperazin-1-yl)-2-(2,6-dioxopiperidin-3-yl)isoindoline-1,3-dione,
5-(4-((7S)-8-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-7-methyl-8-azaspiro[4.5]decan-2-yl)piperazin-1-yl)-2-(2,6-dioxopiperidin-3-yl)isoindoline-1,3-dione,
5-(4-((S)-3-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2-methyl-3-azaspiro[5.5]undecan-9-yl)piperazin-1-yl)-2-(2,6-dioxopiperidin-3-yl)isoindoline-1,3-dione,
5-((S)-4-((R)-2-(tert-butyl)-3-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-azaspiro[5.5]undecan-9-yl)-2-methylpiperazin-1-yl)-2-(2,6-dioxopiperidin-3-yl)isoindoline-1,3-dione,
3-(5-((R)-1-((S)-7-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-6-methyl-7-azaspiro[3.5]nonan-2-yl)pyrrolidin-2-yl)-1-oxoisoindolin-2-yl)piperidine-2,6-dione,
5-(5-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)hexahydrocyclopenta[c]pyrrol-2(1H)-yl)-2-(2,6-dioxopiperidin-3-yl)isoindoline-1,3-dione,
5-(5-((2S)-1-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2-methylpiperidin-4-yl)hexahydropyrrolo[3,4-c]pyrrol-2(1H)-yl)-2-(2,6-dioxopiperidin-3-yl)isoindoline-1,3-dione,
5-((S)-3-((4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-4-methylpiperidin-1-yl)methyl)pyrrolidin-1-yl)-2-(2,6-dioxopiperidin-3-yl)isoindoline-1,3-dione,
5-(3-(((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)methyl)azetidin-1-yl)-2-(2,6-dioxopiperidin-3-yl)isoindoline-1,3-dione,
5-((R)-3-(((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)methyl)pyrrolidin-1-yl)-2-(2,6-dioxopiperidin-3-yl)isoindoline-1,3-dione,
5-(4-(((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)methyl)piperidin-1-yl)-2-(2,6-dioxopiperidin-3-yl)isoindoline-1,3-dione,
5-(4-(((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-isopropylpiperazin-1-yl)methyl)piperidin-1-yl)-2-(2,6-dioxopiperidin-3-yl)isoindoline-1,3-dione,
5-(3-(1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)azetidin-1-yl)-2-(2,6-dioxopiperidin-3-yl)isoindoline-1,3-dione,
5-((S)-3-(1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)pyrrolidin-1-yl)-2-(2,6-dioxopiperidin-3-yl)isoindoline-1,3-dione,
5-(4-(1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)piperidin-1-yl)-2-(2,6-dioxopiperidin-3-yl)isoindoline-1,3-dione,
5-(((2'S)-1′-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-2′-methyl-[1,4′-bipiperidin]-4-yl)ethynyl)-2-(2,6-dioxopiperidin-3-yl)isoindoline-1,3-dione,
5-((4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclohexyl)ethynyl)-2-(2,6-dioxopiperidin-3-yl)isoindoline-1,3-dione,
5-((4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-isopropylpiperazin-1-yl)cyclohexyl)ethynyl)-2-(2,6-dioxopiperidin-3-yl)isoindoline-1,3-dione,
5-(((1S,3R)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclopentyl)ethynyl)-2-(2,6-dioxopiperidin-3-yl)isoindoline-1,3-dione,
5-((3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)ethynyl)-2-(2,6-dioxopiperidin-3-yl)isoindoline-1,3-dione,
5-(((1S,2R)-2-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclopropyl)ethynyl)-2-(2,6-dioxopiperidin-3-yl)isoindoline-1,3-dione,
5-((S)-1-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)pyrrolidin-3-yl)-2-(2,6-dioxopiperidin-3-yl)isoindoline-1,3-dione,
5-((S)-1-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclohexyl)pyrrolidin-3-yl)-2-(2,6-dioxopiperidin-3-yl)isoindoline-1,3-dione,
5-(1-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)azetidin-3-yl)-2-(2,6-dioxopiperidin-3-yl)isoindoline-1,3-dione,
5-(4-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)piperazin-1-yl)-2-(2,6-dioxopiperidin-3-yl)isoindoline-1,3-dione,
5-((R)-1-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)pyrrolidin-2-yl)-2-(2,6-dioxopiperidin-3-yl)isoindoline-1,3-dione,
5-((S)-1-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclohexyl)pyrrolidin-2-yl)-2-(2,6-dioxopiperidin-3-yl)isoindoline-1,3-dione,
3-(6-((3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)ethynyl)-1-oxoisoindolin-2-yl)piperidine-2,6-dione,
3-(5-((3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)ethynyl)-1-oxoisoindolin-2-yl)piperidine-2,6-dione,
4-((3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)ethynyl)-2-(2,6-dioxopiperidin-3-yl)isoindoline-1,3-dione,
3-(4-((3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)ethynyl)-1-oxoisoindolin-2-yl)piperidine-2,6-dione,
3-(7-((3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)ethynyl)-1-oxoisoindolin-2-yl)piperidine-2,6-dione,
3-(5-(((1S,2R)-2-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclopropyl)ethynyl)-1-oxoisoindolin-2-yl)piperidine-2,6-dione,
3-(6-((S)-1-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)pyrrolidin-3-yl)-1-oxoisoindolin-2-yl)piperidine-2,6-dione,
3-(5-((S)-1-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclohexyl)pyrrolidin-3-yl)-1-oxoisoindolin-2-yl)piperidine-2,6-dione,
3-(5-(1-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)azetidin-3-yl)-1-oxoisoindolin-2-yl)piperidine-2,6-dione,
3-(5-(4-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)piperazin-1-yl)-1-oxoisoindolin-2-yl)piperidine-2,6-dione,
3-(5-((R)-1-(3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclobutyl)pyrrolidin-2-yl)-1-oxoisoindolin-2-yl)piperidine-2,6-dione,
3-(6-((S)-1-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)cyclohexyl)pyrrolidin-2-yl)-1-oxoisoindolin-2-yl)piperidine-2,6-dione,
5-((S)-7-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-6-methyl-2,7-diazaspiro[3.5]nonan-2-yl)-2-(2,6-dioxopiperidin-3-yl)isoindoline-1,3-dione,
5-((S)-7-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-6-methyl-2,7-diazaspiro[3.5]nonan-2-yl)-2-(2,6-dioxopiperidin-3-yl)isoindoline-1,3-dione,
5-((5R,7S)-8-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-7-methyl-2,8-diazaspiro[4.5]decan-2-yl)-2-(2,6-dioxopiperidin-3-yl)isoindoline-1,3-dione,
5-((5S,7S)-8-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-7-methyl-2,8-diazaspiro[4.5]decan-2-yl)-2-(2,6-dioxopiperidin-3-yl)isoindoline-1,3-dione,
5-(9-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3,9-diazaspiro[5.5]undecan-3-yl)-2-(2,6-dioxopiperidin-3-yl)isoindoline-1,3-dione,
5-((S)-9-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-8-methyl-3,9-diazaspiro[5.5]undecan-3-yl)-2-(2,6-dioxopiperidin-3-yl)isoindoline-1,3-dione,
5-((2S,6R)-3-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-methyl-3,9-diazaspiro[5.6]dodecan-9-yl)-2-(2,6-dioxopiperidin-3-yl)isoindoline-1,3-dione,
5-((2S,6S)-3-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-methyl-3,9-diazaspiro[5.6]dodecan-9-yl)-2-(2,6-dioxopiperidin-3-yl)isoindoline-1,3-dione,
5-(3-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methyl piperazin-1-yl)azetidin-1-yl)-2-(2,6-dioxopiperidin-3-yl)isoindoline-1,3-dione,
5-(3-((S)-3-(tert-butyl)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)piperazin-1-yl)azetidin-1-yl)-2-(2,6-dioxopiperidin-3-yl)isoindoline-1,3-dione,
5-((S)-3-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methylpiperazin-1-yl)pyrrolidin-1-yl)-2-(2,6-dioxopiperidin-3-yl)isoindoline-1,3-dione,
5-((R)-3-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methylpiperazin-1-yl)pyrrolidin-1-yl)-2-(2,6-dioxopiperidin-3-yl)isoindoline-1,3-dione,
5-(4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methyl piperazin-1-yl)piperidin-1-yl)-2-(2,6-dioxopiperidin-3-yl)isoindoline-1,3-dione,
5-(4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-isopropylpiperazin-1-yl)-2-isopropylpiperidin-1-yl)-2-(2,6-dioxopiperidin-3-yl)isoindoline-1,3-dione,
5-((S)-4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methylpiperazin-1-yl)azepan-1-yl)-2-(2,6-dioxopiperidin-3-yl)isoindoline-1,3-dione,
5-((R)-4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methylpiperazin-1-yl)azepan-1-yl)-2-(2,6-dioxopiperidin-3-yl)isoindoline-1,3-dione,
5-(3-((2S)-1-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-methylpiperidin-4-yl)azetidin-1-yl)-2-(2,6-dioxopiperidin-3-yl)isoindoline-1,3-dione,
5-(3-((2R)-1-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-isopropylpiperidin-4-yl)azetidin-1-yl)-2-(2,6-dioxopiperidin-3-yl)isoindoline-1,3-dione,
5-((3R)-3-((2S)-1-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-methylpiperidin-4-yl)pyrrolidin-1-yl)-2-(2,6-dioxopiperidin-3-yl)isoindoline-1,3-dione,
5-((3S)-3-((2S)-1-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-methylpiperidin-4-yl)pyrrolidin-1-yl)-2-(2,6-dioxopiperidin-3-yl)isoindoline-1,3-dione,
5-((2'S)-1′-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2′-methyl-[4,4′-bipiperidin]-1-yl)-2-(2,6-dioxopiperidin-3-yl)isoindoline-1,3-dione,
5-((4S)-4-((2S)-1-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-methylpiperidin-4-yl)azepan-1-yl)-2-(2,6-dioxopiperidin-3-yl)isoindoline-1,3-dione,
5-((4R)-4-((2S)-1-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-methylpiperidin-4-yl)azepan-1-yl)-2-(2,6-dioxopiperidin-3-yl)isoindoline-1,3-dione,
5-(4-((2S)-1-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-2-methylpiperidin-4-yl)piperazin-1-yl)-2-(2,6-dioxopiperidin-3-yl)isoindoline-1,3-dione,
5-(3-(4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-4-methylpiperidin-1-yl)azetidin-1-yl)-2-(2,6-dioxopiperidin-3-yl)isoindoline-1,3-dione,
5-((S)-1-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methylpiperazin-1-yl)-6-azaspiro[2.5]octan-6-yl)-2-(2,6-dioxopiperidin-3-yl)isoindoline-1,3-dione,
5-((1S)-1-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-isopropylpiperazin-1-yl)-5-isopropyl-6-azaspiro[2.5]octan-6-yl)-2-(2,6-dioxopiperidin-3-yl)isoindoline-1,3-dione,
5-((R)-1-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methylpiperazin-1-yl)-6-azaspiro[2.5]octan-6-yl)-2-(2,6-dioxopiperidin-3-yl)isoindoline-1,3-dione,
5-(2-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methyl piperazin-1-yl)-7-azaspiro[3.5]nonan-7-yl)-2-(2,6-dioxopiperidin-3-yl)isoindoline-1,3-dione,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-4-(1-(2-((S)-2,6-dioxopiperidin-3-yl)-1,3-dioxoisoindolin-5-yl)piperidin-4-yl)-2-methylpiperazin-1-yl)phenyl)propionamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-4-(1-(2-((S)-2,6-dioxopiperidin-3-yl)-1,3-dioxoisoindolin-5-yl)piperidin-4-yl)-2-methylpiperazin-1-yl)phenyl)cyclopropanecarboxamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-4-(1-(2-((S)-2,6-dioxopiperidin-3-yl)-1,3-dioxoisoindolin-5-yl)piperidin-4-yl)-2-methylpiperazin-1-yl)phenyl)acetamide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-4-(1-(2-((S)-2,6-dioxopiperidin-3-yl)-1,3-dioxoisoindolin-5-yl)piperidin-4-yl)-2-methylpiperazin-1-yl)phenyl)isobutyramide,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-((S)-4-(1-(2-((S)-2,6-dioxopiperidin-3-yl)-1,3-dioxoisoindolin-5-yl)piperidin-4-yl)-2-methylpiperazin-1-yl)phenyl)tetrahydro-2H-pyran-4-carboxamide,
5-(4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(methylamino)phenyl)-3-methylpiperazin-1-yl)piperidin-1-yl)-2-((S)-2,6-dioxopiperidin-3-yl)isoindoline-1,3-dione,
5-(4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-methoxyphenyl)-3-methylpiperazin-1-yl)piperidin-1-yl)-2-((S)-2,6-dioxopiperidin-3-yl)isoindoline-1,3-dione,
5-(4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-methylphenyl)-3-methylpiperazin-1-yl)piperidin-1-yl)-2-((S)-2,6-dioxopiperidin-3-yl)isoindoline-1,3-dione,
5-(4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-(trifluoromethyl)phenyl)-3-methylpiperazin-1-yl)piperidin-1-yl)-2-((S)-2,6-dioxopiperidin-3-yl)isoindoline-1,3-dione,
5-(4-((S)-4-(4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)-2-fluorophenyl)-3-methylpiperazin-1-yl)piperidin-1-yl)-2-((S)-2,6-dioxopiperidin-3-yl)isoindoline-1,3-dione,
5-(4-((S)-4-(2-chloro-4-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-methyl-3-oxo-3,4-dihydropyrazin-2-yl)amino)phenyl)-3-methylpiperazin-1-yl)piperidin-1-yl)-2-((S)-2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
5-(2-((S)-3-(tert-butyl)-4-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)piperazin-1-yl)-6-methyl-7-azaspiro[3.5]nonan-7-yl)-2-(2,6-dioxopiperidin-3-yl)isoindoline-1,3-dione,
5-((S)-2-((S)-4-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-3-methylpiperazin-1-yl)-6-methyl-7-azaspiro[3.5]nonan-7-yl)-2-(2,6-dioxopiperidin-3-yl)isoindoline-1,3-dione,
5-((S)-2-((S)-4-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-3-methylpiperazin-1-yl)-8-azaspiro[4.5]decan-8-yl)-2-(2,6-dioxopiperidin-3-yl)isoindoline-1,3-dione,
5-((R)-2-((S)-4-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-3-methylpiperazin-1-yl)-8-azaspiro[4.5]decan-8-yl)-2-(2,6-dioxopiperidin-3-yl)isoindoline-1,3-dione,
5-(9-((S)-4-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-3-methylpiperazin-1-yl)-3-azaspiro[5.5]undecan-3-yl)-2-(2,6-dioxopiperidin-3-yl)isoindoline-1,3-dione,
5-(4-((1R,3R,5S)-6-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-5-methyl-6-azaspiro[2.5]octan-1-yl)piperazin-1-yl)-2-(2,6-dioxopiperidin-3-yl)isoindoline-1,3-dione,
5-((S)-4-((1R,3R,5R)-5-(tert-butyl)-6-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-6-azaspiro[2.5]octan-1-yl)-2-methylpiperazin-1-yl)-2-(2,6-dioxopiperidin-3-yl)isoindoline-1,3-dione,
5-(4-((1R,3S,5S)-6-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-5-methyl-6-azaspiro[2.5]octan-1-yl)piperazin-1-yl)-2-(2,6-dioxopiperidin-3-yl)isoindoline-1,3-dione,
5-(4-((S)-7-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-6-methyl-7-azaspiro[3.5]nonan-2-yl)piperazin-1-yl)-2-(2,6-dioxopiperidin-3-yl)isoindoline-1,3-dione,
5-((2S,3R)-4-((R)-6-(tert-butyl)-7-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-7-azaspiro[3.5]nonan-2-yl)-2,3-dimethylpiperazin-1-yl)-2-(2,6-dioxopiperidin-3-yl)isoindoline-1,3-dione,
2-(2,6-dioxopiperidin-3-yl)-5-(4-((7S)-8-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-7-methyl-8-azaspiro[4.5]decan-2-yl)piperazin-1-yl)isoindoline-1,3-dione,
2-(2,6-dioxopiperidin-3-yl)-5-(4-((S)-3-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-2-methyl-3-azaspiro[5.5]undecan-9-yl)piperazin-1-yl)isoindoline-1,3-dione,
5-((S)-4-((R)-2-(tert-butyl)-3-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-azaspiro[5.5]undecan-9-yl)-2-methylpiperazin-1-yl)-2-(2,6-dioxopiperidin-3-yl)isoindoline-1,3-dione,
3-(5-((R)-1-((S)-7-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-6-methyl-7-azaspiro[3.5]nonan-2-yl)pyrrolidin-2-yl)-1-oxoisoindolin-2-yl)piperidine-2,6-dione,
2-(2,6-dioxopiperidin-3-yl)-5-(5-((S)-4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)hexahydrocyclopenta[c]pyrrol-2(1H)-yl)isoindoline-1,3-dione,
2-(2,6-dioxopiperidin-3-yl)-5-(5-((2S)-1-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-2-methylpiperidin-4-yl)hexahydropyrrolo[3,4-c]pyrrol-2(1H)-yl)isoindoline-1,3-dione,
2-(2,6-dioxopiperidin-3-yl)-5-((S)-3-((4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-4-methylpiperidin-1-yl)methyl)pyrrolidin-1-yl)isoindoline-1,3-dione,
2-(2,6-dioxopiperidin-3-yl)-5-(3-(((S)-4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)methyl)azetidin-1-yl)isoindoline-1,3-dione,
2-(2,6-dioxopiperidin-3-yl)-5-(4-((S)-4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)isoindoline-1,3-dione,
5-(4-((S)-4-(6-((1,3′-dimethyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
5-((R)-3-(((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)methyl)pyrrolidin-1-yl)-2-(2,6-dioxopiperidin-3-yl)isoindoline-1,3-dione,
5-(4-(((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)methyl)piperidin-1-yl)-2-(2,6-dioxopiperidin-3-yl)isoindoline-1,3-dione,
5-(4-(((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-isopropylpiperazin-1-yl)methyl)piperidin-1-yl)-2-(2,6-dioxopiperidin-3-yl)isoindoline-1,3-dione,
5-(3-(1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)cyclobutyl)azetidin-1-yl)-2-(2,6-dioxopiperidin-3-yl)isoindoline-1,3-dione,
5-((S)-3-(1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)cyclobutyl)pyrrolidin-1-yl)-2-(2,6-dioxopiperidin-3-yl)isoindoline-1,3-dione,
5-(4-(1-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)cyclobutyl)piperidin-1-yl)-2-(2,6-dioxopiperidin-3-yl)isoindoline-1,3-dione,
5-(((2'S)-1′-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-2′-methyl-[1,4′-bipiperidin]-4-yl)ethynyl)-2-(2,6-dioxopiperidin-3-yl)isoindoline-1,3-dione,
5-((4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)cyclohexyl)ethynyl)-2-(2,6-dioxopiperidin-3-yl)isoindoline-1,3-dione,
5-((4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-isopropylpiperazin-1-yl)cyclohexyl)ethynyl)-2-(2,6-dioxopiperidin-3-yl)isoindoline-1,3-dione,
5-(((1S,3R)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)cyclopentyl)ethynyl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)pyrimidin-4-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-6-(trifluoromethyl)-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
2′-(7,7-dimethyl-1-oxo-1,3,4,6,7,8-hexahydro-2H-cyclopenta[4,5]pyrrolo[1,2-a]pyrazin-2-yl)-5-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)-3′-(hydroxymethyl)-[3,4′-bipyridine]-6-carboxamide,
2′-(7,7-dimethyl-1-oxo-1,3,4,6,7,8-hexahydro-2H-cyclopenta[4,5]pyrrolo[1,2-a]pyrazin-2-yl)-4-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)-3′-(hydroxymethyl)-[2,4′-bipyridine]-5-carboxamide,
5-(4-((S)-4-(6-((2-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-5-oxo-5,6,7,8-tetrahydro-1,6-naphthyridin-4-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((6-amino-2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-6-(methylamino)-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((1-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-oxo-4,5,6,7-tetrahydro-1H-pyrrolo[3,2-c]pyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-methyl-[4,4′-bipyridin]-2-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(4-((4-(2-(7,7-dimethyl-1-oxo-1,3,4,6,7,8-hexahydro-2H-cyclopenta[4,5]pyrrolo[1,2-a]pyrazin-2-yl)-3-methylpyridin-4-yl)pyrimidin-2-yl)amino)phenyl)-3-methylpiperazin-1-yl)piperidin-1-yl)-2-(2,6-dioxopiperidin-3-yl)isoindoline-1,3-dione,
2-(2,6-dioxopiperidin-3-yl)-5-(4-((S)-4-(6-((6-(3-(hydroxymethyl)-2-(1-oxo-3,4,5,6,7,8-hexahydrobenzo[4,5]thieno[2,3-c]pyridin-2(1H)-yl)pyridin-4-yl)pyrimidin-4-yl)amino)pyridin-3-yl)-3-methylpiperazin-1-yl)piperidin-1-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-methyl-6-(trifluoromethyl)-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
5-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)-3′-(hydroxymethyl)-2′-(1-oxo-3,4,5,6,7,8-hexahydrobenzo[4,5]thieno[2,3-c]pyridin-2(1H)-yl)-[3,4′-bipyridine]-6-carboxamide,
4-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)-3′-methyl-2′-(1-oxo-3,4,5,6,7,8-hexahydrobenzo[4,5]thieno[2,3-c]pyridin-2(1H)-yl)-[2,4′-bipyridine]-5-carboxamide,
5-(4-((S)-4-(6-((2-(2-(7,7-dimethyl-1-oxo-1,3,4,6,7,8-hexahydro-2H-cyclopenta[4,5]pyrrolo[1,2-a]pyrazin-2-yl)-3-methylpyridin-4-yl)-5-oxo-5,6,7,8-tetrahydro-1,6-naphthyridin-4-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((6-amino-3′-(hydroxymethyl)-2′-(1-oxo-3,4,5,6,7,8-hexahydrobenzo[4,5]thieno[2,3-c]pyridin-2(1H)-yl)-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
2-(2,6-dioxopiperidin-3-yl)-5-(4-((S)-3-methyl-4-(6-((3′-methyl-6-(methylamino)-2′-(1-oxo-3,4,5,6,7,8-hexahydrobenzo[4,5]thieno[2,3-c]pyridin-2(1H)-yl)-[3,4′-bipyridin]-5-yl)amino)pyridin-3-yl)piperazin-1-yl)piperidin-1-yl)isoindoline-1,3-dione,
5-(4-((S)-4-(6-((1-(2-(7,7-dimethyl-1-oxo-1,3,4,6,7,8-hexahydro-2H-cyclopenta[4,5]pyrrolo[1,2-a]pyrazin-2-yl)-3-methylpyridin-4-yl)-4-oxo-4,5,6,7-tetrahydro-1H-pyrrolo[3,2-c]pyridin-3-yl)amino)pyridin-3-yl)-3-methylpiperazin-1-yl)piperidin-1-yl)-2-(2,6-dioxopiperidin-3-yl)isoindoline-1,3-dione,
(5aR,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
(5aR,7R)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
(5aS,7R)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
(5aS,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
(S)-3-((5aR,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)piperidine-2,6-dione,
((S)-3-((5aR,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1,3-dioxo-1,3,5,5a,6,7,8,9-octahydro-2H-pyrido[1′,2′:4,5][1,4]oxazino[2,3-e]isoindol-2-yl)-2,6-dioxopiperidin-1-yl)methyl dihydrogen phosphate,
((S)-3-((5aR,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)-2,6-dioxopiperidin-1-yl)methyl (2-(phosphonooxy)ethyl)(3-((phosphonooxy)methyl)pyridin-2-yl)carbamate,
7-((S)-4-(6-((2′-(7,7-dimethyl-1-oxo-1,3,4,6,7,8-hexahydro-2H-cyclopenta[4,5]pyrrolo[1,2-a]pyrazin-2-yl)-1,3′-dimethyl-6-oxo-1,6-dihydro-[3,4′-bipyridin]-5-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
2-(2,6-dioxopiperidin-3-yl)-7-((S)-4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-5,5a,6,7,8,9-hexahydro-1H-pyrido[1′,2′:4,5][1,4]oxazino[2,3-e]isoindole-1,3(2H)-dione,
7-((S)-4-(6-((1,3′-dimethyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-[4,4′-bipyridin]-2-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
5-((5-((2S)-4-(2-(2,6-dioxopiperidin-3-yl)-1,3-dioxo-2,3,5,5a,6,7,8,9-octahydro-1H-pyrido[1′,2′:4,5][1,4]oxazino[2,3-e]isoindol-7-yl)-2-methylpiperazin-1-yl)pyridin-2-yl)amino)-3′-(hydroxymethyl)-2′-(1-oxo-3,4,5,6,7,8-hexahydrobenzo[4,5]thieno[2,3-c]pyridin-2(1H)-yl)-[3,4′-bipyridine]-6-carboxamide,
4-((5-((2S)-4-(2-(2,6-dioxopiperidin-3-yl)-1,3-dioxo-2,3,5,5a,6,7,8,9-octahydro-1H-pyrido[1′,2′:4,5][1,4]oxazino[2,3-e]isoindol-7-yl)-2-methylpiperazin-1-yl)pyridin-2-yl)amino)-3′-methyl-2′-(1-oxo-3,4,5,6,7,8-hexahydrobenzo[4,5]thieno[2,3-c]pyridin-2(1H)-yl)-[2,4′-bipyridine]-5-carboxamide,
7-((S)-4-(6-((2-(2-(7,7-dimethyl-1-oxo-1,3,4,6,7,8-hexahydro-2H-cyclopenta[4,5]pyrrolo[1,2-a]pyrazin-2-yl)-3-methylpyridin-4-yl)-5-oxo-5,6,7,8-tetrahydro-1,6-naphthyridin-4-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
7-((S)-4-(6-((6-amino-3′-(hydroxymethyl)-2′-(1-oxo-3,4,5,6,7,8-hexahydrobenzo[4,5]thieno[2,3-c]pyridin-2(1H)-yl)-[3,4′-bipyridin]-5-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
2-(2,6-dioxopiperidin-3-yl)-7-((S)-3-methyl-4-(6-((3′-methyl-6-(methylamino)-2′-(1-oxo-3,4,5,6,7,8-hexahydrobenzo[4,5]thieno[2,3-c]pyridin-2(1H)-yl)-[3,4′-bipyridin]-5-yl)amino)pyridin-3-yl)piperazin-1-yl)-5,5a,6,7,8,9-hexahydro-1H-pyrido[1′,2′:4,5][1,4]oxazino[2,3-e]isoindole-1,3(2H)-dione,
7-((S)-4-(6-((1-(2-(7,7-dimethyl-1-oxo-1,3,4,6,7,8-hexahydro-2H-cyclopenta[4,5]pyrrolo[1,2-a]pyrazin-2-yl)-3-methylpyridin-4-yl)-4-oxo-4,5,6,7-tetrahydro-1H-pyrrolo[3,2-c]pyridin-3-yl)amino)pyridin-3-yl)-3-methylpiperazin-1-yl)-2-(2,6-dioxopiperidin-3-yl)-5,5a,6,7,8,9-hexahydro-1H-pyrido[1′,2′:4,5][1,4]oxazino[2,3-e]isoindole-1,3(2H)-dione,
(3aR,5S)-5-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-12-(2,6-dioxopiperidin-3-yl)-3,3a,4,5,6,7-hexahydro-2H,11H-pyrido[1′,2′:4,5][1,4]oxazepino[2,3-e]isoindole-11,13(12H)-dione,
(3aS,5S)-5-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-12-(2,6-dioxopiperidin-3-yl)-3,3a,4,5,6,7-hexahydro-2H,11H-pyrido[1′,2′:4,5][1,4]oxazepino[2,3-e]isoindole-11,13(12H)-dione,
(3aR,5R)-5-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-12-(2,6-dioxopiperidin-3-yl)-3,3a,4,5,6,7-hexahydro-2H,11H-pyrido[1′,2′:4,5][1,4]oxazepino[2,3-e]isoindole-11,13(12H)-dione,
(3aS,5R)-5-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-12-(2,6-dioxopiperidin-3-yl)-3,3a,4,5,6,7-hexahydro-2H,11H-pyrido[1′,2′:4,5][1,4]oxazepino[2,3-e]isoindole-11,13(12H)-dione,
3-((5aR,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)piperidine-2,6-dione,
3-((5aR,7R)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)piperidine-2,6-dione,
3-((5aS,7R)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)piperidine-2,6-dione,
3-((5aS,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)piperidine-2,6-dione,
(S)-3-((5aR,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)piperidine-2,6-dione,
((S)-3-((5aR,7S)-7-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)-2,6-dioxopiperidin-1-yl)methyl dihydrogen phosphate,
3-(7-((S)-4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-1-oxo-1,3,5,5a,6,7,8,9-octahydro-2H-pyrido[1′,2′:4,5][1,4]oxazino[2,3-e]isoindol-2-yl)piperidine-2,6-dione,
3-((3aR,5S)-5-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-11-oxo-3,3a,4,5,6,7,11,13-octahydro-2H,12H-pyrido[1′,2′:4,5][1,4]oxazepino[2,3-e]isoindol-12-yl)piperidine-2,6-dione,
3-((3aS,5S)-5-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-11-oxo-3,3a,4,5,6,7,11,13-octahydro-2H,12H-pyrido[1′,2′:4,5][1,4]oxazepino[2,3-e]isoindol-12-yl)piperidine-2,6-dione,
3-((3aR,5R)-5-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-11-oxo-3,3a,4,5,6,7,11,13-octahydro-2H,12H-pyrido[1′,2′:4,5][1,4]oxazepino[2,3-e]isoindol-12-yl)piperidine-2,6-dione,
3-((3aS,5R)-5-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-11-oxo-3,3a,4,5,6,7,11,13-octahydro-2H,12H-pyrido[1′,2′:4,5][1,4]oxazepino[2,3-e]isoindol-12-yl)piperidine-2,6-dione,
(3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione,
(3S,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione,
(3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione,
(3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione,
(S)-3-((3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-oxo-1,2,3,4,4a,5,8,10-octahydro-9H-pyrido[1′,2′:4,5][1,4]oxazino[2,3-f]isoindol-9-yl)piperidine-2,6-dione,
3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-methyl-[4,4′-bipyridin]-2-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione,
3-((S)-4-(4-((4-(2-(7,7-dimethyl-1-oxo-1,3,4,6,7,8-hexahydro-2H-cyclopenta[4,5]pyrrolo[1,2-a]pyrazin-2-yl)-3-methylpyridin-4-yl)pyrimidin-2-yl)amino)phenyl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione,
9-(2,6-dioxopiperidin-3-yl)-3-((S)-4-(6-((6-(3-(hydroxymethyl)-2-(1-oxo-3,4,5,6,7,8-hexahydrobenzo[4,5]thieno[2,3-c]pyridin-2(1H)-yl)pyridin-4-yl)pyrimidin-4-yl)amino)pyridin-3-yl)-3-methylpiperazin-1-yl)-1,2,3,4,4a,5-hexahydro-8H-pyrido[1′,2′:4,5][1,4]oxazino[2,3-f]isoindole-8,10(9H)-dione,
(3S,4aR)-3-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-10-(2,6-dioxopiperidin-3-yl)-2,3,4,4a,5,6-hexahydro-1H,9H-pyrido[1′,2′:4,5][1,4]oxazepino[2,3-f]isoindole-9,11(10H)-dione,
(3R,4aR)-3-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-10-(2,6-dioxopiperidin-3-yl)-2,3,4,4a,5,6-hexahydro-1H,9H-pyrido[1′,2′:4,5][1,4]oxazepino[2,3-f]isoindole-9,11(10H)-dione,
(3S,4aS)-3-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-10-(2,6-dioxopiperidin-3-yl)-2,3,4,4a,5,6-hexahydro-1H,9H-pyrido[1′,2′:4,5][1,4]oxazepino[2,3-f]isoindole-9,11(10H)-dione,
(3R,4aS)-3-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-10-(2,6-dioxopiperidin-3-yl)-2,3,4,4a,5,6-hexahydro-1H,9H-pyrido[1′,2′:4,5][1,4]oxazepino[2,3-f]isoindole-9,11(10H)-dione,
3-((3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-oxo-1,2,3,4,4a,5,8,10-octahydro-9H-pyrido[1′,2′:4,5][1,4]oxazino[2,3-f]isoindol-9-yl)piperidine-2,6-dione,
3-((3S,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-oxo-1,2,3,4,4a,5,8,10-octahydro-9H-pyrido[1′,2′:4,5][1,4]oxazino[2,3-f]isoindol-9-yl)piperidine-2,6-dione,
3-((3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-oxo-1,2,3,4,4a,5,8,10-octahydro-9H-pyrido[1′,2′:4,5][1,4]oxazino[2,3-f]isoindol-9-yl)piperidine-2,6-dione,
3-((3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-oxo-1,2,3,4,4a,5,8,10-octahydro-9H-pyrido[1′,2′:4,5][1,4]oxazino[2,3-f]isoindol-9-yl)piperidine-2,6-dione,
(S)-3-((3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-oxo-1,2,3,4,4a,5,8,10-octahydro-9H-pyrido[1′,2′:4,5][1,4]oxazino[2,3-f]isoindol-9-yl)piperidine-2,6-dione,
3-((3S,4aR)-3-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-oxo-2,3,4,4a,5,6,9,11-octahydro-1H,10H-pyrido[1′,2′:4,5][1,4]oxazepino[2,3-f]isoindol-10-yl)piperidine-2,6-dione,
3-((3R,4aR)-3-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-oxo-2,3,4,4a,5,6,9,11-octahydro-1H,10H-pyrido[1′,2′:4,5][1,4]oxazepino[2,3-f]isoindol-10-yl)piperidine-2,6-dione,
3-((3S,4aS)-3-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-oxo-2,3,4,4a,5,6,9,11-octahydro-1H,10H-pyrido[1′,2′:4,5][1,4]oxazepino[2,3-f]isoindol-10-yl)piperidine-2,6-dione,
3-((3R,4aS)-3-((R)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-oxo-2,3,4,4a,5,6,9,11-octahydro-1H,10H-pyrido[1′,2′:4,5][1,4]oxazepino[2,3-f]isoindol-10-yl)piperidine-2,6-dione,
(3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione,
(3-((3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-8,10-dioxo-1,2,3,4,4a,5,8,10-octahydro-9H-pyrido[1′,2′:4,5][1,4]oxazino[2,3-f]isoindol-9-yl)-2,6-dioxopiperidin-1-yl)methyl dihydrogen phosphate,
1-((S)-3-((3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-8,10-dioxo-1,2,3,4,4a,5,8,10-octahydro-9H-pyrido[1′,2′:4,5][1,4]oxazino[2,3-f]isoindol-9-yl)-2,6-dioxopiperidin-1-yl)ethyl dihydrogen phosphate,
(3S,4aR)-9-(2,6-dioxopiperidin-3-yl)-3-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-1,2,3,4,4a,5-hexahydro-8H-pyrido[1′,2′:4,5][1,4]oxazino[2,3-f]isoindole-8,10(9H)-dione,
(3-((3S,4aR)-3-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-8,10-dioxo-1,2,3,4,4a,5,8,10-octahydro-9H-pyrido[1′,2′:4,5][1,4]oxazino[2,3-f]isoindol-9-yl)-2,6-dioxopiperidin-1-yl)methyl dihydrogen phosphate,
1-((S)-3-((3S,4aR)-3-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-8,10-dioxo-1,2,3,4,4a,5,8,10-octahydro-9H-pyrido[1′,2′:4,5][1,4]oxazino[2,3-f]isoindol-9-yl)-2,6-dioxopiperidin-1-yl)ethyl dihydrogen phosphate,
(3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione,
(3-((3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-8,10-dioxo-1,2,3,4,4a,5,8,10-octahydro-9H-pyrido[1′,2′:4,5][1,4]oxazino[2,3-f]isoindol-9-yl)-2,6-dioxopiperidin-1-yl)methyl dihydrogen phosphate,
1-((S)-3-((3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-8,10-dioxo-1,2,3,4,4a,5,8,10-octahydro-9H-pyrido[1′,2′:4,5][1,4]oxazino[2,3-f]isoindol-9-yl)-2,6-dioxopiperidin-1-yl)ethyl dihydrogen phosphate,
(3R,4aR)-9-(2,6-dioxopiperidin-3-yl)-3-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-1,2,3,4,4a,5-hexahydro-8H-pyrido[1′,2′:4,5][1,4]oxazino[2,3-f]isoindole-8,10(9H)-dione,
(3-((3R,4aR)-3-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-8,10-dioxo-1,2,3,4,4a,5,8,10-octahydro-9H-pyrido[1′,2′:4,5][1,4]oxazino[2,3-f]isoindol-9-yl)-2,6-dioxopiperidin-1-yl)methyl dihydrogen phosphate,
1-((S)-3-((3R,4aR)-3-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-8,10-dioxo-1,2,3,4,4a,5,8,10-octahydro-9H-pyrido[1′,2′:4,5][1,4]oxazino[2,3-f]isoindol-9-yl)-2,6-dioxopiperidin-1-yl)ethyl dihydrogen phosphate,
(10R,11aS)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-8,9,10,11,11a,12-hexahydropyrido[2′,1′:3,4][1,4]oxazepino[7,6-e]isoindole-1,3(2H,6H)-dione,
(10R,11aR)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-8,9,10,11,11a,12-hexahydropyrido[2′,1′:3,4][1,4]oxazepino[7,6-e]isoindole-1,3(2H,6H)-dione,
(10S,11aR)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-8,9,10,11,11a,12-hexahydropyrido[2′,1′:3,4][1,4]oxazepino[7,6-e]isoindole-1,3(2H,6H)-dione,
(10S,11aS)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-8,9,10,11,11a,12-hexahydropyrido[2′,1′:3,4][1,4]oxazepino[7,6-e]isoindole-1,3(2H,6H)-dione,
3-((10R,11aS)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,8,9,10,11,11a,12-octahydropyrido[2′,1′:3,4][1,4]oxazepino[7,6-e]isoindol-2(1H)-yl)piperidine-2,6-dione,
3-((10R,11aR)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,8,9,10,11,11a,12-octahydropyrido[2′,1′:3,4][1,4]oxazepino[7,6-e]isoindol-2(1H)-yl)piperidine-2,6-dione,
3-((10S,11aR)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,8,9,10,11,11a,12-octahydropyrido[2′,1′:3,4][1,4]oxazepino[7,6-e]isoindol-2(1H)-yl)piperidine-2,6-dione,
3-((10S,11aS)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,8,9,10,11,11a,12-octahydropyrido[2′,1′:3,4][1,4]oxazepino[7,6-e]isoindol-2(1H)-yl)piperidine-2,6-dione,
(6aS,8S)-8-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-6,6a,7,8,9,10-hexahydropyrido[2′,1′:3,4][1,4]oxazepino[6,7-f]isoindole-1,3(2H,12H)-dione,
(6aS,8R)-8-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-6,6a,7,8,9,10-hexahydropyrido[2′,1′:3,4][1,4]oxazepino[6,7-f]isoindole-1,3(2H,12H)-dione,
(6aR,8S)-8-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-6,6a,7,8,9,10-hexahydropyrido[2′,1′:3,4][1,4]oxazepino[6,7-f]isoindole-1,3(2H,12H)-dione,
(6aR,8R)-8-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-6,6a,7,8,9,10-hexahydropyrido[2′,1′:3,4][1,4]oxazepino[6,7-f]isoindole-1,3(2H,12H)-dione,
(10R,11aR)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-8,9,10,11,11a,12-hexahydropyrido[1,2-b]pyrrolo[3,4-f]isoquinoline-1,3(2H,6H)-dione,
(10R,11aS)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-8,9,10,11,11a,12-hexahydropyrido[1,2-b]pyrrolo[3,4-f]isoquinoline-1,3(2H,6H)-dione,
(10S,11aR)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-8,9,10,11,11a,12-hexahydropyrido[1,2-b]pyrrolo[3,4-f]isoquinoline-1,3(2H,6H)-dione,
(10S,11aS)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-8,9,10,11,11a,12-hexahydropyrido[1,2-b]pyrrolo[3,4-f]isoquinoline-1,3(2H,6H)-dione,
(9R,10aR)-9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-7,8,9,10,10a,11-hexahydropyrido[1,2-b]pyrrolo[3,4-g]isoquinoline-1,3(2H,5H)-dione,
(9S,10aR)-9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-7,8,9,10,10a,11-hexahydropyrido[1,2-b]pyrrolo[3,4-g]isoquinoline-1,3(2H,5H)-dione,
(9R,10aS)-9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-7,8,9,10,10a,11-hexahydropyrido[1,2-b]pyrrolo[3,4-g]isoquinoline-1,3(2H,5H)-dione,
(9S,10aS)-9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-(2,6-dioxopiperidin-3-yl)-7,8,9,10,10a,11-hexahydropyrido[1,2-b]pyrrolo[3,4-g]isoquinoline-1,3(2H,5H)-dione,
3-((6aS,8S)-8-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,6a,7,8,9,10,12-octahydropyrido[2′,1′:3,4][1,4]oxazepino[6,7-f]isoindol-2(1H)-yl)piperidine-2,6-dione,
3-((6aS,8R)-8-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,6a,7,8,9,10,12-octahydropyrido[2′,1′:3,4][1,4]oxazepino[6,7-f]isoindol-2(1H)-yl)piperidine-2,6-dione,
3-((6aR,8S)-8-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,6a,7,8,9,10,12-octahydropyrido[2′,1′:3,4][1,4]oxazepino[6,7-f]isoindol-2(1H)-yl)piperidine-2,6-dione,
3-((6aR,8R)-8-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,6a,7,8,9,10,12-octahydropyrido[2′,1′:3,4][1,4]oxazepino[6,7-f]isoindol-2(1H)-yl)piperidine-2,6-dione,
3-((10R,11aR)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,8,9,10,11,11a,12-octahydropyrido[1,2-b]pyrrolo[3,4-f]isoquinolin-2(1H)-yl)piperidine-2,6-dione,
3-((10R,11aS)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,8,9,10,11,11a,12-octahydropyrido[1,2-b]pyrrolo[3,4-f]isoquinolin-2(1H)-yl)piperidine-2,6-dione,
3-((10S,11aR)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,8,9,10,11,11a,12-octahydropyrido[1,2-b]pyrrolo[3,4-f]isoquinolin-2(1H)-yl)piperidine-2,6-dione,
3-((10S,11aS)-10-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-3,6,8,9,10,11,11a,12-octahydropyrido[1,2-b]pyrrolo[3,4-f]isoquinolin-2(1H)-yl)piperidine-2,6-dione,
3-((9R,10aR)-9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-1,5,7,8,9,10,10a,11-octahydropyrido[1,2-b]pyrrolo[3,4-g]isoquinolin-2(3H)-yl)piperidine-2,6-dione,
3-((9S,10aR)-9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,5,7,8,9,10,10a,11-octahydropyrido[1,2-b]pyrrolo[3,4-g]isoquinolin-2(3H)-yl)piperidine-2,6-dione,
3-((9R,10aS)-9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-3-oxo-1,5,7,8,9,10,10a,11-octahydropyrido[1,2-b]pyrrolo[3,4-g]isoquinolin-2(3H)-yl)piperidine-2,6-dione,
3-((9S,10aS)-9-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-1-oxo-1,5,7,8,9,10,10a,11-octahydropyrido[1,2-b]pyrrolo[3,4-g]isoquinolin-2(3H)-yl)piperidine-2,6-dione,
(3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-(2,6-dioxopiperidin-3-yl)-1,2,3,4,4a,5-hexahydro-7H-pyrido[1,2-d]pyrrolo[3′,4′:4,5]thieno[3,2-b][1,4]oxazine-7,9(8H)-dione,
(3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-(2,6-dioxopiperidin-3-yl)-1,2,3,4,4a,5-hexahydro-7H-pyrido[1,2-d]pyrrolo[3′,4′:4,5]thieno[3,2-b][1,4]oxazine-7,9(8H)-dione,
(3S,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-(2,6-dioxopiperidin-3-yl)-1,2,3,4,4a,5-hexahydro-7H-pyrido[1,2-d]pyrrolo[3′,4′:4,5]thieno[3,2-b][1,4]oxazine-7,9(8H)-dione,
(3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-8-(2,6-dioxopiperidin-3-yl)-1,2,3,4,4a,5-hexahydro-7H-pyrido[1,2-d]pyrrolo[3′,4′:4,5]thieno[3,2-b][1,4]oxazine-7,9(8H)-dione,
3-((3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-7-oxo-1,2,3,4,4a,5,7,9-octahydro-8H-pyrido[1,2-d]pyrrolo[3′,4′:4,5]thieno[3,2-b][1,4]oxazin-8-yl)piperidine-2,6-dione,
3-((3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-oxo-1,2,3,4,4a,5,7,9-octahydro-8H-pyrido[1,2-d]pyrrolo[3′,4′:4,5]thieno[3,2-b][1,4]oxazin-8-yl)piperidine-2,6-dione,
3-((3S,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-7-oxo-1,2,3,4,4a,5,7,9-octahydro-8H-pyrido[1,2-d]pyrrolo[3′,4′:4,5]thieno[3,2-b][1,4]oxazin-8-yl)piperidine-2,6-dione,
3-((3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-oxo-1,2,3,4,4a,5,7,9-octahydro-8H-pyrido[1,2-d]pyrrolo[3′,4′:4,5]thieno[3,2-b][1,4]oxazin-8-yl)piperidine-2,6-dione
5-(4-((3S)-4-(6-((5-(3-(7,7-dimethyl-1-oxo-1,3,4,6,7,8-hexahydro-2H-cyclopenta[4,5]pyrrolo[1,2-a]pyrazin-2-yl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((3S)-4-(6-((5-(3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-((R)-3-(7,7-dimethyl-1-oxo-1,3,4,6,7,8-hexahydro-2H-cyclopenta[4,5]pyrrolo[1,2-a]pyrazin-2-yl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((S)-3-(7,7-dimethyl-1-oxo-1,3,4,6,7,8-hexahydro-2H-cyclopenta[4,5]pyrrolo[1,2-a]pyrazin-2-yl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2R,3R)-3-(7,7-dimethyl-1-oxo-1,3,4,6,7,8-hexahydro-2H-cyclopenta[4,5]pyrrolo[1,2-a]pyrazin-2-yl)-2-methylpiperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2S,3R)-3-(7,7-dimethyl-1-oxo-1,3,4,6,7,8-hexahydro-2H-cyclopenta[4,5]pyrrolo[1,2-a]pyrazin-2-yl)-2-methylpiperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2R,3S)-3-(7,7-dimethyl-1-oxo-1,3,4,6,7,8-hexahydro-2H-cyclopenta[4,5]pyrrolo[1,2-a]pyrazin-2-yl)-2-methylpiperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2S,3S)-3-(7,7-dimethyl-1-oxo-1,3,4,6,7,8-hexahydro-2H-cyclopenta[4,5]pyrrolo[1,2-a]pyrazin-2-yl)-2-methylpiperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2S,3R)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2R,3R)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2S,3S)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2R,3S)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2R,3S)-3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2S,3S)-3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
-((2R,3R)-3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-((2S,3R)-3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-3-((5-((2S)-4-(1-(2-(2,6-dioxopiperidin-3-yl)-1,3-dioxoisoindolin-5-yl)piperidin-4-yl)-2-methylpiperazin-1-yl)pyridin-2-yl)amino)picolinamide,
5-(4-((S)-4-(6-((5-((R)-3-(7,7-dimethyl-1-oxo-1,3,4,6,7,8-hexahydro-2H-cyclopenta[4,5]pyrrolo[1,2-a]pyrazin-2-yl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((S)-3-(7,7-dimethyl-1-oxo-1,3,4,6,7,8-hexahydro-2H-cyclopenta[4,5]pyrrolo[1,2-a]pyrazin-2-yl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3R)-3-(7,7-dimethyl-1-oxo-1,3,4,6,7,8-hexahydro-2H-cyclopenta[4,5]pyrrolo[1,2-a]pyrazin-2-yl)-2-methylpiperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3R)-3-(7,7-dimethyl-1-oxo-1,3,4,6,7,8-hexahydro-2H-cyclopenta[4,5]pyrrolo[1,2-a]pyrazin-2-yl)-2-methylpiperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3S)-3-(7,7-dimethyl-1-oxo-1,3,4,6,7,8-hexahydro-2H-cyclopenta[4,5]pyrrolo[1,2-a]pyrazin-2-yl)-2-methylpiperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3S)-3-(7,7-dimethyl-1-oxo-1,3,4,6,7,8-hexahydro-2H-cyclopenta[4,5]pyrrolo[1,2-a]pyrazin-2-yl)-2-methylpiperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3R)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3R)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3S)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3S)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3S)-3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3S)-3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3R)-3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3R)-3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3S)-3-(7,7-dimethyl-1-oxo-1,3,4,6,7,8-hexahydro-2H-cyclopenta[4,5]pyrrolo[1,2-a]pyrazin-2-yl)-2-((S)-1-hydroxyethyl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3S)-3-(7,7-dimethyl-1-oxo-1,3,4,6,7,8-hexahydro-2H-cyclopenta[4,5]pyrrolo[1,2-a]pyrazin-2-yl)-2-((R)-1-hydroxyethyl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3R)-3-(7,7-dimethyl-1-oxo-1,3,4,6,7,8-hexahydro-2H-cyclopenta[4,5]pyrrolo[1,2-a]pyrazin-2-yl)-2-((S)-1-hydroxyethyl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3R)-3-(7,7-dimethyl-1-oxo-1,3,4,6,7,8-hexahydro-2H-cyclopenta[4,5]pyrrolo[1,2-a]pyrazin-2-yl)-2-((R)-1-hydroxyethyl)piperidin-1-yl)-2-methoxypyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((R)-3-(7,7-dimethyl-1-oxo-1,3,4,6,7,8-hexahydro-2H-cyclopenta[4,5]pyrrolo[1,2-a]pyrazin-2-yl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((S)-3-(7,7-dimethyl-1-oxo-1,3,4,6,7,8-hexahydro-2H-cyclopenta[4,5]pyrrolo[1,2-a]pyrazin-2-yl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3R)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3R)-3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2R,3R)-3-(7,7-dimethyl-1-oxo-1,3,4,6,7,8-hexahydro-2H-cyclopenta[4,5]pyrrolo[1,2-a]pyrazin-2-yl)-2-((R)-1-hydroxyethyl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((5-((2S,3R)-3-(7,7-dimethyl-1-oxo-1,3,4,6,7,8-hexahydro-2H-cyclopenta[4,5]pyrrolo[1,2-a]pyrazin-2-yl)-2-((R)-1-hydroxyethyl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((3S)-4-(6-((5-(3-(7,7-dimethyl-1-oxo-1,3,4,6,7,8-hexahydro-2H-cyclopenta[4,5]pyrrolo[1,2-a]pyrazin-2-yl)-2-(1-hydroxyethyl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((3S)-4-(6-((5-(3-(7,7-dimethyl-1-oxo-1,3,4,6,7,8-hexahydro-2H-cyclopenta[4,5]pyrrolo[1,2-a]pyrazin-2-yl)-2-(2-hydroxypropan-2-yl)piperidin-1-yl)-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)piperidin-1-yl)-2-((S)-2,6-dioxopiperidin-3-yl)isoindoline-1,3-dione,
5-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione,
5-((2S,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione,
5-((2R,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione,
5-((2R,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione,
2-(2,6-dioxopiperidin-3-yl)-5-((2S,4S)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)isoindoline-1,3-dione,
2-(2,6-dioxopiperidin-3-yl)-5-((2S,4R)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)isoindoline-1,3-dione,
2-(2,6-dioxopiperidin-3-yl)-5-((2R,4S)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)isoindoline-1,3-dione,
2-(2,6-dioxopiperidin-3-yl)-5-((2R,4R)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)isoindoline-1,3-dione,
3-(5-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-1-oxoisoindolin-2-yl)piperidine-2,6-dione,
3-(5-((2S,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-1-oxoisoindolin-2-yl)piperidine-2,6-dione,
3-(5-((2R,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-1-oxoisoindolin-2-yl)piperidine-2,6-dione,
3-(5-((2R,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-1-oxoisoindolin-2-yl)piperidine-2,6-dione,
3-(5-((2S,4S)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)-1-oxoisoindolin-2-yl)piperidine-2,6-dione,
3-(5-((2S,4R)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)-1-oxoisoindolin-2-yl)piperidine-2,6-dione,
3-(5-((2R,4S)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)-1-oxoisoindolin-2-yl)piperidine-2,6-dione,
3-(5-((2R,4R)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)-1-oxoisoindolin-2-yl)piperidine-2,6-dione,
5-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-2-((S)-2,6-dioxopiperidin-3-yl)isoindoline-1,3-dione,
5-((2S,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-2-((S)-2,6-dioxopiperidin-3-yl)isoindoline-1,3-dione,
5-((2R,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-2-((S)-2,6-dioxopiperidin-3-yl)isoindoline-1,3-dione,
5-((2R,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-2-((S)-2,6-dioxopiperidin-3-yl)isoindoline-1,3-dione,
2-((S)-2,6-dioxopiperidin-3-yl)-5-((2S,4S)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)isoindoline-1,3-dione,
2-((S)-2,6-dioxopiperidin-3-yl)-5-((2S,4R)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)isoindoline-1,3-dione,
2-((S)-2,6-dioxopiperidin-3-yl)-5-((2R,4S)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)isoindoline-1,3-dione,
2-((S)-2,6-dioxopiperidin-3-yl)-5-((2R,4R)-4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)-2-methylpiperidin-1-yl)isoindoline-1,3-dione,
N-(5-((6-(2-(7,7-dimethyl-1-oxo-1,3,4,6,7,8-hexahydro-2H-cyclopenta[4,5]pyrrolo[1,2-a]pyrazin-2-yl)-3-(hydroxymethyl)pyridin-4-yl)-4-(methyl-d3)-3-oxo-3,4-dihydropyrazin-2-yl)amino)-2-((S)-4-(1-(2-((S)-2,6-dioxopiperidin-3-yl)-1,3-dioxoisoindolin-5-yl)piperidin-4-yl)-2-methylpiperazin-1-yl)phenyl)propionamide,
3-((3-(4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)phenyl)amino)piperidine-2,6-dione,
3-((4-(4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)phenyl)amino)piperidine-2,6-dione,
3-(3-(4-((S)-4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)phenoxy)piperidine-2,6-dione,
3-(4-(4-((S)-4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)phenoxy)piperidine-2,6-dione,
3-(4-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)phenyl)piperidine-2,6-dione,
1-(4-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)phenyl)dihydropyrimidine-2,4(1H,3H)-dione,
3-(3-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)phenyl)piperidine-2,6-dione,
1-(3-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)phenyl)dihydropyrimidine-2,4(1H,3H)-dione,
N-(2,6-dioxopiperidin-3-yl)-4-(4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)benzamide,
3-(6-(4-((S)-4-(6-((3′-(hydroxymethyl)-1-(methyl-d3)-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)-3-methyl-2-oxo-2,3-dihydro-1H-benzo[d]imidazol-1-yl)piperidine-2,6-dione,
1-(5-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-(methyl-d3)-6-oxo-1,6-dihydro-[3,4′-bipyridin]-5-yl)amino)pyridin-3-yl)-3-methylpiperazin-1-yl)-2-methylpiperidin-1-yl)-1-methyl-1H-indazol-3-yl)dihydropyrimidine-2,4(1H,3H)-dione,
3-(6-(4-((S)-4-(6-((3′-(hydroxymethyl)-1-methyl-6-oxo-2′-(1-oxo-3,4,5,6,7,8-hexahydrobenzo[4,5]thieno[2,3-c]pyridin-2(1H)-yl)-1,6-dihydro-[3,4′-bipyridin]-5-yl)amino)pyridin-3-yl)-3-methylpiperazin-1-yl)piperidin-1-yl)-2-methyl-3-oxo-2,3-dihydro-1H-indazol-1-yl)piperidine-2,6-dione,
3-(6-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-1-methyl-3-oxo-1,3-dihydro-2H-indazol-2-yl)piperidine-2,6-dione,
3-(4-((2S,4S)-4-((3S)-4-(6-((5-(3-(7,7-dimethyl-1-oxo-1,3,4,6,7,8-hexahydro-2H-cyclopenta[4,5]pyrrolo[1,2-a]pyrazin-2-yl)-2-(hydroxymethyl)piperidin-1-yl)-1-methyl-2-oxo-1,2-dihydropyridin-3-yl)amino)pyridin-3-yl)-3-methylpiperazin-1-yl)-2-methylpiperidin-1-yl)phenyl)piperidine-2,6-dione,
3-((3-(4-((3S)-3-methyl-4-(6-((1-methyl-5-(2-methyl-3-(1-oxo-3,4,5,6,7,8-hexahydrobenzo[4,5]thieno[2,3-c]pyridin-2(1H)-yl)piperidin-1-yl)-2-oxo-1,2-dihydropyridin-3-yl)amino)pyridin-3-yl)piperazin-1-yl)piperidin-1-yl)phenyl)amino)piperidine-2,6-dione
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)-2-(trifluoromethyl)pyridin-3-yl)-3-methylpiperazin-1-yl)piperidin-1-yl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)-2-methylpyridin-3-yl)-3-methylpiperazin-1-yl)piperidin-1-yl)-2-(2,6-dioxopiperidin-3-yl)isoindoline-1,3-dione,
5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)-2-(methylamino)pyridin-3-yl)-3-methylpiperazin-1-yl)piperidin-1-yl)-2-(2,6-dioxopiperidin-3-yl)isoindoline-1,3-dione,
N-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)-3-((2S)-4-(1-(2-(2,6-dioxopiperidin-3-yl)-1,3-dioxoisoindolin-5-yl)piperidin-4-yl)-2-methylpiperazin-1-yl)pyridin-2-yl)propionamide,
N-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)-3-((2S)-4-(1-(2-(2,6-dioxopiperidin-3-yl)-1,3-dioxoisoindolin-5-yl)piperidin-4-yl)-2-methylpiperazin-1-yl)pyridin-2-yl)isobutyramide,

Compounds of the invention may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or S configuration and both of these configurations are within the scope of the invention.

A modified compound of any one of such compounds including a modification having an improved (e.g., enhanced, greater) pharmaceutical solubility, stability, bioavailability and/or therapeutic index as compared to the unmodified compound is also contemplated. The examples of modifications include but not limited to the prodrug derivatives, and the deuterium-enriched compounds. For example:
Prodrug derivatives: prodrugs, upon administration to a subject, will converted in vivo into active compounds of the present invention [Nature Reviews of Drug Discovery, 2008, Volume 7, p255]. It is noted that in many instances, the prodrugs themselves also fall within the scope of the range of compounds according to the present invention. The prodrugs of the compounds of the present invention can be prepared by standard organic reaction, for example, by reacting with a carbamylating agent (e.g., 1,1-acyloxyalkylcarbonochloridate, para-nitrophenyl carbonate, or the like) or an acylating agent. Further examples of methods and strategies of making prodrugs are described in Bloorganic and Medicinal Chemistry Letters, 1994, Vol. 4, p. 1985.
Deuterium-enriched compounds: deuterium (D or ²H) is a stable, non-radioactive isotope of hydrogen and has an atomic weight of 2.0144. Hydrogen naturally occurs as a mixture of the isotopes ^{X}H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their nonenriched counterparts.

It should be recognized that the compounds of the present invention may be present and optionally administered in the form of salts, and solvates. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, 2-hydroxyethanesulfonate, iodide, isethionate, iso-butyrate, lactate, lactobionate, malonate, mandelate, metaphosphate, methanesulfonate, methylbenzoate, monohydrogenphosphate, 2-naphthalenesulfonate, nicotinate, oxalate, oleate, pamoate, pectinate, persulfate, phenylacetate, 3-phenylpropionate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

In one aspect, a pharmaceutically acceptable salt is a hydrochloride salt, hydrobromide salt, methanesulfonate, toluenesulfonate, acetate, fumarate, sulfate, bisulfate, succinate, citrate, phosphate, maleate, nitrate, tartrate, benzoate, biocarbonate, carbonate, sodium hydroxide salt, calcium hydroxide salt, potassium hydroxide salt, tromethamine salt, or mixtures thereof.

Compounds of the present invention that comprise tertiary nitrogen-containing groups may be quaternized with such agents as (C₁₋₄) alkyl halides, e.g., methyl, ethyl, iso-propyl and tert-butyl chlorides, bromides and iodides; di-(C₁₋₄) alkyl sulfates, e.g., dimethyl, diethyl and diamyl sulfates; alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water- and oil-soluble compounds of the invention.

Amine oxides, also known as amine-N-oxide and N-oxide, of anti-cancer agents with tertiary nitrogen atoms have been developed as prodrugs [Mol Cancer Therapy. 2004 March; 3(3):233-44]. Compounds of the present invention that comprise tertiary nitrogen atoms may be oxidized by such agents as hydrogen peroxide (H₂O), Caro's acid or peracids like meta-Chloroperoxybenzoic acid (mCPBA) to from amine oxide.

The invention encompasses pharmaceutical compositions comprising the compound of the present invention and pharmaceutical excipients, as well as other conventional pharmaceutically inactive agents. Any inert excipient that is commonly used as a carrier or diluent may be used in compositions of the present invention, such as sugars, polyalcohols, soluble polymers, salts and lipids. Sugars and polyalcohols which may be employed include, without limitation, lactose, sucrose, mannitol, and sorbitol. Illustrative of the soluble polymers which may be employed are polyoxyethylene, poloxamers, polyvinylpyrrolidone, and dextran. Useful salts include, without limitation, sodium chloride, magnesium chloride, and calcium chloride. Lipids which may be employed include, without limitation, fatty acids, glycerol fatty acid esters, glycolipids, and phospholipids.

In addition, the pharmaceutical compositions may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmellose sodium, crospovidone, guar gum, sodium starch glycolate, Primogel), buffers (e.g., tris-HCL, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g., sodium lauryl sulfate), permeation enhancers, solubilizing agents (e.g., glycerol, polyethylene glycerol, cyclodextrins), a glidant (e.g., colloidal silicon dioxide), anti-oxidants (e.g., ascorbic acid, sodium metabisulfite, butylated hydroxyanisole), stabilizers (e.g., hydroxypropyl cellulose, hydroxypropylmethyl cellulose), viscosity increasing agents (e.g., carbomer, colloidal silicon dioxide, ethyl cellulose, guar gum), sweeteners (e.g., sucrose, aspartame, citric acid), flavoring agents (e.g., peppermint, methyl salicylate, or orange flavoring), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate, methyl cellulose, hydroxyethyl cellulose, carboxymethylcellulose sodium), polymer coatings (e.g., poloxamers or poloxamines), coating and film forming agents (e.g., ethyl cellulose, acrylates, polymethacrylates) and/or adjuvants.

In one embodiment, the pharmaceutical compositions are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, the invention encompasses pharmaceutical compositions comprising any solid or liquid physical form of the compound of the invention. For example, the compounds can be in a crystalline form, in amorphous form, and have any particle size. The particles may be micronized, or may be agglomerated, particulate granules, powders, oils, oily suspensions or any other form of solid or liquid physical form.

When compounds according to the present invention exhibit insufficient solubility, methods for solubilizing the compounds may be used. Such methods are known to those of skill in this art, and include, but are not limited to, pH adjustment and salt formation, using co-solvents, such as ethanol, propylene glycol, polyethylene glycol (PEG) 300, PEG 400, DMA (10-30%), DMSO (10-20%), NMP (10-20%), using surfactants, such as polysorbate 80, polysorbate 20 (1-10%), cremophor EL, Cremophor RH40, Cremophor RH60 (5-10%), Pluronic F68/Poloxamer 188 (20-50%), Solutol HS15 (20-50%), Vitamin E TPGS, and d-α-tocopheryl PEG 1000 succinate (20-50%), using complexation such as HPβCD and SBEβCD (10-40%), and using advanced approaches such as micelle, addition of a polymer, nanoparticle suspensions, and liposome formation.

A wide variety of administration methods may be used in conjunction with the compounds of the present invention. Compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds according to the invention may also be administered or coadministered in slow release dosage forms. Compounds may be in gaseous, liquid, semi-liquid or solid form, formulated in a manner suitable for the route of administration to be used. For oral administration, suitable solid oral formulations include tablets, capsules, pills, granules, pellets, sachets and effervescent, powders, and the like. Suitable liquid oral formulations include solutions, suspensions, dispersions, emulsions, oils and the like. For parenteral administration, reconstitution of a lyophilized powder is typically used.

As used herein, “Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Aliphatic” means a moiety characterized by a straight or branched chain arrangement of constituent carbon atoms and may be saturated or partially unsaturated with one or more double or triple bonds.

The term “alkyl” refers to a straight or branched hydrocarbon containing 1-20 carbon atoms (e.g., C₁-C₁₀). Examples of alkyl include, but are not limited to, methyl, methylene, ethyl, ethylene, n-propyl, i-propyl, n-butyl, i-butyl, and t-butyl. Preferably, the alkyl group has one to ten carbon atoms. More preferably, the alkyl group has one to four carbon atoms.

The term “alkenyl” refers to a straight or branched hydrocarbon containing 2-20 carbon atoms (e.g., C₂-C₁₀) and one or more double bonds. Examples of alkenyl include, but are not limited to, ethenyl, propenyl, and allyl.

Preferably, the alkylene group has two to ten carbon atoms. More preferably, the alkylene group has two to four carbon atoms.

The term “alkynyl” refers to a straight or branched hydrocarbon containing 2-20 carbon atoms (e.g., C₂-C₁₀) and one or more triple bonds. Examples of alkynyl include, but are not limited to, ethynyl, 1-propynyl, 1- and 2-butynyl, and 1-methyl-2-butynyl. Preferably, the alkynyl group has two to ten carbon atoms. More preferably, the alkynyl group has two to four carbon atoms.

The term “alkylamino” refers to an —N(R)-alkyl in which R can be H, alkyl, alkenyl, alkynyl, cycloalkyl, cycloalkenyl, heterocycloalkyl, heterocycloalkenyl, aryl, or heteroaryl.

“Alkoxy” means an oxygen moiety having a further alkyl substituent.

“Alkoxycarbonyl” means an alkoxy group attached to a carbonyl group.

“Oxoalkyl” means an alkyl, further substituted with a carbonyl group. The carbonyl group may be an aldehyde, ketone, ester, amide, acid or acid chloride.

The term “cycloalkyl” refers to a saturated hydrocarbon ring system having 3 to 30 carbon atoms (e.g., C₃-C₁₂, C₃-C₈, C₃-C₆). Examples of cycloalkyl include, but are not limited to, cyclopropyl, cyclobutyl, cyclopentyl, cyclohexyl, cycloheptyl, and cyclooctyl. The term “cycloalkenyl” refers to a non-aromatic hydrocarbon ring system having 3 to 30 carbons (e.g., C₃-C₁₂) and one or more double bonds. Examples include cyclopentenyl, cyclohexenyl, and cycloheptenyl.

The term “heterocycloalkyl” refers to a nonaromatic 5-8 membered monocyclic, 8-12 membered bicyclic, or 11-14 membered tricyclic ring system having one or more heteroatoms (such as O, N, S, P, or Se). Examples of heterocycloalkyl groups include, but are not limited to, piperazinyl, pyrrolidinyl, dioxanyl, morpholinyl, and tetrahydrofuranyl.

The term “heterocycloalkenyl” refers to a nonaromatic 5-8 membered monocyclic, 8-12 membered bicyclic, or 11-14 membered tricyclic ring system having one or more heteroatoms (such as O, N, S, P, or Se) and one or more double bonds.

The term “aryl” refers to a 6-carbon monocyclic, 10-carbon bicyclic, 14-carbon tricyclic aromatic ring system. Examples of aryl groups include, but are not limited to, phenyl, naphthyl, and anthracenyl.

The term “heteroaryl” refers to an aromatic 5-8 membered monocyclic, 8-12 membered bicyclic, or 11-14 membered tricyclic ring system having one or more heteroatoms (such as O, N, S, P, or Se). Examples of heteroaryl groups include pyridyl, furyl, imidazolyl, benzimidazolyl, pyrimidinyl, thienyl, quinolinyl, indolyl, and thiazolyl.

Spiroalkyl refers to a compound comprising two saturated cyclic alkyl rings sharing only one common atom (also known as a spiro atom), with no heteroatom and no unsaturated bonds on any of the rings. In one embodiment, the spiroalkyl is bicyclic. In another embodiment, the spiroalikyl has more than two cycles. In certain embodiments, the spiroalkyl compound is a polyspiro compound connected by two or more spiroatoms making up three or more rings. In certain embodiments, one of the rings of the bicyclic spiroalkyl has 3, 4, 5, 6, 7, or 8 atoms, including the common spito atom. In one embodiment, the spiroalkyl is a 5 to 20 membered, 5 to 14 membered, or 5 to 10 membered polycyclic spiroalkyl group. Representative examples of spiroalkyl include, but are not limited to the following groups:

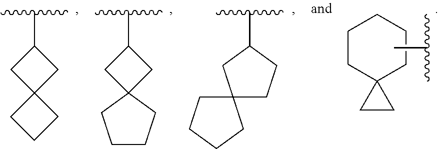

Spiroheterocyclyl refers to a compound comprising two non-saturated rings sharing only one common atom (also known as a spiro atom), with at least one heteroatom on one of the two rings, such as a polycyclic heterocyclyl group with rings connected through one common carbon atom. The common atom can be carbon (C), silicon, or nitrogen (such as a positively charged quaternary nitrogen atom). The heteroatoms can comprise nitrogen, quaternary nitrogen, oxidized nitrogen (e.g., NO), oxygen, silicon, and sulfur, including sulfoxide and sulfone, and the remaining ring atoms are C. In addition, one or more of the rings may contain one or more double bonds. In one embodiment, the spiro heterocyclyl is bicyclic, with heteroatom(s) on either one or both cycles. In certain embodiments, one of the rings of the bicyclic spiro heterocyclyl has 3, 4, 5, 6, 7, or 8 atoms, including the common spito atom. In certain embodiments, the spiro heterocyclic compound is a polyspiro compound connected by two or more spiroatoms making up three or more rings. In one embodiment, the spiro heterocyclyl is a 5 to 20 membered, 5 to 14 membered, or 5 to 10 membered polycyclic heterocyclyl group. Representative examples of spiro heterocyclyl include, but are not limited to the following groups:

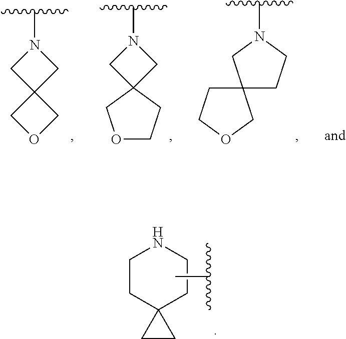

Fused heterocyclyl refers to a polycyclic heterocyclyl group, wherein each ring in the group shares an adjacent pair of atoms (such as carbon atoms) with another ring in the group, wherein one or more rings can contain one or more double bonds (such as a phenyl ring, in a two-ring fused heterocyclyl), and wherein said rings have one or more heteroatoms, which can be nitrogen, quaternary nitrogen, oxidized nitrogen (e.g., NO), oxygen, and sulfur, including sulfoxide and sulfone, and the remaining ring atoms are C. In certain embodiments, the fused heterocyclyl is bicyclic (such as a phenyl ring fused to a N-containing heterocycle). In certain embodiments, the fused heterocyclyl contains more than two rings, at least two of which share an adjacent pair of atoms. In one embodiment, the fused heterocyclyl is a 5 to 20 membered, 5 to 16 membered, or 5 to 10 membered (e.g., 9-membered) polycyclic heterocyclyl group. Representative examples of fused heterocyclyl include, but are not limited to the following groups:

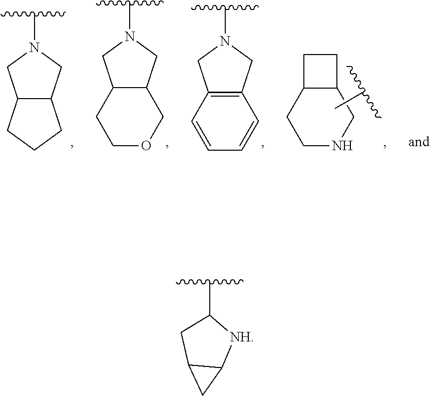

Bridged heterocyclyl refers to a compound having at least two rings sharing three or more common ring atoms, separating the two bridgehead atoms by a bridge containing at least one atom, wherein at least one ring atom is a heteroatom. The bridgehead atoms are the atoms from which three bonds radiate and where the rings meet. The rings of the bridged heterocyclyl can have one or more double bonds, and the ring heteroatom(s) can be nitrogen, quaternary nitrogen, oxidized nitrogen (e.g., NO), oxygen, and sulfur, including sulfoxide and sulfone as ring atoms, while the remaining ring atoms are C. In one embodiment, the bridged heterocyclyl is bicyclic. In one embodiment, the bridged heterocyclyl is a 5 to 20 membered, 5 to 16 membered, or 5 to 10 membered polycyclic heterocyclyl group. Representative examples of bridged heterocyclyl include, but are not limited to the following groups:

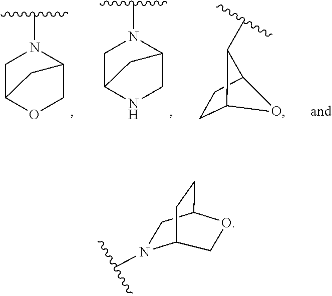

“Amino” means a nitrogen moiety having two further substituents where each substituent has a hydrogen or carbon atom alpha bonded to the nitrogen. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aromatic” means a moiety wherein the constituent atoms make up an unsaturated ring system, all atoms in the ring system are sp2 hybridized and the total number of pi electrons is equal to 4n+2. An aromatic ring may be such that the ring atoms are only carbon atoms or may include carbon and non-carbon atoms (see Heteroaryl).

“Carbamoyl” means the radical —OC(O)NRₐR_{b} where Rₐ and R_{b} are each independently two further substituents where a hydrogen or carbon atom is alpha to the nitrogen. It is noted that carbamoyl moieties may include protected derivatives thereof. Examples of suitable protecting groups for carbamoyl moieties include acetyl, tert-butoxycarbonyl, benzyloxycarbonyl, and the like. It is noted that both the unprotected and protected derivatives fall within the scope of the invention.

“Carbonyl” means the radical —C(O)—. It is noted that the carbonyl radical may be further substituted with a variety of substituents to form different carbonyl groups including acids, acid halides, amides, esters, and ketones.

“Carboxy” means the radical —C(O)O—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Formyl” means the radical —CH═O.

“Formimino” means the radical —HC═NH.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like.

“Hydroxy” means the radical —OH.

“Imine derivative” means a derivative comprising the moiety —C(═NR)—, wherein R comprises a hydrogen or carbon atom alpha to the nitrogen.

“Isomers” mean any compound having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.”

“Nitro” means the radical —NO₂.

“Protected derivatives” means derivatives of compounds in which a reactive site are blocked with protecting groups. Protected derivatives are useful in the preparation of pharmaceuticals or in themselves may be active as inhibitors. A comprehensive list of suitable protecting groups can be found in T. W. Greene, Protecting Groups in Organic Synthesis, 3rd edition, Wiley & Sons, 1999.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position. The term “unsubstituted” means that a given moiety may consist of only hydrogen substituents through available valencies (unsubstituted).

If a functional group is described as being “optionally substituted,” the function group may be either (1) not substituted, or (2) substituted. If a carbon of a functional group is described as being optionally substituted with one or more of a list of substituents, one or more of the hydrogen atoms on the carbon (to the extent there are any) may separately and/or together be replaced with an independently selected optional substituent.

“Sulfide” means —S—R wherein R is H, alkyl, carbocycle, heterocycle, carbocycloalkyl or heterocycloalkyl. Particular sulfide groups are mercapto, alkylsulfide, for example methylsulfide (—S-Me); arylsulfide, e.g., phenylsulfide; aralkylsulfide, e.g., benzylsulfide.

“Sulfinyl” means the radical —S(O)—. It is noted that the sulfinyl radical may be further substituted with a variety of substituents to form different sulfinyl groups including sulfinic acids, sulfinamides, sulfinyl esters, and sulfoxides.

“Sulfonyl” means the radical —S(O)(O)—. It is noted that the sulfonyl radical may be further substituted with a variety of substituents to form different sulfonyl groups including sulfonic acids, sulfonamides, sulfonate esters, and sulfones.

“Thiocarbonyl” means the radical —C(S)—. It is noted that the thiocarbonyl radical may be further substituted with a variety of substituents to form different thiocarbonyl groups including thioacids, thioamides, thioesters, and thioketones.

“Animal” includes humans, non-human mammals (e.g., non-human primates, rodents, mice, rats, hamsters, dogs, cats, rabbits, cattle, horses, sheep, goats, swine, deer, and the like) and non-mammals (e.g., birds, and the like).

“Bioavailability” as used herein is the fraction or percentage of an administered dose of a drug or pharmaceutical composition that reaches the systemic circulation intact. In general, when a medication is administered intravenously, its bioavailability is 100%. However, when a medication is administered via other routes (e.g., orally), its bioavailability decreases (e.g., due to incomplete absorption and first-pass metabolism). Methods to improve the bioavailability include prodrug approach, salt synthesis, particle size reduction, complexation, change in physical form, solid dispersions, spray drying, and hot-melt extrusion.

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means organic or inorganic salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

“Pharmaceutically acceptable carrier” means a non-toxic solvent, dispersant, excipient, adjuvant, or other material which is mixed with the compounds of the present invention in order to form a pharmaceutical composition, i.e., a dose form capable of administration to the patient. Examples of pharmaceutically acceptable carrier includes suitable polyethylene glycol (e.g., PEG400), surfactant (e.g., Cremophor), or cyclopolysaccharide (e.g., hydroxypropyl-β-cyclodextrin or sulfobutyl ether β-cyclodextrins), polymer, liposome, micelle, nanosphere, etc.

“Pharmacophore,” as defined by The International Union of Pure and Applied Chemistry, is an ensemble of steric and electronic features that is necessary to ensure the optimal supramolecular interactions with a specific biological target and to trigger (or block) its biological response. For example, Camptothecin is the pharmacophore of the well known drug topotecan and irinotecan. Mechlorethamine is the pharmacophore of a list of widely used nitrogen mustard drugs like Melphalan, Cyclophosphamide, Bendamustine, and so on.

“Prodrug” means a compound that is convertible in vivo metabolically into an active pharmaceutical according to the present invention. For example, an inhibitor comprising a hydroxyl group may be administered as an ester that is converted by hydrolysis in vivo to the hydroxyl compound.

“Stability” in general refers to the length of time a drug retains its properties without loss of potency. Sometimes this is referred to as shelf life. Factors affecting drug stability include, among other things, the chemical structure of the drug, impurity in the formulation, pH, moisture content, as well as environmental factors such as temperature, oxidization, light, and relative humidity. Stability can be improved by providing suitable chemical and/or crystal modifications (e.g., surface modifications that can change hydration kinetics; different crystals that can have different properties), excipients (e.g., anything other than the active substance in the dosage form), packaging conditions, storage conditions, etc.

“Therapeutically effective amount” of a composition described herein is meant an amount of the composition which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the composition described above may range from about 0.1 mg/kg to about 500 mg/kg, preferably from about 0.2 to about 50 mg/kg. Effective doses will also vary depending on route of administration, as well as the possibility of co-usage with other agents. It will be understood, however, that the total daily usage of the compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

As used herein, the term “treating” refers to administering a compound to a subject that has a neoplastic or immune disorder, or has a symptom of or a predisposition toward it, with the purpose to cure, heal, alleviate, relieve, alter, remedy, ameliorate, improve, or affect the disorder, the symptoms of or the predisposition toward the disorder. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents.

A “subject” refers to a human and a non-human animal. Examples of a non-human animal include all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

“Combination therapy” includes the administration of the subject compounds of the present invention in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, or non-drug therapies, preferably compounds that are able to enhance the effect of the compounds of the invention. The compounds of the invention can be administered simultaneously (as a single preparation or separate preparation) or sequentially to the other therapies. In general, a combination therapy envisions administration of two or more drugs/treatments during a single cycle or course of therapy.

In one embodiment, the compounds of the invention are administered in combination with one or more of traditional chemotherapeutic agents. The traditional chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as Nitrogen Mustards (e.g., Bendamustine, Cyclophosphamide, Melphalan, Chlorambucil, Isofosfamide), Nitrosureas (e.g., Carmustine, Lomustine and Streptozocin), ethylenimines (e.g., thiotepa, hexamethylmelanine), Alkylsulfonates (e.g., Busulfan), Hydrazines and Triazines (e.g., Altretamine, Procarbazine, Dacarbazine and Temozolomide), and platinum based agents (e.g., Carboplatin, Cisplatin, and Oxaliplatin); plant alkaloids such as Podophyllotoxins (e.g., Etoposide and Tenisopide), Taxanes (e.g., Paclitaxel and Docetaxel), Vinca alkaloids (e.g., Vincristine, Vinblastine and Vinorelbine); anti-tumor antibiotics such as Chromomycins (e.g., Dactinomycin and Plicamycin), Anthracyclines (e.g., Doxorubicin, Daunorubicin, Epirubicin, Mitoxantrone, and Idarubicin), and miscellaneous antibiotics such as Mitomycin and Bleomycin; anti-metabolites such as folic acid antagonists (e.g., Methotrexate), pyrimidine antagonists (e.g., 5-Fluorouracil, Foxuridine, Cytarabine, Capecitabine, and Gemcitabine), purine antagonists (e.g., 6-Mercaptopurine and 6-Thioguanine) and adenosine deaminase inhibitors (e.g., Cladribine, Fludarabine, Nelarabine and Pentostatin); topoisomerase inhibitors such as topoisomerase I inhibitors(Topotecan, Irinotecan), topoisomerase II inhibitors (e.g., Amsacrine, Etoposide, Etoposide phosphate, Teniposide), and miscellaneous anti-neoplastics such as ribonucleotide reductase inhibitors (Hydroxyurea), adrenocortical steroid inhibitor (Mitotane), anti-microtubule agents (Estramustine), and retinoids (Bexarotene, Isotretinoin, Tretinoin (ATRA).

In one aspect of the invention, the compounds may be administered in combination with one or more targeted anti-cancer agents that modulate protein kinases involved in various disease states. Examples of such kinases may include, but are not limited ABL1, ABL2/ARG, ACK1, AKT1, AKT2, AKT3, ALK, ALK1/ACVRL1, ALK2/ACVR1, ALK4/ACVR1B, ALK5/TGFBR1, ALK6/BMPR1B, AMPK(A1/B1/G1), AMPK(A1/B1/G2), AMPK(A1/B1/G3), AMPK(A1/B2/G1), AMPK(A2/B1/G1), AMPK(A2/B2/G1), AMPK(A2/B2/G2), ARAF, ARK5/NUAK1, ASK1/MAP3K5, ATM, Aurora A, Aurora B, Aurora C, AXL, BLK, BMPR2, BMX/ETK, BRAF, BRK, BRSK1, BRSK2, BTK, CAMK1a, CAMK1b, CAMK1d, CAMK1g, CAMKIIa, CAMKIIb, CAMKIId, CAMKIIg, CAMK4, CAMKK1, CAMKK2, CDC7-DBF4, CDK1-cyclin A, CDK1-cyclin B, CDK1-cyclin E, CDK2-cyclin A, CDK2-cyclin A1, CDK2-cyclin E, CDK3-cyclin E, CDK4-cyclin D1, CDK4-cyclin D3, CDK5-p25, CDK5-p35, CDK6-cyclin D1, CDK6-cyclin D3, CDK7-cyclin H, CDK9-cyclin K, CDK9-cyclin T1, CHK1, CHK2, CK1a1, CK1d, CK1epsilon, CK1g1, CK1g2, CK1g3, CK2a, CK2a2, c-KIT, CLK1, CLK2, CLK3, CLK4, c-MER, c-MET, COT1/MAP3K8, CSK, c-SRC, CTK/MATK, DAPK1, DAPK2, DCAMKL1, DCAMKL2, DDR1, DDR2, DLK/MAP3K12, DMPK, DMPK2/CDC42BPG, DNA-PK, DRAK1/STK17A, DYRK1/DYRK1A, DYRK1B, DYRK2, DYRK3, DYRK4, EEF2K, EGFR, EIF2AK1, EIF2AK2, EIF2AK3, EIF2AK4/GCN2, EPHA1, EPHA2, EPHA3, EPHA4, EPHA5, EPHA6, EPHA7, EPHA8, EPHB1, EPHB2, EPHB3, EPHB4, ERBB2/HER2, ERBB4/HER4, ERK1/MAPK3, ERK2/MAPK1, ERK5/MAPK7, FAK/PTK2, FER, FES/FPS, FGFR1, FGFR2, FGFR3, FGFR4, FGR, FLT1NEGFR1, FLT3, FLT4/VEGFR3, FMS, FRK/PTK5, FYN, GCK/MAP4K2, GRK1, GRK2, GRK3, GRK4, GRK5, GRK6, GRK7, GSK3a, GSK3b, Haspin, HCK, HGK/MAP4K4, HIPK1, HIPK2, HIPK3, HIPK4, HPK1/MAP4K1, IGF1R, IKKa/CHUK, IKKb/IKBKB, IKKe/IKBKE, IR, IRAK1, IRAK4, IRR/INSRR, ITK, JAK1, JAK2, JAK3, JNK1, JNK2, JNK3, KDRNEGFR2, KHS/MAP4K5, LATS1, LATS2, LCK, LCK2/ICK, LKB1, LIMK1, LOK/STK10, LRRK2, LYN, LYNB, MAPKAPK2, MAPKAPK3, MAPKAPK5/PRAK, MARK1, MARK2/PAR-1Ba, MARK3, MARK4, MEK1, MEK2, MEKK1, MEKK2, MEKK3, MELK, MINK/MINK1, MKK4, MKK6, MLCK/MYLK, MLCK2/MYLK2, MLK1/MAP3K9, MLK2/MAP3K10, MLK3/MAP3K11, MNK1, MNK2, MRCKa/, CDC42BPA, MRCKb/, CDC42BPB, MSK1/RPS6KA5, MSK2/RPS6KA4, MSSK1/STK23, MST1/STK4, MST2/STK3, MST3/STK24, MST4, mTOR/FRAP1, MUSK, MYLK3, MYO3b, NEK1, NEK2, NEK3, NEK4, NEK6, NEK7, NEK9, NEK11, NIK/MAP3K14, NLK, OSR1/OXSR1, P38a/MAPK14, P38b/MAPK11, P38d/MAPK13, P38g/MAPK12, P70S6K/RPS6KB1, p70S6Kbl, RPS6KB2, PAK1, PAK2, PAK3, PAK4, PAK5, PAK6, PASK, PBK/TOPK, PDGFRa, PDGFRb, PDK1/PDPK1, PDK1/PDHK1, PDK2/PDHK2, PDK3/PDHK3, PDK4/PDHK4, PHKg1, PHKg2, PI3Ka, (p110a/p85a), PI3Kb, (p110b/p85a), PI3Kd, (p110d/p85a), PI3Kg(p120g), PIM1, PIM2, PIM3, PKA, PKAcb, PKAcg, PKCa, PKCb1, PKCb2, PKCd, PKCepsilon, PKCeta, PKCg, PKCiota, PKCmu/PRKD1, PKCnu/PRKD3, PKCtheta, PKCzeta, PKD2/PRKD2, PKG1a, PKG1b, PKG2/PRKG2, PKN1/PRK1, PKN2/PRK2, PKN3/PRK3, PLK1, PLK2, PLK3, PLK4/SAK, PRKX, PYK2, RAF1, RET, RIPK2, RIPK3, RIPK5, ROCK1, ROCK2, RON/MST1R, ROS/ROS1, RSK1, RSK2, RSK3, RSK4, SGK1, SGK2, SGK3/SGKL, SIK1, SIK2, SLK/STK2, SNARK/NUAK2, SRMS, SSTK/TSSK6, STK16, STK22D/TSSK1, STK25/YSK1, STK32b/YANK2, STK32c/YANK3, STK33, STK38/NDR1, STK38L/NDR2, STK39/STLK3, SRPK1, SRPK2, SYK, TAK1, TAOK1, TAOK2/TA01, TAOK3/JIK, TBK1, TEC, TESK1, TGFBR2, TIE2/TEK, TLK1, TLK2, TNIK, TNK1, TRKA, TRKB, TRKC, TRPM7/CHAK1, TSSK2, TSSK3/STK22C, TTBK1, TTBK2, TTK, TXK, TYK1/LTK, TYK2, TYRO3/SKY, ULK1, ULK2, ULK3, VRK1, VRK2, WEE1, WNK1, WNK2, WNK3, YES/YES1, ZAK/MLTK, ZAP70, ZIPK/DAPK3, KINASE, MUTANTS, ABL1(E255K), ABL1(F3171), ABL1(G250E), ABL1(H396P), ABL1(M351T), ABL1(Q₂₅₂H), ABL1(T3151), ABL1(Y253F), ALK (C1156Y), ALK(L1196M), ALK (F1174L), ALK (R1275Q), BRAF(V599E), BTK(E41 K), CHK2(1157T), c-Kit(A829P), c-KIT(D816H), c-KIT(D816V), c-Kit(D820E), c-Kit(N822K), C-Kit (T6701), c-Kit(V559D), c-Kit(V559DN654A), c-Kit(V559D/T6701), C-Kit (V560G), c-KIT(V654A), C-MET(D1228H), C-MET(D1228N), C-MET(F12001), c-MET(M1250T), C-MET(Y1230A), C-MET(Y1230C), C-MET(Y1230D), C-MET(Y1230H), c-Src(T341M), EGFR(G719C), EGFR(G719S), EGFR(L858R), EGFR(L861Q), EGFR(T790M), EGFR, (L858R,T790M), EGFR(d746-750/T790M), EGFR(d746-750), EGFR(d747-749/A750P), EGFR(d747-752/P753S), EGFR(d752-759), FGFR1(V561M), FGFR2(N549H), FGFR3(G697C), FGFR3(K650E), FGFR3(K650M), FGFR4(N535K), FGFR4(V550E), FGFR4(V550L), FLT3(D835Y), FLT3(ITD), JAK2 (V617F), LRRK2 (G2019S), LRRK2 (12020T), LRRK2 (R1441C), p38a(T106M), PDGFRa(D842V), PDGFRa(T6741), PDGFRa(V561D), RET(E762Q), RET(G691S), RET(M918T), RET(R749T), RET(R813Q), RET(V804L), RET(V804M), RET(Y791F), TIF2(R849W), TIF2(Y897S), and TIF2(Y1108F).

In another aspect of the invention, the subject compounds may be administered in combination with one or more targeted anti-cancer agents that modulate non-kinase biological targets, pathway, or processes. Such targets pathways, or processes include but not limited to heat shock proteins (e.g.HSP90), poly-ADP (adenosine diphosphate)-ribose polymerase (PARP), hypoxia-inducible factors(HIF), proteasome, Wnt/Hedgehog/Notch signaling proteins, TNF-alpha, matrix metalloproteinase, farnesyl transferase, apoptosis pathway (e.g Bcl-xL, Bcl-2, Bcl-w), histone deacetylases (HDAC), histone acetyltransferases (HAT), and methyltransferase (e.g histone lysine methyltransferases, histone arginine methyltransferase, DNA methyltransferase, etc).

In another aspect of the invention, the compounds of the invention are administered in combination with one or more of other anti-cancer agents that include, but are not limited to, gene therapy, RNAi cancer therapy, chemoprotective agents (e.g., amfostine, mesna, and dexrazoxane), drug-antibody conjugate(e.g brentuximab vedotin, ibritumomab tioxetan), cancer immunotherapy such as Interleukin-2, cancer vaccines(e.g., sipuleucel-T) or monoclonal antibodies (e.g., Bevacizumab, Alemtuzumab, Rituximab, Trastuzumab, etc).

In another aspect of the invention, the subject compounds are administered in combination with radiation therapy or surgeries. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

In certain embodiments, the compounds of the invention are administered in combination with one or more of radiation therapy, surgery, or anti-cancer agents that include, but are not limited to, DNA damaging agents, antimetabolites, topoisomerase inhibitors, anti-microtubule agents, kinase inhibitors, epigenetic agents, HSP90 inhibitors, PARP inhibitors, BCL-2 inhibitor, drug-antibody conjugate, and antibodies targeting VEGF, HER2, EGFR, CD50, CD20, CD30, CD33, etc.

In certain embodiments, the compounds of the invention are administered in combination with one or more of abarelix, abiraterone acetate, aldesleukin, alemtuzumab, altretamine, anastrozole, asparaginase, bendamustine, bevacizumab, bexarotene, bicalutamide, bleomycin, bortezombi, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate, radiation therapy, or surgery.

In certain embodiments, the compounds of the invention are administered in combination with one or more anti-inflammatory agent. Anti-inflammatory agents include but are not limited to NSAIDs, non-specific and COX-2 specific cyclooxgenase enzyme inhibitors, gold compounds, corticosteroids, methotrexate, tumor necrosis factor receptor (TNF) receptors antagonists, immunosuppressants and methotrexate. Examples of NSAIDs include, but are not limited to, ibuprofen, flurbiprofen, naproxen and naproxen sodium, diclofenac, combinations of diclofenac sodium and misoprostol, sulindac, oxaprozin, diflunisal, piroxicam, indomethacin, etodolac, fenoprofen calcium, ketoprofen, sodium nabumetone, sulfasalazine, tolmetin sodium, and hydroxychloroquine. Examples of NSAIDs also include COX-2 specific inhibitors such as celecoxib, valdecoxib, lumiracoxib and/or etoricoxib.

In some embodiments, the anti-inflammatory agent is a salicylate. Salicylates include by are not limited to acetylsalicylic acid or aspirin, sodium salicylate, and choline and magnesium salicylates. The anti-inflammatory agent may also be a corticosteroid. For example, the corticosteroid may be cortisone, dexamethasone, methylprednisolone, prednisolone, prednisolone sodium phosphate, or prednisone.

In additional embodiments the anti-inflammatory agent is a gold compound such as gold sodium thiomalate or auranofin.

The invention also includes embodiments in which the anti-inflammatory agent is a metabolic inhibitor such as a dihydrofolate reductase inhibitor, such as methotrexate or a dihydroorotate dehydrogenase inhibitor, such as leflunomide.

Other embodiments of the invention pertain to combinations in which at least one anti-inflammatory compound is an anti-C5 monoclonal antibody (such as eculizumab or pexelizumab), a TNF antagonist, such as entanercept, or infliximab, which is an anti-TNF alpha monoclonal antibody.

In certain embodiments, the compounds of the invention are administered in combination with one or more immunosuppressant agents.

In some embodiments, the immunosuppressant agent is glucocorticoid, methotrexate, cyclophosphamide, azathioprine, mercaptopurine, leflunomide, cyclosporine, tacrolimus, and mycophenolate mofetil, dactinomycin, anthracyclines, mitomycin C, bleomycin, or mithramycin, or fingolimod.

The invention further provides methods for the prevention or treatment of a neoplastic disease, autoimmune and/or inflammatory disease. In one embodiment, the invention relates to a method of treating a neoplastic disease, autoimmune and/or inflammatory disease in a subject in need of treatment comprising administering to said subject a therapeutically effective amount of a compound of the invention. In one embodiment, the invention further provides for the use of a compound of the invention in the manufacture of a medicament for halting or decreasing a neoplastic disease, autoimmune and/or inflammatory disease.

In one embodiment, the neoplastic disease is a B-cell malignancy includes but not limited to B-cell lymphoma, lymphoma (including Hodgkin's lymphoma and non-Hodgkin's lymphoma), hairy cell lymphoma, small lymphocytic lymphoma (SLL), mantle cell lymphoma (MCL), and diffuse large B-cell lymphoma (DLBCL), multiple myeloma, chronic and acute myelogenous leukemia and chronic and acute lymphocytic leukemia.

The autoimmune and/or inflammatory diseases that can be affected using compounds and compositions according to the invention include, but are not limited to allergy, Alzheimer's disease, acute disseminated encephalomyelitis, Addison's disease, ankylosing spondylitis, antiphospholipid antibody syndrome, asthma, atherosclerosis, autoimmune hemolytic anemia, autoimmune hemolytic and thrombocytopenic states, autoimmune hepatitis, autoimmune inner ear disease, bullous pemphigoid, coeliac disease, chagas disease, chronic obstructive pulmonary disease, chronic Idiopathic thrombocytopenic purpura (ITP), churg-strauss syndrome, Crohn's disease, dermatomyositis, diabetes mellitus type 1, endometriosis, Goodpasture's syndrome (and associated glomerulonephritis and pulmonary hemorrhage), graves' disease, guillain-barré syndrome, hashimoto's disease, hidradenitis suppurativa, idiopathic thrombocytopenic purpura, interstitial cystitis, irritable bowel syndrome, lupus erythematosus, morphea, multiple sclerosis, myasthenia gravis, narcolepsy, neuromyotonia, Parkinson's disease, pemphigus vulgaris, pernicious anaemia, polymyositis, primary biliary cirrhosis, psoriasis, psoriatic arthritis, rheumatoid arthritis, schizophrenia, septic shock, scleroderma, Sjogren's disease, systemic lupus erythematosus (and associated glomerulonephritis), temporal arteritis, tissue graft rejection and hyperacute rejection of transplanted organs, vasculitis (ANCA-associated and other vasculitides), vitiligo, and wegener's granulomatosis.

It should be understood that the invention is not limited to the particular embodiments shown and described herein, but that various changes and modifications may be made without departing from the spirit and scope of the invention as defined by the claims.

The compounds according to the present invention may be synthesized according to a variety of reaction schemes. Necessary starting materials may be obtained by standard procedures of organic chemistry. The compounds and processes of the present invention will be better understood in connection with the following representative synthetic schemes and examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

The typical starting material

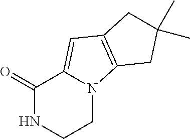

(CAS 1346674-23-4) is commercially available. However, the reported route, e.g. in WO 2013067274, to this intermediate entails at least 7 synthetic steps. The synthesis not only is long, it also includes several reagents and solvents that are toxic or hazardous and present environmental liabilities. We describe herein in Scheme 1, a new more efficient, and cost-effective route (three synthetic steps) focused on the use of sustainable chemistry:

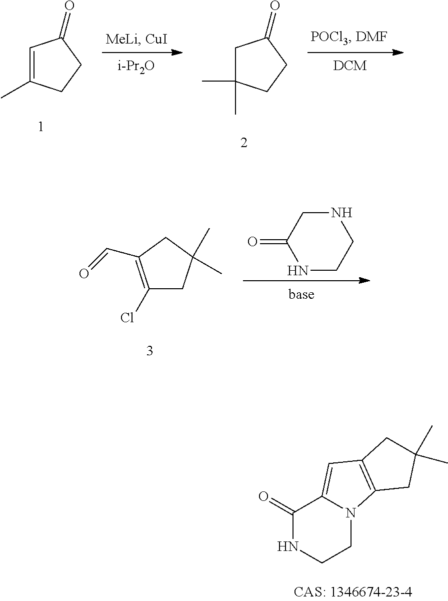

In Scheme 1, the starting material 3-methylcyclopent-2-en-1-one is converted to 3,3-dimethylcyclopentan-1-one by standard organic reaction with high yield, which can further be converted to intermediate 3. Finally, intermediate 3 can react with piperazin-2-one to yield the target molecule of CAS 1346674-23-4.

Also, the target compounds can be synthesized by alternative methods but not limited to the above procedures.

The intermediate

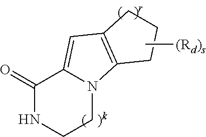

in which each of k, r, and s, independently, is 0, 1, 2, or 3, can be made by the method similar to Scheme 1, by using different starting material and reagents.

The intermediate

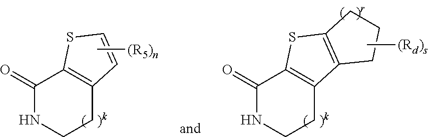

in which each of k, r, and s, independently, is 0, 1, 2, or 3, can be made by the method similar to those disclosure in the WO/2013/067260, WO/2013/067274, WO/2013/067277, WO/2015/000949.

The intermediate

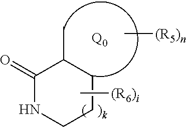

can be made by the method similar to Scheme 1, by using different starting material and reagents, or by the standard organic reactions.

The intermediate

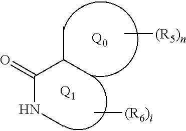

can be made by the method similar to Scheme 1 by using different starting material and reagents, or by the standard organic reactions.

The intermediate

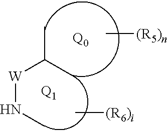

in which W is C(O) or S(O2) can be made by the method similar to Scheme 1 by using different starting material and reagents, or by the standard organic reactions.

The intermediate

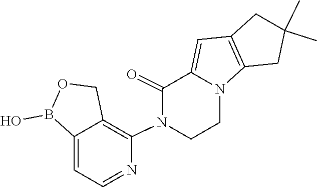 [SMILES: CC1(C)Cc2cc3n(c2C1)CCN(c1nccc2c1COB2O)C3=O]

can be made by the Scheme 2 described below.

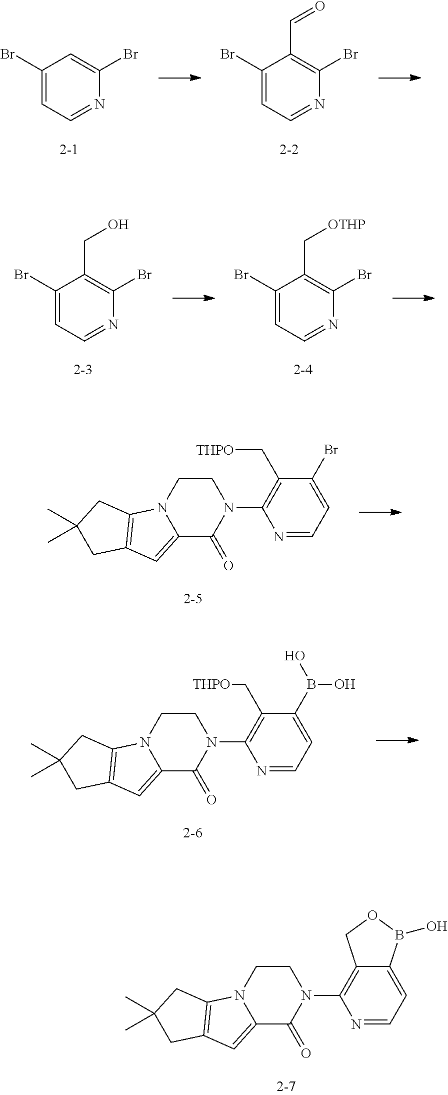

In Scheme 2, the starting material 2,4-dibromopyridine is converted to 2,4-dibromonicotinaldehyde by standard organic reaction with high yield, which can further be reduced to the alcohol intermediate 2-3. After that, the OH group of intermediate 2-3 can be protected by the THP to form the intermediate 2-4, which can react with 7,7-dimethyl-3,4,7,8-tetrahydro-2H-cyclopenta[4,5]pyrrolo[1,2-a]pyrazin-1(6H)-one (CAS 1346674-23-4) to afford the intermediate 2-5. Next, intermediate 2-5 can be converted to the intermediate 2-6, which can undergo a ring closure reaction to yield the intermediate 2-7.

Also, the target compounds can be synthesized by alternative methods but not limited to the above procedures.

The intermediate

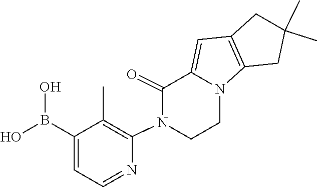 [SMILES: Cc1c(B(O)O)ccnc1N1CCn2c(cc3c2CC(C)(C)C3)C1=O]

can be made by the Scheme 3 described below.

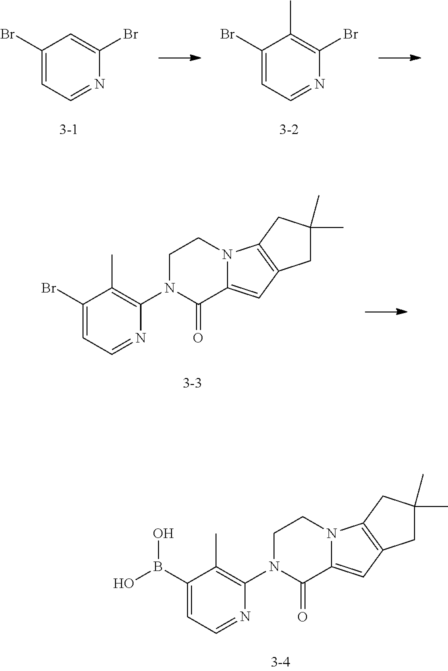

In Scheme 3, the starting material 2,4-dibromopyridine is converted to intermediate 3-2 by standard organic reaction with high yield, which can further react with 7,7-dimethyl-3,4,7,8-tetrahydro-2H-cyclopenta[4,5]pyrrolo[1,2-a]pyrazin-1(6H)-one (CAS 1346674-23-4) to afford the intermediate 3-3. Finally, 3-3 can be converted to target borate intermediate 3-4 by standard organic reaction.

Also, the target compounds can be synthesized by alternative methods but not limited to the above procedures.

The intermediate

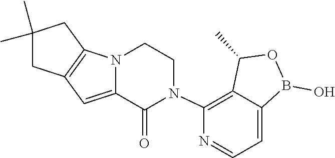 [SMILES: C[C@@H]1OB(O)c2ccnc(N3CCn4c(cc5c4CC(C)(C)C5)C3=O)c21]

can be made by the Scheme 4 described below.

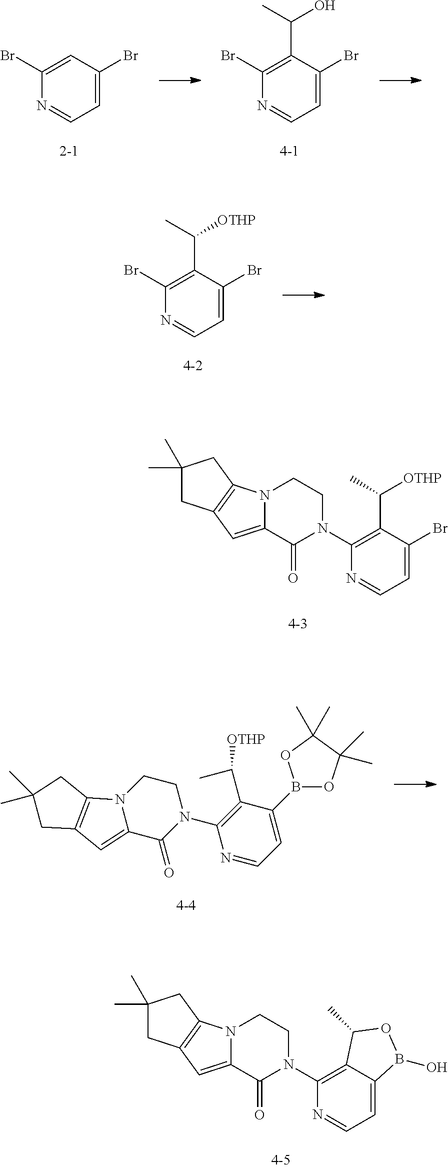

In Scheme 4, the starting material 2,4-dibromopyridine is converted to intermediate 4-1 by standard organic reaction, which can be protected by the THP and further under chiral separation to afford the intermediate 4-2. Next, intermediate 4-2 can react with 7,7-dimethyl-3,4,7,8-tetrahydro-2H-cyclopenta[4,5]pyrrolo[1,2-a]pyrazin-1(6H)-one (CAS 1346674-23-4) to give the intermediate 4-3. Finally, intermediate 4-3 can be converted to the intermediate 4-4, which can undergoes a ring closure reaction to yield the intermediate 4-5.

Also, the target compounds can be synthesized by alternative methods but not limited to the above procedures.

The intermediate

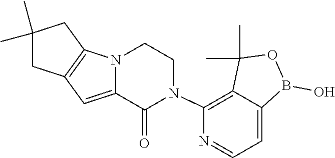 [SMILES: CC1(C)Cc2cc3n(c2C1)CCN(c1nccc2c1C(C)(C)OB2O)C3=O]

can be made by the Scheme 5 described below.

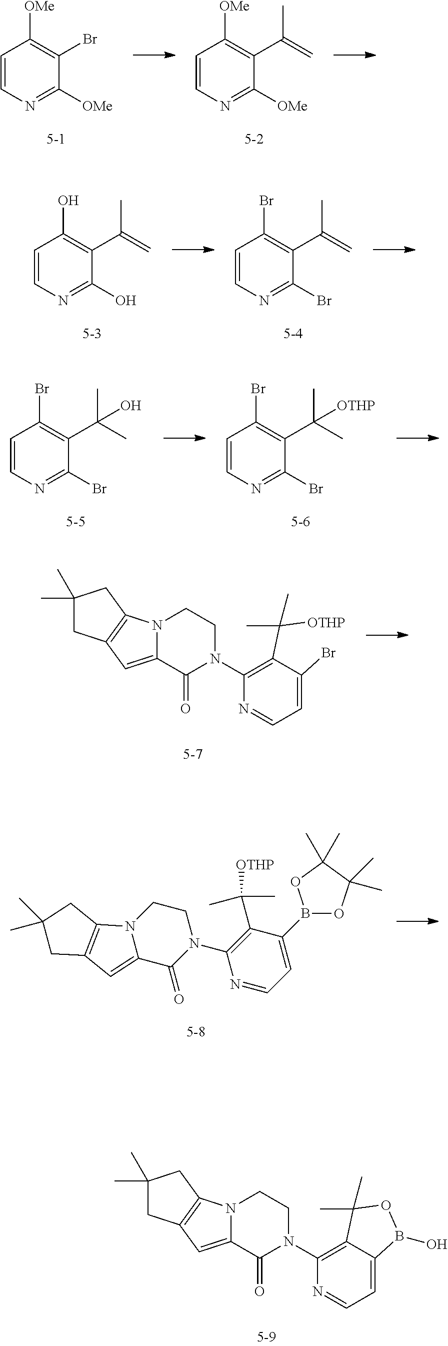

In Scheme 5, the starting material 5-1 can be prepared by conventional procedures using appropriate compounds and reagents. The starting material 5-1 can be converted to intermediate 5-2 through Suzuki coupling reaction. After that, the intermediate 5-2 is deprotected to give intermediate 5-3, which can be converted to intermediate 5-4 through a conventional reaction. The intermediate 5-4 is converted to intermediate 5-5 by standard organic reaction, which can be protected by the THP to afford 5-6. Next, intermediate 5-6 can react with 7,7-dimethyl-3,4,7,8-tetrahydro-2H-cyclopenta[4,5]pyrrolo[1,2-a]pyrazin-1(6H)-one (CAS 1346674-23-4) to afford the intermediate 5-7. Finally, intermediate 5-8 is prepared from the intermediate 5-7, and the intermediate 5-8 can undergo a ring closure reaction to yield the intermediate 5-9.

Also, the target compounds can be synthesized by alternative methods but not limited to the above procedures.

The intermediate

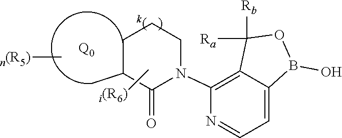

can be made by the method similar to Scheme 1-5, by using different starting material and reagents, or by the standard organic reactions.

The intermediate

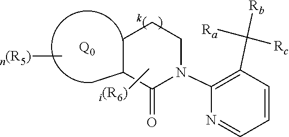

can be made by the method similar to Scheme 1-5, by using different starting material and reagents, or by the standard organic reactions.

The intermediate

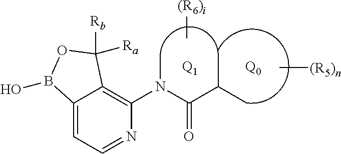

can be made by the method similar to Scheme 1-5 by using appropriate starting material and reagents, or by the standard organic reactions.

The intermediate

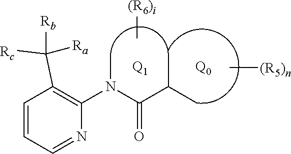

can be made by the method similar to Scheme 1-5 by using appropriate starting material and reagents, or by the standard organic reactions.

An approach to synthesize compounds of

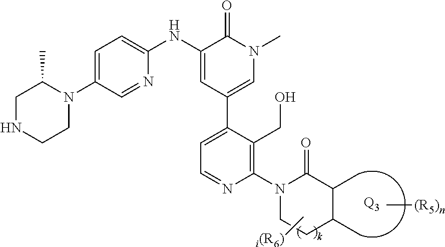

is described in Scheme 6-1.

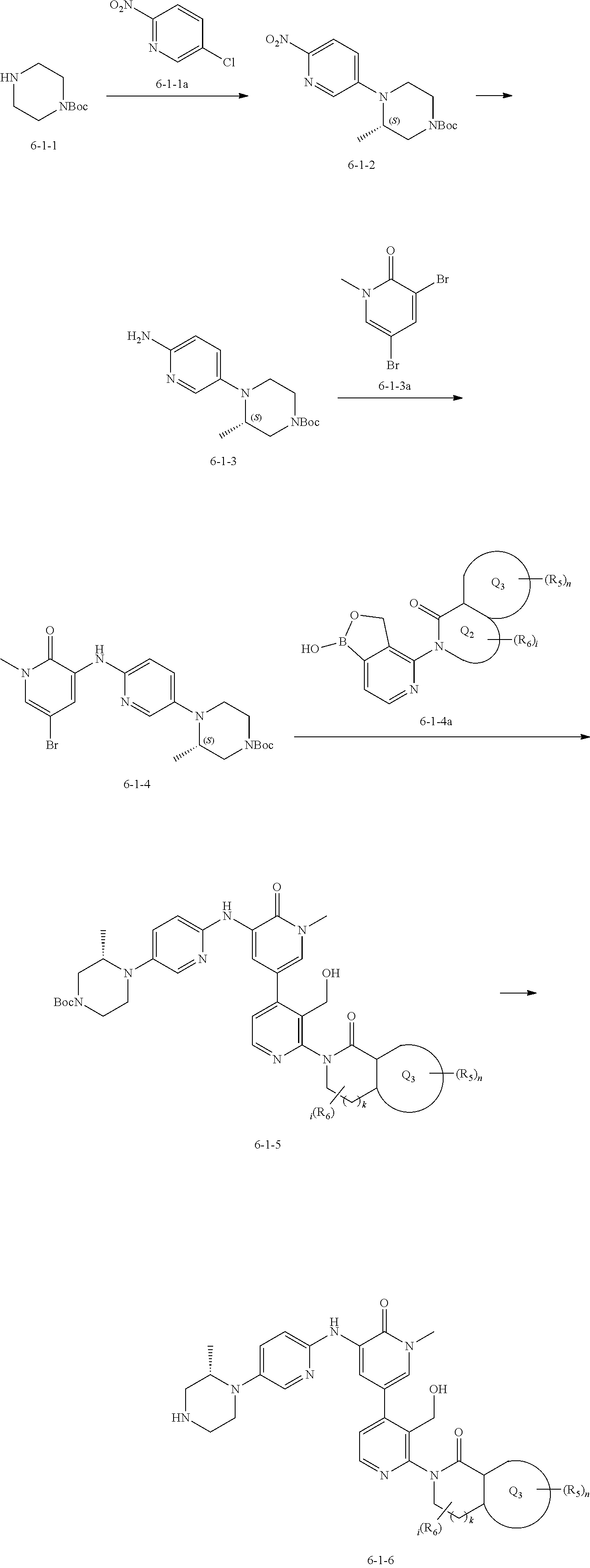

In Scheme 6-1, the starting material 6-1-1 can react with 6-1-1a to afford 6-1-2, which is reduced to give analine 6-1-3. The intermediate 6-1-3 can couple with 6-1-3a to yield 6-1-4. Next, the intermediate 6-1-4 can go through a Suzuki coupling with 6-1-4a to give 6-1-5, which is deprotected to afford the intermediate 6-1-6.

Also, the target compounds can be synthesized by alternative methods but not limited to the above procedures.

An approach to synthesize compounds of

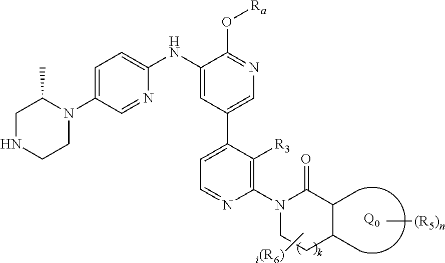

is described in Scheme 6-2.

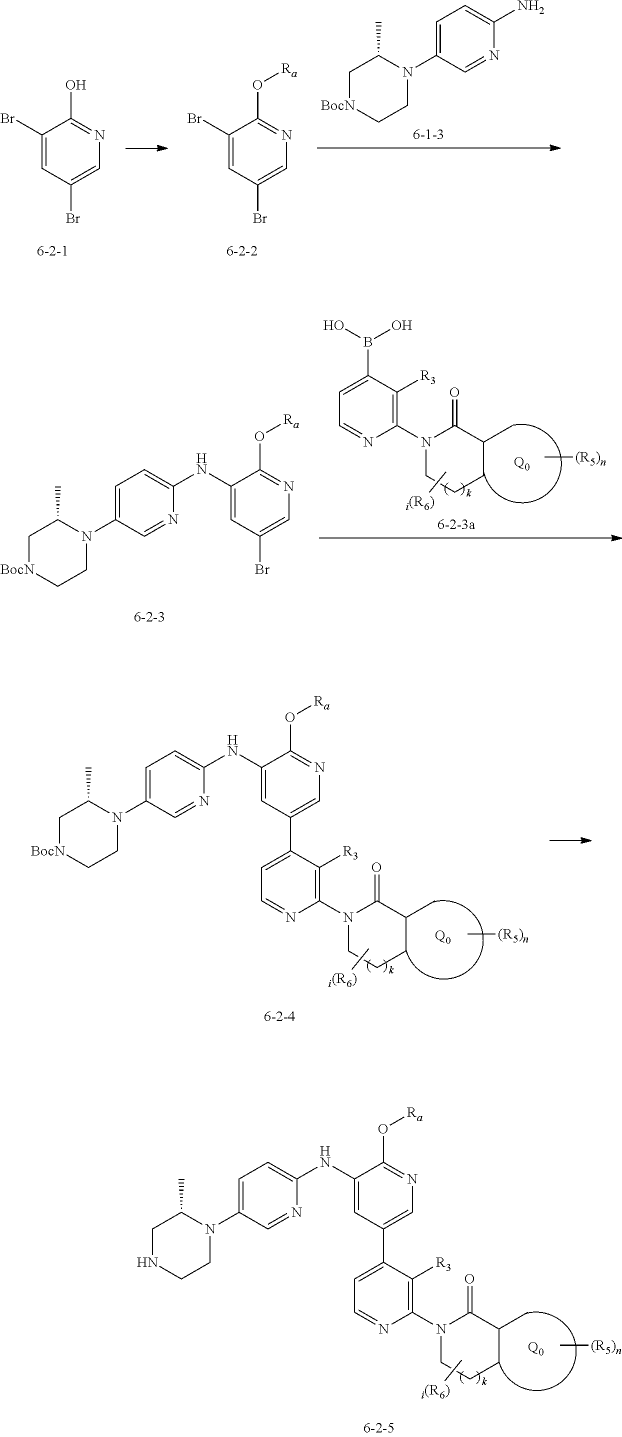

In Scheme 6-2, the starting material 6-2-1 can be converted to 6-2-2 readily, which can undergo a coupling reaction with compound 6-1-3 to generate intermediate 6-2-3. Next, 6-2-3 undergoes a Suzuki coupling reaction with 6-2-3a to give the intermediate 6-2-4, which can be deprotected to afford the intermediate 6-2-5.

Also, the target compounds can be synthesized by alternative methods but not limited to the above procedures.

The intermediate

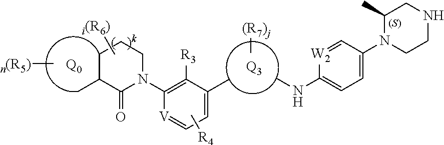

can be made by the method similar to Scheme 6-1 and 6-2 by using appropriate starting material and reagents, or by the standard organic reactions.

The intermediate

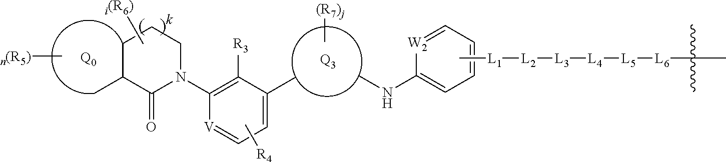

can be made by the method similar to Scheme 6-1 and 6-2 by using appropriate starting material and reagents, or by the standard organic reactions.

An approach to synthesize compounds of

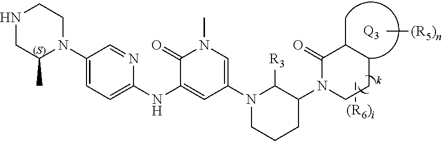

is described in Scheme 6-3.

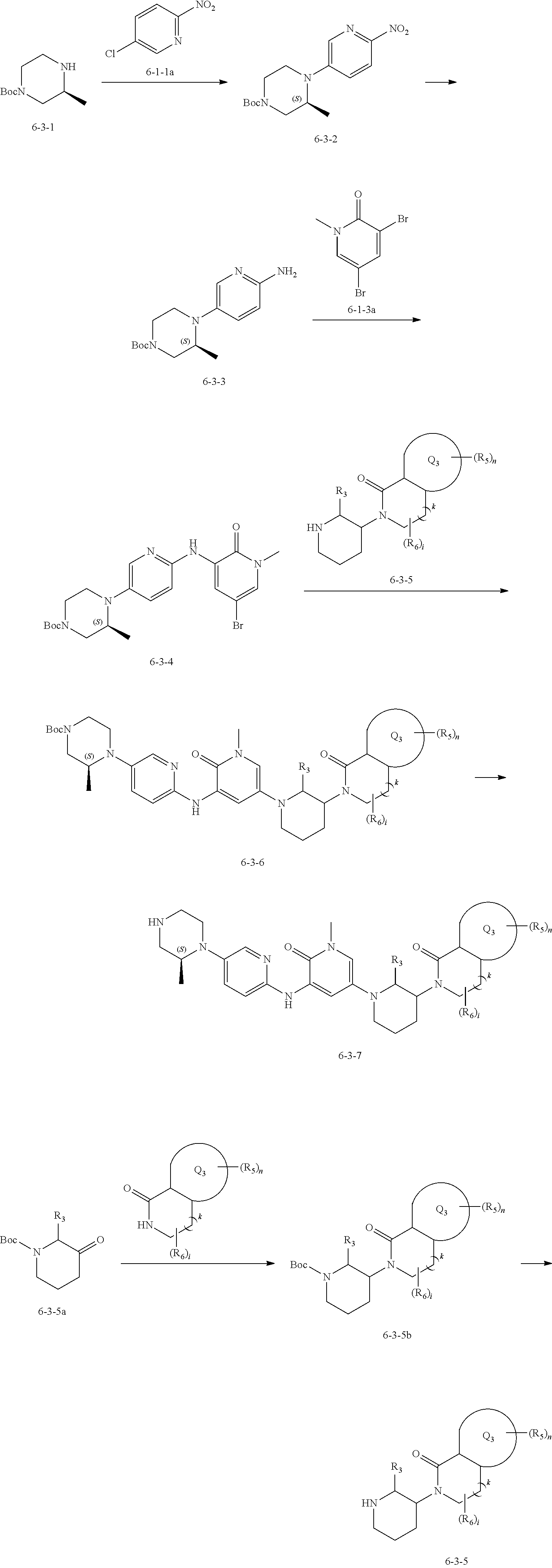

In Scheme 6-3, the starting material 6-3-1 can react with 6-1-1a to afford intermediate 6-3-2, which is reduced to give analine 6-3-3. Next, intermediate 6-3-3 can couple with 6-1-3a to yield 6-3-4. The intermediate 6-3-5 can be prepared through a 2-step sequence of conventional reactions from 6-3-5a. Finally, 6-3-4 can go through a coupling reaction with 6-3-5 to give 6-3-6, which is deprotected to afford intermediate 6-3-7.

Also, the target compounds can be synthesized by alternative methods but not limited to the above procedures.

The intermediate

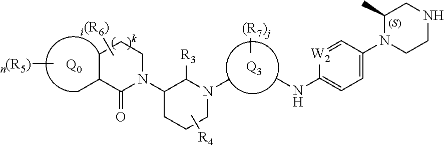

can be made by the method similar to Scheme 6-2 by using appropriate starting material and reagents, or by the standard organic reactions.

The intermediate

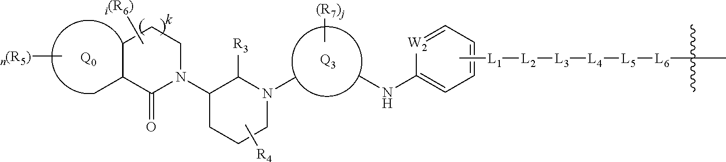

can be made by the method similar to Scheme 6-2 by using appropriate starting material and reagents, or by the standard organic reactions.

Also, the target compounds can be synthesized by alternative methods but not limited to the above procedures.

The intermediate

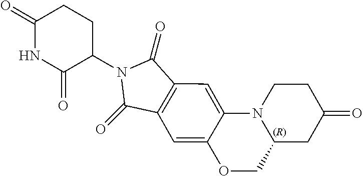

can be made by the Scheme 7 described below.

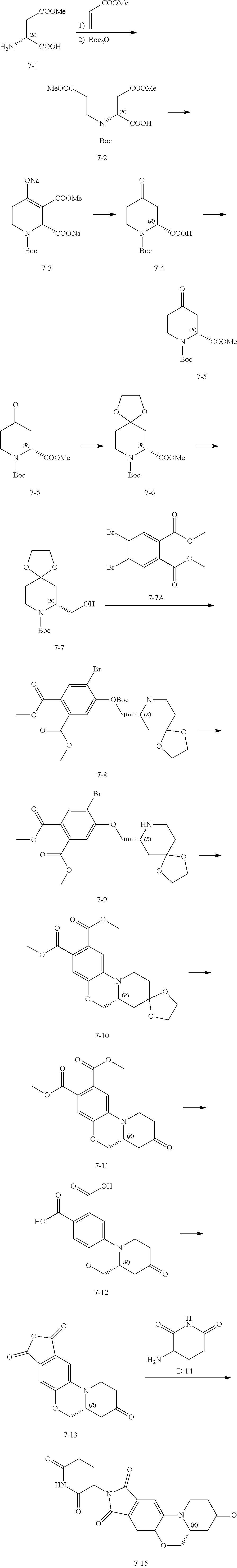

In Scheme 7, the starting material 7-1 can go through a conventional reaction to afford 7-2. The intermediate 7-2 can be converted to 7-3 through an intramolecular cycliztion, which is decarboxylated to afford 7-4. The intermediate 7-4 can be converted to 7-5 by a standard organic reaction, which is protected to give 7-6. After that, the intermediate 7-6 can be reduced to afford the intermediate 7-7. And 7-7 can undergo a coupling reaction with 7-7A to afford the intermediate 7-8. The intermediate 7-8 is deprotected to afford 7-9, which can be further converted to 7-10 through an intramolecular coupling reaction. Next, the intermediate 7-10 can be converted to 7-12 through a 2-step sequence of deprotection and hydrolysis reaction. Finally, 7-12 can be converted to the in termediate 7-13 readily, which can react with 7-14 to yield the intermediate 7-15.

Also, the target compounds can be synthesized by alternative methods but not limited to the above procedures.

The intermediate

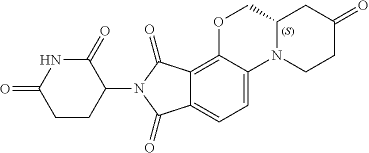

can be made by the Scheme 8 described below.

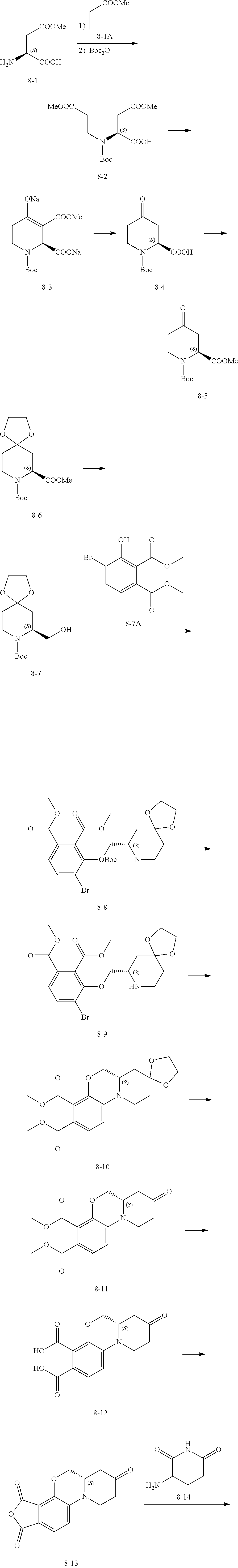

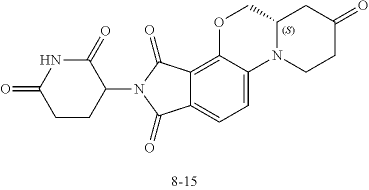

In Scheme 8, the starting material 8-1 can go through a conventional reaction to afford 8-2. The intermediate 8-2 can be converted to 8-3 through an intramolecular cycliztion, which is decarboxylated to afford 8-4. The intermediate 8-4 can be converted to 8-5 by a standard organic reaction, which is protected to give 8-6. After that, the intermediate 8-6 can be reduced to afford the intermediate 8-7. 8-7 can undergo a coupling reaction with 8-7A to afford the intermediate 8-8. The intermediate 8-8 is deprotected to afford 8-9, which can be further converted to 8-10 through an intramolecular coupling reaction. Next, the intermediate 8-10 can be converted to 8-12 through a 2-step sequence of deprotection and hydrolysis reaction. Finally, 8-12 can be converted to the in termediate 8-13 readily, which can react with 8-14 to yield the intermediate 8-15.

Also, the target compounds can be synthesized by alternative methods but not limited to the above procedures.

The intermediate

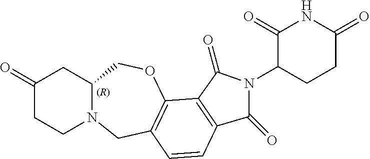

can be made by the Scheme 9 described below.

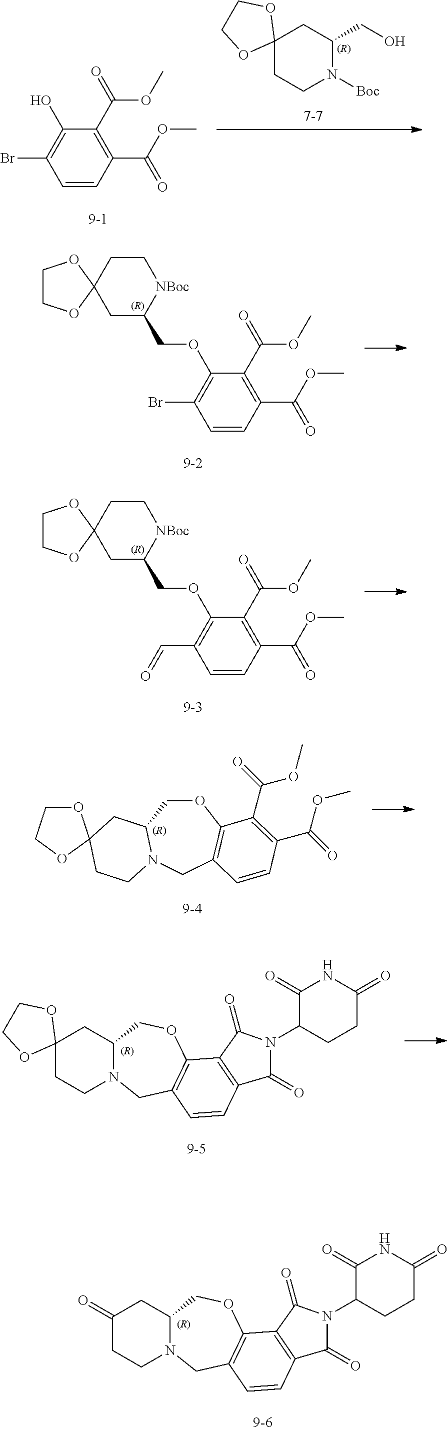

In Scheme 9, the starting material 9-1 can be prepared by conventional procedures using appropriate compounds and reagents. The starting material 9-1 can be converted to intermediate 9-2 readily, which is converted to 9-3 through a literate known condition. After that, the intermediate 9-3 is converted to 9-4 via a sequence of deprotection and reductive amination reaction. Next, the intermediate 9-5 is prepared from 9-4 through a convertional reaction. Finally, intermediate 9-5 is deprotected to give the target compounds 9-6.

Also, the target compounds can be synthesized by alternative methods but not limited to the above procedures.

The intermediate

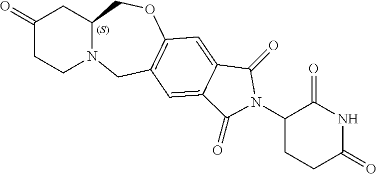

can be made by the Scheme 10 described below.

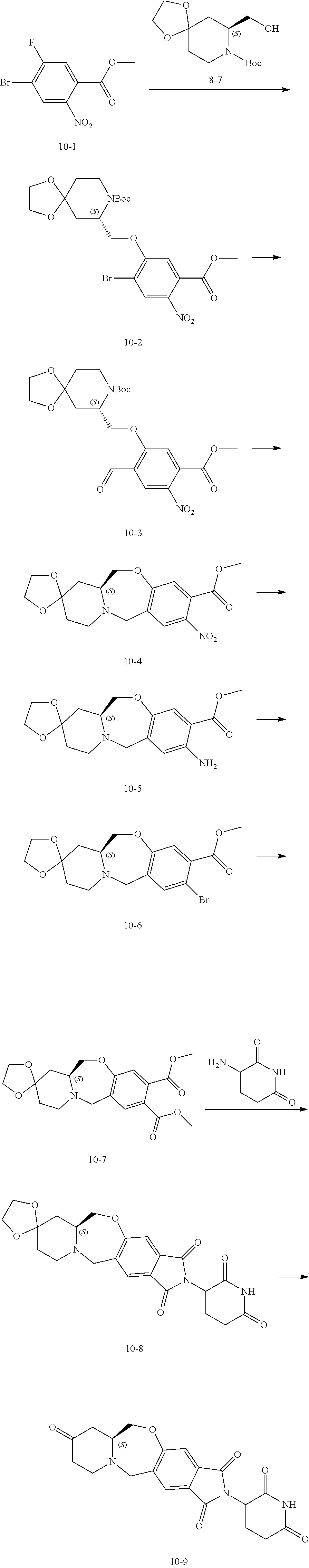

In Scheme 10, the starting material 10-1 can be prepared by conventional procedures using appropriate compounds and reagents. The starting material 10-1 can be converted to intermediate 10-2 via a SN_{Ar} reaction, which can be converted to 10-3 through a literate known condition. After that, the intermediate 10-3 is converted to 10-4 through a 2-step sequence of deprotection and reductive amination reaction. Next, the intermediate 10-4 is reduced to give 10-5, which is converted to 10-7 though a 2-step sequence conventional reaction. Finally, intermediate 10-8 can be prepared from 10-7 readily, which is deprotected to give the target compounds 10-9.

Also, the target compounds can be synthesized by alternative methods but not limited to the above procedures.

The intermediate

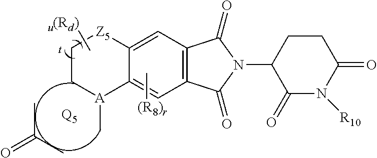

can be made by the method similar to Scheme 7-10 by using appropriate starting material and reagents, or by the standard organic reactions.

The intermediate

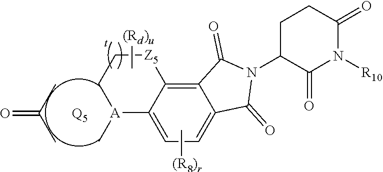

can be made by the method similar to Scheme 7-10 by using appropriate starting material and reagents, or by the standard organic reactions.

The intermediate

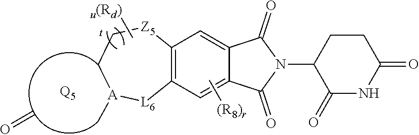

can be made by the method similar to Scheme 7-10 by using appropriate starting material and reagents, or by the standard organic reactions.

The intermediate

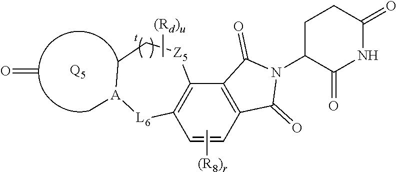

can be made by the method similar to Scheme 7-10 by using appropriate starting material and reagents, or by the standard organic reactions.

The intermediate

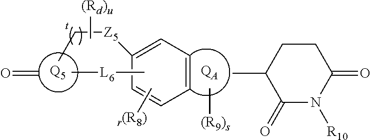

can be made by the method similar to Scheme 7-10 by using appropriate starting material and reagents, or by the standard organic reactions.

The intermediate

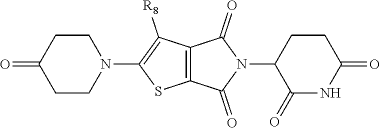

can be made by the Scheme 11 described below.

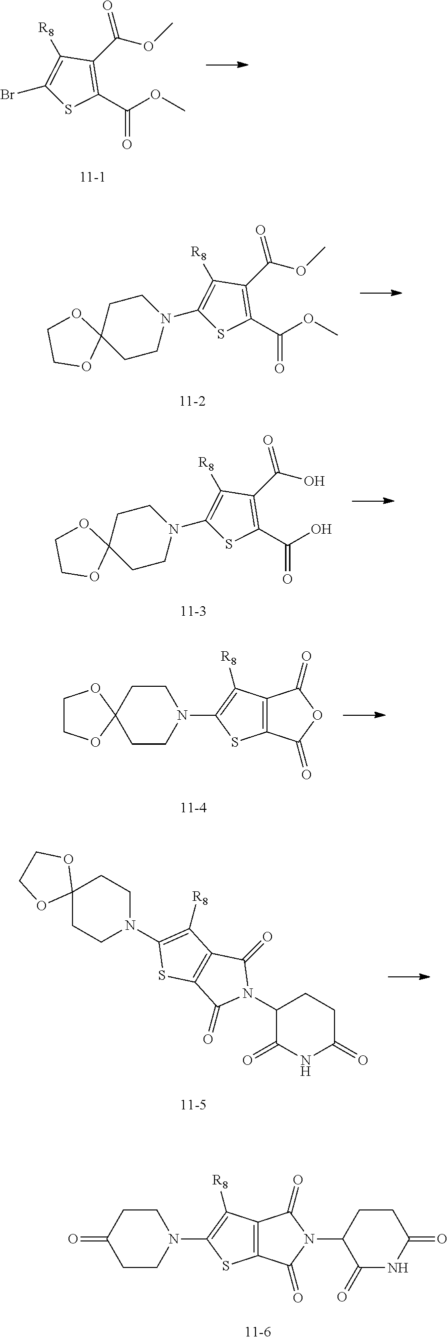

In Scheme 11, the starting material 11-1 can be prepared by conventional procedures using appropriate compounds and reagents. The starting material 11-1 can be converted to 11-2 through a coupling reaction, which can be hydrolyzed into di-carboxylic acid 11-3 readily. Next, 11-3 is dehydrated to give the acid anhydride 11-4, which can be converted to 11-5. Finally, the intermediate 11-5 is deprotected to give the target compounds 11-6.

Also, the target compounds can be synthesized by alternative methods but not limited to the above procedures.

An approach to synthesize compounds of

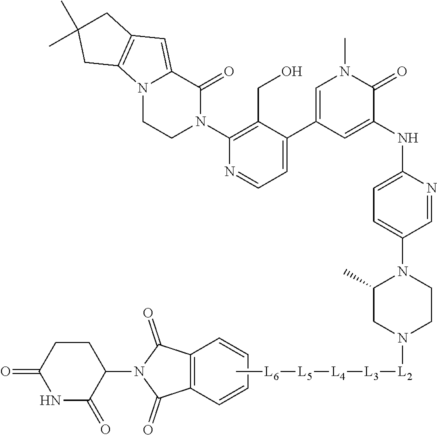

is described in Scheme A. L₂, L₃, L₄, L₅, and L₆ in general Scheme A are the same as those described in the Summary section above.

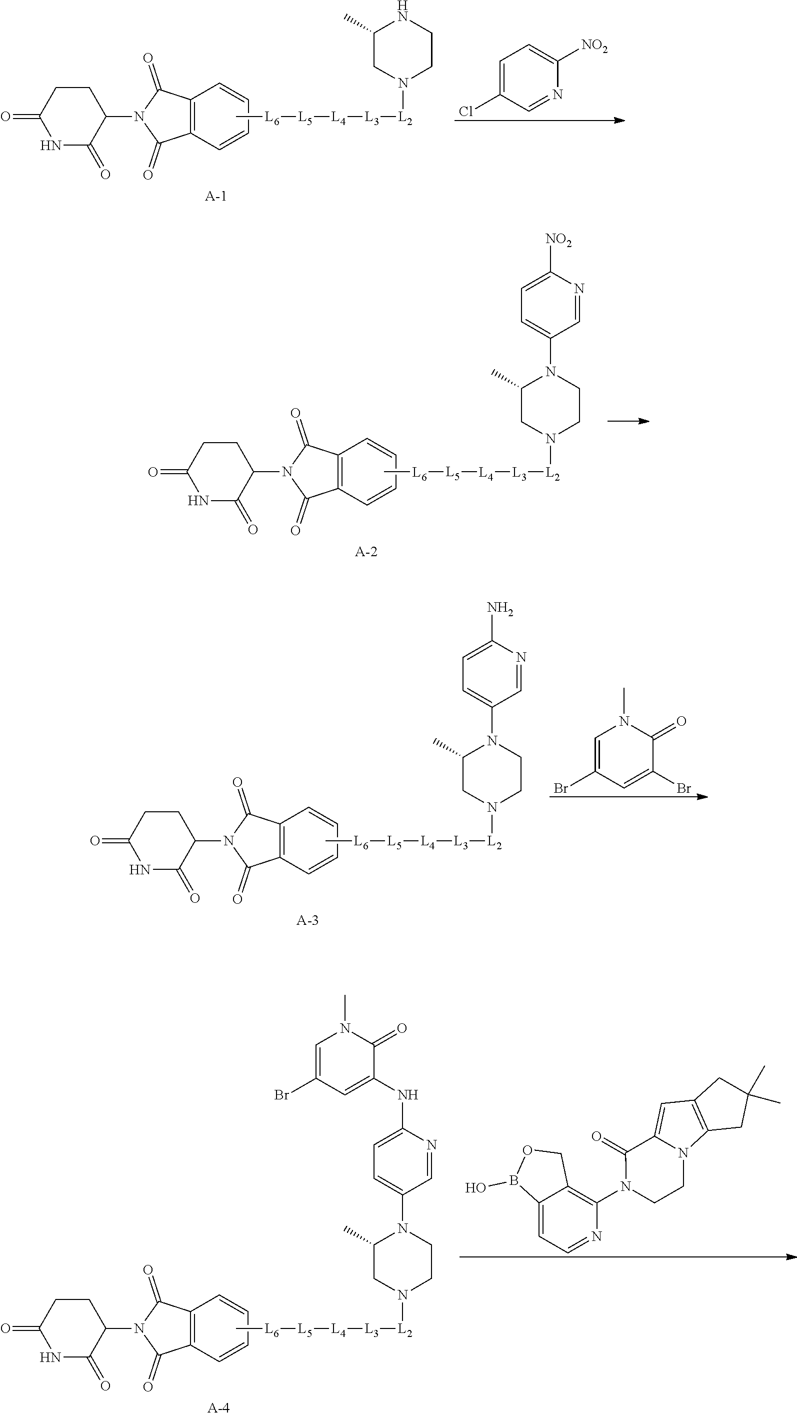

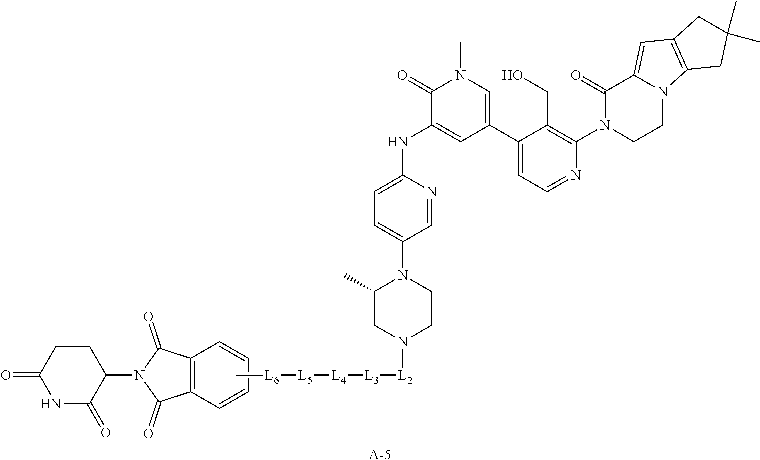

In Scheme A, the starting material A-1 can be prepared by conventional procedures using appropriate starting material and reagents. A-1 can react with 5-chloro-2-nitropyridine to form the A-2, which can be reduced to afford the intermediate A-3. A-3 can couple with 3,5-dibromo-1-methylpyridin-2(1H)-one to yield intermediate A-4, which can react with 2-(1-hydroxy-1,3-dihydro-[1,2]oxaborolo[4,3-c]pyridin-4-yl)-7,7-dimethyl-3,4,7,8-tetrahydro-2H-cyclopenta[4,5]pyrrolo[1,2-a]pyrazin-1(6H)-one to afford the target compound A-5.

Also, the target compounds can be synthesized by alternative methods but not limited to the above procedures.

An approach to synthesize compounds of

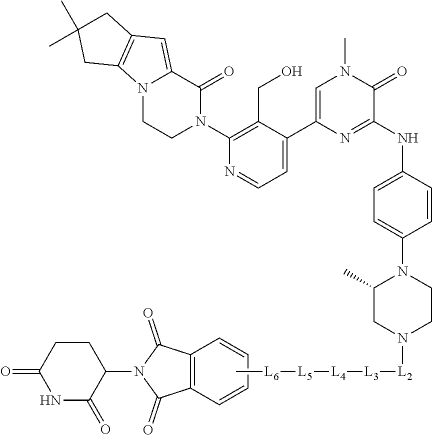

is described in Scheme B.

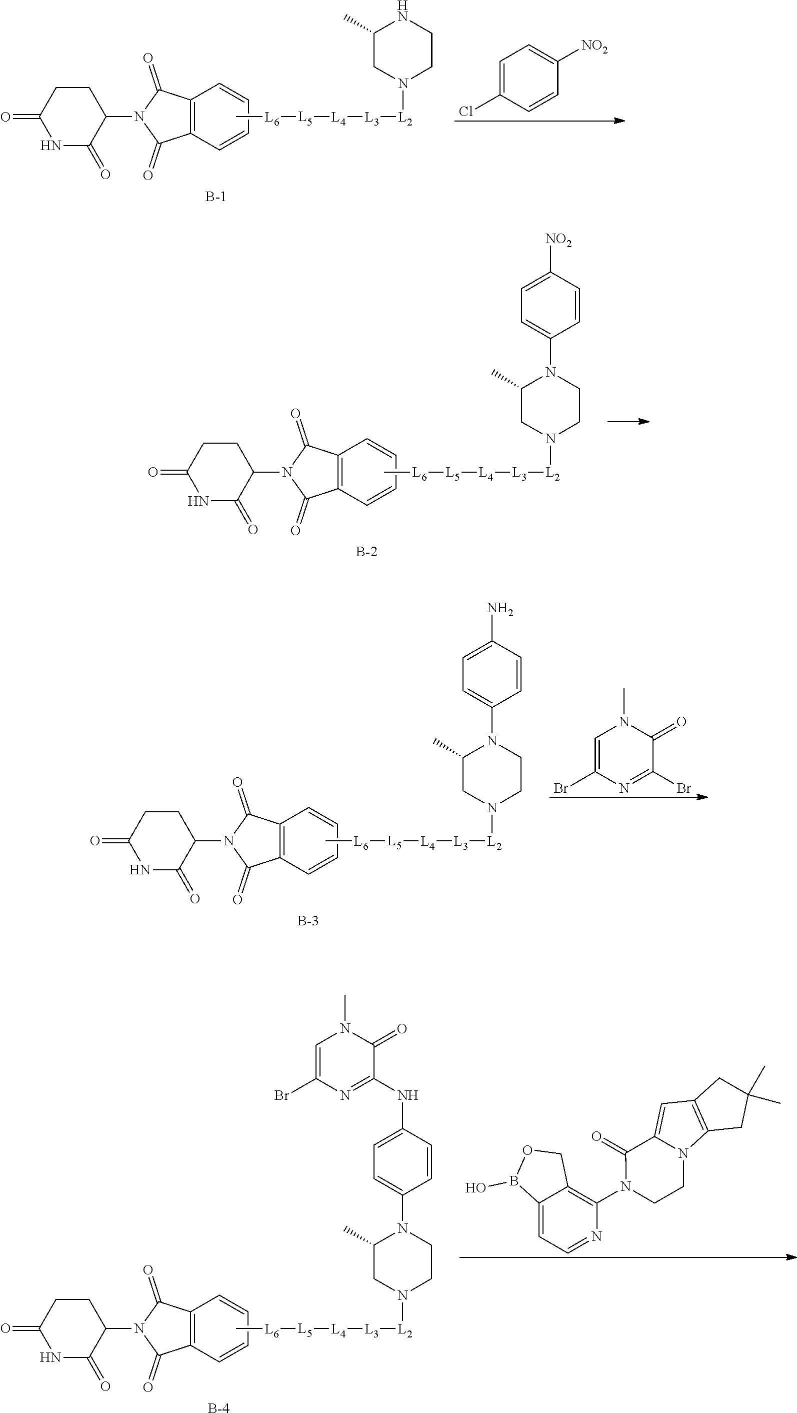

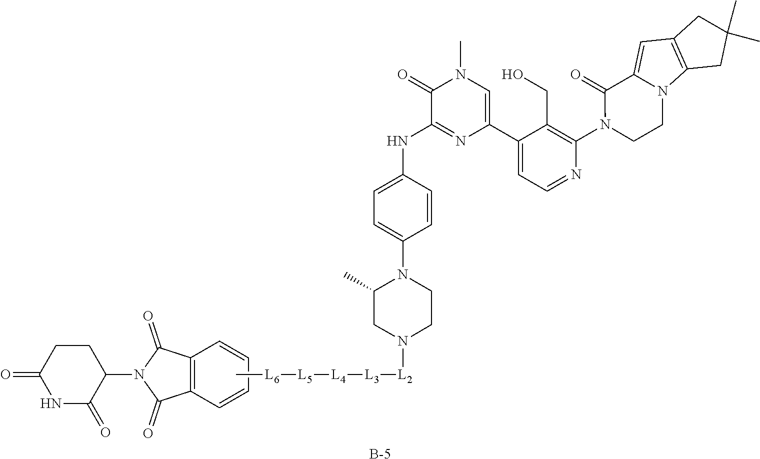

In Scheme B, the starting material B-1 can be prepared by conventional procedures using appropriate starting material and reagents. B-1 can react with 1-chloro-4-nitrobenzene to form the B-2 which can be reduced to afford the intermediate B-3. B-3 can couple with 3,5-dibromo-1-methylpyrazin-2(1H)-one to yield intermediate B-4, which can react with 2-(1-hydroxy-1,3-dihydro-[1,2]oxaborolo[4,3-c]pyridin-4-yl)-7,7-dimethyl-3,4,7,8-tetrahydro-2H-cyclopenta[4,5]pyrrolo[1,2-a]pyrazin-1(6H)-one to afford the target compound B-5.

Also, the target compounds can be synthesized by alternative methods but not limited to the above procedures.

An approach to synthesize compounds of

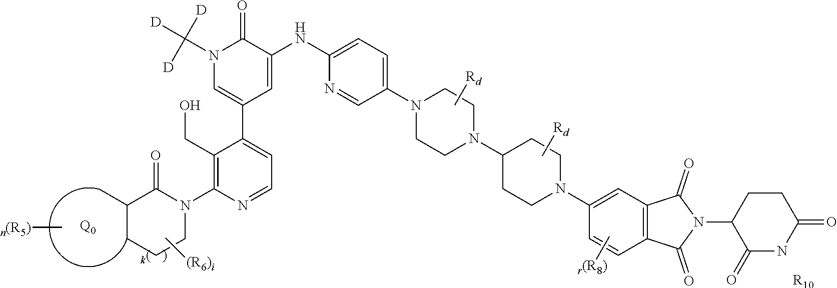

is described in Scheme B-1.

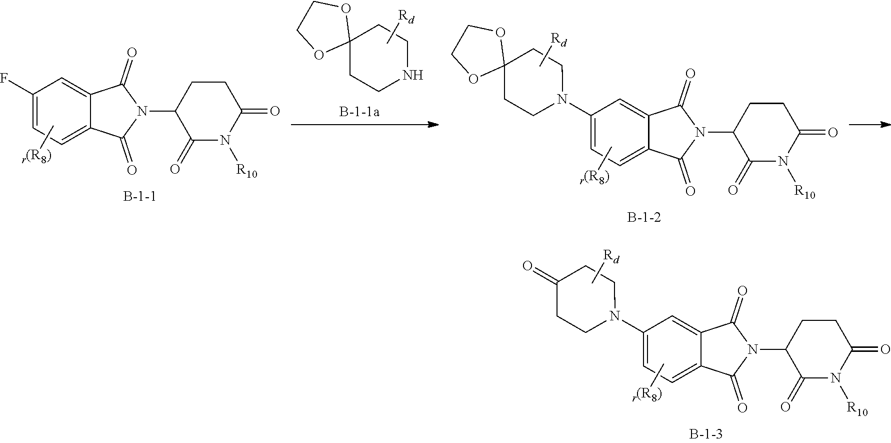

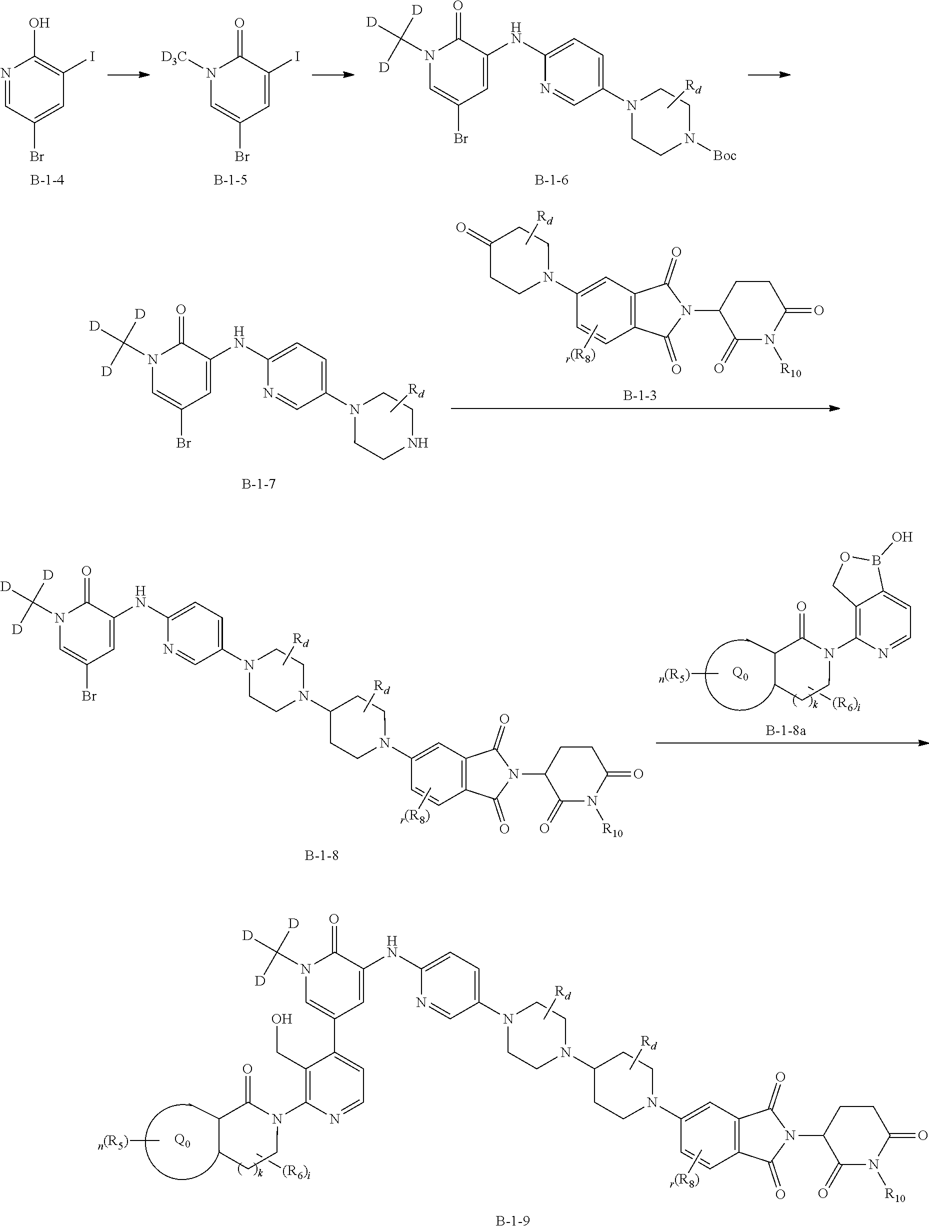

In Scheme B-1, the starting material B-1-1 can be prepared by conventional procedures using appropriate starting material and reagents. B-1-1 can react with B-1-1a to form the intermediate B-1-2, which can be deprotected to afford the intermediate B-1-3. After that, B-1-4 can be converted to B-1-5 readily, which is converted to B-1-6 through Buchwald coupling reaction. Next, the intermediate B-1-6 is deprotected to give B-1-7, which can react with B-1-3 to afford the intermediate B-1-8. Finally, the intermediate B-1-8 can couple with B-1-8a to generate the target compound B-1-9.

Also, the target compounds can be synthesized by alternative methods but not limited to the above procedures.

An approach to synthesize compounds of

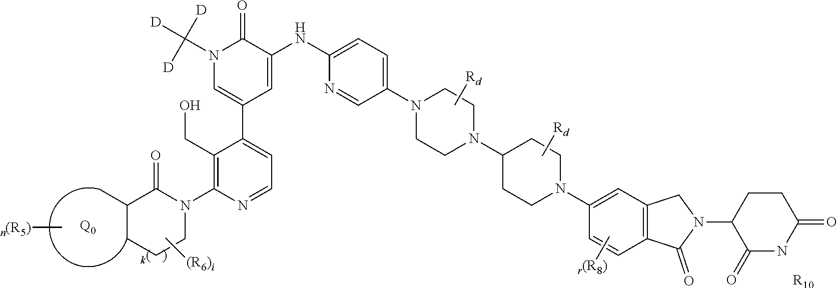

is described in Scheme B-2.

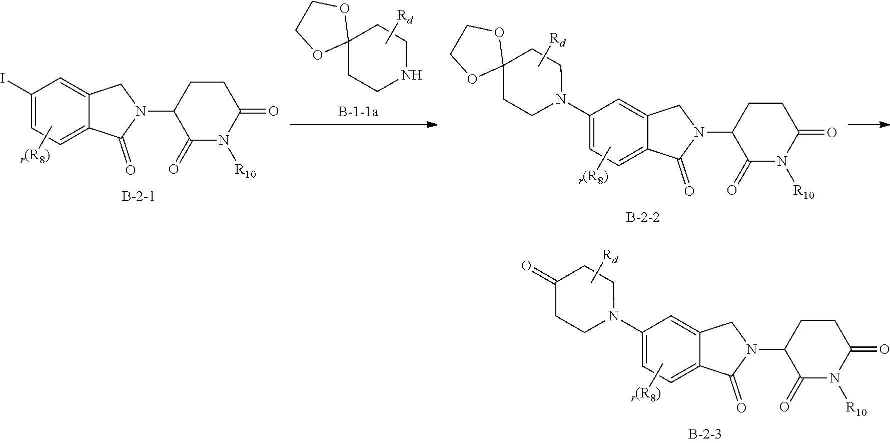

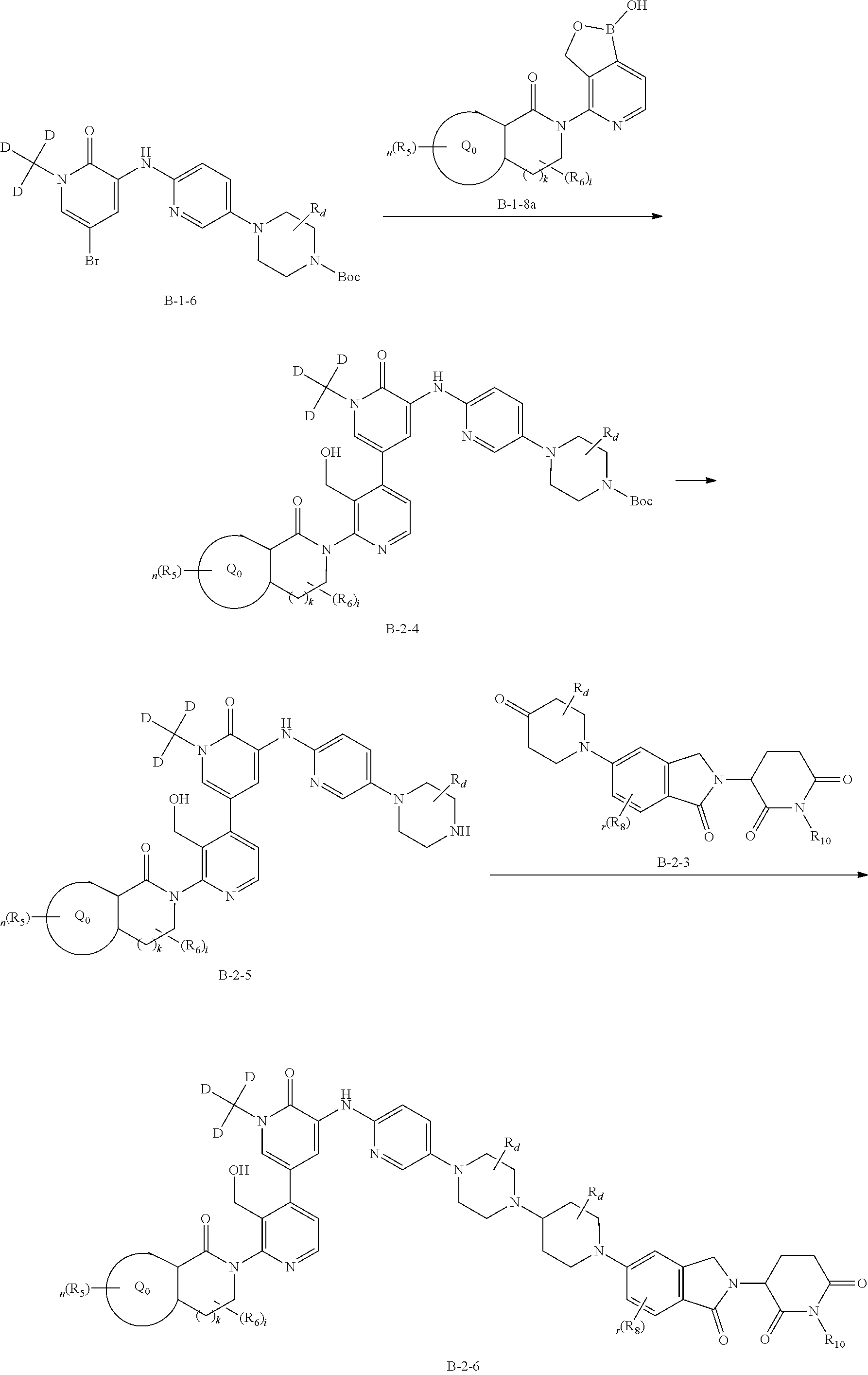

In Scheme B-2, the starting material B-2-1 can be prepared by conventional procedures using appropriate starting material and reagents. B-2-1 can react with B-1-1a to form the intermediate B-2-2, which can be deprotected to afford the intermediate B-2-3. After that, B-1-6 can couple with B-1-8a to give B-2-4 readily, which is deprotected to afford B-2-5. Finally, the intermediate B-2-5 can react with B-2-3 to generate the target compound B-2-6.

Also, the target compounds can be synthesized by alternative methods but not limited to the above procedures.

An approach to synthesize compounds of

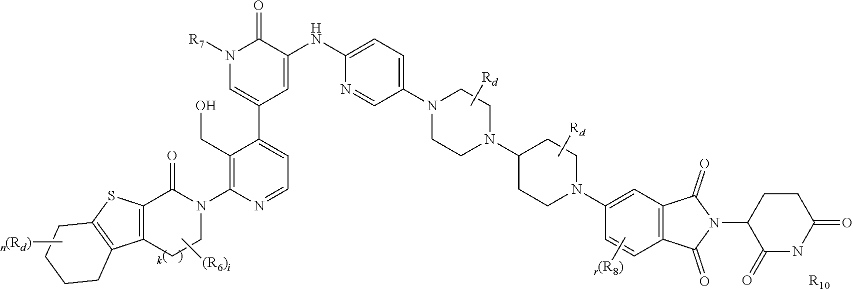

is described in Scheme B-3.

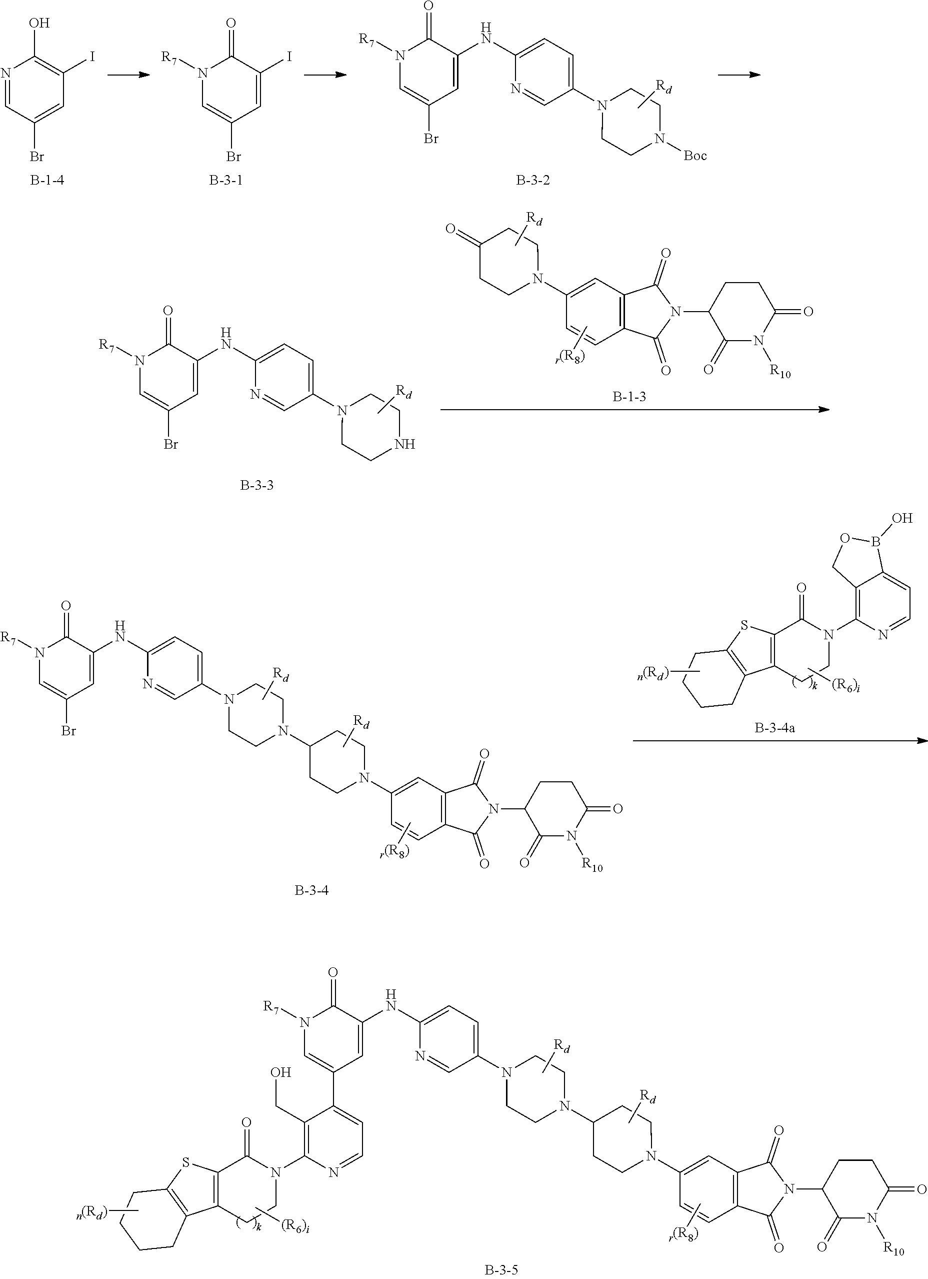

In Scheme B-3, the starting material B-1-4 can be prepared by conventional procedures using appropriate starting material and reagents. B-1-4 can be converted to B-3-1 readily, which is converted to B-3-2 through Buchwald coupling reaction. After that, B-3-2 is deprotected to give B-3-3, which can react with B-1-3 to afford the intermediate B-3-4. Finally, the intermediate B-3-4 can couple with B-3-4a to generate the target compound B-3-5.

Also, the target compounds can be synthesized by alternative methods but not limited to the above procedures.

An approach to synthesize compounds of

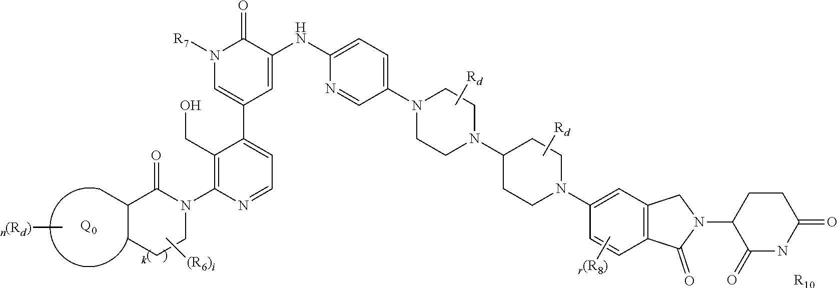

is described in Scheme B-4.

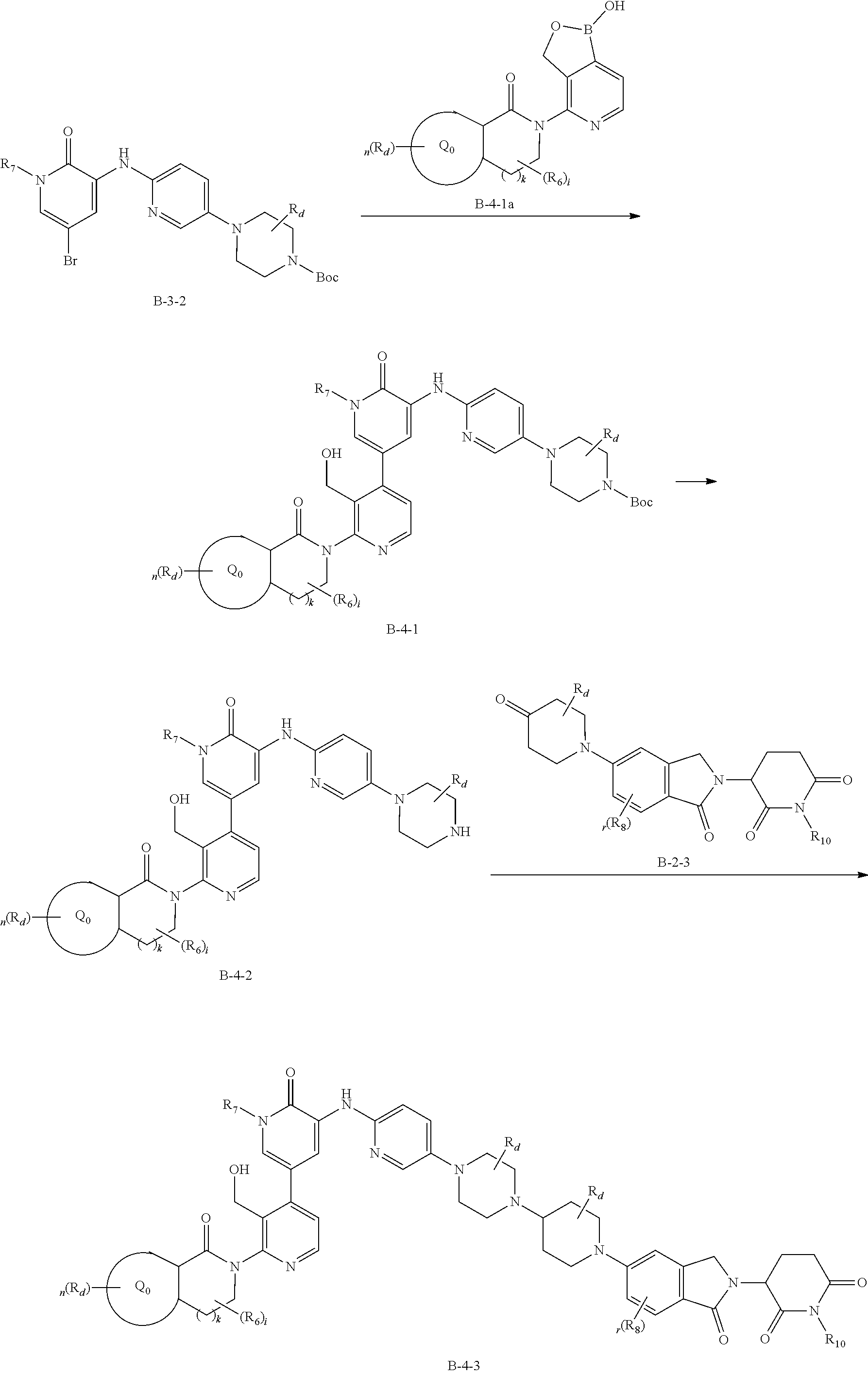

In Scheme B-4, the starting material B-3-2 can be prepared by conventional procedures using appropriate starting material and reagents. B-3-2 can couple with B-4-1a to give B-4-1, which is deprotected to afford the intermediate B-4-2. Finally, the intermediate B-4-2 can react with B-2-3 to generate the target compound B-4-3.

Also, the target compounds can be synthesized by alternative methods but not limited to the above procedures.

An approach to synthesize compounds of

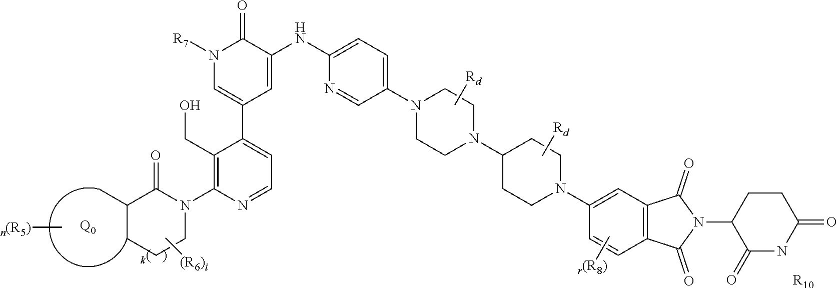

is described in Scheme B-5.

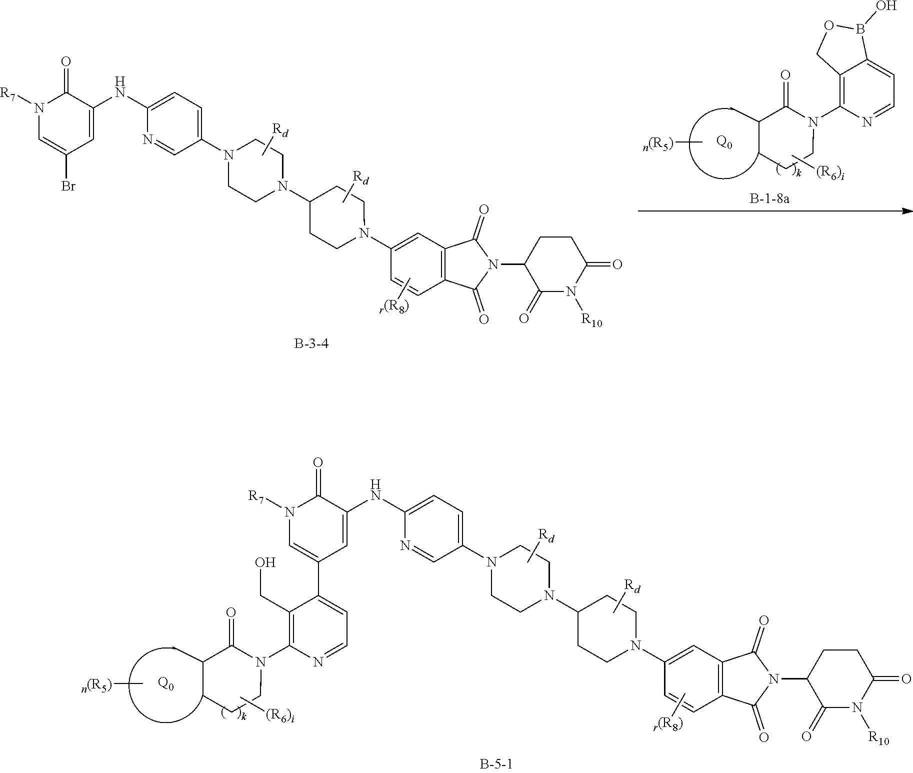

In Scheme B-5, the starting material B-3-4 can be prepared by conventional procedures using appropriate starting material and reagents. B-3-4 can couple with B-1-8a to to generate the target compound B-5-1.

Also, the target compounds can be synthesized by alternative methods but not limited to the above procedures.

An approach to synthesize compounds of

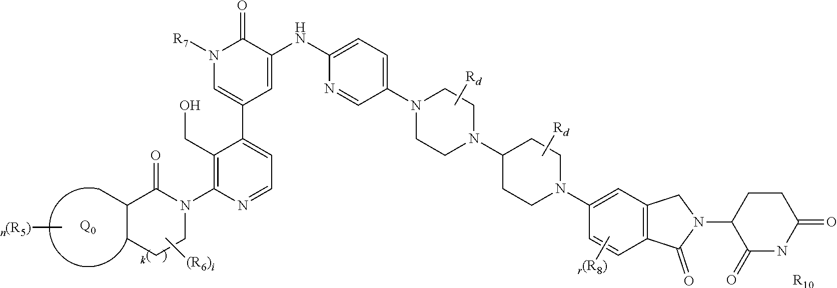

is described in Scheme B-6.

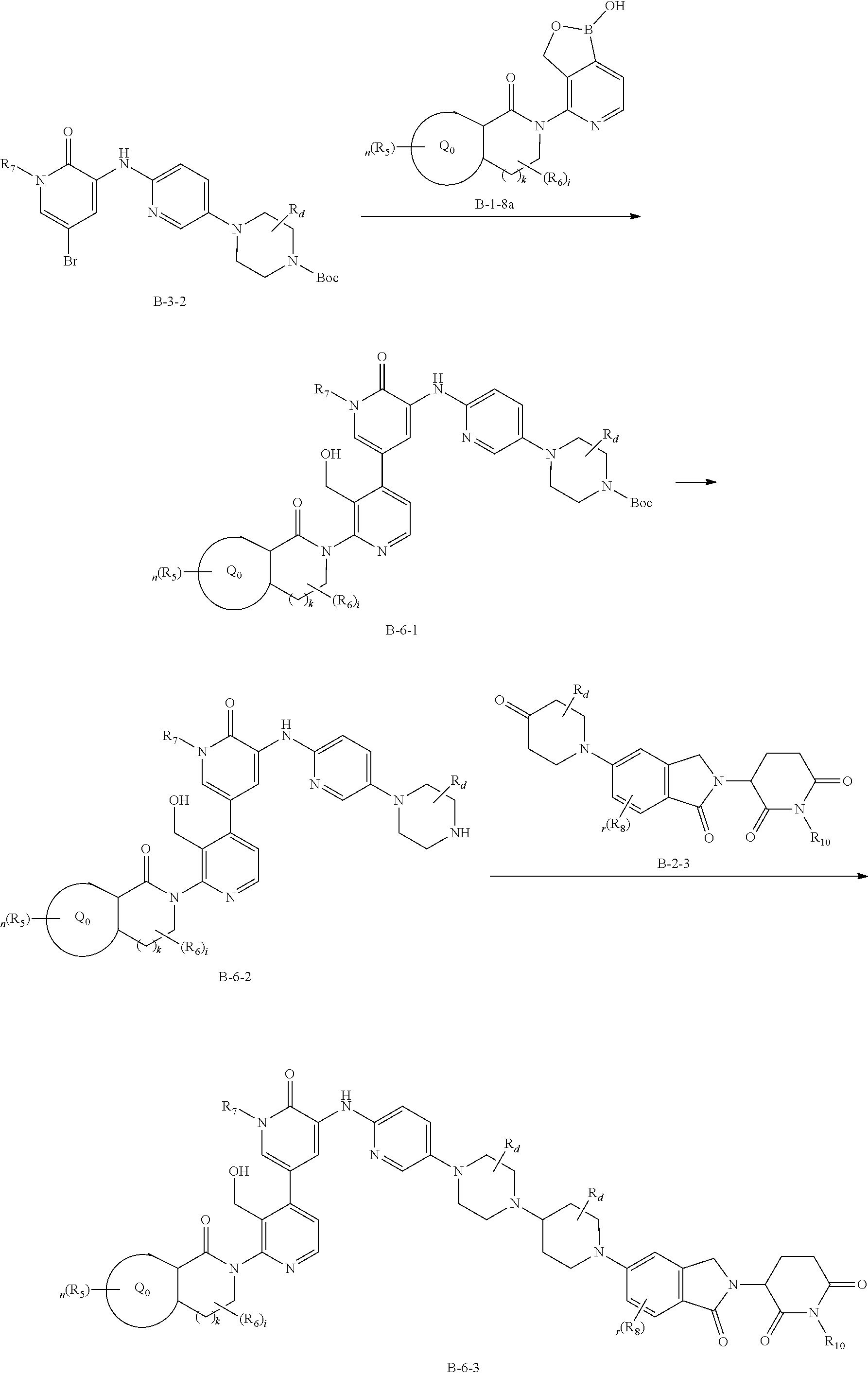

In Scheme B-6, the starting material B-3-2 can be prepared by conventional procedures using appropriate starting material and reagents. B-3-2 can couple with B-1-8a to give B-6-1, which is deprotected to afford the intermediate B-6-2. Finally, the intermediate B-6-2 can react with B-2-3 to generate the target compound B-6-3.

Also, the target compounds can be synthesized by alternative methods but not limited to the above procedures.

An approach to synthesize compounds of

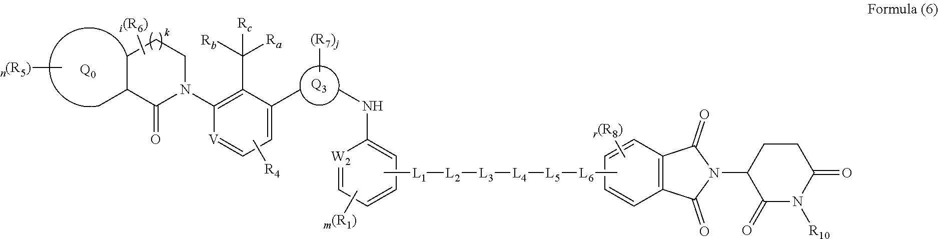

is described in

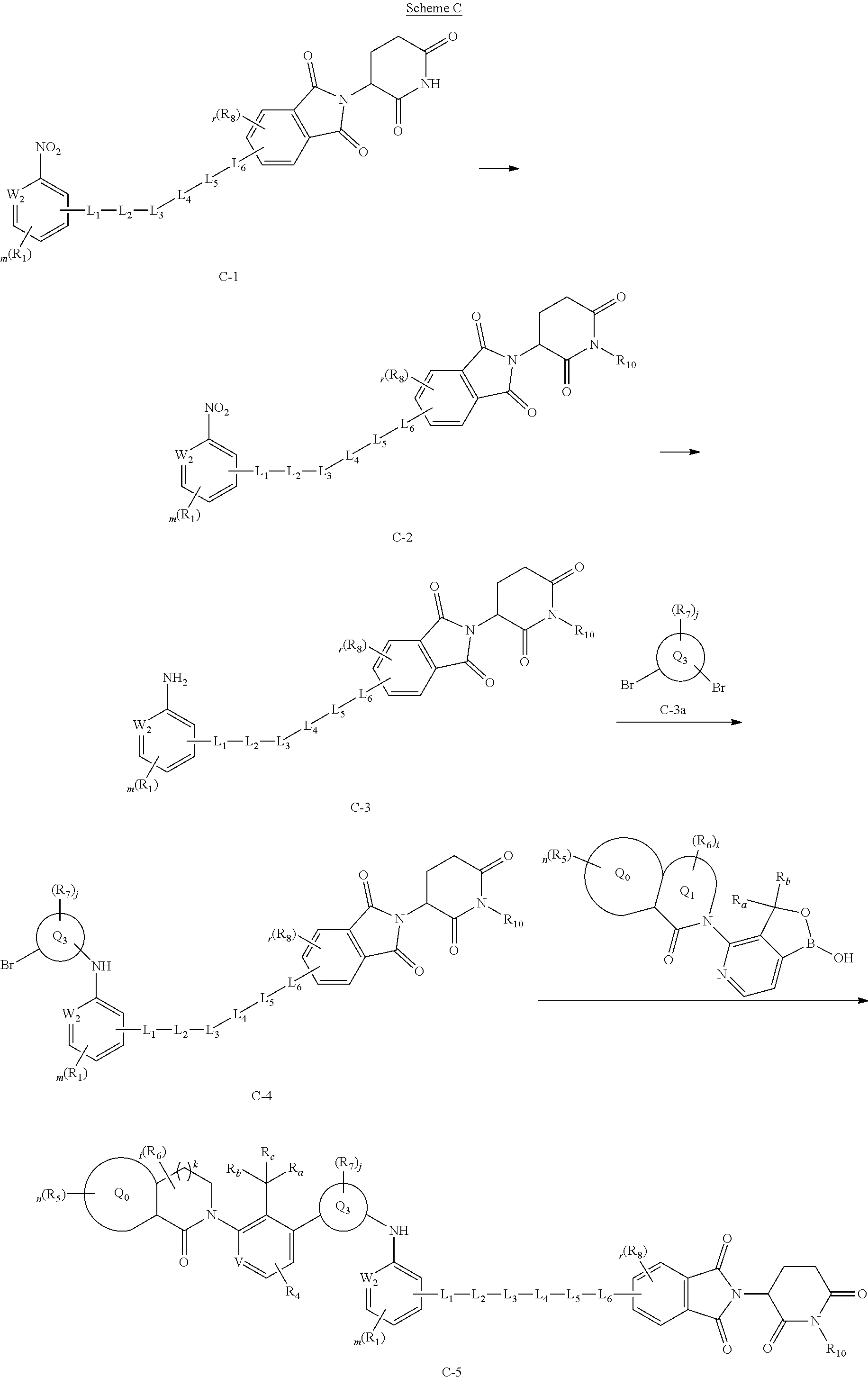

In Scheme C, the starting material C-1 can be prepared by conventional procedures using appropriate compounds and reagents. The starting material C-1 can be converted to C-2 through a conventional reaction. After that, the intermediate C-2 can be reduced to C-3 readily, which can react with C-3a to give C-4. Finally, C-4 can be converted to the target compounds C-5 through a Suzuki coupling reaction.

Also, the target compounds can be synthesized by alternative methods but not limited to the above procedures.

The compounds of

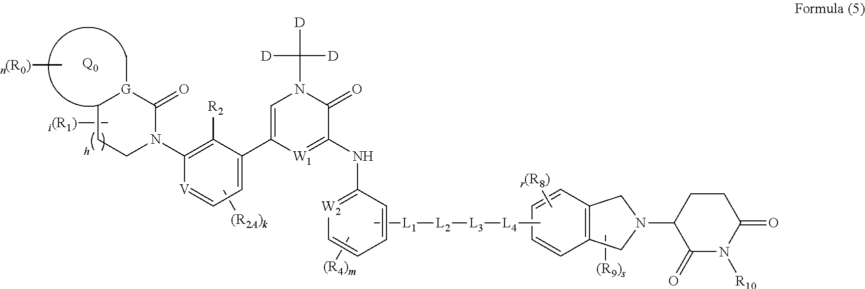

can be made by the method similar to Scheme A, B, B1, B2, B3, B4, B5, B6, C by using different starting material, intermediates, and reagents.

The compounds of

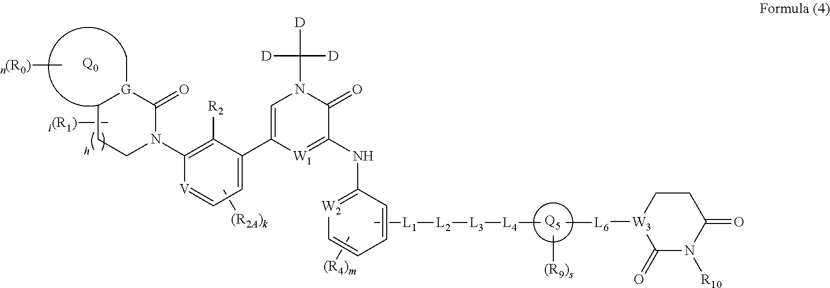

can be made by the method similar to Scheme A, B, B1, B2, B3, B4, B5, B6, C by using different starting material, intermediates, and reagents.

The compounds of

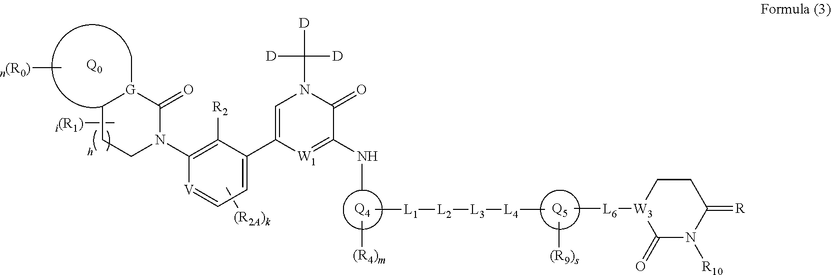

can be made by the method similar to Scheme A, B, B1, B2, B3, B4, B5, B6, C by using different starting material, intermediates, and reagents.

The compounds of

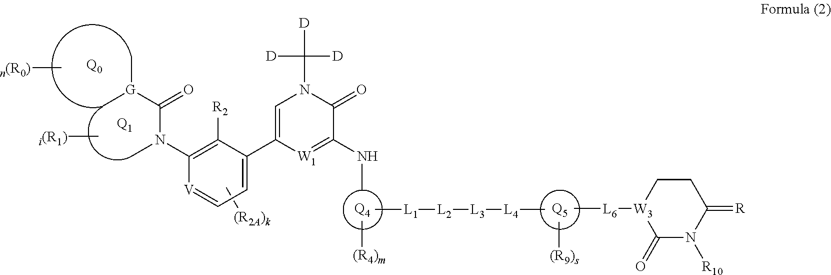

can be made by the method similar to Scheme A, B, B1, B2, B3, B4, B5, B6, C by using different starting material, intermediates, and reagents.

The compounds of

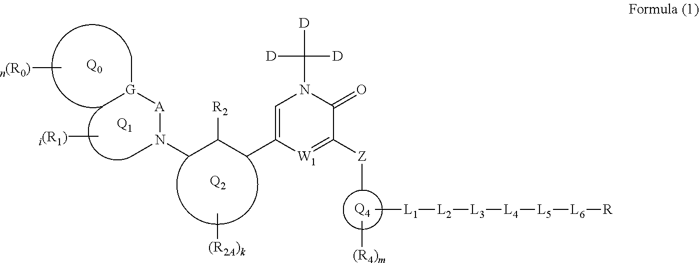

can be made by the method similar to Scheme A, B, B1, B2, B3, B4, B5, B6, C by using different starting material, intermediates, and reagents.

The compounds of

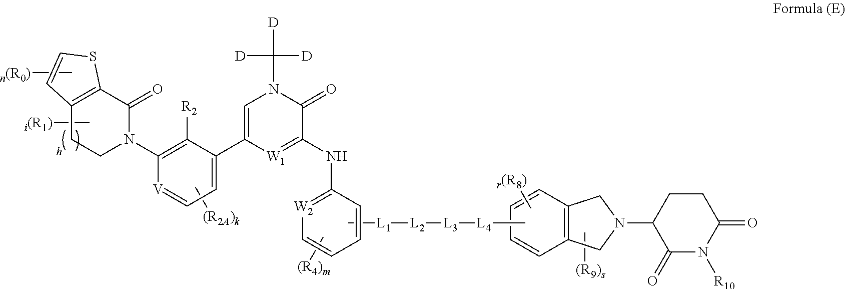

can be made by the method similar to Scheme A, B, B1, B2, B3, B4, B5, B6, C by using different starting material, intermediates, and reagents.

The compounds of

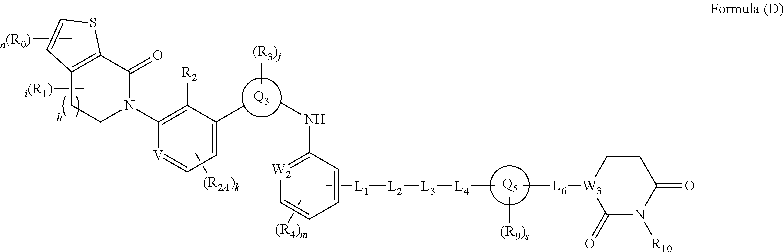

can be made by the method similar to Scheme A, B, B1, B2, B3, B4, B5, B6, C by using different starting material, intermediates, and reagents.

The compounds of

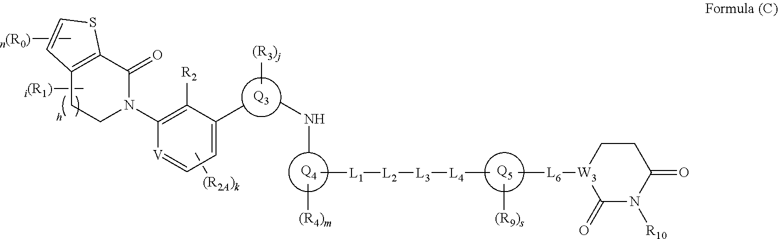

can be made by the method similar to Scheme A, B, B1, B2, B3, B4, B5, B6, C by using different starting material, intermediates, and reagents.

The compounds of

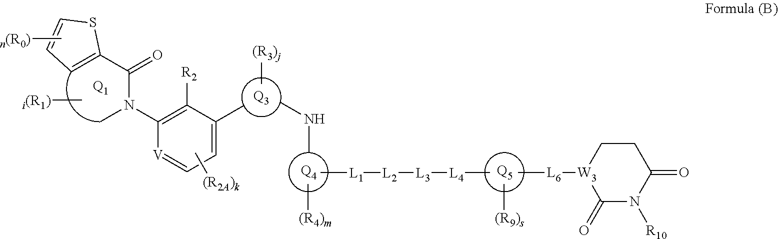

can be made by the method similar to Scheme A, B, B1, B2, B3, B4, B5, B6, C by using different starting material, intermediates, and reagents.

The compounds of

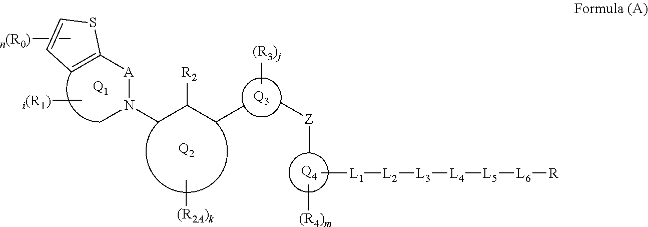

can be made by the method similar to Scheme A, B, B1, B2, B3, B4, B5, B6, C by using different starting material, intermediates, and reagents.

An approach to synthesize compounds of

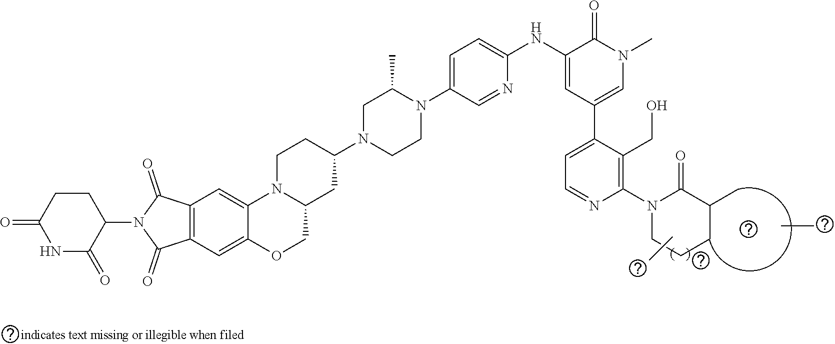

is described in Scheme D. Q₃, R₅, R₆, i, j, and k in general Scheme D are the same as those described in the Summary section above.

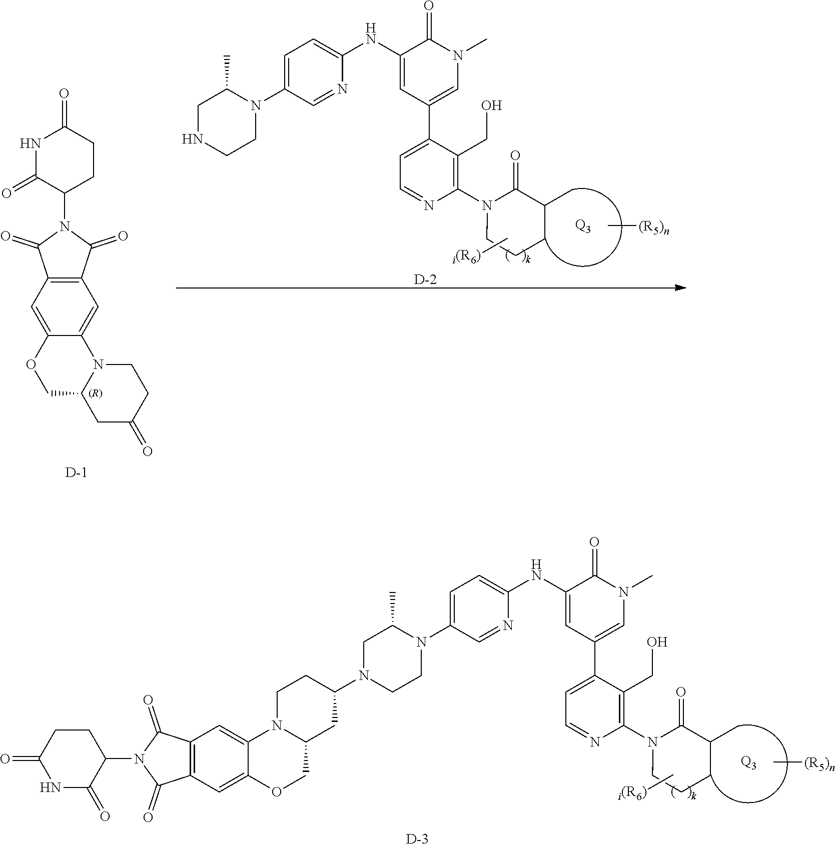

In Scheme 0, the reductive amination of D-1 and D-2 under the corresponding conditions can afford the target compounds 0-3.

Also, the target compounds can be synthesized by alternative methods but not limited to the above procedures.

An approach to synthesize compounds of

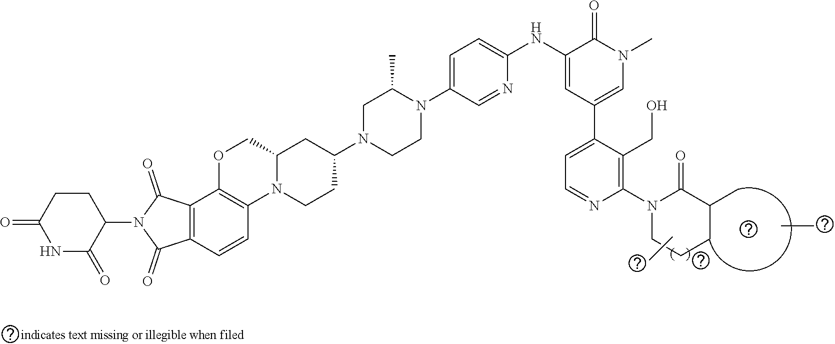

is described in Scheme E.

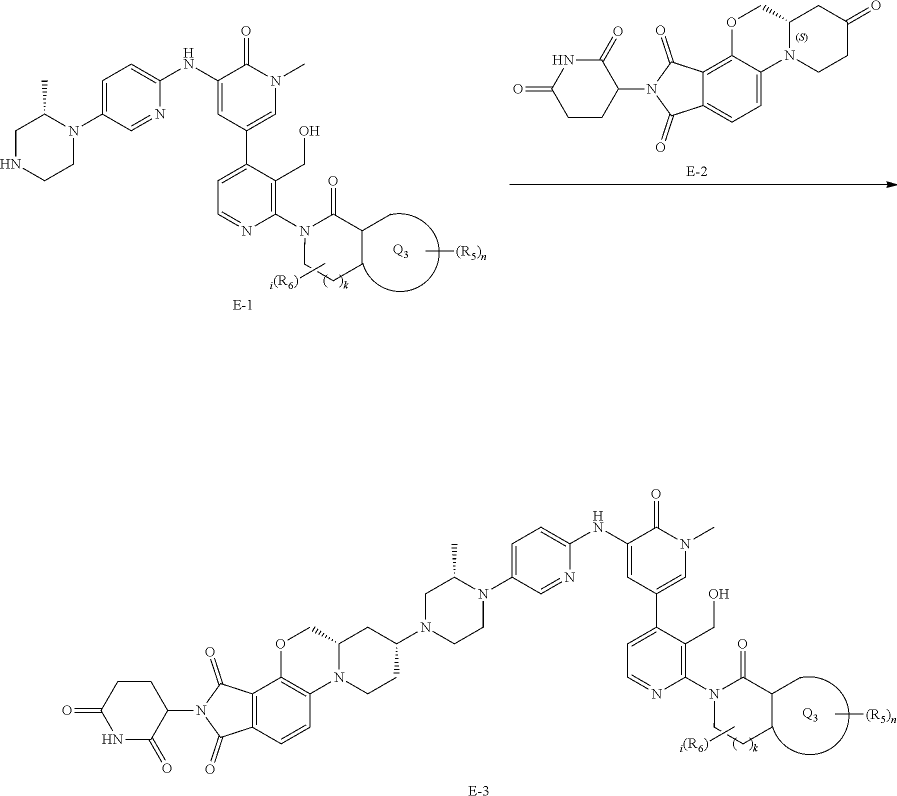

In Scheme E, E-1 can react with E-2 to afford E-3 via a reductive amination.

Also, the target compounds can be synthesized by alternative methods but not limited to the above procedures.

An approach to synthesize compounds of

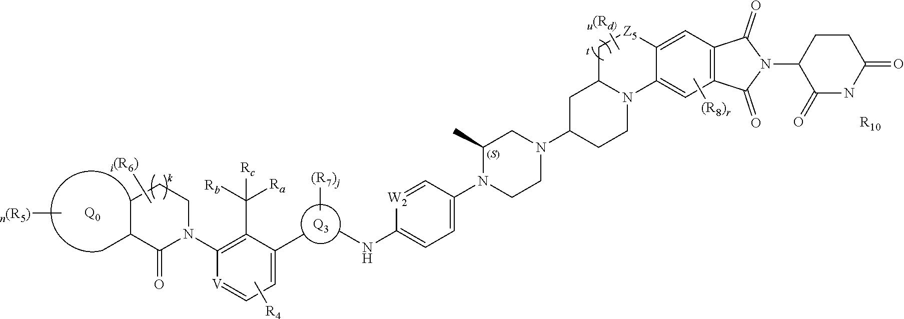

is described in Scheme F.

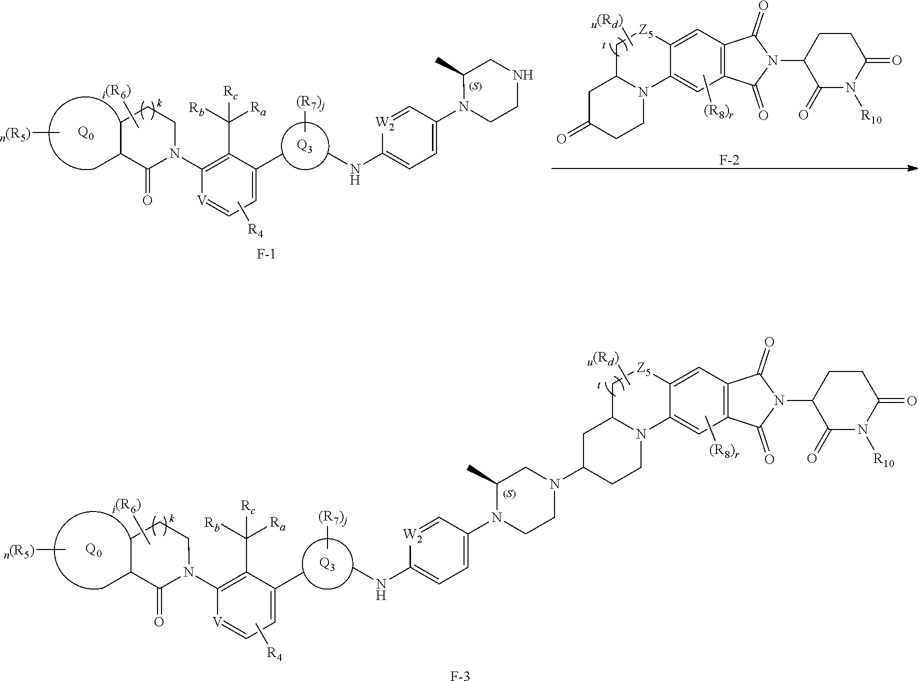

In Scheme F, the reductive amination of F-1 and F-2 under the corresponding conditions can afford the target compounds F-3.

Also, the target compounds can be synthesized by alternative methods but not limited to the above procedures.

An approach to synthesize compounds of

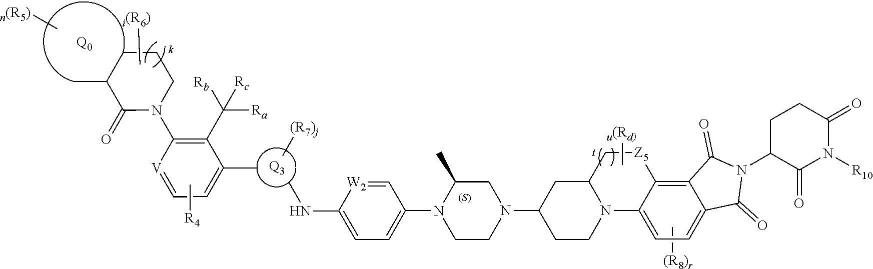

is described in Scheme G.

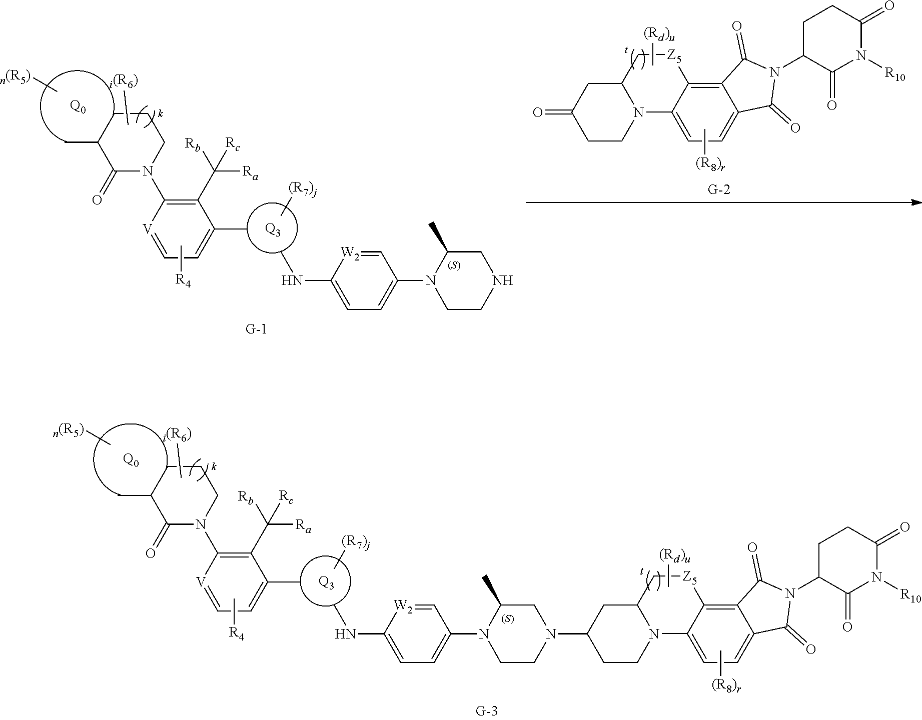

In Scheme G, the reductive amination of G-1 and G-2 under the corresponding conditions can afford the target compounds G-3.

Also, the target compounds can be synthesized by alternative methods but not limited to the above procedures.

An approach to synthesize compounds of

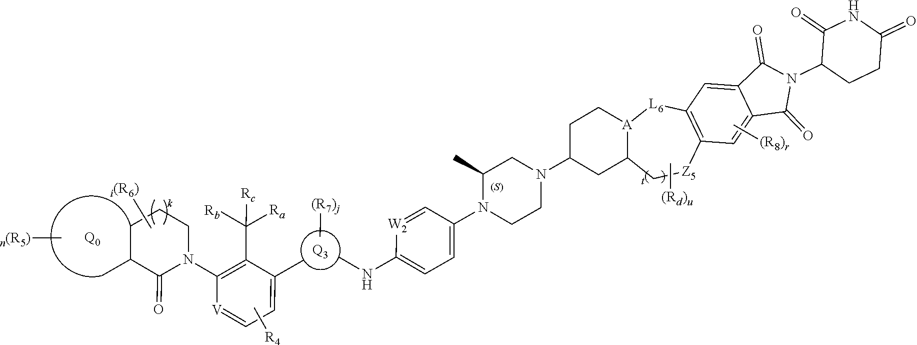

is described in Scheme H.

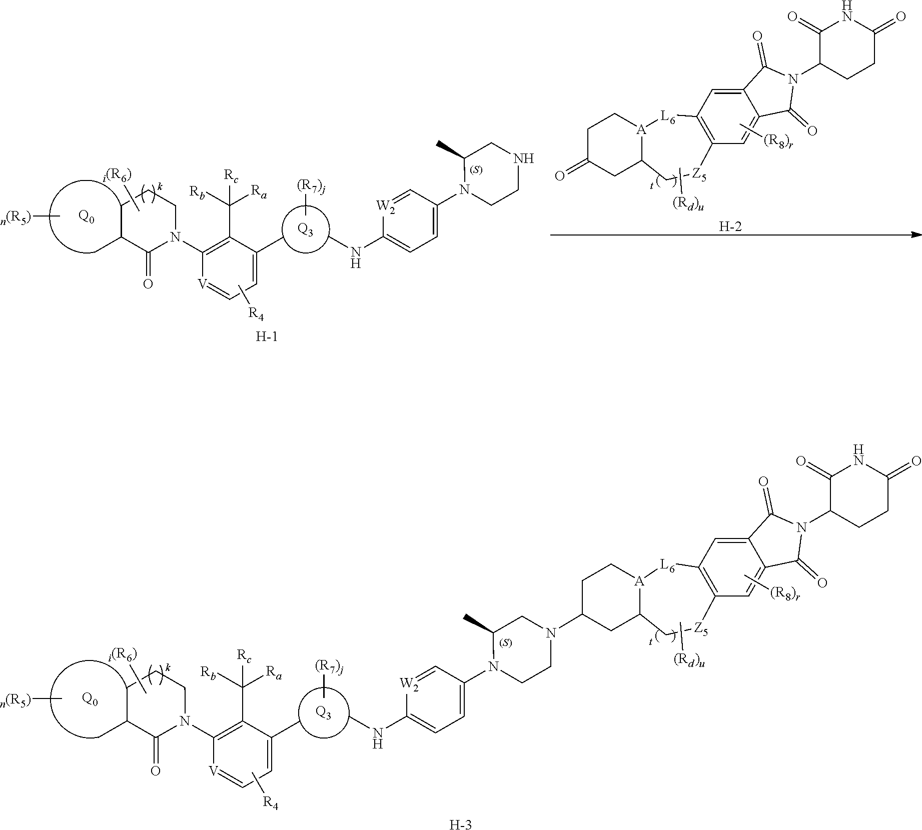

In Scheme H, the reductive amination of H-1 and H-2 under the corresponding conditions can afford the target compounds H-3.

Also, the target compounds can be synthesized by alternative methods but not limited to the above procedures.

An approach to synthesize compounds of

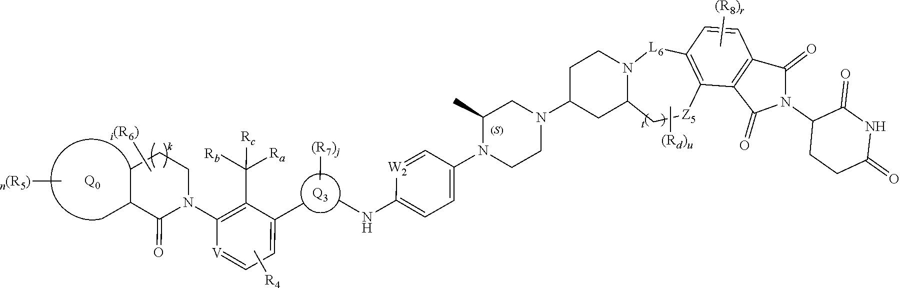

is described in Scheme I.

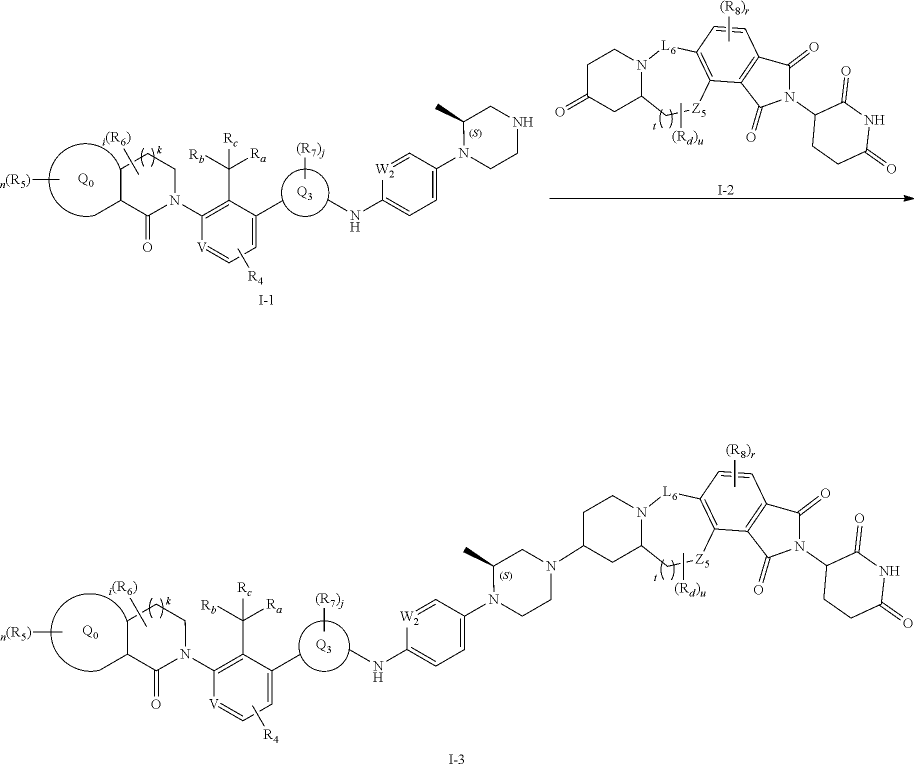

In Scheme I, the reductive amination of I-1 and 1-2 under the corresponding conditions can afford the target compounds 1-3.

Also, the target compounds can be synthesized by alternative methods but not limited to the above procedures.

The compounds of

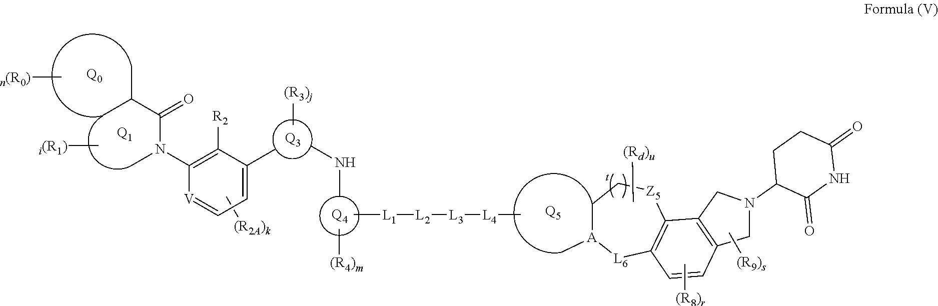

can be made by the method similar to Scheme D-I by using different starting material, intermediates, and reagents.

The compounds of

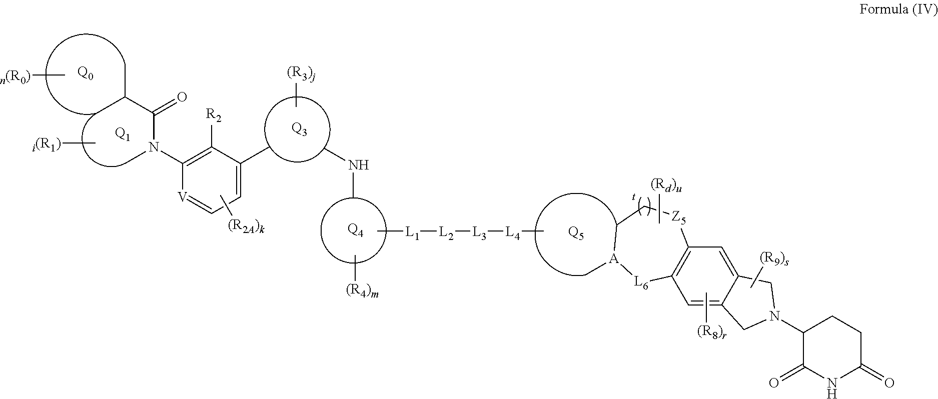

can be made by the method similar to Scheme D-I by using different starting material, intermediates, and reagents.

The compounds of

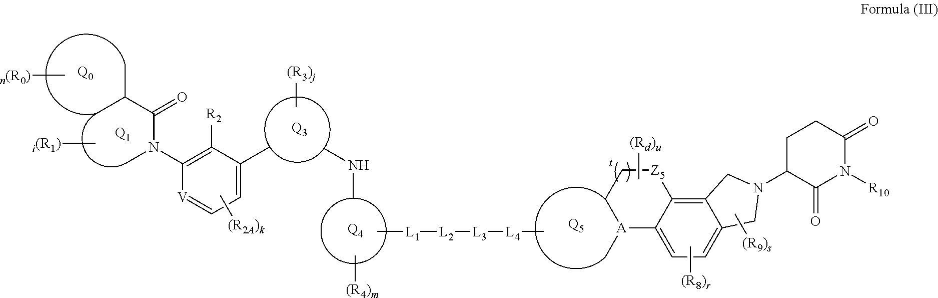

can be made by the method similar to Scheme D-I by using different starting material, intermediates, and reagents.

The compounds of

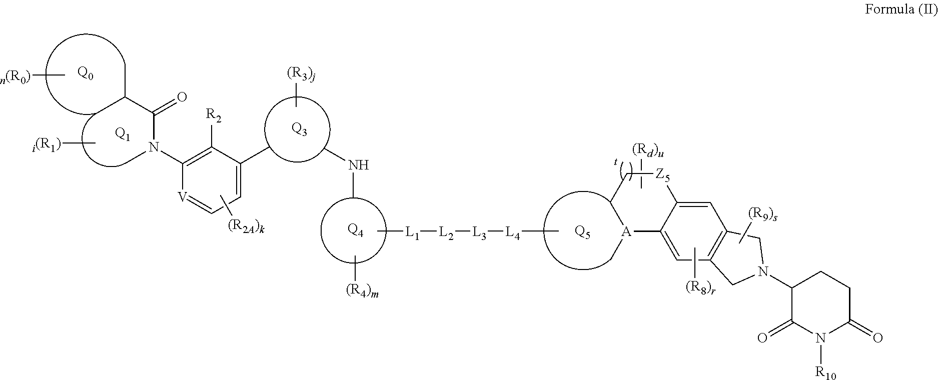

can be made by the method similar to Scheme D-I by using different starting material, intermediates, and reagents.

The compounds of

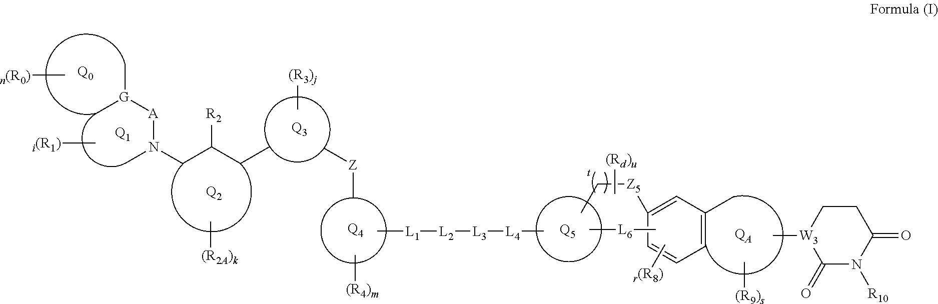

can be made by the method similar to Scheme D-I by using different starting material, intermediates, and reagents.

The compounds of

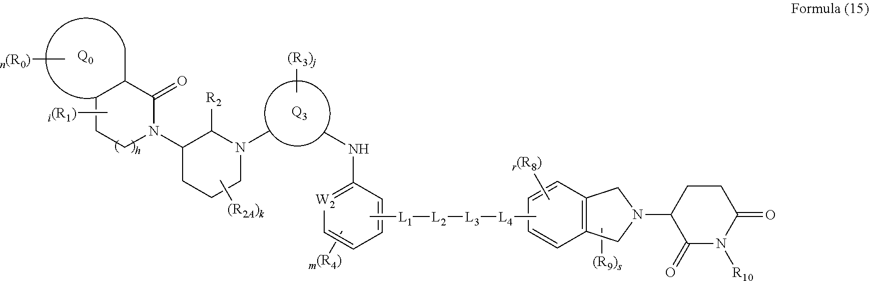

can be made by the method similar to Scheme D-I by using different starting material, intermediates, and reagents.

The compounds of

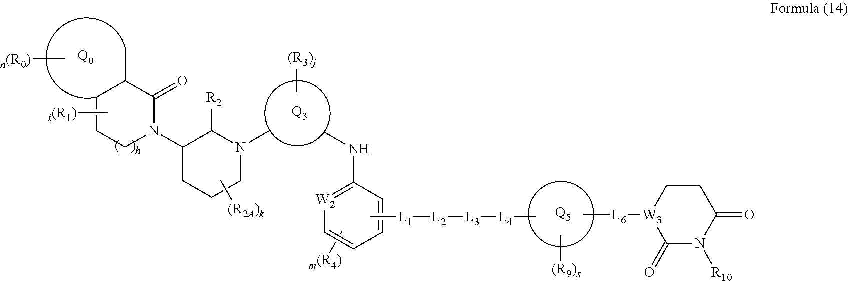

can be made by the method similar to Scheme D-I by using different starting material, intermediates, and reagents.

The compounds of

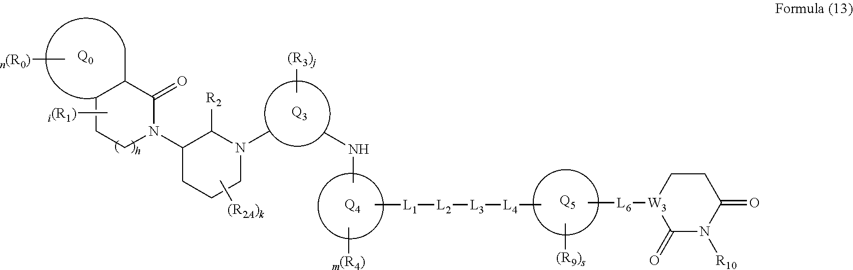

can be made by the method similar to Scheme D-I by using different starting material, intermediates, and reagents.

The compounds of

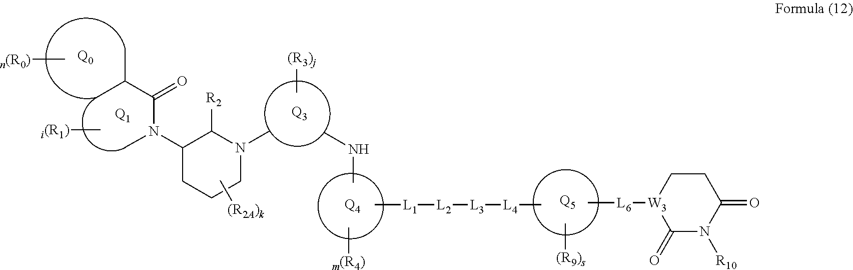

can be made by the method similar to Scheme D-I by using different starting material, intermediates, and reagents.

The compounds of

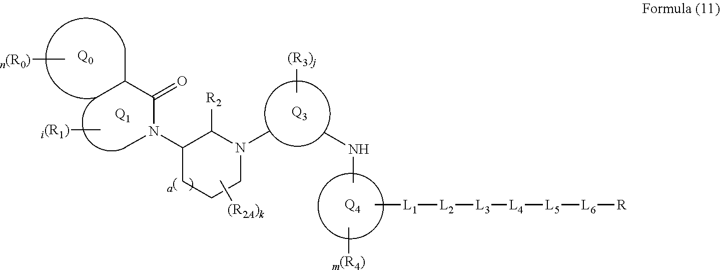

can be made by the method similar to Scheme D-I by using different starting material, intermediates, and reagents.

The compounds of

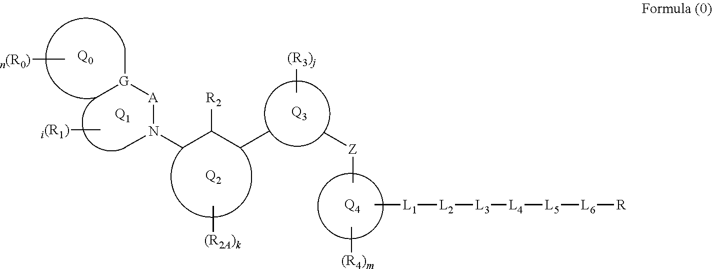

can be made by the method similar to Scheme A to Scheme I by using different starting material, intermediates, and reagents.

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Where NMR data are presented, ¹H spectra were obtained on XL400 (400 MHz) and are reported as ppm down field from Me₄Si with number of protons, multiplicities, and coupling constants in Hertz indicated parenthetically. Where HPLC data are presented, analyses were performed using an Agilent 1100 system. Where LC/MS data are presented, analyses were performed using an Applied Biosystems API-100 mass spectrometer and Shimadzu SCL-10A LC column:

### Example INT_1: Preparation of 7,7-dimethyl-3,4,7,8-tetrahydro-2H-cyclopenta[4,5]pyrrolo[1,2-a]pyrazin-1(6H)-one

Synthesis of [(3,3-dimethylcyclopent-1-en-1-yl)oxy]trimethylsilane: Into a 10-L 4-necked round-bottom flask purged and maintained under an inert atmosphere of nitrogen, were placed CuCl (20.6 g, 208 mmol, 0.05 eq), LiCl (17.6 g, 416.1 mmol, 0.1 eq) and THE (2.5 L). After that, 3-methyl-2-cyclopenten-1-one (400.0 g, 4161.0 mmol, 1.0 eq) was added at −5 to 5° C., followed by the addition of TMSCI (474.7 g, 4369.1 mm ol, 1.1 eq) dropwise with stirring at −5 to 5° C. To the above mixture was added MeMgCI (1670.0 mL, 14495.1 mmol, 3.5 eq) dropwise with stirring at −5 to 10° C. The reaction was stirred for 2 hours at −5 to 10° C. The reaction mixture was then quenched by the addition of MeOH (34 mL) and then diluted with NH₄Cl (5 L). The reaction mixture was filtered and the filtrate was extracted with petroleum ether (3×5 L). The combined organic phase was dried over anhydrous sodium sulfate. After filtration, the filtrate was concentrated to give [(3,3-dimethylcyclopent-1-en-1-yl)oxy]trimethylsilane (780.2 g, crude) as yellow oil. GC-MS (ES, m/z) M: 184.

Synthesis of 3,3-dimethylcyclopentanone: Into a 20-L 4-necked round-bottom flask, were placed [(3,3-dimethylcyclopent-1-en-1-yl)oxy]trimethylsilane (780.0 g, 4231.0 mmol, 1.0 eq), CH₂Cl₂ (7.8 L) and water (30.5 g, 1692.4 mmol, 0.4 eq). After that, POCl₃ (214.1 g, 1396.3 mmol, 0.3 eq) was added dropwise with stirring at 25 to 30° C. The reaction was stirred for 0.5 hour at 25° C. The crude product formed in solution was used for next step directly. GC-MS (ES, m/z) M: 112.

Synthesis of 3,3-dimethylcyclopentanone: Into a 20-L 4-necked round-bottom flask, were placed 3,3-dimethylcyclopentan-1-one in DCM (7.80 L) from the previous step. After that, DMF (619.0 g, 8.5 mol, 2.0 eq) was added dropwise with stirring at 25° C., followed by the addition of POCl₃ (1362 g, 17.8 mol, 2.1 eq) dropwise with stirring at 40° C. The reaction was stirred overnight at 40° C. The reaction mixture was then quenched by the addition of K₃PO₄ (2000 g) in water (5 L). The resulting solution was extracted with dichloromethane (3×10 L) and the combined organic phase was dried over anhydrous sodium sulfate. After filtration, the filtrate was concentrated under vacuumto give the crude product 2-chloro-4,4-dimethylcyclopent-1-ene-1-carbaldehyde (530 g) as a brown solid. GC-MS (ES, m/z): 158.

Synthesis of 4,4-dimethyl-1,10-diazatricyclo[6.4.0.0\hat{ }[2,6]]dodeca-2(6),7-dien-9-one: Into a 5-L 4-necked round-bottom flask, were placed 2-chloro-4,4-dimethylcyclopent-1-ene-1-carbaldehyde (474.0 g, 2988.1 mmol, 1.0 eq), DMF (3 L), piperazin-2-one (299.2 g, 2988.1 mmol, 1.0 eq) and DIEA (463.4 g, 3585.7 mmol, 1.2 eq). The reaction was stirred overnight at 115° C. The reaction mixture was cooled down to 25° C. The resulting mixture was partitioned between water (2 L) and petroleum ether (2 L). The organic phase was dried over anhydrous sodium sulfate. After filtration, the filtrate was concentrated under vacuum. Finally, 4,4-dimethyl-1,10-diazatricyclo[6.4.0.0\hat{ }[2,6]]dodeca-2(6),7-dien-9-one (30.0 g, 37.7%) was obtained as a grey solid. LC-MS (ES, m/z) M+1: 205.

### Example INT_2: Preparation of 2,4-dibromo-3-(((tetrahydro-2H-pyran-2-yl)oxy)methyl)pyridine

Synthesis of 2,4-dibromopyridine-3-carbaldehyde: Into a 1000-mL 3-necked round-bottom flask, were placed 2,4-dibromopyridine (40.0 g, 168.9 mmol, 1.0 eq) and THE (400 mL). After that, LDA (2M in hexane, 126.6 mL, 1.5 eq) was added dropwise with stirring at −78° C. The reaction was stirred for 2 hours at −78° C., then DMF (16.0 g, 219.5 mmol, 1.3 eq) was added dropwise with stirring at −78° C. The reaction was stirred for 1 hour at −78° C. The reaction mixture was quenched by NH₄Cl/HOAc (1:1, 500 mL). The resulting solution was extracted with ethyl acetate (3×3500 mL) and the combined organic was dried over anhydrous sodium sulfate. After filtration, the filtrate was concentrated under vacuum. The crude residue was applied onto a silica gel column and eluted with ethyl acetate/petroleum ether=1:1 to give 2,4-dibromopyridine-3-carbaldehyde as a white solid 24.4 g, 54.5%). LC-MS (ES, m/z) M+1: 264.

Synthesis of (2,4-dibromopyridin-3-yl)methanol: Into a 100-mL round-bottom flask, were placed 2,4-dibromopyridine-3-carbaldehyde (2.0 g, 7.6 mmol, 1.0 eq) andEtOH (30 mL). After that, NaBH₄ (286 mg, 7.6 mmol, 1.0 eq) was added in portions at 0° C. The reaction was stirred for 3 hours at θ° C. The reaction mixture was quenched by the addition of water (30 mL). The resulting solution was extracted with ethyl acetate (3×30 mL) and the combined organic phase was dried over anhydrous sodium sulfate. After filtration, the filtrate was concentrated under vacuum. The crude residue was applied onto a silica gel column and eluted with ethyl acetate/petroleum ether=1:1 to give (2,4-dibromopyridin-3-yl)methanol as a light yellow solid (1.4 g, 69.5%). LC-MS (ES, m/z) M+1: 266.

Synthesis of 2,4-dibromo-3-[(oxan-2-yloxy)methyl]pyridine: Into a 100-mL round-bottom flask, were placed (2,4-dibromopyridin-3-yl)methanol (1.4 g, 5.2 mmol, 1.0 eq), DCM (30 mL), PPTS (132 mg, 0.5 mmol, 0.1 eq) and DHP (662 mg, 7.9 mmol, 1.5 eq). The reaction was stirred overnight at 45° C. The reaction was then quenched by water (30 mL). The resulting solution was extracted with dichloromethane (3×30 mL) and the combined organic phase was dried over anhydrous sodium sulfate. After filtration, the filtrate was concentrated under vacuum. The crude residue was applied onto a silica gel column and eluted with ethyl acetate/petroleum ether=1:1 to give 2,4-dibromo-3-[(oxan-2-yloxy)methyl]pyridine as colorless oil (1.5 g, 80.0%). LC-MS (ES, m/z) M+1: 350.

### Example INT_3: Preparation of 2-(1-hydroxy-1,3-dihydro-[1,2]oxaborolo[4,3-c]pyridin-4-yl)-7,7-dimethyl-3,4,7,8-tetrahydro-2H-cyclopenta[4,5]pyrrolo[1,2-a]pyrazin-1(6H)-one

Synthesis of 10-[4-bromo-3-[(oxan-2-yloxy)methyl]pyridin-2-yl]-4,4-dimethyl-1,10-diazatricyclo[6.4.0.0\hat{ }[2,6]]dodeca-2(6),7-dien-9-one: Into a 100-mL round-bottom flask purged and maintained under an inert atmosphere of nitrogen, were placed 4,4-dimethyl-1,10-diazatricyclo[6.4.0.0\hat{ }[2,6]]dodeca-2(6),7-dien-9-one (1.0 g, 4.9 mmol, 1.0 eq), dioxane (40 mL), Cs₂CO₃ (3.2 g, 9.8 mmol, 2 eq), 2,4-dibromo-3-[(oxan-2-yloxy)methyl]pyridine (1.7 g, 4.9 mmol, 1.0 eq), Pd₂(dba)₃(448 mg, 0.5 mmol, 0.1 eq) and XantPhos (283 mg, 0.5 mmol, 0.1 eq). The reaction was stirred for 3 hours at 100° C. The reaction mixture was cooled down to 25° C. and quenched by the addition of water (40 mL). The resulting solution was extracted with ethyl acetate (3×40 mL) and the combined organic phase was dried over anhydrous sodium sulfate. After filtration, the filtrate was concentrated under vacuum. The crude residue was applied onto a silica gel column and eluted with ethyl acetate/petroleum ether=1:1 to give 10-[4-bromo-3-[(oxan-2-yloxy)methyl]pyridin-2-yl]-4,4-dimethyl-1,10-diazatricyclo[6.4.0.0\hat{ }[2,6]]dodeca-2(6),7-dien-9-one (900 mg, 38.7%) as a brown solid. LC-MS (ES, m/z) M+1: 474.

Synthesis of 2-[4,4-dimethyl-9-oxo-1,10-diazatricyclo[6.4.0.0\hat{ }[2,6]]dodeca-2(6),7-dien-10-yl]-3-[(oxan-2-yloxy)methyl]pyridin-4-ylboronic acid: Into a 100-mL round-bottom flask purged and maintained under an inert atmosphere of nitrogen, were placed 10-[4-bromo-3-[(oxan-2-yloxy)methyl]pyridin-2-yl]-4,4-dimethyl-1,10-diazatricyclo[6.4.0.0\hat{ }[2,6]]dodeca-2(6),7-dien-9-one (1.0 g, 2.1 mmol, 1.0 eq), dioxane (10 mL), bis(pinacolato)diboron (1.3 g, 5.3 mmol, 2.5 eq), KOAc (620 mg, 6.3 mmol, 3.0 eq), Pd(dppf)Cl₂ (172 mg, 0.2 mmol, 0.1 eq). The reaction was stirred for 2 hours at 100° C. The reaction mixture was filtered and the filtrate was concentrated under vacuum to give 2-[4,4-dimethyl-9-oxo-1,10-diazatricyclo[6.4.0.0\hat{ }[2,6]]dodeca-2(6),7-dien-10-yl]-3-[(oxan-2-yloxy)methyl]pyridin-4-ylboronic acid (920 mg, crude) as brown oil. LC-MS (ES, m/z) M+1: 440.

Synthesis of 10-[1-hydroxy-3H-[1,2]oxaborolo[4,3-c]pyridin-4-yl]-4,4-dimethyl-1,10-diazatricyclo[6.4.0.0\hat{ }[2,6]]dodeca-2(6),7-dien-9-one: Into a 100-mL round-bottom flask, were placed 2-[4,4-dimethyl-9-oxo-1,10-diazatricyclo[6.4.0.0\hat{ }[2,6]]dodeca-2(6),7-dien-10-yl]-3-[(oxan-2-yloxy)methyl]pyridin-4-ylboronic acid (920 mg, 1.0 eq, crude), dioxane (10 mL) and HCl (6 M, 10 mL). The reaction was stirred for 1 hour at 25° C. The resulting mixture was concentrated under vacuum. The crude product was purified by Flash-Prep-HPLC using the following conditions: Column, C18 reversed phase column; mobile phase, water (0.05% NH₃·H₂O) and CH₃CN (5% CH₃CN up to 30% in 15 min); Flow rate: 60 mL/min; Detector, 254/220 nm. Finally, 10-[1-hydroxy-3H-[1,2]oxaborolo[4,3-c]pyridin-4-yl]-4,4-dimethyl-1,10-diazatricyclo[6.4.0.0\hat{ }[2,6]]dodeca-2(6),7-dien-9-one (350 mg) was obtained as a light yellow solid. LC-MS (ES, m/z) M+1: 338.

### Example INT_4: Preparation of 2-(2,6-dioxopiperidin-3-yl)-5-iodoisoindoline-1,3-dione

Synthesis of 4-iodobenzene-1,2-dicarboxylic acid: To a stirred mixture of 4-iodo-1,2-dimethylbenzene (30.0 g, 129.3 mmol, 1.0 eq) and Pyridine (270 mL) in water (500 mL) was added KMnO₄ (200.0 g, 1.3 mol, 10.0 eq) in portions at 25° C. The reaction was stirred for 24 hours at 100° C. The resulting mixture was filtered at a high temperature and the filter cake was washed with 1M NaOH (aq.). The filtrate was acidified to pH=1 with conc. HCl. The resulting mixture was extracted with EtOAc (3×200 mL). The combined organic phase was washed with brine (1×200 mL), dried over anhydrous Na₂SO₄. After filtration, the filtrate was concentrated under vacuum. The filtrate was concentrated under vacuum to give 4-iodobenzene-1,2-dicarboxylic acid (28 g, 74.2%) as a light brown solid. ¹HNMR (300 MHz, DMSO-d₆) δ 13.37 (bs, 2H), 8.17-7.76 (m, 2H), 7.48 (d, J=8.0 Hz, 1H).

Synthesis of 5-iodo-2-benzofuran-1,3-dione: Into a 1 L round-bottom flask were added 4-iodobenzene-1,2-dicarboxylic acid (28.0 g, 0.1 mol, 1.0 eq) and acetic anhydride (300 mL) at 25° C. The reaction was stirred for 12 hours at 100° C. The resulting mixture was concentrated under vacuum to give 5-iodo-2-benzofuran-1,3-dione (19 g, 72.3%) as a brown solid. ¹HNMR (400 MHz, DMSO-d₆) δ 8.46 (d, J=1.4 Hz, 1H), 8.37 (dd, J=7.9, 1.4 Hz, 1H), 7.83 (d, J=7.9 Hz, 1H).

Synthesis of 2-(2,6-dioxopiperidin-3-yl)-5-iodoisoindole-1,3-dione: To a stirred mixture of 5-iodo-2-benzofuran-1,3-dione (19.0 g, 69.3 mmol, 1.0 eq), 3-aminopiperidine-2,6-dione (17.8 g, 138.7 mmol, 2.0 eq), NaOAc (11.4 g, 138.7 mmol, 2.0 eq) in AcOH (150 mL) at 25° C. The reaction was stirred for 12 hours at 115° C. After that, the reaction was quenched with water (100 mL) at 25° C. The precipitated solids were collected by filtration and washed with water (3×100 mL). The resulting solid was dried under infrared light to give 2-(2,6-dioxopiperidin-3-yl)-5-iodoisoindole-1,3-dione (23 g, 86.4%) as a black solid. ¹HNMR (400 MHz, DMSO-d₆) δ 11.13 (s, 1H), 8.31-8.25 (m, 2H), 7.70 (d, J=8.2 Hz, 1H), 5.16 (dd, J=12.8, 5.4 Hz, 1H), 2.89 (ddd, J=17.0, 13.8, 5.4 Hz, 1H), 2.66-2.51 (m, 2H), 2.06 (dd, J=13.0, 5.3, 2.2 Hz, 1H).

### Example INT_5: Preparation of tert-butyl (S)-4-(6-aminopyridin-3-yl)-3-methylpiperazine-1-carboxylate

Synthesis of tert-butyl (3S)-3-methyl-4-(6-nitropyridin-3-yl)piperazine-1-carboxylate: Into a 500 mL round-bottom flask were added tert-butyl (3S)-3-methylpiperazine-1-carboxylate (19.7 g, 98.4 mmol, 1.0 eq), 5-bromo-2-nitropyridine (20.0 g, 98.4 mmol, 1.0 eq), Cs₂CO₃ (96.1 g, 295.1 mmol, 3.0 eq), Pd₂(dba)₃·CHCl₃ (10.2 g, 9.8 mmol, 0.1 eq), XantPhos (5.7 g, 9.8 mmol, 0.1 eq) and 1,4-dioxane (200 mL). The reaction was stirred for 16 hours at 100° C. under nitrogen atmosphere. The resulting mixture was filtered and the filter cake was washed with CH₂Cl₂ (50 mL). The resulting mixture was diluted with water (100 mL) and then extracted with CH₂Cl₂ (3×50 mL). The combined organic phase was washed with brine (1×50 mL), dried over anhydrous Na₂SO₄. After filtration, the filtrate was concentrated under vacuum. The crude residue was purified by a flash column (silica gel, petroleum ether/ethyl acetate=1:1) to give tert-butyl (3S)-3-methyl-4-(6-nitropyridin-3-yl)piperazine-1-carboxylate (14 g, 44.0%) as a brown solid. ¹HNMR (400 MHz, DMSO-d₆) δ 8.24-8.14 (m, 2H), 7.43 (dd, J=9.2, 3.0 Hz, 1H), 4.32 (br, 1H), 3.94 (bs, 1H), 3.80 (dt, J=12.4, 3.3 Hz, 2H), 3.30-3.10 (m, 2H), 3.09 (bs, 1H), 1.43 (s, 9H), 1.09 (d, J=6.4 Hz, 3H).

Synthesis of tert-butyl (3S)-4-(6-aminopyridin-3-yl)-3-methylpiperazine-1-carboxylate: Into a 500 mL round-bottom flask were added tert-butyl (3S)-3-methyl-4-(6-nitropyridin-3-yl)piperazine-1-carboxylate (14.0 g, 43.4 mmol, 1.0 eq) and Pd/C (1.5 g, 13.9 mmol, 0.3 eq) in EtOH (140 mL) was stirred for 6 hours at 25° C. under hydrogen atmosphere. The resulting mixture was filtered and the filter cake was washed with EtOH (50 mL). The filtrate was concentrated under vacuum to give tert-butyl (3S)-4-(6-aminopyridin-3-yl)-3-methylpip erazine-1-carboxylate (9 g, 71.3%) as a brown solid. ¹HNMR (300 MHz, DMSO-d₆) δ 7.62 (d, J=3.0 Hz, 1H), 7.22 (dd, J=8.7, 3.0 Hz, 1H), 6.42 (d, J=8.7 Hz, 1H), 5.49-5.55 (bs, 2H), 3.58-3.42 (m, 1H), 3.42-3.32 (bs, 3H), 3.18 (bs, 1H), 2.87-2.78 (m, 2H), 1.42 (s, 9H), 0.77 (d, J=6.0 Hz, 3H).

### Example INT_6: Preparation of tert-butyl (3S)-4-{6-[(5-bromo-1-methyl-2-oxopyridin-3-yl)amino]pyridin-3-yl}-3-methylpiperazine-1-carboxylate

Synthesis of tert-butyl (3S)-4-{6-[(5-bromo-1-methyl-2-oxopyridin-3-yl)amino]pyridin-3-yl}-3-me thylpiperazine-1-carboxylate: Into a 250 mL round-bottom flask were added 3,5-dibromo-1-methylpyridin-2-one (7.3 g, 27.4 mmol, 1.0 eq), tert-butyl (3S)-4-(6-aminopyridin-3-yl)-3-methylpiperazine-1-carboxylate (8.0 g, 27.4 mmol, 1.0 eq), Cs₂CO₃ (17.8 g, 54.7 mmol, 2.0 eq), Xantphos Pd 4G (2.6 g, 2.7 mmol, 0.1 eq) and 1,4-dioxane (100 mL). The reaction was stirred for 16 hours at 100° C. under nitrogen atmosphere. The reaction was cooled down to 25° C. and concentrated under vacuum. The resulting mixture was diluted with water (30 mL), extracted with CH₂Cl₂ (3×30 mL). The combined organic phase was washed with brine (1×30 mL), dried over anhydrous Na₂SO₄. After filtration, the filtrate was concentrated under vacuum. The crude residue was purified by a flash column (silica gel, petroleum ether/ethyl acetate=1:1) to give tert-butyl (3S)-4-{6-[(5-bromo-1-methyl-2-oxopyridin-3-yl)amino]pyridin-3-yl}-3-methylpiperazine-1-carboxylate (8 g, 61.7%) as a white solid. ¹HNMR (300 MHz, DMSO-d₆) δ 8.63-8.55 (m, 2H), 7.93 (d, J=2.7 Hz, 1H), 7.46 (d, J=2.7 Hz, 1H), 7.39 (dd, J=9.0, 3.0 Hz, 1H), 7.28 (d, J=9.0 Hz, 1H), 3.41-3.34 (bs, 3H), 3.15-3.10 (m, 4H), 3.09 (d, J=12.9 Hz, 1H), 2.95-2.89 (m, 2H), 1.43 (s, 9H), 0.85 (d, J=6.3 Hz, 3H).

### Example INT_7: Preparation of 2-(2,6-dioxopiperidin-3-yl)-5-(4-oxopiperidin-1-yl)isoindoline-1,3-dione

Synthesis of 2-(2,6-dioxopiperidin-3-yl)-5-(4-oxopiperidin-1-yl)isoindole-1,3-dione: Into a 50 mL round-bottom flask were added 4-piperidinone (1.0 g, 10.1 mmol, 1.5 eq), 2-(2,6-dioxopiperidin-3-yl)-5-fluoroisoindole-1,3-dione (2.0 g, 7.2 mmol, 0.7 eq), DIEA (4.3 g, 33.2 mmol, 3.3 eq) and NMP (7 mL) at 25° C. The reaction was stirred for 18 hours at 100° C. The resulting mixture was diluted with EtOAc (10 mL). The precipitated solids were collected by filtration and washed with EtOAc (3×5 mL). The combined organic phase was washed with water (3×5 mL), dried over anhydrous Na₂SO₄. After filtration, the filtrate was concentrated under vacuum. The crude residue was purified by silica gel column chromatography, eluted with CH₂Cl₂/MeOH=10:1 to give 2-(2,6-dioxopiperidin-3-yl)-5-(4-oxopiperidin-1-yl)isoindole-1,3-dione (700 mg, 29.3%) as a yellow solid. ¹HNMR (300 MHz, DMSO-d₆) δ 11.08 (s, 1H), 7.71 (d, J=8.5 Hz, 1H), 7.38 (d, J=2.3 Hz, 1H), 7.29 (dd, J=8.6, 2.4 Hz, 1H), 5.08 (dd, J=12.6, 5.4 Hz, 1H), 3.86 (t, J=6.1 Hz, 3H), 3.30 (d, J=7.0 Hz, 1H), 2.70-2.55 (m, 3H), 2.18 (t, J=8.1 Hz, 2H), 2.12-1.98 (m, 1H), 1.98-1.82 (m, 2H).

### Example INT_8: Preparation of 5-(4-((S)-4-(6-((5-bromo-1-methyl-2-oxo-1,2-dihydropyridin-3-yl)amino)pyridin-3-yl)-3-methylpiperazin-1-yl)piperidin-1-yl)-2-(2,6-dioxopiperidin-3-yl)isoindoline-1,3-dione

Synthesis of 5-bromo-1-methyl-3-({5-[(2S)-2-methylpiperazin-1-yl]pyridin-2-yl}amino)pyridin-2-one: Into a 100 mL round-bottom flask were added tert-butyl (3S)-4-{6-[(5-bromo-1-methyl-2-oxopyridin-3-yl)amino]pyridin-3-yl}-3-methylpiperazine-1-carboxylate (2.0 g, 4.2 mmol, 1.0 eq) and 4 M HCl in EtOAc (10 mL) at 25° C. The reaction was stirred for 1 hour at 25° C. The precipitated solids were collected by filtration and washed with EtOAc (3×5 mL) to give 5-bromo-1-methyl-3-({5-[(2S)-2-methylpiperazin-1-yl]pyridin-2-yl}amino)pyridin-2-one (1.5 g, 94.9%) as a yellow green solid. LC-MS: (ES, m/z): M+1: 378/380.

Synthesis of 5-{4-[(3S)-4-{6-[(5-bromo-1-methyl-2-oxopyridin-3-yl)amino]pyridin-3-yl}-3-methylpiperazin-1-yl]piperidin-1-yl}-2-(2,6-dioxopiperidin-3-yl)isoindole-1,3-dione: A solution of 5-bromo-1-methyl-3-({5-[(2S)-2-methylpiperazin-1-yl]pyridin-2-yl}amino)pyridin-2-one (1.0 g, 2.6 mmol, 1.0 eq) in DCE (10 mL) was treated with 2-(2,6-dioxopiperidin-3-yl)-5-(4-oxopiperidin-1-yl)isoindole-1,3-dione (750 mg, 2.1 mmol, 0.8 eq) for 30 min at 25° C. After that, NaBH(AcO)₃ (2.2 g, 10.6 mmol, 4.0 eq) was added in portions at 0° C. The reaction was stirred overnight at 45° C. The reaction was quenched with water (5 mL) at 0° C., and then extracted with CHCl₃ (3×5 mL). The combined organic phase was washed with brine (1×5 mL), dried over anhydrous Na₂SO₄. After filtration, the filtrate was concentrated under vacuum. The residue was purified by silica gel column chromatography, eluted with CH₂Cl₂/MeOH=10:1 to give 5-{4-[(3S)-4-{6-[(5-bromo-1-methyl-2-oxopyridin-3-yl)amino]pyridin-3-yl}-3-methylpiperazin-1-yl]piperidin-1-yl}-2-(2,6-dioxopiperidin-3-yl)isoindole-1,3-dione (480 mg, 25.3%) as an orange solid. LC-MS: (ES, m/z): M+1: 717/719.

### Example INT_9: Preparation of (S)-2-(3′-(hydroxymethyl)-1-methyl-5-((5-(2-methylpiperazin-1-yl)pyridin-2-yl)amino)-6-oxo-1,6-dihydro-[3,4′-bipyridin]-2′-yl)-7,7-dimethyl-3,4,7,8-tetrahydro-2H-cyclopenta[4,5]pyrrolo[1,2-a]pyrazin-1(6H)-one

Synthesis of tert-butyl (3S)-4-{6-[(2′-{4,4-dimethyl-9-oxo-1,10-diazatricyclo[6.4.0.0\hat{ }{2,6}]dodeca-2(6),7-dien-10-yl}-3′-(hydroxymethyl)-1-methyl-6-oxo-[3,4′-bipyridin]-5-yl)amino]pyridin-3-yl}-3-methylpiperazine-1-carboxylate: Into a 250 mL round-bottom flask were added 10-{1-hydroxy-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one (7.1 g, 20.9 mmol, 2.0 eq), tert-butyl (3S)-4-{6-[(5-bromo-1-methyl-2-oxopyridin-3-yl)amino]pyridin-3-yl}-3-methylpiperazine-1-carboxylate (5.0 g, 10.5 mmol, 1.0 eq), K₂CO₃ (4.3 g, 31.4 mmol, 3.0 eq), Pd(DtBPF)Cl₂ (0.7 g, 1.0 mmol, 0.1 eq) and 1,4-dioxane/water (50 mL/5 mL). The reaction was stirred for 1.5 hours at 90° C. under nitrogen atmosphere. The resulting mixture was cooled down to 25° C. and concentrated under vacuum. The crude residue was purified by a flash column (silica gel, petroleum ether/ethyl acetate=1:1) to give tert-butyl (3S)-4-{6-[(2′-{4,4-dimethyl-9-oxo-1,10-diazatricyclo[6.4.0.0\hat{ }{2,6}]dodeca-2(6),7-dien-10-yl}-3′-(hydroxymethyl)-1-methyl-6-oxo-[3,4′-bipyridin]-5-yl)amino]pyridin-3-yl}-3-methylpiperazine-1-carboxylate (5 g, 67.5%) as a white solid. LC-MS: (ES, m/z): M+1: 709.

Synthesis of 10-[3′-(hydroxymethyl)-1-methyl-5-({5-[(2S)-2-methylpiperazin-1-yl]pyridin-2-yl}amino)-6-oxo-[3,4′-bipyridin]-2′-yl]-4,4-dimethyl-1,10-diazatricyclo[6.4.0.0\hat{ }{2,6}]dodeca-2(6),7-dien-9-one: Into a 250 mL round-bottom flask were added tert-butyl (3S)-4-{6-[(2′-{4,4-dimethyl-9-oxo-1,10-diazatricyclo[6.4.0.0\hat{ }{2,6}]dodeca-2(6),7-dien-10-yl}-3′-(hydroxymethyl)-1-methyl-6-oxo-[3,4′-bipyridin]-5-yl)amino]pyridin-3-yl}-3-methylpiperazine-1-carboxylate (5.0 g, 7.1 mmol, 1.0 eq), trifluoroacetic acid (5 mL) and CH₂Cl₂ (50 mL). The reaction was stirred overnight at 25° C. After that, the pH of the reaction mixture was adjusted to 9 with saturated NaHCO₃ (aq.). The resulting mixture was extracted with CH₂Cl₂ (2×100 mL). The combined organic phase was washed with brine (1×100 mL), dried over anhydrous Na₂SO₄. After filtration, the filtrate was concentrated under vacuum to give 10-[3′-(hydroxymethyl)-1-methyl-5-({5-[(2S)-2-methylpiperazin-1-yl]pyridin-2-yl}amino)-6-oxo-[3,4′-bipyridin]-2′-yl]-4,4-dimethyl-1,10-diazatricyclo[6.4.0.0\hat{ }{2,6}]dodeca-2(6),7-dien-9-one (4 g, 93.4%) as a white solid. LC-MS: (ES, m/z): M+1: 609.

### Example INT_10: Preparation of (3-(7,7-dimethyl-1-oxo-1,3,4,6,7,8-hexahydro-2H-cyclopenta[4,5]pyrrolo[1,2-a]pyrazin-2-yl)-2-methylphenyl)boronic Acid

Synthesis of 2,4-dibromo-3-methylpyridine: Into a 1000-mL 3-necked round-bottom flask purged and maintained under an inert atmosphere of nitrogen, were placed diisopropylamine (19.1 g, 190.0 mmol, 1.5 eq), THE (300 mL). After that, butyllithium (12.3 g, 190.0 mmol, 1.5 eq) was added at −30° C. and the reaction was stirred for 30 minutes. To the above mixture was added 2,4-dibromopyridine (30.0 g, 126.6 mmol, 1.0 eq) at −70° C. and stirred for other 30 minutes, followed by the addition of Mel (27.0 g, 190.0 mmol, 1.5 eq) at −70° C. and the reaction was stirred for 30 minutes at −70° C. The reaction was then quenched by the addition of 300 mL of NH₄Cl, extracted with ethyl acetate (3×200 mL) and the organic phase were combined. The organic phase was washed with water (2×100 ml) and brine (1×100 mL). The mixture was dried over anhydrous sodium sulfate and concentrated in vacuum. The residue was applied onto a silica gel column and eluted with ethyl acetate/petroleum ether=1:10 to give 2,4-dibromo-3-methylpyridine (20 g) as a brown solid. LC-MS: (ES, m/z): M+1: 250.

Synthesis of 10-(4-bromo-3-methylpyridin-2-yl)-4,4-dimethyl-1,10-diazatricyclo[6.4.0.0\hat{ }[2,6]]dodeca-2(6),7-dien-9-one: Into a 100-mL round-bottom flask purged and maintained under an inert atmosphere of nitrogen, were placed 2,4-dibromo-3-methylpyridine (5.0 g, 20.0 mmol, 1.0 eq), 4,4-dimethyl-1,10-diazatricyclo[6.4.0.0\hat{ }[2,6]]dodeca-2(6),7-dien-9-one (4.1 g, 20.0 mmol, 1.0 eq), XantPhos PD G2 (1.8 g, 2.0 mmol, 0.1 eq), dioxane (50 mL, 590.0 mmol, 29.6 eq) and Cs₂CO₃ (19.5 g, 59.8 mmol, 3.0 eq). The reaction was stirred for 3 hours at 100° C. The resulting mixture was concentrated. The crude residue was applied onto a silica gel column and eluted with ethyl acetate/petroleum ether=1:1 to give 10-(4-bromo-3-methylpyridin-2-yl)-4,4-dimethyl-1,10-diazatricyclo[6.4.0.0\hat{ }[2,6]]dodeca-2(6),7-dien-9-one (2.5 g, 33.5%) as a brown solid. LC-MS (ES, m/z): M+1: 374.

Synthesis of 2-[4,4-dimethyl-9-oxo-1,10-diazatricyclo[6.4.0.0\hat{ }[2,6]]dodeca-2(6),7-dien-10-yl]-3-methylpyridin-4-ylboronic acid: Into a 50-mL round-bottom flask purged and maintained under an inert atmosphere of nitrogen, were placed 10-(4-bromo-3-methylpyridin-2-yl)-4,4-dimethyl-1,10-diazatricyclo[6.4.0.0\hat{ }[2,6]]dodeca-2(6),7-dien-9-one (1.0 g, 2.7 mmol, 1.0 eq), bis(pinacolato)diboron (1.0 g, 4.0 mmol, 1.5 eq), dioxane (15 mL), KOAc (0.5 g, 5.3 mmol, 2.0 eq) and Pd(dppf)Cl₂ (195 mg, 0.3 mmol, 0.1 eq). The reaction was stirred for 2 hours at 100° C. The resulting mixture was concentrated. The crude residue was purified by Flash-Prep-HPLC using the following conditions (CombiFlash-1): Column, C18 silica gel; mobile phase, A: 0.1% NH₃·H₂O in water; B: MeCN; Gradient: 35%-70% B in 9 min; Detector, 220 nm. Finally, 2-[4,4-dimethyl-9-oxo-1,10-diazatricyclo[6.4.0.0\hat{ }[2,6]]dodeca-2(6),7-dien-10-yl]-3-methylpyridin-4-ylboronic acid (450 mg, 49.6%) was obtained as a white solid. LC-MS (ES, m/z): M+1: 340.

### Example INT_11: Preparation of 3,4,5,6,7,8-hexahydrobenzo[4,5]thieno[2,3-c]pyridin-1(2H)-one

Synthesis of N-(methoxymethyl)-N-methyl-4, 5, 6, 7-tetrahydro-1-benzothiophene-2-carboxamide: Into a 250-mL 3-necked round-bottom flask purged and maintained under an inert atmosphere of nitrogen, were placed 4,5,6,7-tetrahydro-1-benzothiophene-2-carboxylic acid (8.0 g, 43.9 mmol, 1.0 eq), DMF (193 mg, 2.2 mmol, 0.05 eq) and DCM (150 ml). After that, oxalyl chloride (6.1 g, 48.4 mmol, 1.1 eq) was added dropwise with stirring at 0° C. The reaction was stirred for 1 hour at 0° C. The mixture was concentrated, and then dissolved in DCM (5 mL). To the above mixture was added TEA (13.3 g, 131.9 mmol, 3.0 eq) and N, O-dimethylhydroxylamine HCl salt (4.3 g, 43.9 mmol, 1.0 eq) at 0° C. The reaction was stirred for 2 hours at 0° C. The resulting solution was diluted with water (100 mL) and extracted with dichloromethane (3×150 mL). The organic phase was combined and then washed with water (2×100 ml) and brine (1×100 mL). The mixture was dried over anhydrous sodium sulfate and concentrated. The crude residue was applied onto a silica gel column and eluted with ethyl acetate/petroleum ether=1:10 to give N-(methoxymethyl)-N-methyl-4, 5, 6, 7-tetrahydro-1-benzothiophene-2-carboxamide (9 g) as a white solid. LC-MS: (ES, m/z): M+1: 226.

Synthesis of 3-chloro-1-(4, 5, 6, 7-tetrahydro-1-benzothiophen-2-yl) propan-1-one: Into a 250-mL 3-necked round-bottom flask purged and maintained under an inert atmosphere of nitrogen, were placed N-methoxy-N-methyl-4,5,6,7-tetrahydro-1-benzothiophene-2-carboxamide (8.0 g, 35.6 mmol, 1.0 eq) and THE (40 mL). After that, bromo(ethenyl) magnesium (1 M in THF) (160 mL, 142.2 mmol, 4.0 eq) was added dropwise with stirring at −10° C. The reaction was stirred for 3 hours at 0° C. The reaction was then quenched by the addition of 40 mL of 2 M HCl (aq). The resulting solution was extracted with ethyl acetate (2×100 mL) and the organic phase were combined. The resulting mixture was washed with water (2×100 ml) and brine (1×100 mL). The mixture was dried over anhydrous sodium sulfate and concentrated. The resulting solution was diluted with 80 mL of DCM. The residue was dissolved in 40 mL of 2 M HCl (gas) in Et₂O and stirred for 3 hours at 25° C. Then the solution was concentrated and the residue was applied onto a silica gel column and eluted with ethyl acetate/petroleum ether=1:5 to give 3-chloro-1-(4, 5, 6, 7-tetrahydro-1-benzothiophen-2-yl) propan-1-one (2.3 g) as a yellow oil. LC-MS: (ES, m/z): M+1: 229.

Synthesis of 1, 2, 5, 6, 7, 8-hexahydro-3H-benzo[b]cyclopenta[d]thiophen-3-one: Into a 100-mL round-bottom flask, were placed 3-chloro-1-(4, 5, 6, 7-tetrahydro-1-benzothiophen-2-yl) propan-1-one (2.3 g, 10.1 mmol, 1.0 eq) and H₂SO₄ (20 mL). The reaction was stirred for 16 hours at 95° C. The reaction mixture was cooled down to 0° C. The resulting solution was diluted with water (50 mL), extracted with ethyl acetate (2×50 mL) and the organic phase were combined. The resulting mixture was washed with brine (1×50 ml). The mixture was dried over anhydrous sodium sulfate and concentrated. The crude residue was applied onto a silica gel column and eluted with ethyl acetate/petroleum ether=1:5 to give 1, 2, 5, 6, 7, 8-hexahydro-3H-benzo[b]cyclopenta[d]thiophen-3-one (0.8 g) as brown oil. LC-MS: (ES, m/z): M+1: 193.

Synthesis of (Z)-1, 2, 5, 6, 7, 8-hexahydro-3H-benzo[b]cyclopenta[d]thiophen-3-one oxime: Into a 100-mL 3-necked round-bottom flask purged and maintained under an inert atmosphere of nitrogen, were placed NH₂OH·HCl (1.41 g, 20.3 mmol, 5.0 eq), MeOH (30 mL). After that, NaOAc (1.7 g, 20.3 mmol, 5.0 eq) was added at 0° C. and the reaction was stirred for 30 minutes at 0° C. To the above mixture was added 1, 2, 5, 6, 7, 8-hexahydro-3H-benzo[b]cyclopenta[d]thiophen-3-one (780 mg, 4.1 mmol, 1.0 eq) at 0° C. The reaction was stirred for 18 hours at 0° C. The mixture was diluted with DCM (60 mL), then washed with water (2×30 mL) and brine (1×50 mL). The organic phase was combined and dried over anhydrous sodium sulfate and concentrated. The crude residue was applied onto a silica gel column and eluted with ethyl acetate/petroleum ether=1:1 to give (Z)-1, 2, 5, 6, 7, 8-hexahydro-3H-benzo[b]cyclopenta[d]thiophen-3-one oxime (300 mg) as brown oil. LC-MS: (ES, m/z): M+1: 208.

Synthesis of 3, 4, 5, 6, 7, 8-hexahydrobenzo [4,5]thieno [2, 3-c]pyridin-1(2H)-one: Into a 50-mL round-bottom flask purged and maintained under an inert atmosphere of nitrogen, were placed (Z)-1, 2, 5, 6, 7, 8-hexahydro-3H-benzo[b]cyclopenta[d]thiophen-3-one oxime (295 mg, 1.4 mmol, 1.0 eq) and PPA (6 mL). The reaction was stirred for 18 hours at 80° C. The reaction mixture was cooled down to 0° C. The resulting solution was diluted with water (20 mL) and extracted with ethyl acetate (2×50 mL) and the organic phase were combined. The resulting mixture was washed with brine (1×50 ml), dried over anhydrous sodium sulfate and concentrated in vacuum. The residue was applied onto a silica gel column and eluted with dichloromethane/methanol=5:1 to give 3, 4, 5, 6, 7, 8-hexahydrobenzo [4, 5]thieno [2, 3-c]pyridin-1(2H)-one (260 mg) as an off-white solid. LC-MS: (ES, m/z): M+1: 208.

### Example INT_12: Preparation of tert-butyl (3S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(1-hydroxyethyl)-1-methyl-6-oxo-1,6-dihydro-[3,4′-bipyridin]-5-yl)amino)pyridin-3-yl)-3-methylpiperazine-1-carboxylate

Synthesis of 1-(2,4-dibromopyridin-3-yl)ethanol: Into a 250 mL 3-necked round-bottom flask were added 2,4-dibromopyridine (15.0 g, 63.3 mmol, 1.0 eq) and THE (100 mL) at −78° C. After that, to the above mixture was added LDA (2 M in THF) (47 mL, 94.9 mmol, 1.5 eq) dropwise over 30 min at −78° C. under nitrogen atmosphere. The resulting mixture was stirred for additional 1 hour at −78° C. To the above mixture was added acetaldehyde (8.3 g, 189.9 mmol, 3.0 eq) dropwise at −78° C. The resulting mixture was stirred for an additional 1 hour at −78° C.-0° C. The reaction was quenched by the addition of sat. NH₄Cl (aq.) (50 mL) at 0° C. and extracted with EtOAc (3×50 mL). The combined organic layers were washed with brine (50 mL), dried over anhydrous Na₂SO₄. After filtration, the filtrate was concentrated under vacuum. The crude residue was purified by a flash column (silica gel, petroleum ether/ethyl acetate=2:1) to give 1-(2,4-dibromopyridin-3-yl)ethanol as an orange oil (12.0 g, 67.5%). ¹HNMR (400 MHz, DMSO-d₆) δ 8.10 (d, J=5.2 Hz, 1H), 7.73 (d, J=5.2 Hz, 1H), 5.51 (d, J=4.0 Hz, 1H), 5.36 (m, 1H), 1.47 (d, J=6.8 Hz, 3H).

Synthesis of 2,4-dibromo-3-[1-(oxan-2-yloxy)ethyl]pyridine: Into a 100 mL 3-necked round-bottom flask were placed 1-(2,4-dibromopyridin-3-yl)ethanol (12.0 g, 42.7 mmol, 1.0 eq), DHP (5.0 g, 64.1 mmol, 1.5 eq), CH₂Cl₂ (100 mL) and PPTS (1.0 g, 4.3 mmol, 0.1 eq). The resulting mixture was stirred for 4 hours at 50° C. The reaction was quenched by the addition of water (20 mL) at 0° C. and extracted with CH₂Cl₂ (3×30 mL). The combined organic layers were washed with brine (30 mL), dried over anhydrous Na₂SO₄. After filtration, the filtrate was concentrated under vacuum. The crude residue was purified by a flash column (silica gel, petroleum ether/ethyl acetate=3:1) to give 2,4-dibromo-3-[1-(oxan-2-yloxy)ethyl]pyridine as an orange oil (15.0 g, 96.2%). LC-MS (ESI, m/z) M+1: 364/366/368.

Synthesis of 10-{4-bromo-3-[1-(oxan-2-yloxy)ethyl]pyridin-2-yl}-4,4-dimethyl-1,10-diazatricyclo[6.4.0.0\hat{ }{2,6}]dodeca-2(6),7-dien-9-one: Into a 250 mL 3-necked round-bottom flask were placed 2,4-dibromo-3-[1-(oxan-2-yloxy)ethyl]pyridine (8.0 g, 21.9 mmol, 1.0 eq), 4,4-dimethyl-1,10-diazatricyclo[6.4.0.0\hat{ }{2,6}]dodeca-2(6),7-dien-9-one (4.5 g, 21.9 mmol, 1.0 eq), 1,4-dioxane (100 mL), Cul (1.6 g, 8.8 mmol, 0.4 eq), 1,10-phenanthroline (2.4 g, 13.1 mmol, 0.6 eq) and K₂CO₃ (9.1 g, 65.7 mmol, 3.0 eq) at 25° C. under nitrogen atmosphere. The resulting mixture was stirred for 30 hours at 110° C. under nitrogen atmosphere. The precipitated solids were collected by filtration and washed with CH₂Cl₂ (3×20 mL). The resulting mixture was diluted with water (30 mL) and extracted with CH₂Cl₂ (3×30 mL). The combined organic layers were washed with brine (30 mL), dried over anhydrous Na₂SO₄. After filtration, the filtrate was concentrated under vacuum. The crude residue was purified by a flash column (silica gel, petroleum ether/ethyl acetate=1:1) to give 10-{4-bromo-3-[1-(oxan-2-yloxy)ethyl]pyridin-2-yl}-4,4-dimethyl-1,10-diazatricyclo[6.4.0.0\hat{ }{2,6}]dodeca-2(6),7-dien-9-one as a brown solid (3.4 g, 31.8%). LC-MS (ESI, m/z) M+1: 488/490.

Synthesis of 4,4-dimethyl-10-{3-[1-(oxan-2-yloxy)ethyl]-4-(4,4,5,5-tetramethyl-1,3,2-dioxaborolan-2-yl)pyridin-2-yl}-1,10-diazatricyclo[6.4.0.0\hat{ }{2,6}]dodeca-2(6),7-dien-9-one: Into a 250 mL 3-necked round-bottom flask were placed 10-{4-bromo-3-[1-(oxan-2-yloxy)ethyl]pyridin-2-yl}-4,4-dimethyl-1,10-diazatricyclo[6.4.0.0\hat{ }{2,6}]dodeca-2(6),7-dien-9-one (3.4 g, 7.0 mmol, 1.0 eq), bis(pinacolato)diboron (4.4 g, 17.4 mmol, 2.5 eq), 1,4-dioxane (20 mL), Pd(dppf)Cl₂ (500 mg, 0.7 mmol, 0.1 eq) and KOAc (2.0 g, 20.9 mmol, 3.0 eq) at 25° C. under nitrogen atmosphere. The resulting mixture was stirred for 2 hours at 100° C. under nitrogen atmosphere. The reaction was quenched by the addition of water (20 mL) and extracted with CH₂Cl₂ (3×20 mL). The combined organic layers were washed with brine (20 mL), dried over anhydrous Na₂SO₄. After filtration, the filtrate was concentrated under vacuum. The crude residue was purified by reverse phase flash using the following conditions: column, C18 silica gel; mobile phase, CH₃CN in water (0.05% TFA), 30% to 70% gradient in 10 min; detector, UV 254 nm to give 4,4-dimethyl-10-{3-[1-(oxan-2-yloxy)ethyl]-4-(4,4,5,5-tetramethyl-1,3,2-dioxaborolan-2-yl)pyridin-2-yl}-1,10-diazatricyclo[6.4.0.0\hat{ }{2,6}]dodeca-2(6),7-dien-9-one as a brown solid (1.1 g, 32.2%). LC-MS (ESI, m/z) M+1: 536.

Synthesis of 10-{1-hydroxy-3-methyl-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one: Into a 50 mL round-bottom flask were added 4,4-dimethyl-10-{3-[1-(oxan-2-yloxy)ethyl]-4-(4,4,5,5-tetramethyl-1,3,2-dioxaborolan-2-yl)pyridin-2-yl}-1,10-diazatricyclo[6.4.0.0\hat{ }{2,6}]dodeca-2(6),7-dien-9-one (1.1 g, 2.1 mmol, 1.0 eq) and HCl in 1,4-dioxane (2 M, 10 mL) at 25° C. The resulting mixture was stirred for 4 hours at 25° C. The resulting mixture was concentrated under vacuum to give 10-{1-hydroxy-3-methyl-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one as a brown solid (650 mg, 90.1%). LC-MS (ES, m/z) M+1: 352.

### Example INT_13: Preparation of dimethyl 4-bromo-5-hydroxyphthalate

Synthesis of 1-bromo-2-methoxy-4,5-dimethylbenzene: Into a 3000 mL 4-necked round-bottom flask were added CH₂Cl₂ (1000 mL), MeOH (1000 mL), Bu₄NBr₃ (177.0 g, 367.0 mmol, 1.0 eq) at 25° C. After that, 4-methoxy-1,2-dimethyl-benzene (50.0 g, 367.1 mmol, 1.0 eq) was added in portions. The reaction mixture was stirred for 16 hours at 25° C. The resulting mixture was diluted with water (2000 mL) and then extracted with CH₂Cl₂ (2×1000 mL). The combined organic layers were washed with HCl (0.5 M, 500 mL) and brine (500 mL), dried over anhydrous Na₂SO₄. After filtration, the filtrate was concentrated under vacuum to give 1-bromo-2-methoxy-4,5-dimethylbenzene as a light yellow solid (78 g, 98.7%).

Synthesis of 4-bromo-5-methoxyphthalic acid: Into a 1000 mL 3-necked round-bottom flask were added 1-bromo-2-methoxy-4,5-dimethylbenzene (26.0 g, 120.8 mmol, 1.0 eq), water (300 mL), NaOH (9.6 g, 240.0 mmol, 2.0 eq) and KMnO₄ (94.8 g, 599.8 mmol, 5.0 eq). The reaction mixture was stirred for 3 hours at 100° C. The resulting mixture was filtered and the filter cake was washed with water (200 mL). The filtrate was acidified to pH=1 with 4 M HCl and then extracted with CH₂Cl₂ (4×300 mL). The combined organic layers were washed with brine (300 mL), dried over anhydrous Na₂SO₄. After filtration, the filtrate was concentrated under vacuum to give 4-bromo-5-methoxyphthalic acid as a white solid (44.0 g, 66.1%).

Synthesis of dimethyl 4-bromo-5-methoxyphthalate: Into a 2000 mL round-bottom flask were added 4-bromo-5-methoxyphthalic acid (44.0 g, 159.9 mmol, 1 eq), MeOH (800 mL) and H₂SO₄ (1.6 g, 16.3 mmol, 0.1 eq). The reaction mixture was stirred for 16 hours at 60° C. The resulting mixture was diluted with water (1000 mL), and then extracted with EtOAc (3×500 mL). The combined organic layers were washed with brine (500 mL), dried over anhydrous Na₂SO₄. After filtration, the filtrate was concentrated under vacuum to give dimethyl 4-bromo-5-methoxyphthalate as a colorless oil (35.0 g, 72.1%).

Synthesis of dimethyl 4-bromo-5-hydroxyphthalate: Into a 1000 mL 3-necked round-bottom flask were added dimethyl 4-bromo-5-methoxyphthalate (35.0 g, 115.4 mmol, 1.0 eq), CH₂Cl₂ (300 mL) and AlCl₃ (30.8 g, 231.0 mmol, 2.0 eq). The resulting mixture was stirred for 16 hours at 25° C. The reaction was quenched by the addition of water (1000 mL) and then extracted with CH₂Cl₂ (3×300 mL). The combined organic layers were washed with brine (300 mL), dried over anhydrous Na₂SO₄. After filtration, the filtrate was concentrated under vacuum. The crude residue was purified by a flash column (silica gel, ethyl acetate/petroleum ether=1:1) to give dimethyl 4-bromo-5-hydroxyphthalate as a white solid (22 g, 65.9%).

### Example 24: Preparation of (3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dion

Synthesis of (S)-2-((tert-butoxycarbonyl)(3-methoxy-3-oxopropyl)amino)-4-methoxy-4-oxobutanoic acid: Into a 2 L 3-necked round-bottom flask were added (S)-2-amino-4-methoxy-4-oxobutanoic acid (250.0 g, 1359.3 mmol, 1.0 eq) and water (750 mL) at 25° C. After that, triethylamine (344.1 g, 3400.5 mmol, 2.5 eq) was added dropwise at 0° C., followed by the addition of methyl acrylate (175.8 g, 2042.0 mmol, 1.5 eq) at 0° C. The reaction mixture was stirred for 4 hours at 25° C. The resulting mixture was washed with hexane (2×500 mL). And then to the aqueous layer were added t-BuOH (250 mL) and Boc₂O (371.1 g, 1702.2 mol, 1.25 eq). The resulting mixture was stirred for additional 4 hours at 25° C. The mixture was extracted with hexane (3×500 mL) and then acidified to pH=3 with 6 M HCl. The resulting mixture was extracted with ethyl acetate (3×400 mL). The combined organic layers were washed with brine (500 mL), dried over anhydrous Na₂SO₄. After filtration, the filtrate was concentrated under vacuum to give (S)-2-((tert-butoxycarbonyl)(3-methoxy-3-oxopropyl)amino)-4-methoxy-4-oxobutanoic acid as a brown oil (200.0 g, 44.1%). LC-MS (ESI, m/z) M-Boc+1: 234.

Synthesis of 2-methylpropan-2-aminium (S)-1-(tert-butoxycarbonyl)-4-oxopiperidine-2-carboxylate: Into a 2 L 4-necked round-bottom flask were added (S)-2-((tert-butoxycarbonyl)(3-methoxy-3-oxopropyl)amino)-4-methoxy-4-oxobutanoic acid (200.0 g, 0.6 mol, 1.0 eq), tetrahydrofuran (800 mL) at 25° C. After that, NaOMe (324.0 g, 1.8 mol, 3.0 eq., 30% in MeOH) was added dropwise at 10° C. The resulting mixture was stirred for 3 hours at 70° C. The resulting mixture was concentrated under vacuum. The residue was diluted with water (600 mL), and the resulting mixture was stirred for additional 20 hours at 100° C. The aqueous layer was extracted with ethyl acetate (2×500 mL). The aqueous layer was acidified to pH=3 with 6 M HCl and then extracted with ethyl acetate (2×500 mL). The combined organic layers were washed with brine (500 mL), dried over anhydrous Na₂SO₄. After filtration, to the filtrate was added 2-methylpropan-2-amine (43.9 g, 600.0 mmol, 1.0 eq) dropwise at 25° C. The resulting mixture was stirred for an additional 30 min at 25° C. The precipitated solids were collected by filtration, and the filter cake was dissolved in i-PrOH (500 mL). The mixture was stirred for 30 min at 80° C. After filtration, the filter cake was washed with i-PrOH (100 mL) to give 2-methylpropan-2-aminium (S)-1-(tert-butoxycarbonyl)-4-oxopiperidine-2-carboxylate as a white solid (80.0 g, 42.1%).

Synthesis of (S)-1-(tert-butoxycarbonyl)-4-oxopiperidine-2-carboxylic acid: Into a 500 mL 3-necked round-bottom flask were added 2-methylpropan-2-aminium (S)-1-(tert-butoxycarbonyl)-4-oxopiperidine-2-carboxylate (80.0 g, 250.0 mmol, 1.0 eq), water (200 mL) and HCl (22 mL, 270.0 mmol, 1.1 eq) at 0° C. The resulting mixture was stirred for 30 min at 25° C. The resulting mixture was extracted with ethyl acetate (3×200 mL). The combined organic layers were washed with brine (300 mL), dried over anhydrous Na₂SO₄. After filtration, the filtrate was concentrated under vacuum to give (S)-1-(tert-butoxycarbonyl)-4-oxopiperidine-2-carboxylic acid as a colorless oil (40.0 g, 65.0%). ¹HNMR (300 MHz, DMSO-d₆) δ 12.91 (s, 1H), 4.90-4.55 (m, 1H), 3.98-3.80 (m, 1H), 3.69-3.21 (m, 2H), 3.01-2.78 (m, 1H), 2.60-2.55 (m, 2H), 1.42 (s, 9H).

Synthesis of 1-(tert-butyl) 2-methyl (S)-4-oxopiperidine-1,2-dicarboxylate: Into a 1000 mL 3-necked round-bottom flask were added (S)-1-(tert-butoxycarbonyl)-4-oxopiperidine-2-carboxylic acid (50.0 g, 205.5 mmol, 1.0 eq), DMF (300 mL), and Cs₂CO₃ (40.0 g, 122.7 mmol, 0.6 eq) at 25° C. After that, CH₃I (35.0 g, 246.5 mmol, 1.2 eq) was added dropwise at 25° C. The reaction mixture was stirred for additional 12 hours at 25° C. The resulting mixture was quenched by the addition of water (300 mL) and then extracted with ethyl acetate (2×300 mL). The combined organic layers were washed with brine (300 mL), dried over anhydrous Na₂SO₄. After filtration, the filtrate was concentrated under vacuum. The crude residue was purified by a flash column (silica gel, ethyl acetate/petroleum ether=1:2) to give 1-(tert-butyl) 2-methyl (S)-4-oxopiperidine-1,2-dicarboxylate as a colorless oil (35.0 g, 66.1%). ¹HNMR (300 MHz, DMSO-d₆) δ 5.00-4.66 (m, 1H), 3.96-3.80 (m, 1H), 3.67-3.47 (m, 2H), 2.90 (s, 3H), 2.64-2.53 (m, 1H), 2.53-2.24 (m, 2H), 1.41 (s, 9H).

Synthesis of 8-(tert-butyl) 7-methyl (S)-1,4-dioxa-8-azaspiro[4.5]decane-7,8-dicarboxylate: Into a 500 mL round-bottom flask were added 1-(tert-butyl) 2-methyl (S)-4-oxopiperidine-1,2-dicarboxylate (20.0 g, 77.7 mmol, 1.0 eq), toluene (300 mL), ethylene glycol (9.7 g, 156.3 mmol, 2.0 eq) and TsCl (3.0 g, 15.7 mmol, 0.2 eq) at 25° C. The reaction mixture was stirred for 5 hours at 120° C. The resulting mixture was concentrated under vacuum. The resulting mixture was diluted with water (60 mL) and then extracted with ethyl acetate (3×100 mL). The combined organic layers were washed with brine (100 mL), dried over anhydrous Na₂SO₄. After filtration, the filtrate was concentrated under vacuum. The crude residue was purified by a flash column (silica gel, petroleum ether/ethyl acetate=4:1) to give 8-(tert-butyl) 7-methyl (S)-1,4-dioxa-8-azaspiro[4.5]decane-7,8-dicarboxylate as a colorless oil (15.0 g, 64.0%). ¹HNMR (400 MHz, DMSO-d₆) δ 4.84-4.64 (m, 1H), 3.97-3.80 (m, 4H), 3.79-3.72 (m, 1H), 3.65 (s, 3H), 3.24-2.97 (m, 1H), 2.30-2.17 (m, 1H), 1.87-1.72 (m, 1H), 1.71-1.49 (m, 2H), 1.39 (s, 9H).

Synthesis of tert-butyl (S)-7-(hydroxymethyl)-1,4-dioxa-8-azaspiro[4.5]decane-8-carboxylate: Into a 500 mL 3-necked round-bottom flask were added 8-(tert-butyl) 7-methyl (S)-1,4-dioxa-8-azaspiro[4.5]decane-7,8-dicarboxylate (15.0 g, 49.8 mmol, 1.0 eq) and THE (250 mL) at 0° C. After that, LiAlH₄ (3.8 g, 99.6 mmol, 2.0 eq) was added in portions. The reaction mixture was stirred for 3 hours at 0° C. and then quenched by the addition of water (3.8 mL), 15% NaOH (3.8 mL) and water (11.4 mL) at 0° C. The resulting mixture was filtered and the filter cake was washed with tetrahydrofuran (200 mL). The filtrate was concentrated under vacuum. The crude residue was purified by a flash column (silica gel, ethyl acetate/petroleum ether=1:1) to give tert-butyl (S)-7-(hydroxymethyl)-1,4-dioxa-8-azaspiro[4.5]decane-8-carboxylate as a colorless oil (6.2 g, 45.5%). ¹HNMR (300 MHz, DMSO-d₆) δ 4.59-4.49 (m, 1H), 4.21-4.07 (m, 1H), 3.97-3.76 (m, 6H), 3.55-3.42 (m, 2H), 3.00-2.78 (m, 1H), 1.87-1.75 (m, 1H), 1.67-1.53 (m, 2H), 1.40 (s, 9H).

Synthesis of (S)-(1,4-dioxa-8-azaspiro[4.5]decan-7-yl)methanol: Into a 250 mL 3-necked round-bottom flask were added tert-butyl (S)-7-(hydroxymethyl)-1,4-dioxa-8-azaspiro[4.5]decane-8-carboxylate (12.0 g, 43.9 mmol, 1.0 eq), CH₂Cl₂ (100 mL) and TFA (20 mL) at 25° C. The resulting mixture was concentrated under vacuum to afford (S)-(1,4-dioxa-8-azaspiro[4.5]decan-7-yl)methanol as a light yellow oil (10 g, crude). LC-MS (ESI, m/z) M+1: 174.

Synthesis of (S)-tetrahydro-3H-spiro[[1,2,3]oxathiazolo[3,4-a]pyridine-5,2′-[1,3]dioxolane]1,1-dioxide: Into a 250 mL 3-necked round-bottom flask were added (S)-(1,4-dioxa-8-azaspiro[4.5]decan-7-yl)methanol (9.0 g, 51.9 mmol, 1.0 eq), CH₂Cl₂ (90 mL) and TEA (10.5 g, 103.9 mmol, 2.0 eq) at 25° C. After that, SO₂Cl₂ (7.0 g, 51.9 mmol, 1.0 eq) was added dropwise at 0° C. The reaction mixture was stirred for 3 hours at 25° C. The resulting mixture was concentrated under vacuum to afford (S)-tetrahydro-3H-spiro[[1,2,3]oxathiazolo[3,4-a]pyridine-5,2′-[1,3]dioxolane]1,1-dioxide as an off-white solid (24.0 g, crude).

Synthesis of (S)-7-((2-bromo-4,5-bis(methoxycarbonyl)phenoxy)methyl)-1,4-dioxa-8-azaspiro[4.5]decane-8-sulfonic acid: Into a 500 mL round-bottom flask were added (S)-tetrahydro-3H-spiro[[1,2,3]oxathiazolo[3,4-a]pyridine-5,2′-[1,3]dioxolane]1,1-dioxide (24.0 g, 102.0 mmol, 1 eq), CH₃CN (250 mL), 1,2-dimethyl 4-bromo-5-hydroxyphthalate (14.7 g, 51.0 mmol, 0.5 eq) and K₂CO₃ (28.2 g, 204.0 mmol, 2.0 eq) at 25° C. The reaction mixture was stirred for 16 hours at 50° C. The resulting mixture was concentrated under vacuum. The residue was purified by reverse flash chromatography using the following conditions: column, C18 silica gel; mobile phase, CH₃CN in water (0.5% TFA), 10% to 80% gradient in 10 min; detector, UV 254 nm to afford (S)-7-((2-bromo-4,5-bis(methoxycarbonyl)phenoxy)methyl)-1,4-dioxa-8-azaspiro[4.5]decane-8-sulfonic acid as a colorless oil (5.5 g, 10.2%). LC-MS (ESI, m/z) M+1: 524/526.

Synthesis of (S)-4-((1,4-dioxa-8-azaspiro[4.5]decan-7-yl)methoxy)-5-bromophthalate: Into a 250 mL round-bottom flask were added (S)-7-((2-bromo-4,5-bis(methoxycarbonyl)phenoxy)methyl)-1,4-dioxa-8-azaspiro[4.5]decane-8-sulfonic acid (5.5 g, 10.5 mmol, 1.0 eq), CH₂Cl₂ (50 mL) and TFA (0.25 g, 2.2 mmol, 0.2 eq) at 25° C. The resulting mixture was stirred for 16 hours at 25° C. The reaction was quenched by the addition of water (20 mL) and then extracted with CH₂Cl₂ (3×30 mL). The combined organic layers were washed with sat. NaHCO₃ (30 mL) and brine (30 mL), dried over anhydrous Na₂SO₄. After filtration, the filtrate was concentrated under vacuum. The crude residue was purified by a flash column (silica gel, petroleum ether: ethyl acetate=1:1) to afford dimethyl (S)-4-((1,4-dioxa-8-azaspiro[4.5]decan-7-yl)methoxy)-5-bromophthalate as a brown solid (2.4 g, 51.5%). LC-MS (ESI, m/z) M+1: 444.

Synthesis of dimethyl (S)-6a,7,9,10-tetrahydro-6H-spiro[benzo[b]pyrido[1,2-d][1,4]oxazine-8,2′-[1,3]dioxolane]-2,3-dicarboxylate: Into a 100 mL round-bottom flask were added 1,2-dimethyl 4-bromo-5-[(7S)-1,4-dioxa-8-azaspiro[4.5]decan-7-ylmethoxy]phthalate (2.3 g, 5.1 mmol, 1.0 eq), dioxane (25 mL), Cs₂CO₃ (3.4 g, 10.2 mmol, 2.0 eq) and A-Phos-PdCl₂ (0.4 g, 0.5 mmol, 0.1 eq) at 25° C. The resulting mixture was stirred for 2 hours at 100° C. under nitrogen atmosphere. The resulting mixture was diluted with water (50 mL) and then extracted with EtOAc (3×50 mL). The combined organic layers were washed with brine (30 mL), dried over anhydrous Na₂SO₄. After filtration, the filtrate was concentrated under vacuum. The crude residue was purified by a flash column (silica gel, petroleum ether/ethyl acetate=1:1) to afford 12′,13′-dimethyl dimethyl (S)-6a,7,9,10-tetrahydro-6H-spiro[benzo[b]pyrido[1,2-d][1,4]oxazine-8,2′-[1,3]dioxolane]-2,3-dicarboxylate as a colorless oil (1.0 g, 53.1%). LC-MS (ESI, m/z) M+1: 364. ¹HNMR (300 MHz, DMSO-d₆) δ 7.13 (s, 1H), 7.06 (s, 1H), 4.33-4.24 (m, 1H), 4.06-3.93 (m, 2H), 3.94-3.89 (m, 2H), 3.78 (s, 3H), 3.73 (s, 3H), 3.42-3.24 (m, 1H), 2.90-2.83 (m, 1H), 1.77-1.72 (m, 3H), 1.47-1.42 (m, 2H), 1.22-1.16 (m, 1H).

Synthesis of dimethyl (S)-8-oxo-6,6a,7,8,9,10-hexahydrobenzo[b]pyrido[1,2-d][1,4]oxazine-2,3-dicarboxylate: Into a 100 mL round-bottom flask were added dimethyl (S)-6a,7,9,10-tetrahydro-6H-spiro[benzo[b]pyrido[1,2-d][1,4]oxazine-8,2′-[1,3]dioxolane]-2,3-dicarboxylate (1.0 g, 2.7 mmol, 1.0 eq), CH₂Cl₂ (10 mL), water (1 mL) and TFA (2 mL) at 25° C. The resulting mixture was stirred for 12 hours at 25° C. The reaction mixture was quenched by the addition of water (20 mL) and then extracted with CH₂Cl₂ (3×20 mL). The combined organic layers were washed with brine (2×20 mL), dried over anhydrous Na₂SO₄. After filtration, the filtrate was concentrated under vacuum to afford dimethyl (S)-8-oxo-6,6a,7,8,9,10-hexahydrobenzo[b]pyrido[1,2-d][1,4]oxazine-2,3-dicarboxylate as a colorless oil (0.9 g, crude). LC-MS (ESI, m/z) M+1: 320.

Synthesis of dimethyl (6aS,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate and dimethyl (6aS,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate: Into a 100 mL round-bottom flask were added dimethyl (S)-8-oxo-6,6a,7,8,9,10-hexahydrobenzo[b]pyrido[1,2-d][1,4]oxazine-2,3-dicarboxylate (0.9 g, 2.8 mmol, 1.0 eq), DCE (15 mL) and 5-bromo-1-methyl-3-({5-[(2S)-2-methylpiperazin-1-yl]pyridin-2-yl}amino)pyridin-2-one (1.1 g, 2.8 mmol, 1.0 eq) at 25° C. After that, NaBH(OAc)₃ (1.2 g, 5.6 mmol, 2.0 eq) was added in portions. The reaction mixture was stirred for 24 hours at 25° C. The resulting mixture was quenched by the addition of MeOH (5 mL), diluted with water (20 mL) and then extracted with CH₂Cl₂ (3×20 mL). The combined organic layers were washed with brine (20 mL), dried over anhydrous Na₂SO₄. After filtration, the filtrate was concentrated under vacuum. The residue was purified by reverse flash chromatography using the following conditions: column, C18 silica gel; mobile phase, CH₃CN in Water (0.5% NH₃·H₂O), 40% to 950% gradient in 10 min; detector, UV 254 nm to afford dimethyl dimethyl (6aS,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate as a light yellow solid (500 mg, 26.0%) and dimethyl (6aS,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate as a light yellow solid (230 mg 11.9%). Dimethyl (6aS,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate. LC-MS (ESI, m/z) M+1: 681/683. ¹HNMR (300 MHz, Chloroform-d) δ 8.61 (d, J=2.4 Hz, 1H), 8.04 (d, J=2.7 Hz, 1H), 7.81 (s, 1H), 7.34-7.28 (m, 1H), 7.18 (s, 1H), 7.07 (s, 1H), 6.95 (d, J=2.6 Hz, 1H), 6.77 (d, J=8.9 Hz, 1H), 4.26 (dd, J=10.9, 2.9 Hz, 1H), 4.05-3.99 (m, 1H), 3.96 (s, 3H), 3.84 (s, 3H), 3.60 (s, 3H), 3.49 (s, 1H), 3.14-3.06 (m, 3H), 2.78 (m, 4H), 2.60 (s, 1H), 2.53-2.48 (m, 1H), 2.07-1.99 (m, 1H), 1.92-1.88 (m, 1H), 1.65-1.57 (m, 1H), 1.40-1.18 (m, 2H), 0.99 (d, J=6.3 Hz, 3H). Dimethyl (6aS,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate. LC-MS (ESI, m/z) M+1: 681/683. ¹HNMR (300 MHz, Chloroform-d) δ 8.59 (d, J=2.4 Hz, 1H), 8.01 (d, J=2.7 Hz, 1H), 7.79 (s, 1H), 7.33-7.25 (m, 1H), 7.18 (s, 1H), 7.02 (s, 1H), 6.95 (d, J=2.6 Hz, 1H), 6.77 (d, J=8.9 Hz, 1H), 4.20 (dd, J=10.9, 2.9 Hz, 1H), 4.01-3.96 (m, 1H), 3.90 (s, 3H), 3.82 (s, 3H), 3.61 (s, 3H), 3.52 (s, 1H), 3.14-3.06 (m, 3H), 2.83 (m, 4H), 2.60 (s, 1H), 2.29-2.22 (m, 1H), 2.05-1.99 (m, 1H), 1.84-1.76 (m, 1H), 1.42-1.34 (m, 1H), 1.32-1.24 (m, 2H), 1.01 (d, J=6.3 Hz, 3H).

Synthesis of (6aS,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid: Into a 40 mL vial were added dimethyl (6aS,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate (200 mg, 0.3 mmol, 1.0 eq), THE (2 mL), water (2 mL) and NaOH (70 mg, 1.8 mmol, 6.0 eq) at 25° C. The reaction mixture was stirred for 6 hours at 70° C. The resulting mixture was acidified to pH=5 with 3 M HCl and then concentrated under vacuum to give (6aS,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid as a yellow solid (230 mg, crude). LC-MS (ESI, m/z) M+1: 653/655.

Synthesis of (3R,4aS)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione: Into a 40 mL vial were added (6aS,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid (230 mg, 0.4 mmol, 1.0 eq), HOAc (5 mL), KOAc (104 mg, 1.05 mmol, 3.0 eq) and 3-aminopiperidine-2,6-dione hydrochloride (116 mg, 0.7 mmol, 2.0 eq) at 25° C. The reaction mixture was stirred for 16 hours at 100° C. The resulting mixture was concentrated under vacuum. The reaction was quenched by the addition of water (10 mL) and then extracted with CH₂Cl₂ (3×10 mL). The combined organic layers were washed with brine (10 mL), dried over anhydrous Na₂SO₄. After filtration, the filtrate was concentrated under vacuum. The crude residue was purified by a flash column (silica gel, petroleum ether/ethyl acetate=1:2) to afford (3R,4aS)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione as a yellow solid (160 mg, 60.9%). LC-MS (ESI, m/z) M+1: 745/747.

Synthesis of (3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dion: Into a 5 mL sealed tube were added (3R,4aS)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione (150 mg, 0.2 mmol, 1.0 eq), dioxane (3 mL), 10-{1-hydroxy-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one (68 mg, 0.2 mmol, 1.0 eq), K₂CO₃ (55 mg, 0.4 mmol, 2.0 eq), Pd(DtBPF)Cl₂ (13 mg, 0.02 mmol, 0.1 eq) and water (0.3 mL) at 25° C. The reaction mixture was stirred for 2 hours at 110° C. under nitrogen atmosphere in microwave. The resulting mixture was quenched by the addition of water (10 mL) and then extracted with CH₂Cl₂ (3×10 mL). The combined organic layers were washed with brine (10 mL), dried over anhydrous Na₂SO₄. After filtration, the filtrate was concentrated under vacuum. The residue was purified by reverse flash chromatography using the following conditions: column, C18 silica gel; mobile phase, CH₃CN in Water (0.5% NH₃·H₂O), 10% to 80% gradient in 10 min; detector, UV 254 nm to afford (3R,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione as a yellow solid (16 mg, 8.15%). LC-MS (ESI, m/z) M+1: 976. ¹HNMR (300 MHz, Chloroform-d) δ 8.65 (d, J=2.4 Hz, 1H), 8.48 (d, J=4.9 Hz, 1H), 8.10 (s, 1H), 7.95 (s, 1H) 7.83-7.81 (d, J=26.5 Hz, 2H), 7.33 (d, J=5.0 Hz, 1H), 7.29 (d, J=8.0 Hz, 1H), 7.26-7.25 (m, 1H), 7.19 (s, 1H), 6.84-6.82 (m, 2H), 5.06 (dd, J=12.4, 5.4 Hz, 1H), 4.91-4.89 (m, 1H), 4.62-4.42 (m, 2H), 4.33-4.30 (m, 2H), 4.17-4.14 (m, 2H), 4.08-4.02 (m, 2H), 3.85 (s, 1H), 3.71 (s, 3H), 3.45 (s, 1H), 3.25 (s, 1H), 3.05 (s, 2H), 2.97-2.79 (m, 7H), 2.76-2.57 (m, 6H), 2.10-2.00 (m, 2H), 1.99-1.90 (m, 1H), 1.36-1.32 (m, 1H), 1.27 (s, 6H), 0.96 (d, J=6.3 Hz, 3H).

### Example 25: Preparation of (3S,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione

Synthesis of (6aS,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid: Into a 40 mL vial were added dimethyl dimethyl (6aS,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate (200 mg, 0.3 mmol, 1.0 eq), THE (2 mL), water (2 mL) and NaOH (70 mg, 1.8 mmol, 6.0 eq) at 25° C. The reaction mixture was stirred for 6 hours at 70° C. The resulting mixture was acidified to pH=5 with 3 M HCl. The resulting mixture was concentrated under vacuum to afford (6aS,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid as a yellow solid (230 mg, crude %). LC-MS (ESI, m/z) M+1: 653/655.

Synthesis of (3S,4aS)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione: Into a 40 mL vial were added (6aS,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid (230 mg, 0.4 mmol, 1.0 eq), HOAc (5 mL), KOAc (104 mg, 1.05 mmol, 3.0 eq) and 3-aminopiperidine-2,6-dione hydrochloride (116 mg, 0.7 mmol, 2.0 eq) at 25° C. The reaction mixture was stirred for 16 hours at 100° C. The resulting mixture was concentrated under vacuum. The residue was quenched by the addition of water (10 mL) and then extracted with CH₂Cl₂ (3×10 mL). The combined organic layers were washed with brine (10 mL), dried over anhydrous Na₂SO₄. After filtration, the filtrate was concentrated under reduced pressure. The crude residue was purified by a flash column (silica gel, petroleum ether/ethyl acetate=1:2) to afford (3S,4aS)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione as a yellow solid (150 mg, 57.16%). LC-MS (ESI, m/z) M+1: 745/747.

Synthesis of (3S,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione: Into a 5 mL sealed tube were added (3S,4aS)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione (150 mg, 0.2 mmol, 1.0 eq), dioxane (3 mL), 10-{1-hydroxy-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one (68 mg, 0.2 mmol, 1.0 eq), K₂CO₃ (55 mg, 0.4 mmol, 2.0 eq), Pd(DtBPF)Cl₂ (13 mg, 0.02 mmol, 0.1 eq) and water (0.3 mL) at 25° C. The resulting mixture was stirred for 2 hours at 110° C. under nitrogen atmosphere in microwave. The reaction was quenched by the addition of water (10 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.5% NH₃·H₂O), 10% to 80% gradient in 10 min; detector, UV 254 nm to afford (3S,4aS)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione as a yellow solid (16 mg, 8.1%). LC-MS (ESI, m/z) M+1: 976. ¹HNMR (300 MHz, Chloroform-d) δ 8.64(d, J=2.4 Hz, 1H), 8.46 (d, J=4.9 Hz, 1H), 8.16 (s, 1H), 7.94 (s, 1H) 7.83-7.81 (d, J=26.5 Hz, 2H), 7.33 (d, J=5.0 Hz, 1H), 7.26 (d, J=8.0 Hz, 1H), 7.20-7.17 (m, 2H), 6.84-6.82 (m, 2H), 5.06 (dd, J=12.4, 5.4 Hz, 1H), 4.91-4.89 (m, 1H), 4.62-4.42 (m, 2H), 4.33-4.30 (m, 2H), 4.17-4.14 (m, 2H), 4.02-3.99 (m, 1H), 3.85 (s, 1H), 3.71 (s, 3H), 3.45 (s, 3H), 3.25-3.05 (m, 3H), 2.97-2.69 (m, 5H), 2.67-2.50 (m, 7H), 2.30-2.25 (m, 1H), 2.13-2.09 (m, 1H), 1.99-1.90 (m, 1H), 1.36-1.32 (m, 1H), 1.27 (s, 6H), 0.97 (d, J=6.3 Hz, 3H).

### Example 26: Preparation of (3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione

Synthesis of (R)-2-((tert-butoxycarbonyl)(3-methoxy-3-oxopropyl)amino)-4-methoxy-4-oxobutanoic acid: Into a 2 L 3-necked round-bottom flask were added (S)-2-amino-4-methoxy-4-oxobutanoic acid (250.0 g, 1359.3 mmol, 1.0 eq), water (750 mL) at 25° C. After that, triethylamine (344.1 g, 3400.5 mmol, 2.5 eq) was added dropwise at 0° C., followed by the addition of methyl acrylate (175.8 g, 2042.0 mmol, 1.5 eq) at 0° C. The reaction mixture was stirred for 4 hours at 25° C. The resulting mixture was extracted with hexane (2×500 mL). To the aqueous layer was added t-BuOH (250 mL) and Boc₂O (371.1 g, 1702.2 mol, 1.25 eq). The resulting mixture was stirred for additional 4 hours at 25° C. The mixture was acidified to pH=3 with 6 M HCl and then extracted with ethyl acetate (3×400 mL). The combined organic layers were washed with brine (500 mL), dried over anhydrous Na₂SO₄. After filtration, the filtrate was concentrated under vacuum to give (S)-2-((tert-butoxycarbonyl)(3-methoxy-3-oxopropyl)amino)-4-methoxy-4-oxobutanoic acid as a brown oil (200.0 g, 44.1%). LC-MS (ESI, m/z) M-Boc+1: 234.

Synthesis of 2-methylpropan-2-aminium (R)-1-(tert-butoxycarbonyl)-4-oxopiperidine-2-carboxylate: Into a 2 L 4-necked round-bottom flask were added (R)-2-((tert-butoxycarbonyl)(3-methoxy-3-oxopropyl)amino)-4-methoxy-4-oxobutanoic acid (200.0 g, 0.6 mol, 1.0 eq), tetrahydrofuran (800 mL) at 25° C. After that, NaOMe (324.0 g, 1.8 mol, 3.0 eq., 30% in MeOH) was added dropwise at 10° C. The reaction mixture was stirred for 3 hours at 70° C. The resulting mixture was concentrated under vacuum, and then the residue was diluted with water (600 mL). After stirring for additional 20 hours at 100° C., the aqueous layer was extracted with ethyl acetate (2×500 mL) and then acidified to pH=3 with 6 M HCl. The resulting mixture was extracted with ethyl acetate (2×500 mL). The combined organic layers were washed with brine (500 mL), dried over anhydrous Na₂SO₄. After filtration, to the filtrate was added 2-methylpropan-2-amine (43.9 g, 600.0 mmol, 1.0 eq) dropwise at 25° C. The resulting mixture was stirred for additional 30 min at 25° C. The precipitated solids were collected by filtration and then the filter cake was dissolved in i-PrOH (500 mL). The mixture was stirred for 30 min at 80° C. and allowed to cool down to 5° C. After filtration, the filter cake was dried in vacuo to give 2-methylpropan-2-aminium (R)-1-(tert-butoxycarbonyl)-4-oxopiperidine-2-carboxylate as a white solid (80.0 g, 42.1%).

Synthesis of (R)-1-(tert-butoxycarbonyl)-4-oxopiperidine-2-carboxylic acid: Into a 500 mL 3-necked round-bottom flask were added 2-methylpropan-2-aminium (R)-1-(tert-butoxycarbonyl)-4-oxopiperidine-2-carboxylate (80.0 g, 250.0 mmol, 1.0 eq), water (200 mL) and HCl (22 mL, 270.0 mmol, 1.1 eq) at 0° C. The reaction mixture was stirred for 30 min at 25° C. The resulting mixture was extracted with ethyl acetate (3×200 mL). The combined organic layers were washed with brine (300 mL), dried over anhydrous Na₂SO₄. After filtration, the filtrate was concentrated under vacuum to give (R)-1-(tert-butoxycarbonyl)-4-oxopiperidine-2-carboxylic acid as a colorless oil (40.0 g, 65.0%). ¹HNMR (300 MHz, DMSO-d₆) δ 12.91 (s, 1H), 4.90-4.55 (m, 1H), 3.98-3.80 (m, 1H), 3.69-3.21 (m, 2H), 3.01-2.78 (m, 1H), 2.60-2.55 (m, 2H), 1.42 (s, 9H).

Synthesis of 1-(tert-butyl) 2-methyl (R)-4-oxopiperidine-1,2-dicarboxylate: Into a 1000 mL 3-necked round-bottom flask were added (R)-1-(tert-butoxycarbonyl)-4-oxopiperidine-2-carboxylic acid (50.0 g, 205.5 mmol, 1.0 eq), DMF (300 mL), and Cs₂CO₃ (40.0 g, 122.7 mmol, 0.6 eq) at 25° C. After that, CH₃I (35.0 g, 246.5 mmol, 1.2 eq) was added dropwise at 25° C. The reaction mixture was stirred for additional 12 hours at 25° C. The resulting mixture was quenched by the addition of water (300 mL) and then extracted with ethyl acetate (2×300 mL). The combined organic layers were washed with brine (300 mL), dried over anhydrous Na₂SO₄. After filtration, the filtrate was concentrated under vacuum. The crude residue was purified by a flash column (silica gel, ethyl acetate/petroleum ether=1:2) to give 1-(tert-butyl) 2-methyl (R)-4-oxopiperidine-1,2-dicarboxylate as a colorless oil (35.0 g, 66.1%). ¹HNMR (300 MHz, DMSO-d₆) δ 5.00-4.66 (m, 1H), 3.96-3.80 (m, 1H), 3.67-3.47 (m, 2H), 2.90 (s, 3H), 2.64-2.53 (m, 1H), 2.53-2.24 (m, 2H), 1.41 (s, 9H).

Synthesis of 8-(tert-butyl) 7-methyl (R)-1,4-dioxa-8-azaspiro[4.5]decane-7,8-dicarboxylate: Into a 500 mL round-bottom flask were added 1-(tert-butyl) 2-methyl (R)-4-oxopiperidine-1,2-dicarboxylate (20.0 g, 77.7 mmol, 1.0 eq), toluene (300 mL), ethylene glycol (9.7 g, 156.3 mmol, 2.0 eq) and TsCI (3.0 g, 15.7 mmol, 0.2 eq) at 25° C. The resulting mixture was stirred for 5 hours at 120° C. The resulting mixture was concentrated under vacuum. The residue was diluted with water (60 mL) and then extracted with ethyl acetate (3×100 mL). The combined organic layers were washed with brine (100 mL), dried over anhydrous Na₂SO₄. After filtration, the filtrate was concentrated under vacuum. The crude residue was purified by a flash column (silica gel, petroleum ether/ethyl acetate=4:1) to give 8-(tert-butyl) 7-methyl (R)-1,4-dioxa-8-azaspiro[4.5]decane-7,8-dicarboxylate as a colorless oil (15.0 g, 64.0%). ¹HNMR (400 MHz, DMSO-d₆) δ 4.84-4.64 (m, 1H), 3.97-3.80 (m, 4H), 3.79-3.72 (m, 1H), 3.65 (s, 3H), 3.24-2.97 (m, 1H), 2.30-2.17 (m, 1H), 1.87-1.72 (m, 1H), 1.71-1.49 (m, 2H), 1.39 (s, 9H).

Synthesis of tert-butyl (R)-7-(hydroxymethyl)-1,4-dioxa-8-azaspiro[4.5]decane-8-carboxylate: Into a 500 mL 3-necked round-bottom flask were added 8-(tert-butyl) 7-methyl (R)-1,4-dioxa-8-azaspiro[4.5]decane-7,8-dicarboxylate (15.0 g, 49.8 mmol, 1.0 eq) and THE (250 mL) at 0° C. After that, LiAlH₄ (3.8 g, 99.6 mmol, 2.0 eq) was added in portions. The resulting mixture was stirred for 3 hours at 0° C. The reaction mixture was quenched by the addition of water (3.8 mL), 15% NaOH (3.8 mL) and water (11.4 mL) at 0° C. The resulting mixture was filtered, the filter cake was washed with tetrahydrofuran (200 mL). The filtrate was concentrated under vacuum. The crude residue was purified by a flash column (silica gel, ethyl acetate/petroleum ether=1:1) to give tert-butyl (R)-7-(hydroxymethyl)-1,4-dioxa-8-azaspiro[4.5]decane-8-carboxylate as a colorless oil (6.2 g, 45.5%). ¹HNMR (300 MHz, DMSO-d₆) δ4.59-4.49 (m, 1H), 4.21-4.07 (m, 1H), 3.97-3.76 (m, 6H), 3.55-3.42 (m, 2H), 3.00-2.78 (m, 1H), 1.87-1.75 (m, 1H), 1.67-1.53 (m, 2H), 1.40 (s, 9H).

Synthesis of (R)-(1,4-dioxa-8-azaspiro[4.5]decan-7-yl)methanol: Into a 250 mL 3-necked round-bottom flask were added tert-butyl (R)-7-(hydroxymethyl)-1,4-dioxa-8-azaspiro[4.5]decane-8-carboxylate (12.0 g, 43.9 mmol, 1.0 eq), CH₂Cl₂ (100 mL) and TFA (20 mL) at 25° C. The resulting mixture was concentrated under vacuum to afford (R)-(1,4-dioxa-8-azaspiro[4.5]decan-7-yl)methanol as a light yellow oil (10 g, crude). LC-MS (ESI, m/z) M+1: 174.

Synthesis of (R)-tetrahydro-3H-spiro[[1,2,3]oxathiazolo[3,4-a]pyridine-5,2′-[1,3]dioxolane]1,1-dioxide: Into a 250 mL 3-necked round-bottom flask were added (R)-(1,4-dioxa-8-azaspiro[4.5]decan-7-yl)methanol (10.0 g, 57.8 mmol, 1.0 eq) and TEA (11.7 g, 115.6 mmol, 2.0 eq) at 25° C. After that, SO₂Cl₂ (7.8 g, 57.8 mmol, 1.0 eq) was added dropwise at 0° C. The reaction mixture was stirred for additional 3 hours at 25° C. The resulting mixture was concentrated under vacuum to afford (R)-tetrahydro-3H-spiro[[1,2,3]oxathiazolo[3,4-a]pyridine-5,2′-[1,3]dioxolane]1,1-dioxide as an off-white solid (26.5 g, crude).

Synthesis of (R)-7-((2-bromo-4,5-bis(methoxycarbonyl)phenoxy)methyl)-1,4-dioxa-8-azaspiro[4.5]decane-8-sulfonic acid: Into a 500 mL round-bottom flask were added (R)-tetrahydro-3H-spiro[[1,2,3]oxathiazolo[3,4-a]pyridine-5,2′-[1,3]dioxolane]1,1-dioxide (26.5 g, 112.6 mmol, 1.0 eq), CH₃CN (250 mL), 1,2-dimethyl 4-bromo-5-hydroxyphthalate (14.7 g, 51.0 mmol, 0.5 eq) and K₂CO₃ (31.1 g, 225.0 mmol, 2.0 eq) at 25° C. The resulting mixture was stirred for 16 hours at 50° C. The resulting mixture was concentrated under vacuum. The residue was purified by reverse flash chromatography using the following conditions: column, C18 silica gel; mobile phase, CH₃CN in water (0.5% TFA), 10% to 80% gradient in 10 min; detector, UV 254 nm to afford (R)-7-((2-bromo-4,5-bis(methoxycarbonyl)phenoxy)methyl)-1,4-dioxa-8-azaspiro[4.5]decane-8-sulfonic acid as a colorless oil (6.5 g, 11.0%). LC-MS (ESI, m/z) M+1: 524/526.

Synthesis of dimethyl dimethyl (R)-4-((1,4-dioxa-8-azaspiro[4.5]decan-7-yl)methoxy)-5-bromophthalate: Into a 250 mL round-bottom flask were added (R)-7-((2-bromo-4,5-bis(methoxycarbonyl)phenoxy)methyl)-1,4-dioxa-8-azaspiro[4.5]decane-8-sulfonic acid (6.1 g, 11.6 mmol, 1.0 eq) and TFA (0.3 g, 2.6 mmol, 0.2 eq) at 25° C. The resulting mixture was stirred for 16 hours at 25° C. The reaction was quenched by the addition of water (20 mL) and then extracted with CH₂Cl₂ (3×30 mL). The combined organic layers were washed with sat. NaHCO₃ (30 mL) and brine (30 mL), dried over anhydrous Na₂SO₄. After filtration, the filtrate was concentrated under vacuum. The crude residue was purified by a flash column (silica gel, petroleum ether: ethyl acetate=1:1) to afford dimethyl (R)-4-((1,4-dioxa-8-azaspiro[4.5]decan-7-yl)methoxy)-5-bromophthalate as a brown solid (2.4 g, 46.4%). LC-MS (ESI, m/z) M+1: 444/446. ¹HNMR (300 MHz, DMSO-d₆) δ 8.01 (s, 1H), 7.35 (s, 1H), 4.13-4.05 (m, 2H), 3.92-3.84 (m, 4H), 3.80 (s, 3H), 3.78 (s, 3H), 3.04-2.98 (m, 2H), 2.72-2.64 (m, 1H), 1.82-1.74 (m, 1H), 1.64-1.57 (m, 1H), 1.49-1.41 (m, 3H).

Synthesis of dimethyl dimethyl (R)-6a,7,9,10-tetrahydro-6H-spiro[benzo[b]pyrido[1,2-d][1,4]oxazine-8,2′-[1,3]dioxolane]-2,3-dicarboxylate: Into a 100 mL round-bottom flask were added dimethyl (R)-4-((1,4-dioxa-8-azaspiro[4.5]decan-7-yl)methoxy)-5-bromophthalate (2.3 g, 5.1 mmol, 1.0 eq), dioxane (25 mL), Cs₂CO₃ (3.4 g, 10.4 mmol, 2.0 eq) and A-Phos-PdCl₂ (0.4 g, 0.5 mmol, 0.1 eq) at 25° C. The reaction mixture was stirred for 2 hours at 100° C. under nitrogen atmosphere. The resulting mixture was diluted with water (50 mL) and then extracted with EtOAc (3×50 mL). The combined organic layers were washed with brine (30 mL), dried over anhydrous Na₂SO₄.

After filtration, the filtrate was concentrated under vacuum. The crude residue was purified by a flash column (silica gel, petroleum ether/ethyl acetate=1:1) to afford dimethyl (R)-6a,7,9,10-tetrahydro-6H-spiro[benzo[b]pyrido[1,2-d][1,4]oxazine-8,2′-[1,3]dioxolane]-2,3-dicarboxylate as a colorless oil (1.2 g, 63.7%). LC-MS (ESI, m/z) M+1: 364. ¹HNMR (300 MHz, DMSO-d₆) δ 7.13 (s, 1H), 7.06 (s, 1H), 4.33-4.25 (m, 2H), 4.03-3.98 (m, 2H), 3.97-3.91 (m, 4H), 3.77 (s, 3H), 3.73 (s, 3H), 3.33-3.25 (m, 1H), 2.88-2.82 (m, 1H), 1.78-1.70 (m, 2H), 1.48-1.42 (m, 1H).

Synthesis of dimethyl (R)-8-oxo-6,6a,7,8,9,10-hexahydrobenzo[b]pyrido[1,2-d][1,4]oxazine-2,3-dicarboxylate: Into a 100 mL round-bottom flask were added dimethyl (R)-6a,7,9,10-tetrahydro-6H-spiro[benzo[b]pyrido[1,2-d][1,4]oxazine-8,2′-[1,3]dioxolane]-2,3-dicarboxylate (1.2 g, 3.3 mmol, 1.0 eq) and TFA (2 mL) at 25° C. The resulting mixture was stirred for 12 hours at 25° C. The reaction was quenched by the addition of water (20 mL) and then extracted with CH₂Cl₂ (3×20 mL). The combined organic layers were washed with brine (2×20 mL), dried over anhydrous Na₂SO₄. After filtration, the filtrate was concentrated under vacuum to afford dimethyl (R)-8-oxo-6,6a,7,8,9,10-hexahydrobenzo[b]pyrido[1,2-d][1,4]oxazine-2,3-dicarboxylate as a colorless oil (1.1 g, crude). LC-MS (ESI, m/z) M+1: 320.

Synthesis of dimethyl (6aR,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate (assumed) and dimethyl (6aR,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate: Into a 100 mL round-bottom flask were added dimethyl (R)-8-oxo-6,6a,7,8,9,10-hexahydrobenzo[b]pyrido[1,2-d][1,4]oxazine-2,3-dicarboxylate (1.1 g, 3.4 mmol, 1.0 eq) and 5-bromo-1-methyl-3-({5-[(2S)-2-methylpiperazin-1-yl]pyridin-2-yl}amino)pyridin-2-one (1.3 g, 3.4 mmol, 1.0 eq) at 25° C. After that, NaBH(OAc)₃ (1.5 g, 6.8 mmol, 2.0 eq) was added in portions. The reaction mixture was stirred for 24 hours at 25° C. The resulting mixture was quenched by the addition of MeOH (5 mL), diluted with water (20 mL) and extracted with CH₂Cl₂ (3×20 mL). The combined organic layers were washed with brine (20 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.5% NH₃·H₂O), 40% to 950% gradient in 10 min; detector, UV 254 nm to afford dimethyl (6aR,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate as a light yellow solid (230 mg, 9.8%), and dimethyl (6aR,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate as a light yellow solid (520 mg, 22.1%). Dimethyl (6aR,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate. LC-MS (ESI, m/z) M+1: 681/683. ¹HNMR (300 MHz, Chloroform-d) δ 8.60 (d, J=2.4 Hz, 1H), 8.03 (d, J=2.7 Hz, 1H), 7.81 (s, 1H), 7.34-7.28 (m, 1H), 7.18 (s, 1H), 7.07 (s, 1H), 6.95 (d, J=2.6 Hz, 1H), 6.77 (d, J=8.9 Hz, 1H), 4.25 (dd, J=10.9, 2.9 Hz, 1H), 4.05-3.99 (m, 1H), 3.96 (s, 3H), 3.84 (s, 3H), 3.59 (s, 3H), 3.49 (s, 1H), 3.14-3.06 (m, 3H), 2.78 (m, 4H), 2.60-2.56 (m, 1H), 2.52-2.47 (m, 1H), 2.06-1.99 (m, 1H), 1.94-1.90 (m, 1H), 1.65-1.57 (m, 1H), 1.35-1.18 (m, 2H), 0.99 (d, J=6.3 Hz, 3H). Dimethyl (6aR,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate. LC-MS (ESI, m/z) M+1: 681/683. ¹HNMR (300 MHz, Chloroform-d) δ 8.59 (d, J=2.4 Hz, 1H), 8.99 (d, J=2.7 Hz, 1H), 7.78 (s, 1H), 7.29-7.27 (m, 1H), 7.19 (s, 1H), 7.00 (s, 1H), 6.94 (d, J=2.6 Hz, 1H), 6.77 (d, J=8.9 Hz, 1H), 4.22 (dd, J=10.9, 2.9 Hz, 1H), 4.15-4.09 (m, 1H), 3.98 (s, 3H), 3.96 (s, 3H), 3.60 (s, 3H), 3.53-3.49 (m, 2H), 3.14-3.06 (m, 2H), 2.83-2.79 (m, 1H), 2.60-2.56 (m, 1H), 2.52-2.47 (m, 1H), 2.23-2.17(m, 1H), 2.06-1.99 (m, 1H), 1.83-1.77 (m, 2H), 1.51-1.46 (m, 1H), 1.35-1.18 (m, 2H), 0.99 (d, J=6.3 Hz, 3H).

Synthesis of (6aR,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid: Into a 40 mL vial were added dimethyl (6aR,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate (200 mg, 0.3 mmol, 1.0 eq) and NaOH (70 mg, 1.8 mmol, 6.0 eq) at 25° C. The reaction mixture was stirred for 6 hours at 70° C. The resulting mixture was acidified to pH=5 with 3 M HCl. The resulting mixture was concentrated under vacuum to afford (6aR,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid as a yellow solid (230 mg, crude). LC-MS (ESI, m/z) M+1: 653/655.

Synthesis of (3R,4aR)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione: Into a 40 mL vial were added (6aR,8R)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid (230 mg, 0.4 mmol, 1.0 eq), HOAc (5 mL), KOAc (104 mg, 1.05 mmol, 3.0 eq) and 3-aminopiperidine-2,6-dione hydrochloride (116 mg, 0.7 mmol, 2.0 eq) at 25° C. The resulting mixture was stirred for 16 hours at 100° C. The resulting mixture was concentrated under vacuum. The residue was diluted with water (10 mL) and then extracted with CH₂Cl₂ (3×10 mL). The combined organic layers were washed with brine (10 mL), dried over anhydrous Na₂SO₄. After filtration, the filtrate was concentrated under vacuum. The crude residue was purified by a flash column (silica gel, petroleum ether/ethyl acetate=1:2) to afford (3R,4aR)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione as a yellow solid (150 mg, 57.1%). LC-MS (ESI, m/z) M+1: 745/747.

Synthesis of (3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione: Into a 5 mL sealed tube were added (3R,4aR)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione (150 mg, 0.2 mmol, 1.0 eq), dioxane (3 mL), 10-{1-hydroxy-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one (68 mg, 0.2 mmol, 1.0 eq), K₂CO₃ (55 mg, 0.4 mmol, 2.0 eq), Pd(DtBPF)Cl₂ (13 mg, 0.01 mmol, 0.1 eq), and water (0.3 mL) at 25° C. The resulting mixture was stirred for 2 hours at 110° C. under nitrogen atmosphere in microwave. The reaction was quenched by the addition of water (10 mL) and then extracted with CH₂Cl₂ (3×10 mL). The combined organic layers were washed with brine (10 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.5% NH₃·H₂O), 10% to 80% gradient in 10 min; detector, UV 254 nm to afford (3R,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione as a yellow solid (16 mg, 8.1%). LC-MS (ESI, m/z) M+1: 976. ¹HNMR (300 MHz, Chloroform-d) δ 8.63(d, J=2.4 Hz, 1H), 8.47 (d, J=4.9 Hz, 1H), 7.97-7.95 (m, 2H), 7.82-7.82 (m, 2H), 7.35 (d, J=5.0 Hz, 1H), 7.29 (s, 1H), 7.19-7.18 (m, 2H), 6.83-6.79 (m, 2H), 5.05 (dd, J=12.4, 5.4 Hz, 1H), 4.92-4.89 (m, 1H), 4.68-4.79 (m, 1H), 4.17-4.15 (m, 2H), 4.14-4.12 (m, 2H), 4.04-4.02 (m, 1H), 3.98 (s, 1H), 3.70 (s, 3H), 3.59-3.56 (m, 3H), 3.11-3.07 (m, 3H), 2.91-2.83 (m, 4H), 2.79-2.71 (m, 8H), 2.56-2.50 (m, 1H), 2.13-2.09 (m, 2H), 1.90-1.70 (m, 1H), 1.57-1.43 (m, 1H), 1.27-1.22 (s, 6H), 0.98 (d, J=6.3 Hz, 3H).

### Example 27: Preparation of (3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione

Synthesis of (6aR,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid (assumed): Into a 40 mL vial were added dimethyl (6aR,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylate (200 mg, 0.3 mmol, 1.0 eq) and NaOH (70 mg, 1.8 mmol, 6.0 eq) at 25° C. The resulting mixture was stirred for 6 hours at 70° C. The resulting mixture was acidified to pH=5 with 3 M HCl. The resulting mixture was concentrated under vacuum to afford (6aR,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid as a yellow solid (230 mg, crude). LC-MS (ESI, m/z) M+1: 653/655.

Synthesis of (3S,4aR)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione: Into a 40 mL vial were added (6aR,8S)-8-((S)-4-(6-((5-bromo-1-methyl-2-oxo-1,2-dihydropyridin-3-yl)amino)pyridin-3-yl)-3-methylpiperazin-1-yl)-6,6a,7,8,9,10-hexahydrobenzo[b]pyrido[1,2-d][1,4]oxazine-2,3-dicarboxylic acid (230 mg, 0.4 mmol, 1.0 eq), HOAc (5 mL), KOAc (104 mg, 1.05 mmol, 3.0 eq) and 3-aminopiperidine-2,6-dione hydrochloride (116 mg, 0.7 mmol, 2.0 eq) at 25° C. The resulting mixture was stirred for 16 hours at 100° C. The resulting mixture was concentrated under vacuum. The reaction was quenched by the addition of water (10 mL) and then extracted with CH₂Cl₂ (3×10 mL). The combined organic layers were washed with brine (10 mL), dried over anhydrous Na₂SO₄. After filtration, the filtrate was concentrated under vacuum. The crude residue was purified by a flash column (silica gel, petroleum ether/tetrahydrofuran=1:2) to afford (3S,4aR)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione as a yellow solid (150 mg, 57.1%). LC-MS (ESI, m/z) M+1: 745/747.

Synthesis of (3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione: Into a 5 mL sealed tube were added (3S,4aR)-3-((S)-4-(6-((5-bromo-1-methyl-2-oxo-1,2-dihydropyridin-3-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione (150 mg, 0.2 mmol, 1.0 eq), dioxane (3 mL), 10-{1-hydroxy-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one (68 mg, 0.2 mmol, 1.0 eq), K₂CO₃ (55 mg, 0.4 mmol, 2.0 eq), Pd(DtBPF)Cl₂ (13 mg, 0.02 mmol, 0.1 eq), and water (0.3 mL) at 25° C. The resulting mixture was stirred for 2 hours at 110° C. under nitrogen atmosphere in microwave. The reaction was quenched by the addition of water (10 mL) and then extracted with DCM (3×10 mL). The combined organic layers were washed with brine (10 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.5% NH₃·H₂O), 10% to 80% gradient in 10 min; detector, UV 254 nm to afford (3S,4aR)-3-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-9-(2,6-dioxopiperidin-3-yl)-1,2,3,4,4a,5-hexahydro-8H-pyrido[1′,2′:4,5][1,4]oxazino[2,3-f]isoindole-8,10(9H)-dione as a yellow solid (24 mg, 12.2%). LC-MS (ESI, m/z) M+1: 976. ¹HNMR (300 MHz, Chloroform-d) δ 8.65(d, J=2.4 Hz, 1H), 8.48 (d, J=4.9 Hz, 1H), 8.17 (s, 1H), 7.97 (s, 1H), 7.85-7.82 (m, 2H), 7.36-7.34 (m, 1H), 7.33-7.30 (m, 1H), 7.25 (s, 1H), 7.19 (s, 1H), 6.83-6.79 (m, 2H), 5.05 (dd, J=12.4, 5.4 Hz, 1H), 4.92-4.89 (m, 1H), 4.28-4.25 (m, 2H), 4.17-4.15 (m, 2H), 4.14-4.12 (m, 2H), 4.04-4.02 (m, 2H), 3.98 (s, 1H), 3.70 (s, 3H), 3.5-3.42 (m, 1H), 3.31-3.24 (m, 1H), 3.12-3.01 (m, 1H), 2.97-2.72 (m, 8H), 2.57 (s, 2H), 2.51-2.45 (m, 3H), 2.13-2.2.09 (m, 3H), 1.27-1.22 (s, 8H), 0.98 (d, J=6.3 Hz, 3H).

### Example 28: Preparation of 5-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione hydrochloride

Synthesis of (S)-2-methylpiperidin-4-one hydrochloride: Into a 50 mL vial were added tert-butyl (S)-2-methyl-4-oxopiperidine-1-carboxylate (3.0 g, 14.1 mmol, 1 eq) and HCl (gas) in 1,4-dioxane (4 M, 10 mL). The resulting mixture was stirred for 4 hours at 25° C. The precipitated solids were collected by filtration and washed with EtOAc (3×5 mL) to give (S)-2-methylpiperidin-4-one hydrochloride as a white solid (1.5 g, 94.2%). LC-MS (ESI, m/z) M+1: 114.

Synthesis of (S)-4-(2-methyl-4-oxopiperidin-1-yl)phthalonitrile: Into a 50 mL round-bottom flask were added (S)-2-methylpiperidin-4-one (1.4 g, 12.4 mmol, 1.5 eq), 4-fluorophthalonitrile (1.2 g, 8.2 mmol, 1.0 eq), Na₂CO₃ (2.6 g, 24.7 mmol, 3.0 eq) and DMSO (20 mL). The resulting mixture was stirred for overnight at 80° C. The reaction was quenched by the addition of water (20 mL) and extracted with CH₂Cl₂ (3×20 mL). The combined organic layers were washed with brine (20 mL), dried over anhydrous Na₂SO₄. After filtration, the filtrate was concentrated under vacuum. The residue was purified by silica gel column chromatography, eluted with ethyl acetate/petroleum ether=2:1 to give (S)-4-(2-methyl-4-oxopiperidin-1-yl)phthalonitrile as a light yellow solid (900 mg, 45.6%). ¹HNMR (300 MHz, DMSO-d6) δ 7.90-7.77 (m, 1H), 7.57 (d, J=2.8 Hz, 1H), 7.27 (dd, J=9.1, 2.8 Hz, 1H), 4.50 (td, J=6.6, 3.5 Hz, 1H), 3.99 (dq, J=12.5, 6.2 Hz, 1H), 3.64 (ddd, J=13.5, 8.1, 5.3 Hz, 1H), 2.87 (dd, J=15.2, 6.2 Hz, 1H), 2.66-2.53 (m, 1H), 2.35 (dd, J=15.2, 3.4 Hz, 1H), 1.24 (s, 1H), 1.19 (d, J=6.6 Hz, 3H).

Synthesis of 4-((2S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalonitrile: Into a 40 mL vial were added (S)-4-(2-methyl-4-oxopiperidin-1-yl)phthalonitrile (900 mg, 3.8 mmol, 1.0 eq), (S)-5-bromo-1-methyl-3-((5-(2-methylpiperazin-1-yl)pyridin-2-yl)amino)pyridin-2(1H)-one hydrochloride (1.4 g, 3.8 mmol, 1.0 eq), NaBH₃CN (1.2 g, 18.8 mmol, 5.0 eq), ZnCl₂ (3.6 g, 26.3 mmol, 7.0 eq) and MeOH(10 mL). The resulting mixture was stirred overnight at 50° C. The reaction was quenched by the addition of water (10 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by silica gel column chromatography, eluted with ethyl acetate/petroleum ether=1:1 to give 4-((2S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalonitrile as a yellow solid (900 mg, 39.8%). LC-MS (ESI, m/z) M+1: 601/603.

Synthesis of 4-((2S,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid and 4-((2S,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid: Into a 40 mL vial were added 4-((2S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalonitrile (900 mg, 1.5 mmol, 1.5 eq) and H₂SO₄/HOAc/H₂O (3 mL/3 mL/3 mL). The resulting mixture was stirred for 2 hours at 110° C. The residue was purified by reverse flash chromatography using the following conditions: column, C18 silica gel; mobile phase, CH₃CN in water (0.1% TFA), 10% to 50% gradient in 10 min; detector, UV 254 nm. Finally, 4-((2S,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid was obtained as a yellow green solid (600 mg, 62.7%) and 4-((2S,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid was obtained as a yellow green solid (120 mg, 12.5%). LC-MS (ESI, m/z) M+1: 639/641.

Synthesis of 5-((2S,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione: Into a 80 mL vial were added 4-((2S,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid (80 mg, 0.1 mmol, 1.0 eq), KOAc (61 mg, 0.6 mmol, 5.0 eq), 3-aminopiperidine-2,6-dione (80 mg, 0.6 mmol, 5.0 eq) and HOAc (1 mL) at 25° C. The resulting mixture was stirred for overnight at 100° C. The resulting mixture was diluted with water (3 mL) and extracted with EtOAc (3×3 mL). The combined organic layers were washed with brine (3 mL), dried over anhydrous Na₂SO₄. After filtration, the filtrate was concentrated under vacuum to give 5-((2S,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione was used in the next step directly without further purification (90 mg, 80.1%). LC-MS (ESI, m/z) M+1: 731/733.

Synthesis of 5-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione hydrochloride: Into an 8 mL vial were added 5-((2S,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione (90 mg, 0.1 mmol, 1.0 eq), 2-(1-hydroxy-1,3-dihydro-[1,2]oxaborolo[4,3-c]pyridin-4-yl)-7,7-dimethyl-3,4,7,8-tetrahydro-2H-cyclopenta[4,5]pyrrolo[1,2-a]pyrazin-1(6H)-one (41 mg, 0.1 mmol, 1.0 eq), Pd(DtBPF)Cl₂ (8 mg, 0.01 mmol, 0.1 eq), K₂CO₃ (34 mg, 0.2 mmol, 2.0 eq) and dioxane (1 mL) at 25° C. The resulting mixture was stirred for 1 hour at 90° C. under nitrogen atmosphere. The reaction was quenched by the addition of water (2 mL) and extracted with CH₂Cl₂ (3×3 mL). The combined organic layers were washed with brine (3 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.1% TFA), 10% to 50% gradient in 10 min; detector, UV 254 nm. Finally, 5-((2S,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione hydrochloride as a yellow solid (9 mg, 7.3%). LC-MS (ESI, m/z) M+1: 962. ¹HNMR (300 MHz, DMSO-d6) δ 11.09 (s, 1H), 9.06 (s, 1H), 8.52 (d, J=5.1 Hz, 1H), 7.99 (s, 1H), 7.73 (s, 3H), 7.43-7.08 (m, 4H), 7.30 (s, 1H), 6.57 (s, 1H), 5.09 (dd, J=10.2, 5.4 Hz, 1H), 4.44 (s, 2H), 4.22 (s, 3H), 3.91-3.70 (m, 3H), 3.68-3.61 (m, 3H), 3.58-3.03 (m, 8H), 3.00-2.82 (m, 2H), 2.71-2.56 (m, 3H), 2.51-2.34 (m, 4H), 2.35-1.86 (m, 4H), 1.55 (d, J=6.3 Hz, 1H), 1.33-1.05 (m, 11H), 0.91 (d, J=5.7 Hz, 2H).

### Example 29: Preparation of 5-((2S,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione hydrochloride

Synthesis of 5-((2S,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione: Into an 80 mL vial were added 4-((2S,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid (80 mg, 0.1 mmol, 1.0 eq), KOAc (61 mg, 0.6 mmol, 5.0 eq), 3-aminopiperidine-2,6-dione (80 mg, 0.6 mmol, 5.0 eq) and HOAc (1 mL) at 25° C. The resulting mixture was stirred for overnight at 100° C. The resulting mixture was diluted with water (3 mL) and then extracted with EtOAc (3×3 mL). The combined organic layers were washed with brine (3 mL), dried over anhydrous Na₂SO₄. After filtration, the filtrate was concentrated under vacuum to give (5-((2S,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione, which was used directly to the next step without further purification (80 mg, 76.4%). LC-MS (ESI, m/z) M+1: 731/733.

Synthesis of 5-((2S,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione hydrochloride: Into an 8 mL vial were added (5-((2S,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione (80 mg, 0.1 mmol, 1.0 eq), 2-(1-hydroxy-1,3-dihydro-[1,2]oxaborolo[4,3-c]pyridin-4-yl)-7,7-dimethyl-3,4,7,8-tetrahydro-2H-cyclopenta[4,5]pyrrolo[1,2-a]pyrazin-1(6H)-one (37 mg, 0.1 mmol, 1.0 eq), Pd(DtBPF)Cl₂ (7 mg, 0.01 mmol, 0.1 eq), K₂CO₃ (30 mg, 0.2 mmol, 2.0 eq) and dioxane (1 mL) at 25° C. The reaction mixture was stirred for 1 hour at 90° C. under nitrogen atmosphere. The resulting mixture was quenched by the addition of water (2 mL) and then extracted with CH₂Cl₂ (3×3 mL). The combined organic layers were washed with brine (3 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.1% TFA), 10% to 50% gradient in 10 min; detector, UV 254 nm. Finally, 5-((2S,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione hydrochloride was obtained as a yellow solid (3 mg, 2.7%). LC-MS (ESI, m/z) M+1: 962. ¹HNMR (300 MHz, DMSO-d6) δ 11.09 (s, 1H), 9.06-8.56 (m, 3H), 8.52 (d, J=5.1 Hz, 1H), 8.08-7.85 (s, 1H), 7.73 (s, 1H), 7.60-7.46 (m, 2H), 7.44-7.19 (s, 3H), 6.57 (s, 1H), 5.09 (dd, J=10.2, 5.4 Hz, 1H), 4.86-4.53 (s, 1H), 4.48 (s, 2H), 4.39-4.13 (m, 4H), 4.08 (m, 1H), 3.96 (m, 1H), 3.66 (m, 3H), 3.28-3.02 (m, 4H), 3.01-2.81 (m, 2H), 2.72-2.60 (m, 3H), 2.42 (m, 2H), 2.15-2.05 (m, 4H), 2.03-1.89 (m, 2H), 1.55 (d, J=6.3 Hz, 1H), 1.41-1.19 (m, 10H), 1.17-1.08 (m, 3H), 0.91 (d, J=5.7 Hz, 3H).

### Example 30: Preparation of 5-((2R,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione

Synthesis of (R)-2-methylpiperidin-4-one hydrochloride: Into a 50 mL vial were added tert-butyl (R)-2-methyl-4-oxopiperidine-1-carboxylate (3.0 g, 14.1 mmol, 1 eq) and 4 M HCl (gas) in 1,4-dioxane (10 mL). The reaction mixture was stirred for 4 hours at 25° C. The precipitated solids were collected by filtration and washed with EtOAc (3×5 mL) to give ((R)-2-methylpiperidin-4-one hydrochloride as a white solid (1.5 g, 94.2%).

Synthesis of (R)-4-(2-methyl-4-oxopiperidin-1-yl)phthalonitrile: Into a 50 mL round-bottom flask were added (R)-2-methylpiperidin-4-one hydrochloride (800 mg, 7.1 mmol, 1.5 eq), 4-fluorobenzene-1,2-dicarbonitrile (689 mg, 4.7 mmol, 1.0 eq), Na₂CO₃ (1498 mg, 14.1 mmol, 3.0 eq) and DMSO (10 mL). The resulting mixture was stirred overnight at 80° C. The reaction mixture was quenched by the addition of water (5 mL) and extracted with CH₂Cl₂ (3×5 mL). The combined organic layers were washed with brine (5 mL), dried over anhydrous Na₂SO₄. After filtration, the filtrate was concentrated under vacuum. The crude residue was purified by a flash column (silica gel, ethyl acetate/petroleum ether=2:1) to give (R)-4-(2-methyl-4-oxopiperidin-1-yl)phthalonitrile as a light yellow solid (500 mg, 44.3%). ¹HNMR (300 MHz, DMSO-d₆) δ 7.83 (d, J=9.0 Hz, 1H), 7.57 (d, J=2.7 Hz, 1H), 7.27 (dd, J=9.0, 2.7 Hz, 1H), 4.50 (td, J=6.6, 3.6 Hz, 1H), 4.08-3.90 (m, 1H), 3.71-3.58 (m, 1H), 2.87 (dd, J=15.3, 6.3 Hz, 1H), 2.51 (dd, J=3.9, 1.8 Hz, 2H), 2.35 (dd, J=15.0, 3.6 Hz, 1H), 1.19 (d, J=6.6 Hz, 3H).

Synthesis of 4-((2R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalonitrile: Into a 40 mL vial were added (R)-4-(2-methyl-4-oxopiperidin-1-yl)phthalonitrile (500 mg, 2.1 mmol, 1.0 eq), (S)-5-bromo-1-methyl-3-((5-(2-methylpiperazin-1-yl)pyridin-2-yl)amino)pyridin-2(1H)-one hydrochloride (790 mg, 2.1 mmol, 1.0 eq), NaBH₃CN (2214 mg, 10.5 mmol, 5.0 eq), ZnCl₂ (1428 mg, 10.5 mmol, 5.0 eq) and MeOH (10 mL). The resulting mixture was stirred overnight at 50° C. The reaction was quenched by the addition of water (3 mL) and then extracted with CH₂Cl₂ (3×3 mL). The combined organic layers were washed with brine (5 mL), dried over anhydrous Na₂SO₄. After filtration, the filtrate was concentrated under vacuum. The crude residue was purified by a flash column (silica gel, ethyl acetate/petroleum ether=1:1) to give 4-((2R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalonitrile as a yellow solid (950 mg, 75.6%). LC-MS (ESI, m/z) M+1: 601/603.

Synthesis of 4-((2R,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid and 4-((2R,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid: Into a 40 mL vial were added 4-((2R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalonitrile (900 mg, 1.5 mmol, 1.5 eq) and H₂SO₄/HOAc/H₂O (3 mL/3 mL/3 mL). The resulting mixture was stirred for 2 hours at 110° C. The resulting mixture was diluted with 15% NaOH aq. (10 mL) and extracted with CH₂Cl₂ (3×10 mL). The combined organic layers were washed with brine (10 mL), dried over anhydrous Na₂SO₄. After filtration, the filtrate was concentrated under vacuum. The residue was purified by reverse flash chromatography using the following conditions: column, C18 silica gel; mobile phase, CH₃CN in H₂O (0.1% TFA), 10% to 50% gradient in 10 min; detector, UV 254 nm to give 4-((2R,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid as a yellow green solid (120 mg, 12.5%) and 4-[(2R,4R)-4-[(3S)-4-{6-[(5-bromo-1-methyl-2-oxopyridin-3-yl)amino]pyridin-3-yl}-3-methylpiperazin-1-yl]-2-methylpiperidin-1-yl]benzene-1,2-dicarboxylic acid as a yellow green solid (600 mg, 62.7%). LC-MS (ESI, m/z) M+1: 639/641.

Synthesis of 5-((2R,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione: Into an 80 mL vial were added 4-((2R,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)phthalic acid (80 mg, 0.1 mmol, 1.0 eq), KOAc (61 mg, 0.6 mmol, 5.0 eq), 3-aminopiperidine-2,6-dione (80 mg, 0.6 mmol, 5.0 eq) and HOAc (1 mL) at 25° C. The resulting mixture was stirred overnight at 100° C. The resulting mixture was diluted with water (3 mL) and extracted with EtOAc (3×3 mL). The combined organic layers were washed with brine (3 mL), dried over anhydrous Na₂SO₄. After filtration, the filtrate was concentrated under vacuum. The crude product 5-((2R,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione was used directly to the next without further purification (80 mg, 87.3%). LC-MS (ESI, m/z) M+1: 731/733.

Synthesis of 5-((2R,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione: Into an 8 mL vial were added 5-((2R,4S)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione (70 mg, 0.1 mmol, 1.0 eq), 10-{1-hydroxy-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one (32 mg, 0.1 mmol, 1.0 eq), Pd(DtBPF)Cl₂ (6 mg, 0.01 mmol, 0.1 eq), K₂CO₃ (40 mg, 0.3 mmol, 3.0 eq) and dioxane/water (1 mL/0.1 mL) at 25° C. The resulting mixture was stirred for 1 hour at 90° C. under nitrogen atmosphere. The reaction mixture was quenched by the addition of water (2 mL) and extracted with CH₂Cl₂ (3×3 mL). The combined organic layers were washed with brine (3 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.1% TFA), 10% to 50% gradient in 10 min; detector, UV 254 nm to give 5-((2R,4S)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione as a yellow solid (20 mg, 21.7%). LC-MS (ESI, m/z) M+1: 962. ¹HNMR (300 MHz, DMSO-d₆) δ 11.09 (s, 1H), 9.04 (s, 1H), 8.59 (s, 1H), 8.51 (d, J=5.1 Hz, 1H), 8.07-7.75 (m, 1H), 7.76-7.53 (m, 3H), 7.46-7.20 (m, 4H), 6.56 (s, 1H), 5.08 (dd, J=12.6, 5.4 Hz, 1H), 4.71 (s, 2H), 4.44 (s, 3H), 4.32-4.06 (m, 11H), 4.10-3.71 (m, 3H), 3.62 (s, 3H), 3.45-3.00 (m, 2H), 2.87 (d, J=13.5 Hz, 2H), 2.57 (d, J=4.8 Hz, 3H), 2.43 (s, 2H), 2.34-1.44 (m, 3H), 1.23 (s, 6H), 1.13 (d, J=6.6 Hz, 3H), 0.91 (d, J=6.0 Hz, 2H).

### Example 31: Preparation of 5-((2R,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione

Synthesis of 5-((2R,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione: Into an 80 mL vial were added 4-[(2R,4R)-4-[(3S)-4-{6-[(5-bromo-1-methyl-2-oxopyridin-3-yl)amino]pyridin-3-yl}-3-methylpiperazin-1-yl]-2-methylpiperidin-1-yl]benzene-1,2-dicarboxylic acid (80 mg, 0.1 mmol, 1.0 eq), KOAc (61 mg, 0.6 mmol, 5.0 eq), 3-aminopiperidine-2,6-dione (80 mg, 0.6 mmol, 5.0 eq) and HOAc (1 mL) at 25° C. The resulting mixture was stirred overnight at 100° C. The resulting mixture was diluted with water (3 mL) and extracted with EtOAc (3×3 mL). The combined organic layers were washed with brine (3 mL), dried over anhydrous Na₂SO₄. After filtration, the filtrate was concentrated under vacuum. The crude product 5-((2R,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione was used in the next step directly without further purification (70 mg, 76.4%). LC-MS (ESI, m/z) M+1: 731/733.

Synthesis of 5-((2R,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione: Into an 8 mL vial were added 5-((2R,4R)-4-((S)-4-(6-((5-bromo-1-methyl-2-oxo-1,2-dihydropyridin-3-yl)amino)pyridin-3-yl)-3-methylpiperazin-1-yl)-2-methylpiperidin-1-yl)-2-(2,6-dioxopiperidin-3-yl)isoindoline-1,3-dione (70 mg, 0.1 mmol, 1.0 eq), 10-{1-hydroxy-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one (32 mg, 0.1 mmol, 1.0 eq), Pd(DtBPF)Cl₂ (6 mg, 0.01 mmol, 0.1 eq), K₂CO₃ (40 mg, 0.3 mmol, 3.0 eq) and dioxane/water (1 mL/0.1 mL) at 25° C. The resulting mixture was stirred for 1 hour at 90° C. under nitrogen atmosphere. The reaction was quenched by the addition of water (2 mL) and extracted with CH₂Cl₂ (3×3 mL). The combined organic layers were washed with brine (3 mL), dried over anhydrous Na₂SO₄. After filtration, the filtrate was concentrated under vacuum. The residue was purified by reverse flash chromatography using the following conditions: column, C18 silica gel; mobile phase, CH₃CN in water (0.1% TFA), 10% to 50% gradient in 10 min; detector, UV 254 nm to give 5-((2R,4R)-4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-1-methyl-6-oxo-1,6-dihydro-[3,4′-bipyridin]-5-yl)amino)pyridin-3-yl)-3-methylpiperazin-1-yl)-2-methylpiperidin-1-yl)-2-(2,6-dioxopiperidin-3-yl)isoindoline-1,3-dione as a yellow solid (12 mg, 13.0%). LC-MS (ESI, m/z) M+1: 962. ¹HNMR (400 MHz, Acetonitrile-d₃) δ 8.81 (s, 1H), 8.42 (d, J=4.8 Hz, 1H), 7.81 (s, 1H), 7.66-7.55 (m, 2H), 7.51 (d, J=8.8 Hz, 1H), 7.28 (d, J=4.8 Hz, 1H), 7.21 (d, J=2.0 Hz, 1H), 7.09 (d, J=8.8 Hz, 2H), 6.59 (s, 1H), 4.86 (dd, J=12.0, 5.2 Hz, 1H), 4.41 (s, 2H), 4.28-4.07 (m, 3H), 3.52 (s, 3H), 3.45-3.33 (m, 1H), 3.22 (s, 3H), 2.71-2.51 (m, 3H), 2.50 (s, 2H), 2.38 (s, 2H), 2.26 (d, J=10.4 Hz, 2H), 2.09-1.96 (m, 3H), 1.86 (m, 10H), 1.52 (d, J=6.8 Hz, 2H), 1.15 (s, 6H), 1.13 (d, J=6.0 Hz, 3H).

### Example 33: Preparation of 5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-6-methoxy-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione

Synthesis of 5-bromo-2-methoxy-3-nitropyridine: Into a 250 mL round-bottom flask were added 5-bromo-2-chloro-3-nitropyridine (4.0 g, 16.8 mmol, 1.0 eq) and CH₃OH (40 mL) at 25° C. After that, NaH (0.6 g, 25.4 mmol, 1.5 eq. 60% w/w) was added in portions over 10 min at 0° C. The resulting mixture was stirred for 12 hours at 70° C. The reaction was quenched with sat. NH₄Cl (aq.) at 0° C. The resulting mixture was extracted with CH₂Cl₂ (3×40 mL). The combined organic layers were washed with brine (40 mL), dried over anhydrous Na₂SO₄. After filtration, the filtrate was concentrated under reduced pressure. The crude residue was purified by a flash column (silica gel, ethyl acetate/petroleum ether=3:1) to give 5-bromo-2-methoxy-3-nitropyridine as a white solid (3.5 g, 89.1%).

Synthesis of 5-bromo-2-methoxypyridin-3-amine: Into a 100 mL 3-necked round-bottom flask were added 5-bromo-2-methoxy-3-nitropyridine (3.5 g, 15.0 mmol, 1.0 eq), CH₃OH (40 mL), NH₄Cl (3.2 g, 60.1 mmol, 4.0 eq) and Zn (2.0 g, 30.0 mmol, 2.0 eq) at 25° C. The resulting mixture was stirred for additional 4 hours at 70° C. The resulting mixture was diluted with water (40 mL) and extracted with CH₂Cl₂ (3×40 mL). The combined organic layers were washed with brine (40 mL), dried over anhydrous Na₂SO₄. After filtration, the filtrate was concentrated under reduced pressure. The crude residue was purified by a flash column (silica gel, dichloromethane/methanol=10:1) to give 5-bromo-2-methoxypyridin-3-amine as a white solid (3.0 g, 98.3%). ¹HNMR (300 MHz, DMSO-d6) δ 7.39 (s, 1H), 7.20-6.63 (m, 1H), 5.63-4.87 (m, 2H), 3.84 (s, 3H).

Synthesis of 5-bromo-3-iodo-2-methoxypyridine: Into a 100 mL 3-necked round-bottom flask were added 5-bromo-2-methoxypyridin-3-amine (3.0 g, 14.8 mmol, 1.0 eq) and water (30 mL) at 25° C. After that, HCl (1.6 g, 44.3 mmol, 3.0 eq) was added dropwise over 10 min at 0° C., followed by the addition of NaNO₂ (1.1 g, 16.3 mmol, 1.1 eq) in water (3 mL). The resulting mixture was stirred for additional 10 min at 0° C. and then added KI (3.7 g, 22.2 mmol, 1.5 eq) at 0° C. The resulting mixture was stirred for 2 hours at 0° C. The resulting mixture was quenched by the addition of aq. Na₂SO₃ (20 mL) and then extracted with CH₂Cl₂ (3×30 mL). The combined organic layers were washed with brine (30 mL), dried over anhydrous Na₂SO₄. After filtration, the filtrate was concentrated under reduced pressure. The crude residue was purified by a flash column (silica gel, ethyl acetate/petroleum ether=1:1) to give 5-bromo-3-iodo-2-methoxypyridine as a grey solid (2.2 g, 47.4%). ¹HNMR (300 MHz, DMSO-d6) δ 8.35 (dq, J=8.1, 2.1 Hz, 1H), 8.29-8.17 (m, 1H), 3.87 (d, J=1.7 Hz, 3H).

Synthesis of tert-butyl (S)-4-(6-((5-bromo-2-methoxypyridin-3-yl)amino)pyridin-3-yl)-3-methylpiperazine-1-carboxylate: Into a 40 mL vial, were added 5-bromo-3-iodo-2-methoxypyridine (700 mg, 2.2 mmol, 1.0 eq), toluene (15 mL), tert-butyl (3S)-4-(6-aminopyridin-3-yl)-3-methylpiperazine-1-carboxylate (717 mg, 2.5 mmol, 1.1 eq), Cs₂CO₃ (1.5 g, 4.5 mmol, 2.0 eq) and 2nd Generation XantPhos precatalyst (99 mg, 0.1 mmol, 0.05 eq) at 25° C. The resulting mixture was stirred for 16 hours at 80° C. under nitrogen atmosphere. The resulting mixture was concentrated under vacuum. The residue was quenched by the addition of water (10 mL) and extracted with ethyl acetate (3×10 mL). The combined organic layers were washed with brine (10 mL), dried over anhydrous Na₂SO₄. After filtration, the filtrate was concentrated under vacuum. The crude residue was purified by a flash column (silica gel, ethyl acetate/petroleum ether=1:1) to give tert-butyl (S)-4-(6-((5-bromo-2-methoxypyridin-3-yl)amino)pyridin-3-yl)-3-methylpiperazine-1-carboxylate as a pink solid (500 mg, 46.8%). LC-MS: (ESI, m/z): M+1: 478/480.

Synthesis of (S)—N-(5-bromo-2-methoxypyridin-3-yl)-5-(2-methylpiperazin-1-yl)pyridin-2-amine hydrochloride: Into a 40 mL vial, were added tert-butyl (3S)-4-{6-[(5-bromo-2-methoxypyridin-3-yl)amino]pyridin-3-yl}-3-methylpiperazine-1-carboxylate (480 mg, 1.0 mmol, 1.0 eq), ethyl acetate(3 mL) and 4 M HCl in dioxane (3 mL) at 25° C. The resulting mixture was stirred for 1 hour at 25° C. The resulting mixture was concentrated under vacuum. The precipitated solids were collected by filtration and washed with EtOAc (3×5 mL) to afford (S)—N-(5-bromo-2-methoxypyridin-3-yl)-5-(2-methylpiperazin-1-yl)pyridin-2-amine hydrochloride as an orange solid (350 mg, 92.2%). LC-MS: (ESI, m/z): M+1: 378/380.

Synthesis of 5-(4-((S)-4-(6-((5-bromo-2-methoxypyridin-3-yl)amino)pyridin-3-yl)-3-methylpiperazin-1-yl)piperidin-1-yl)-2-(2,6-dioxopiperidin-3-yl)isoindoline-1,3-dione: Into a 40 mL vial, were added (S)—N-(5-bromo-2-methoxypyridin-3-yl)-5-(2-methylpiperazin-1-yl)pyridin-2-amine hydrochloride (350 mg, 0.9 mmol, 1.0 eq), 1,2-dichloroethane (10 mL), 2-(2,6-dioxopiperidin-3-yl)-5-(4-oxopiperidin-1-yl)isoindole-1,3-dione (329 mg, 0.9 mmol, 1.0 eq), NaBH(AcO)₃ (392 mg, 1.9 mmol, 2.0 eq) and HOAc (0.2 mL) at 25° C. The resulting mixture was stirred for additional 16 hours at 50° C. After filtration, the filtrate was concentrated under vacuum. The residue was purified by Prep-TLC (tetrahydrofuran/petroleum ether 10:1) to give 5-(4-((S)-4-(6-((5-bromo-2-methoxypyridin-3-yl)amino)pyridin-3-yl)-3-methylpiperazin-1-yl)piperidin-1-yl)-2-(2,6-dioxopiperidin-3-yl)isoindoline-1,3-dione as a grey solid (130 mg, 15.6%). LC-MS: (ESI, m/z): M+1: 717/719.

Synthesis of 5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-6-methoxy-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione: Into an 8 mL vial, were added 5-{4-[(3S)-4-{6-[(5-bromo-2-methoxypyridin-3-yl)amino]pyridin-3-yl}-3-methylpiperazin-1-yl]piperidin-1-yl}-2-(2,6-dioxopiperidin-3-yl)isoindole-1,3-dione (130 mg, 0.1 mmol, 1.0 eq), dioxane (3 mL), 10-{1-hydroxy-3H-[1,2]oxaborolo[4,3-c]pyridin-4-yl}-4,4-dimethyl-1,10-diazatricyclo[6.4.0.0\hat{ }{2,6}]dodeca-2(6),7-dien-9-one (49 mg, 0.1 mmol, 1.0 eq), K₂CO₃ (40 mg, 0.3 mmol, 2.0 eq), Pd(DtBPF)Cl₂ (5 mg, 0.005 mmol, 0.05 eq) and water (0.3 mL) at 25° C. The resulting mixture was stirred for 1 hour at 90° C. under nitrogen atmosphere in microwave. The resulting mixture was quenched by the addition of water (5 mL) and extracted with CH₂Cl₂ (4×5 mL). The combined organic layers were washed with brine (5 mL), dried over anhydrous Na₂SO₄. After filtration, the filtrate was concentrated under vacuum. The residue was purified by Prep-TLC (tetrahydrofuran/petroleum ether 3:1) to give 70 mg crude. The crude product was purified by reverse flash chromatography using the following conditions: column, silica gel; mobile phase, CH₃CN in water (0.05% FA), 10% to 80% gradient in 10 min; detector, UV 254 nm. Finally, 5-(4-((S)-4-(6-((2′-(7,7-dimethyl-1-oxo-1,3,4,6,7,8-hexahydro-2H-cyclopenta[4,5]pyrrolo[1,2-a]pyrazin-2-yl)-3′-(hydroxymethyl)-6-methoxy-[3,4′-bipyridin]-5-yl)amino)pyridin-3-yl)-3-methylpiperazin-1-yl)piperidin-1-yl)-2-(2,6-dioxopiperidin-3-yl)isoindoline-1,3-dione was obtained as a yellow solid (5.5 mg, 4.0%). LC-MS: (ESI, m/z): M+1: 948. ¹HNMR (300 MHz, Methanol-d₄) δ 8.76 (s, 1H), 8.53 (d, J=5.1 Hz, 1H), 7.91 (s, 1H), 7.82 (s, 1H), 7.60 (s, 1H), 7.46-7.55 (m, 2H), 7.39 (s, 1H), 7.28 (s, 1H), 7.03 (s, 1H), 6.78 (s, 1H), 4.60 (s, 1H), 4.33-4.25 (m, 2H), 4.19-4.08 (m, 6H), 3.10-3.04 (m, 5H), 2.91-2.68 (m, 6H), 2.61-2.55 (m, 3H), 2.53-2.32 (m, 3H), 2.26-1.98 (m, 3H), 1.70-1.58 (m, 2H), 1.46-1.27 (m, 11H), 0.97 (d, J=6.0 Hz, 3H).

### Biological Example 1: Binding Constant (K_{d}) Determination

The K_{d} of the compounds were determined by KINOMEscan™ assay, the industry's most comprehensive high-throughput system for screening compounds against large numbers of human kinases. KINOMEscan™ assay is based on a competition binding assay that quantitatively measures the ability of a compound to compete with an immobilized, active-site directed ligand. The assay is performed by combining three components: DNA-tagged kinase; immobilized ligand; and a test compound. The ability of the test compound to compete with the immobilized ligand is measured via quantitative PCR of the DNA tag. The kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17× PBS, 0.05% Tween 20, 6 mM DTT). All reactions were performed in polystyrene 96-well plates in a final volume of 0.135 ml. The assay plates were incubated at room temperature with shaking for 1 hour and the affinity beads were washed with wash buffer (1× PBS, 0.05% Tween 20). The beads were then re-suspended in elution buffer (1× PBS, 0.05% Tween 20, 0.5 μM nonbiotinylated affinity ligand) and incubated at room temperature with shaking for 30 minutes. The kinase concentration in the eluates was measured by qPCR. An 11-point 3-fold serial dilution of each test compound was prepared in 100% DMSO at 100× final test concentration and subsequently diluted to 1× in the assay (final DMSO concentration=1%). Most K_{d} were determined using a compound top concentration=30,000 nM. If the initial Kd determined was <0.5 nM (the lowest concentration tested), the measurement was repeated with a serial dilution starting at a lower top concentration. A K_{d} value reported as 40,000 nM indicates that the K_{d} was determined to be >30,000 nM. Binding constants (K_{d}s) were calculated with a standard dose-response curve using the Hill equation: Response=Background+(Signal−Background)/[1+(K_{d}^{Hill Slope}/Dose^{Hill Slope})]. The Hill Slope was set to −1. Curves were fitted using a non-linear least square fit with the Levenberg-Marquardt algorithm. Such assays, carried out with a range of doses of test compounds, allow the determination of an approximate K_{d} value. Although the K_{d} of the compounds of the present invention vary with structural change as expected, the activity generally exhibited by these agents is in the range of K_{d}=0.1−1000 nM.

### Biological Example 2: Biochemical Enzymatic Assay (IC50) Against WT and C481S BTK

A Caliper-based kinase assay (Caliper Life Sciences, Hopkinton, MA) was used to measure inhibition of WT and C481S Btk kinase activity of a compound of the present disclosure. Ibrutinib and ACP=196 was used as control compounds. Serial dilutions of test compounds were incubated with human recombinant WT BTK or C481S Btk (0.5 nM), ATP (16 μM) and a phosphoacceptor peptide substrate FAM-GEEPLYWSFPAKKK-NH2 (1 μM) at room temperature for 3 h. The reaction was then terminated with EDTA, final concentration 20 mM and the phosphorylated reaction product was quantified on a Caliper Desktop Profiler (Caliper LabChip 3000). Percent inhibition was calculated for each compound dilution and the concentration that produced 50% inhibition was calculated.

### Biological Example 3: BTK HTRF Degradation Assay

Rec-1 cells were obtained from American Type Culture Collection and were grown in RPMI-1640 media (ATCC, 30-2001) supplemented with 10% heat-inactivated FBS (Corning Premium Fetal Bovine Serum from Fisher, MT35015CV). Compounds of the present invention were added to 50,000 Ramos cells in round-bottom 96 well plates with a final DMSO concentration of >0.2% and were incubated at 37° C. 5% C02 for different time. BTK levels were determined using Cisbio Total-BTK HTRF (Homologous Time-Resolved Fluorescence) kit (63ADK064PEG) according to manufacturer's protocol. Briefly, cells were incubated in 1X supplied lysis buffer for 30 minutes. In an opaque white low volume 96 well plate (Cisbio, 66PL96005), cell lysate was combined with two different specific BTK antibodies, one conjugated with Eu3+-Cryptate FRET donor and one conjugated with d2 FRET acceptor. Assay controls include wells containing cell lysate with only the Eu3+-Cryptate FRET donor antibody and wells containing both HTRF antibodies and lysis buffer without cells or control lysate provided by Cisbio. HTRF ratio was calculated as (acceptor signal at 665 nm/donor signal at 620 nm)×104. Background HTRF levels were determined from the control well containing the donor, but no acceptor, antibody. Background HTRF levels were subtracted from all samples. Readouts were reported as HTRF levels relative to HTRF levels of DMSO-treated cells. Four-parameter non-linear regressions were performed in GraphPad Prism 7.02 to obtain DC50 values.

### Biological Example 4: Inhibition of CD69 Surface Expression in Primary Human B Cells (Western Analysis)

Primary human B cells (CD20+, purified by negative selection) were obtained from StemCell Technologies. Prior to experiment, the cells were thawed and washed two times with RPMI growth media supplemented with 10% FBS. The cells were seeded into 24 well plate at a density of 4×105 cells/per a well in a total volume of 500 uL. 6 hr after plating, serial dilutions of NW-1-96 were added. Control wells received DMSO only (0.1%). After 1h pre-incubation with compound, the cells were stimulated for 19 hr with goat anti-human IgM F(ab′)₂ (10 μg/mL; ThermoFisher). After stimulation, the cells were fixed by addition of paraformaldehyde to a final concentration of 4% and incubated for 20 min at room temperature. Fixed cells were collected into Eppendorf tubes, centrifuged at 1,000×g and washed three times with 50 mM Tris pH8.0, 100 mM NaCl. After washes, the cells were re-suspended in 100 μL of 50 mM Tris pH8.0, 100 mM NaCl, 0.1% BSA supplemented with 5ug/mL of FITC-conjugated anti-CD69 antibody (ThermoFisher) and incubated at room temperature for 2 hr. The cells were next washed 3 times with 10 volumes of 50 mM Tris pH8.0, 100 mM NaCl, 0.1% BSA and re-suspended in 150ul of the same buffer. Stained cells were transferred into black 96well plate (100 uL suspension per well) and allowed to sediment for 1 hr. CD69 staining was detected on Synergy Neo2 fluorescent plate reader: 485 nm emission, 528 nm excitation.

### Biological Example 5: HepG2 In Vitro Liver Toxicity Assay

Cell antiproliferation is assayed by PerkinElmer ATPlite™ Luminescence Assay System. Briefly, the liver cancer cell line HepG2 are plated at a density of about 1×10⁴ cells per well in Costar 96-well plates, and are incubated with different concentrations of compounds for about 72 hours in medium supplemented with 5% FBS. One lyophilized substrate solution vial is then reconstituted by adding 5 mL of substrate buffer solution, and is agitated gently until the solution is homogeneous. About 50 μL of mammalian cell lysis solution is added to 100 μL of cell suspension per well of a microplate, and the plate is shaken for about five minutes in an orbital shaker at ˜700 rpm. This procedure is used to lyse the cells and to stabilize the ATP. Next, 50 μL substrate solution is added to the wells and microplate is shaken for five minutes in an orbital shaker at ˜700 rpm. Finally, the luminescence is measured by a PerkinElmer TopCount® Microplate Scintillation Counter. Such assays, carried out with a range of doses of test compounds, allow the determination of the cellular anti-antiproliferative IC₅₀ of the compounds of the present invention.

### Biological Example 6: Human Primary Hepatocyte Cytotoxicity

Cell viability assay is assayed by PerkinElmer ATPlite™ Luminescence Assay System. Briefly, the human primary hepatocyte are plated at a density of about 1×10⁴ cells per well in Costar 96-well plates, and are incubated with different concentrations of compounds for about 72 hours in medium supplemented with 5% FBS. One lyophilized substrate solution vial is then reconstituted by adding 5 mL of substrate buffer solution, and is agitated gently until the solution is homogeneous. About 50 μL of mammalian cell lysis solution is added to 100 μL of cell suspension per well of a microplate, and the plate is shaken for about five minutes in an orbital shaker at −700 rpm. This procedure is used to lyse the cells and to stabilize the ATP. Next, 50 μL substrate solution is added to the wells and microplate is shaken for five minutes in an orbital shaker at −700 rpm. Finally, the luminescence is measured by a PerkinElmer TopCount® Microplate Scintillation Counter. Such assays, carried out with a range of doses of test compounds, allow the determination of the cellular anti-antiproliferative IC₅₀ of the compounds of the present invention.

### Biological Example 7: Mice PK Study

The pharmacokinetics of compounds were evaluated in CD-1 mouse via Intravenous and Oral Administration. The IV dose was administered as a slow bolus in the Jugular vein, and oral doses were administered by gavage. The formulation for IV dosing is 5% DMSO in 20% HPBCD in water, and the PO formulation is 2.5% DMSO, 10% EtOH, 20% Cremphor EL, 67.5% D5W. The PK time point for the IV arm was 5, 15, 30 min, 1, 2, 4, 6, 8, 12, 24 hours post dose, and for PO arm was 15, 30 min, 1, 2, 4, 6, 8, 12, 24 hours post dose. Approximately 0.03 mL blood was collected at each time point. Blood of each sample was transferred into plastic micro centrifuge tubes containing EDTA-K2 and plasma was collected within 15 min by centrifugation at 4000 g for 5 minutes in a 4° C. centrifuge. Plasma samples were stored in polypropylene tubes. The samples were stored in a freezer at −75±15° C. prior to analysis. Concentrations of compounds in the plasma samples were analyzed using a LC-MS/MS method. WinNonlin (Phoenix™, version 6.1) or other similar software was used for pharmacokinetic calculations. The following pharmacokinetic parameters were calculated, whenever possible from the plasma concentration versus time data: IV administration: C₀, CL, V_{d}, T_{1/2}, AUC_{inf}, AUCₗₐₛₜ, MRT, Number of Points for Regression; PO administration: Cₘₐₓ, Tₘₐₓ, T₁/2, AUC_{inf}, AUCₗₐₛₜ, F %, Number of Points for Regression. The pharmacokinetic data was described using descriptive statistics such as mean, standard deviation. Additional pharmacokinetic or statistical analysis was performed at the discretion of the contributing scientist, and was documented in the data summary.

### Biological Example A: Calcium Flux Fluoresence-Based Assay

Calcium flux fluoresence-based assays were performed in a FlexStation 11384 fluorometric imaging plate reader (Molecular Devices) according to manufacturer instructions. In brief, actively growing Ramos cells (ATCC) in RPMI medium supplemented with 10% FBS (Invitrogen) were washed and re-plated in low serum medium at approximately 5×10⁵ cells per 100 μl per well in a96-well plate. Compounds to be assayed were dissolved in DMSO and then diluted in low serum medium to final concentrations ranging from 0 to 10 μM (at a dilution factor of 0.3). The diluted compounds were then added to each well (final DMSO concentration was 0.01%) and incubated at 37 degree in 5% CO₂ incubator for one hour. Afterwards, 100 μl of a calcium-sensitive dye (from the Calcium 3 assay kit, Molecular Devices) was added to each well and incubated for an additional hour. The compound-treated cells were stimulated with a goat anti-human IgM antibody (80ug/ml; Jackson ImmunoResearch) and read in the FlexStation 11384 using a λ_{Ex}=485 nm and λ_{Em}=538 nm for 200 seconds. The relative fluorescence unit (RFU) and the IC₅₀ were recorded and analyzed using a built-in SoftMax program (Molecular devices).

### Biological Example B: Inhibition of B-Cell Activation-B Cell FLIPR Assay in Ramos Cells

Inhibition of B-cell activation by compounds of the present invention is demonstrated by determining the effect of the test compounds on anti-IgM stimulated B cell responses. The B cell FLIPR assay is a cell based functional method of determining the effect of potential inhibitors of the intracellular calcium increase from stimulation by an anti-IgM antibody. Ramos cells (human Burkitt's lymphoma cell line. ATCC-No. CRL-1596) were cultivated in Growth Media (described below). One day prior to assay, Ramos cells were resuspended in fresh growth media (same as above) and set at a concentration of 0.5×10⁶/mL in tissue culture flasks. On day of assay, cells are counted and set at a concentration of 1×10⁶/mLI in growth media supplemented with lμM FLUO-3AM(TefLabs Cat-No. 0116, prepared in anhydrous DMSO and 10% Pluronic acid) in a tissue culture flask, and incubated at 37° C. (5% CO₂) for one h. To remove extracellular dye, cells were collected by centrifugation (5 min, 1000 rpm), resuspended in FLIPR buffer (described below) at 1×10⁶ cells/mL and then dispensed into 96-well poly-D-lysine coated black/clear plates (BD Cat-No. 356692) at 1×10⁵ cells per well. Test compounds were added at various concentrations ranging from 100 μM to 0.03 μM (7 concentrations, details below), and allowed to incubate with cells for 30 min at RT. Ramos cell Ca²⁺ signaling was stimulated by the addition of 10 μg/mL anti-IgM (Southern Biotech, Cat-No. 2020-01) and measured on a FLIPR (Molecular Devices, captures images of 96 well plates using a CCD camera with an argon laser at 480 nM excitation).
Growth Medium: RPMI 1640 medium with L-glutamine (Invitrogen, Cat-No. 61870-010), 10% Fetal Bovine Serum (FBS, Summit Biotechnology Cat-No. FP-100-05); lmM Sodium Pyruvate (Invitrogen Cat. No. 11360-070).
FLIPR buffer: HBSS (Invitrogen, Cat-No. 141175-079), 2 mM CaCl₂ (Sigma Cat-No. C-4901), HEPES (Invitrogen, Cat-No. 15630-080), 2.5 mM Probenecid (Sigma, Cat-No. P-8761), 0.1% BSA (Sigma, Cat-No.A-7906), I lmM Glucose (Sigma, Cat-No.G-7528);
Assay and Analysis: Intracellular increases in calcium were reported using a max-min statistic (subtracting the resting baseline from the peak caused by addition of the stimulatory antibody using a Molecular Devices FLIPR control and statistic exporting software. The IC₅₀ was determined using a nonlinear curve fit (GraphPad Prism).

### Biological Example C: In Vivo Xenograft Studies

Typically, athymic nude mice (CD-1 nu/nu) or SCID mice are obtained at age 6-8 weeks from vendors and acclimated for a minimum 7-day period. The cancer cells are then implanted into the nude mice. Depending on the specific tumor type, tumors are typically detectable about two weeks following implantation. When tumor sizes reach ˜100-200 mm³, the animals with appreciable tumor size and shape are randomly assigned into groups of 8 mice each, including one vehicle control group and treatment groups. Dosing varies depending on the purpose and length of each study, which typically proceeds for about 3-4 weeks. Tumor sizes and body weight are typically measured three times per week. In addition to the determination of tumor size changes, the last tumor measurement is used to generate the tumor size change ratio (T/C value), a standard metric developed by the National Cancer Institute for xenograft tumor evaluation. In most cases, % T/C values are calculated using the following formula: % T/C=100×ΔT/ΔC if ΔT>0. When tumor regression occurred (ΔT<0), however, the following formula is used: % T/T0=100×ΔT/T0. Values of <42% are considered significant.

### Biological Example D: Mouse Collagen-Induced Arthritis (mCIA)

On day 0 mice are injected at the base of the tail or several spots on the back with an emulsion of Type II Collagen (i.d.) in Complete Freund's adjuvant (CFA). Following collagen immunization, animals will develop arthritis at around 21 to 35 days. The onset of arthritis is synchronized (boosted) by systemic administration of collagen in Incomplete Freund's adjuvant (IFA; i.d.) at day 21. Animals are examined daily after day 20 for any onset of mild arthritis (score of 1 or 2; see score description below) which is the signal to boost. Following boost, mice are scored and dosed with candidate therapeutic agents for the prescribed time (typically 2-3 weeks) and dosing frequency, daily (QD) or twice-daily (BID). The developing inflammation of the paws and limb joints is quantified using a scoring system that involves the assessment of the 4 paws following the criteria described below:

### Scoring:

1=swelling and/or redness of paw or one digit.
2=swelling in two or more joints.
3=gross swelling of the paw with more than two joints involved.
4=severe arthritis of the entire paw and digits.

Evaluations are made on day 0 for baseline measurement and starting again at the first signs or swelling for up to three times per week until the end of the experiment. The arthritic index for each mouse is obtained by adding the four scores of the individual paws, giving a maximum score of 16 per animal.

### Biological Example E: Rat Collagen-Induced Arthritis (rCIA)

On day 0, rats are injected with an emulsion of Bovine Type II Collagen in Incomplete Freund's adjuvant (IFA) is injected intradermally (i.d.) on several locations on the back. A booster injection of collagen emulsion is given around day 7, (i.d.) at the base of the tail or alternative sites on the back. Arthritis is generally observed 12-14 days after the initial collagen injection. Animals may be evaluated for the development of arthritis as described below (Evaluation of arthritis) from day 14 onwards. Animals are dosed with candidate therapeutic agents in a preventive fashion starting at the time of secondary challenge and for the prescribed time (typically 2-3 weeks) and dosing frequency, daily (QD) or twice-daily (BID). The developing inflammation of the paws and limb joints is quantified using a scoring system that involves the assessment of the 4 paws following the criteria as described above. Evaluation are made on day 0 for baseline measurement and starting again at the first signs or swelling for up to three times per week until the end of the experiment. The arthritic index for each mouse is obtained by adding the four scores of the individual paws, giving a maximum score of 16 per animal.

### Biological Example F: Rat In Vivo Asthma Model

Male Brown-Norway rats are sensitized i.p. with 100 μg of OA (ovalbumin) in 0.2 ml alum once every week for three weeks (day 0, 7, and 14). On day 21 (one week following last sensitization), the rats are dosed q.d. with either vehicle or compound formulation subcutaneously 0.5 hour before OA aerosol challenge (1% OA for 45 minutes) and terminated 4 or 24 hours after challenge. At time of sacrifice, serum and plasma are collected from all animals for serology and PK, respectively. A tracheal cannula is inserted and the lungs are lavaged 3× with PBS. The BAL fluid is analyzed for total leukocyte number and differential leukocyte counts. Total leukocyte number in an aliquot of the cells (20-100 μ\tilde{l}) is determined by Coulter Counter. For differential leukocyte counts, 50-200 μ\tilde{l} of the sample is centrifuged in a Cytospin and the slide stained with Diff-Quik. The proportions of monocytes, eosinophils, neutrophils and lymphocytes are counted under light microscopy using standard morphological criteria and expressed as a percentage. Representative inhibitors of Btk show decreased total leucocyte count in the BAL of OA sensitized and challenged rats as compared to control levels.

What is claimed is:

1. A compound of Formula (1), or an N-oxide thereof, or a pharmaceutically acceptable salt, solvate, polymorph, tautomer, stereoisomer, an isotopic form, or a prodrug of said compound of Formula (1) or N-oxide thereof: 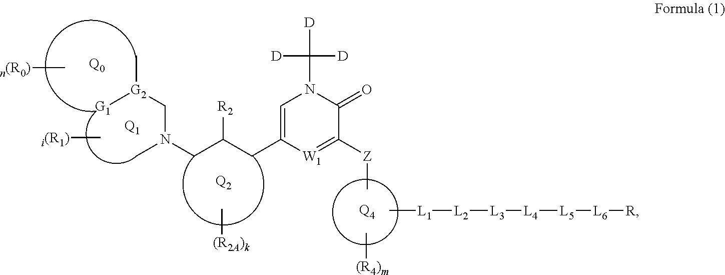
wherein
R is a small molecule (e.g., molecular weight less than about 1,500 Da, 1,200 Da, 900 Da, 500 Da or less) E3 ubiquitin ligase binding moiety that binds an E3 ubiquitin ligase;
each of L₁, L₂, L₃, L₄, L₅, and L₆, independently, is absent, a bond, N(Rₐ), O, S, C(O), S(O₂), OC(O), C(O)O, OSO₂, S(O₂)O, C(O)S, SC(O), C(O)C(O), C(O)N(Rₐ), N(Rₐ)C(O), S(O₂)N(Rₐ), N(Rₐ)S(O₂), OC(O)O, OC(O)S, OC(O)N(Rₐ), N(Rₐ)C(O)O, N(Rₐ)C(O)S, N(Rₐ)C(O)N(Rₐ), alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, cycloalkyl, cycloalkenyl, heterocycloalkyl, spiro-heterocyclic, fused-heterocyclic, bridged-heterocyclic, heterocycloalkenyl, aryl, or heteroaryl is optionally subsitiuted with one or more R_{d};
Q₀ is a 5-9 membered aryl or heteroaryl;
Q₁ is a 5-7 membered heterocycloalkyl;
Q₀ and Q₁ taken together form a fused-heterocyclic with two shared/border atoms between Q₀ and Q₁, including G₁ and G₂, wherein each said shared/border atoms can be carbon or heteroatom;
Q₂ is a 5-9 membered cycloalkyl, heterocycloalkyl, aryl or heteroaryl;
Q₄ is a 5-9 membered aryl or heteroaryl;
A is in Q₁ and is —C(O)—, —P(O)(RₐR_{b})—, or —S(O₂)—;
Z is NH or O;
W₁ is N or CH;
each of R₀, R₁, R_{2A}, and R₄, independently, is H, D, alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, nitro, oxo, cyano, ORₐ, SRₐ, alkyl-Rₐ, NH(CH₂)PRₐ, C(O)Rₐ, S(O)Rₐ, SO₂Rₐ, C(O)ORₐ, OC(O)Rₐ, NR_{b}R_{c}, C(O)N(R_{b})R_{c}, N(R_{b})C(O)R_{c}, —P(O)R_{b}R_{c}, -alkyl-P(O)R_{b}R_{c}, -alkyl-O—P(O)(Rₐ)(R_{b}), -alkyl-OC(O)N(Rₐ)(R_{b}), —S(O)(═N(R_{b}))R_{c}, —N═S(O)R_{b}R_{c}, =NR_{b}, SO₂N(R_{b})R_{c}, or N(R_{b})SO₂R_{c}, in which said cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
R₂ is H, halo, alkyl, —C(RₐR_{b}R_{c}), haloalkyl, or hydroxyalkyl;
two of R₀ groups, taken together with the atom to which they are attached, may optionally form a cycloalkyl, heterocycloalkyl, heterocycloalkenyl, aryl, or heteroaryl, each optionally subsitiuted with one or more R_{d};
two of R₁ groups, taken together with the atom to which they are attached, may optionally form a cycloalkyl, heterocycloalkyl, heterocycloalkenyl, aryl, or heteroaryl, each optionally subsitiuted with one or more R_{d};
two of R_{2A} groups, taken together with the atom to which they are attached, may optionally form a cycloalkyl, heterocycloalkyl, heterocycloalkenyl, aryl, or heteroaryl, each optionally subsitiuted with one or more R_{d};
two of R₄ groups, taken together with the atom to which they are attached, may optionally form a cycloalkyl, heterocycloalkyl, heterocycloalkenyl, aryl, or heteroaryl, each optionally subsitiuted with one or more R_{d};
each of Rₐ, R_{b}, R_{c} and R_{d}, independently, is H, D, alkyl, spiroalkyl, alkenyl, alkynyl, halo, cyano, amine, nitro, hydroxy, ═O, -alkyl-O—P(O)(OH)(OH), C(O)NHOH, C(O)OH, C(O)NH₂, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more Rₑ;
each Rₑ is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl, in which said alkyl, alkoxy, alkoxyalkyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{f}; and
each R_{f} is independently H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl;
Rₐ and R_{b}, taken together with the atom to which they are attached, may optionally form a cycloalkyl, heterocycloalkyl, heterocycloalkenyl, aryl, or heteroaryl, each optionally subsitiuted with one or more Rₑ;
R_{b} and R_{c}, taken together with the atom to which they are attached, may optionally form a cycloalkyl, heterocycloalkyl, heterocycloalkenyl, aryl, or heteroaryl, each optionally subsitiuted with one or more Rₑ;
two of R_{d} groups, taken together with the atom to which they are attached, may optionally form a cycloalkyl, heterocycloalkyl, heterocycloalkenyl, aryl, or heteroaryl, each optionally subsitiuted with one or more Rₑ;
two of Rₑ groups, taken together with the atom to which they are attached, may optionally form a cycloalkyl, heterocycloalkyl, heterocycloalkenyl, aryl, or heteroaryl, each optionally subsitiuted with one or more R_{f};
two of R_{f} groups, taken together with the atom to which they are attached, may optionally form a cycloalkyl, heterocycloalkyl, heterocycloalkenyl, aryl, or heteroaryl, each optionally subsitiuted with one or more H, D, alkyl, spiroalkyl, alkenyl, alkynyl, halo, cyano, amine, nitro, hydroxy, ═O, -alkyl-O—P(O)(OH)(OH), C(O)NHOH, alkoxy, alkoxyalkyl, haloalkyl, hydroxyalkyl, aminoalkyl, alkylcarbonyl, alkoxycarbonyl, alkylcarbonylamino, alkylamino, oxo, halo-alkylamino, cycloalkyl, cycloalkenyl, heterocycloalkyl, heterocycloalkenyl, spiro-heterocyclic, fused-heterocyclic, bridged-heterocyclic, aryl, or heteroaryl; and,
each of i, k, m, n, and p, independently, is 0, 1, 2, 3, or 4.

2. The compound according to claim 1 or an N-oxide thereof, or a pharmaceutically acceptable salt, solvate, polymorph, tautomer, stereoisomer, an isotopic form, or a prodrug thereof, wherein said E3 ubiquitin ligase is Cereblon, Von Hippel-Lindau, mouse double-minute homolog 2, or IAP.

3. The compound according to claim 2 or an N-oxide thereof, or a pharmaceutically acceptable salt, solvate, polymorph, tautomer, stereoisomer, an isotopic form, or a prodrug thereof, wherein the compound is represented by Formula (2): 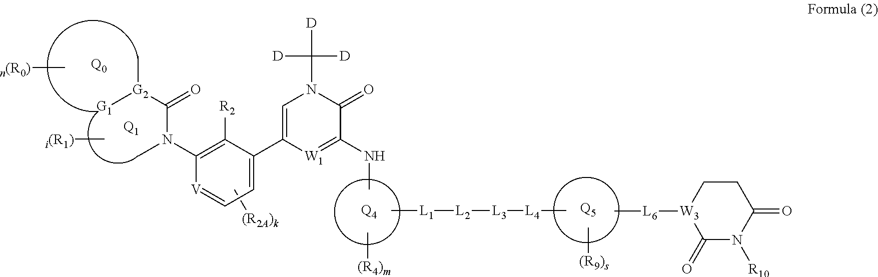
wherein
R₁₀ is H, D, -alkyl-O—P(O)(Rₐ)(R_{b}), or -alkyl-OC(O)—Rₐ;
L₆ is absent, NH, CONH, or 0;
W₁ is N or CH;
W₃ is N or CH;
Q₅ is absent, cycloalkyl, cycloalkenyl, heterocycloalkyl, heterocycloalkenyl, spiro-heterocyclic, fused-heterocyclic, bridged-heterocyclic, aryl, or heteroaryl;
R₉ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
R₉ and L₄ groups, taken together with the atom to which they are attached, may optionally form a cycloalkyl, heterocycloalkyl, heterocycloalkenyl, aryl, or heteroaryl optionally subsitiuted with one or more R_{d};
V is C(Rₐ) or N; and,
s is 0, 1, 2, 3, or 4.

4. The compound according to claim 3 or an N-oxide thereof, or a pharmaceutically acceptable salt, solvate, polymorph, tautomer, stereoisomer, an isotopic form, or a prodrug thereof, wherein the compound is represented by Formula (3): 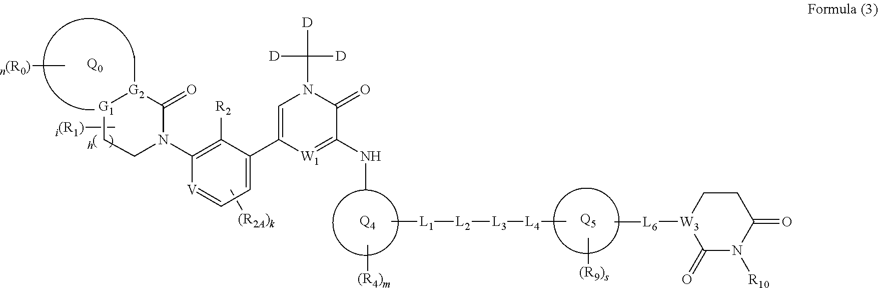
wherein h is 0, 1 or 2; and each of the border atoms between Q₀ and Q₁ including G₁ and G₂, can be carbon or heteroatom.

5. The compound according to claim 4 or an N-oxide thereof, or a pharmaceutically acceptable salt, solvate, polymorph, tautomer, stereoisomer, an isotopic form, or a prodrug thereof, wherein the compound is represented by Formula (4) wherein: 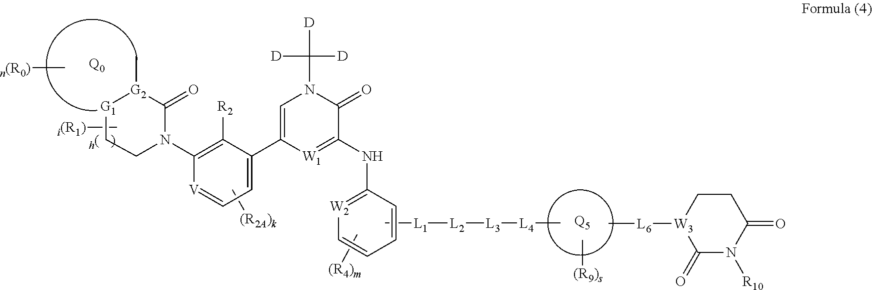
wherein W₁ is CH and W₂ is N, or W₁ is N and W₂ is CH, and each of the border atoms between Q₀ and Q₁ including G₁ and G₂, can be carbon or heteroatom.

6. The compound according to claim 5 or an N-oxide thereof, or a pharmaceutically acceptable salt, solvate, polymorph, tautomer, stereoisomer, an isotopic form, or a prodrug thereof, wherein the compound is represented by Formula (5) wherein: 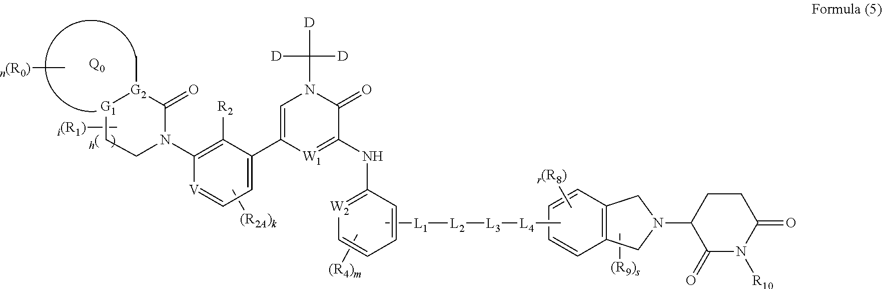
wherein:
R₈ is absent, H, D, alkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, heteroaryl, halo, oxo, cyano, —ORₐ, —SRₐ, -alkyl-Rₐ, -alkyl-O—P(O)(Rₐ)(R_{b}), -alkyl-OC(O)N(Rₐ)(R_{b}), —NH(CH₂)ₚRₐ, —C(O)Rₐ, —S(O)Rₐ, —SO₂Rₐ, —C(O)ORₐ, —OC(O)Rₐ, —NR_{b}R_{c}, —C(O)N(R_{b})R_{c}, —N(R_{b})C(O)R_{c}, in which said alkyl, spiroalkyl, alkenyl, alkynyl, cycloalkyl, cycloalkenyl, heterocycloalkyl, heterocycloalkenyl, spiro-heterocyclic, fused-heterocyclic, bridged-heterocyclic, aryl, or heteroaryl is optionally subsitiuted with one or more R_{d};
each of the border atoms between Q₀ and Q₁ including G₁ and G₂, can be carbon or heteroatom;
R₈ and L₄ groups, taken together with the atom to which they are attached, may optionally form a cycloalkyl, heterocycloalkyl, heterocycloalkenyl, aryl, or heteroaryl optionally subsitiuted with one or more R_{d}; and
r is 0, 1, 2, 3, or 4.

7. A pharmaceutical composition comprising a compound of any one of Formulas (1)-(5), or an N-oxide thereof, as defined in claims 1-6, respectively, or a pharmaceutically acceptable salt, solvate, polymorph, tautomer, stereoisomer, an isotopic form, or a prodrug of said compound of any one of Formulas (1)-(5) or an N-oxide thereof, and a pharmaceutically acceptable diluent or carrier.

8. A method of treating a neoplastic disease, autoimmune disease, and inflammatory disorder, comprising administering to a subject in need thereof an effective amount of a compound of any one of Formulae (1)-(5), or an N-oxide thereof, as defined in claims 1-6, respectively, or a pharmaceutically acceptable salt, solvate, polymorph, tautomer, stereoisomer, an isotopic form, or a prodrug of said compound of any one of Formulae (1)-(5), or N-oxide thereof.
